(19) United States
(12) Patent Application Publication
(10) Pub. No.: US 2026/0337278 A1
(43) Pub. Date: Oct. 29, 2026

(54) BIEPITOPIC TETRAVALENT ANTIBODY TARGETING EGFR

(71) Applicant: SYSTIMMUNE, INC., Redmond, WA (US)

(72) Inventors: Luke A. HELGESON, Sammamish, WA (US); Tyler C. ROZANITIS, Redmond, WA (US); Dennis R. GOULET, Redmond, WA (US); Jahan KHALILI, Kirkland, WA (US); Nicholas S. DAVIS, Seatle, WA (US); Yi ZHU, Redmond, WA (US)

(73) Assignee: SYSTIMMUNE, INC., Redmond, WA (US)

(21) Appl. No.: 19/107,585

(22) PCT Filed: Aug. 30, 2023
(86) PCT No.: PCT/US2023/073194

§ 371 Date: Feb. 28, 2025

Related U.S. Application Data
(60) Provisional application No. 63/402,945, filed on Aug. 31, 2022.

(51) Int. Cl. C07K 16/28; A61K 47/68

(52) U.S. Cl. CPC C07K 16/2863; A61K 47/6803; C07K 2317/24; C07K 2317/31; C07K 2317/34; C07K 2317/35; C07K 2317/55; C07K 2317/565; C07K 2317/622; C07K 2317/64; C07K 2317/732; C07K 2317/92

(57) ABSTRACT

A biepitopic tetravalent antibody having a binding affinity to at least two epitopes of EGFR. The antibody comprises an antibody backbone and a scFv domain linked to the antibody backbone. The two epitopes of EGFR may include an EGFR wild-type (EGFRwt) epitope and an EGFRvIII epitope. The antibody has a stronger binding affinity to the EGFRvIII epitope than to the EGFRwt epitope.

### CROSS REFERENCE TO RELATED APPLICATIONS

This application claims the benefit of the filing date of U.S. Provisional Application Ser. No. 63/402,945 filed Aug. 31, 2022, under 35 U.S.C. 119 (e), the entire disclosures of which are incorporated by reference herein.

### TECHNICAL FIELD

The present disclosure generally relates to the technical field of antibody cancer therapeutics, and more particularly relates to biepitopic tetravalent antibodies.

### BACKGROUND

Glioblastoma multiforme is an aggressive form of cancer accounting for the majority of malignancies originating in the brain and has poor prognosis with an average survival of 14-15 months post diagnosis.¹ A large percentage of glioblastoma cases (34-63%) involve amplification of the EGFR gene.² In addition to overexpression of wild-type EGFR protein, mutant versions of EGFR occur in 63-75% of cases, the most common of which is called EGFRvIII and occurs in 25-64% of cases.² Structurally, EGFRvIII mutation results in a 267-residue deletion of the extracellular domain of the protein, which functionally may induce constitutive signaling due to less efficient endocytosis of the receptor.² These changes cause EGFRvIII-bearing tumors to display increased proliferation, increased angiogenesis, and reduced apoptosis.² In addition to their association with glioblastoma multiforme, wild-type EGFR and mutant EGFRvIII may also be overexpressed in other types of solid tumor, including ovarian, breast, and lung cancers.²

Several antibodies targeting EGFR, including Cetuximab, Panitumumab, and Necitumumab, have been approved by the FDA for treatment of epithelial tumors. Nimotuzumab is also approved in several countries for treatment of solid tumors. The lower affinity of Nimotuzumab for EGFR, compared to Cetuximab or Panitumumab, could help to explain its reduced skin toxicities.³ Meanwhile, antibody-based therapies specifically targeting mutant EGFRvIII have been evaluated in clinical trials, including the monoclonal antibody Depatuxizumab (Phase I) and the antibody-drug conjugate Depatuxizumab mafodotin (Phase 3). In particular, the Phase 3 trial for the EGFRvIII-targeting ADC was halted after not meeting the primary endpoint of overall survival in patients with newly diagnosed glioblastoma.⁴ T cell engagers targeting EGFRvIII and CD3 have also been evaluated preclinically, further highlighting the promise of EGFRvIII as a therapeutic target.^{5,6} While the EGFRvIII-targeting therapies will not be available soon, there is no single therapeutics simultaneously targeting tumor cells expressing oncogenic EGFR that includes both overly expressed (wt) and mutated EGFR (vIII).

A potential shortcoming of Cetuximab- and ABT806-derived antibodies is that the variable regions of these antibodies were discovered in mice and retain non-human sequences. It has been demonstrated that chimeric antibodies may have increased capacity for immunogenicity when compared to humanized or human antibodies.⁷ On the other hand, humanization can not only decrease immunogenicity, but also increase the stability of antibodies by making the variable and constant regions more compatible.⁸

The high affinity of Cetuximab for its antigen, EGFR, may result in on-target, off-tumor binding. As a result, toxicities such as severe skin irritation are often observed. Meanwhile, Nimotuzumab, which binds to EGFR with lower affinity, seems to have fewer of these undesired side effects.⁹ Thus, it may be advantageous to lower the affinity of antibodies like Cetuximab in order to retain their antigen specificity while decreasing binding to low-expressing healthy tissue and thereby increasing the therapeutic index. In the context of a bispecific antibody, weakening affinity may be useful for balancing the relative strengths of the two binding domains, and effectively optimizing the overall specificity of the antibody.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

The disclosure provides biepitopic tetravalent antibodies targeting at least two epitopes of EGFR, and the methods of making and using the antibody.

In one aspect, the application provides a biepitopic tetravalent antibody having a binding affinity to at least two epitopes of EGFR. The antibody may include an antibody backbone, comprising an antibody light chain having an antibody light chain variable (VL) domain, an antibody heavy chain having an antibody heavy chain variable (VH) domain, wherein the antibody VL domain and the antibody VH domain forms a Fab region. The antibody may further include a scFv domain having a scFv light chain variable (VL) domain and scFv heavy chain variable (VH) domain, wherein the scFv domain is linked to at least one end of the antibody light chain or the antibody heavy chain through an inter-domain linker.

In one embodiment, each scFv domain has a structure order of N terminus—VH—linker VL—C—terminus, or N—terminus—VL—linker—VH—C-terminus. In one embodiment, the linker comprises a flexible GS linker having from about 20 amino acids. In one embodiment, the linker comprises an amino acid sequence (Gly-Gly-Gly-Gly-Ser) m, and wherein m is an integer of at least 3. In one embodiment, m is 4.

In one embodiment, the inter-domain linker comprises from about 10 to about 30 amino acids. In one embodiment, the inter-domain linker comprises an amino acid sequence (Gly-Gly-Gly-Gly-Ser) m, and wherein m is an integer of at least 2. In one embodiment, m is an integer from 2 to 6.

In one embodiment, the two epitopes of EGFR comprise an EGFR wild-type (EGFRwt) epitope and an EGFRvIII epitope, and the biepitopic tetravalent antibody has a stronger binding affinity to EGFRvIII than to EGFRwt. In one embodiment, the antibody has a binding affinity to the EGFRwt epitope with a first KD, and a binding affinity to the EGFRvIII epitope with a second KD, wherein the first KD is higher than the second KD.

In one embodiment, the first KD is not less than 1E-11 M, and the second KD is not more than 1E-06 M. In one embodiment, the first KD is between 1E-10 M and 1E-06 M, and the second KD is between 1E-11 M and 1E-07 M. In one embodiment, the first KD is more than 1.1-fold, 1.2-fold, 1.5-fold, 2-fold, 3-fold, 4-fold, 5-fold, 10-fold, 15-fold, 20-fold, 30-fold, 40-fold, 50-fold, 60-fold, or 70-fold higher than the second KD. In one embodiment, the first KD is between 1.1 to 1000-fold stronger than the second KD.

In one embodiment, the antibody has a first ADCC EC50 against cells bearing only EGFRwt and a second ADCC EC50 against cells bearing EGFRwt and EGFRvIII, wherein the first ADCC EC50 is more than the second ADCC EC50. In one embodiment, the first ADCC EC50 is more than 1.1-fold, 1.2-fold, 1.5-fold, 2-fold, 3-fold, 4-fold, 5-fold higher than the second ADCC EC50.

In one embodiment, the EGFRwt epitope comprises the epitope having a binding affinity to Cetuximab. In one embodiment, the EGFRvIII epitope comprises the epitope having a binding affinity to ABT-806.

In one embodiment, the scFv domain of the biepitopic tetravalent antibody has the binding affinity to the EGFRwt epitope and the Fab domain has the binding affinity to the EGFRvIII epitope. In one embodiment, the scFv domain has the binding affinity to the EGFRvIII epitope and the Fab domain has the binding affinity to the EGFRwt epitope.

In one embodiment, the scFv domain is linked to the biepitopic tetravalent antibody light chain at its C-terminus. In one embodiment, the scFv domain is linked to the antibody light chain at its N-terminus. In one embodiment, the scFv domain is linked to the antibody heavy chain at its C-terminus. In one embodiment, the scFv domain is linked to the antibody heavy chain at its N-terminus.

In one embodiment, the antibody backbone may be derived from Cetuximab, humanized Cetuximab, dematured Cetuximab, or humanized dematured Cetuximab (i.e., Cetuximab-derived backbone), or Nimotuzumab, and the scFv domain may comprise a binding domain derived from or ABT-806 or humanized ABT-806.

Alternatively, the antibody backbone may be derived from ABT-806 or humanized ABT-806, and the scFv domain may comprise a binding domain derived from Cetuximab, humanized Cetuximab, dematured Cetuximab, or humanized dematured Cetuximab, or Nimotuzumab. In one embodiment, the humanized ABT-806 is ABT-806 V1, V2, V3, V4, V5, V6, V7, V8, or V9.

### Cetuximab Backbone Biepitopic Tetravalent Antibodies

In one embodiment, the application provides a biepitopic tetravalent, wherein the antibody backbone is derived from Cetuximab, humanized Cetuximab, dematured Cetuximab, or humanized dematured Cetuximab (i.e., Cetuximab-derived backbone). In one embodiment, the scFv domain comprises a binding domain derived from the variable region of ABT-806 or humanized ABT-806.

In one embodiment, the antibody backbone comprises a Cetuximab dematuration mutation (sequential numbering) selected from VH-Y101, VH-Y102, VH-D103, VH-Y104, VL-N92, or a combination thereof. In one embodiment, the Cetuximab dematuration mutation (sequential numbering) comprises VH-Y101A, VH-Y101W, VH-Y102A, VH-D103F, VH-D103W, VH-D103Y, VH-Y104L, VL-N92F, VL-N92K, or a combination thereof.

In one embodiment, the antibody heavy chain comprises an amino acid sequence having at least 80%, 85%, 90%, 95%, 98%, 99% sequence identity to SEQ ID NO. 1, 13, 79, 97, 101, 103, 105, 107, 123, 127, 129, 131, or 133. In one embodiment, the antibody light chain comprises an amino acid sequence having at least 80%, 85%, 90%, 95%, 98%, 99% sequence identity to SEQ ID NO. 3, 81, 83, 99, 109, 125, or 135.

In one embodiment, the antibody backbone comprises humanized Cetuximab. In one embodiment, the antibody VH domain comprises an amino acid sequence having at least 80%, 85%, 90%, 95%, 98%, 99% sequence identity to SEQ ID NO. 237. In one embodiment, the antibody VL domain comprises an amino acid sequence having at least 80%, 85%, 90%, 95%, 98%, 99% sequence identity to SEQ ID NO. 239.

In one embodiment, the antibody backbone comprises dematured Cetuximab. In one embodiment, the antibody VH domain comprises an amino acid sequence having at least 80%, 85%, 90%, 95%, 98%, 99% sequence identity to SEQ ID NO. 247, 251, 253, 259, 261, 263, 265, or 267. In one embodiment, the antibody VL domain comprises an amino acid sequence having at least 80%, 85%, 90%, 95%, 98%, 99% sequence identity to SEQ ID NO. 243 or 269.

In one embodiment, the antibody backbone comprises the humanized dematured Cetuximab. In one embodiment, the antibody VH domain comprises an amino acid sequence having at least 80%, 85%, 90%, 95%, 98%, 99% sequence identity to SEQ ID NO. 245, 249, 255, or 257. In one embodiment, the antibody VL domain comprises an amino acid sequence having at least 80%, 85%, 90%, 95%, 98%, 99% sequence identity to SEQ ID NO. 241.

In one embodiment, the humanized ABT-806 is ABT-806 V1, V2, V3, V4, V5, V6, V7, V8, or V9.

In one embodiment, the scFv domain comprises the variable regions of ABT-806 or humanized ABT-806.

In one embodiment, the scFv VH domain comprises an amino acid sequence having at least 80%, 85%, 90%, 95%, 98%, 99% sequence identity to SEQ ID NO. 209, 225, 229, or 233. In one embodiment, the scFv VL domain comprises an amino acid sequence having at least 80%, 85%, 90%, 95%, 98%, 99% sequence identity to SEQ ID NO. 211, 227, 231, or 235.

In one embodiment, the antibody VH domain comprises 3 complementary determining regions (CDRs) of SEQ ID NO. 201, 237, 245, 247, 249, 251, 253, 255, 257, 259, 261, 263, 265, or 267. In one embodiment, the antibody VL domain comprises 3 CDRs of SEQ ID NO. 203, 239, 241, 243, or 269. In one embodiment, the scFv VH domain comprises 3 CDRs of SEQ ID NO. 209. In one embodiment, the scFv VL domain comprises 3 CDRs of SEQ ID NO. 211.

In one embodiment, the biepitopic tetravalent antibody comprises an amino acid sequence having at least 80%, 85%, 90%, 95%, 98%, 99% sequence identity to SEQ ID NO. 1, 3, 13, 79, 81, 83, 97, 99, 101, 103, 105, 107, 109, 123, 125, 127, 129, 131, 133, or 135.

### ABT-806 Backbone Biepitopic Tetravalent Antibodies

In one embodiment, the application provides a biepitopic tetravalent antibody, wherein the antibody backbone is derived from ABT0806 or humanized ABT-806, and wherein the scFv domain comprises a binding domain derived from the variable region of Cetuximab, humanized Cetuximab, dematured Cetuximab, humanized dematured Cetuximab. In one embodiment, the scFv domain comprises a binding domain derived from Nimotuzumab.

In one embodiment, the antibody heavy chain comprises an amino acid sequence having at least 80%, 85%, 90%, 95%, 98%, 99% sequence identity to SEQ ID NO. 9, 17, 19, 21, 23, 25, 27, 29, 31, 75, 93, 113, 115, 117, 119, 121, 137, 139, 141, 143, 145, 147, or 179.

In one embodiment, the antibody light chain comprises an amino acid sequence having at least 80%, 85%, 90%, 95%, 98%, 99% sequence identity to SEQ ID NO. 11, 33, 35, 37, 39, 41, 43, 77, 91, 95, or 111.

In one embodiment, the antibody backbone comprises the ABT-806, and wherein the antibody VH domain comprises an amino acid sequence having at least 80%, 85%, 90%, 95%, 98%, 99% sequence identity to SEQ ID NO. 209. In one embodiment the VL domain comprises an amino acid sequence having at least 80%, 85%, 90%, 95%, 98%, 99% sequence identity to SEQ ID NO. 211.

In one embodiment, the antibody backbone comprises the humanized ABT-806. In one embodiment, the antibody VH domain comprises an amino acid sequence having at least 80%, 85%, 90%, 95%, 98%, 99% sequence identity to SEQ ID NO. 225, 229, or 233. In one embodiment, the antibody VL domain comprises an amino acid sequence having at least 80%, 85%, 90%, 95%, 98%, 99% sequence identity to SEQ ID NO. 227, 231, or 235.

In one embodiment, the scFv VH domain comprises an amino acid sequence having at least 80%, 85%, 90%, 95%, 98%, 99% sequence identity to SEQ ID NO. 201, 205, 237, 245, 247, 249, 251, 253, 255, 257, 259, 261, 263, 265, or 267.

In one embodiment, the scFv VL domain comprises an amino acid sequence having at least 80%, 85%, 90%, 95%, 98%, 99% sequence identity to SEQ ID NO. 203, 207, 239, 241, 243, or 269.

In one embodiment, the scFv domain comprises a binding domain derived from the variable region of the dematured Cetuximab or the humanized dematured Cetuximab. In one embodiment, the dematured Cetuximab or the humanized dematured Cetuximab comprises a Cetuximab dematuration mutation (sequential numbering) selected from Y101, Y102, D103, Y104, N92, and a combination thereof. In one embodiment, the Cetuximab dematuration mutation (sequential numbering) comprises VH-Y101, VH-Y102, VH-D103, VH-Y104, VL-N92, or a combination thereof. In one embodiment, the Cetuximab dematuration mutation (sequential numbering) comprises VH-Y101A, VH-Y101W, VH-Y102A, VH-D103F, VH-D103W, VH-D103Y, VH-Y104L, VL-N92F, VL-N92K, or a combination thereof.

In one embodiment, the biepitopic tetravalent antibody VH domain comprises 3 complementary determining regions (CDRs) of SEQ ID NO. 209. In one embodiment, the biepitopic tetravalent antibody VL domain comprises 3 CDRs of SEQ ID NO. 211. In one embodiment, the scFv VH domain comprises 3 CDRs of SEQ ID NO. 201, 205, 237, 245, 247, 249, 251, 253, 255, 257, 259, 261, 263, 265, or 267. In one embodiment, the scFv VL domain comprises 3 CDRs of SEQ ID NO. 203, 207, 239, 241, 243, or 269.

In one embodiment, the biepitopic tetravalent antibody comprises an amino acid sequence having at least 80%, 85%, 90%, 95%, 98%, 99% sequence identity to SEQ ID NO. 9, 11, 17, 19, 21, 23, 25, 27, 29, 31, 33, 37, 41, 43, 75, 77, 91, 93, 95, 111, 113, 115, 117, 119, 121, 137, 139, 141, 143, 145, 147, or 179.

### Nimotuzumab Backbone Antibodies

In one embodiment, the application provides the biepitopic tetravalent antibody, wherein the antibody backbone comprises Nimotuzumab, and wherein the scFv domain comprises a binding domain derived from the variable region of ABT-806 or humanized ABT-806. In one embodiment, the scFv domain comprises the variable regions of ABT-806.

In one embodiment, the antibody heavy chain comprises an amino acid sequence having at least 80%, 85%, 90%, 95%, 98%, 99% sequence identity to SEQ ID NO. 5, 15, 45, 47, 49, 51, 53, 55, or 85.

In one embodiment, the antibody light chain comprises an amino acid sequence having at least 80%, 85%, 90%, 95%, 98%, 99% sequence identity to SEQ ID NO. 7, 57, 59, 61, 63, 65, 67, 69, 71, or 73.

In one embodiment, the antibody VH domain comprises an amino acid sequence having at least 80%, 85%, 90%, 95%, 98%, 99% sequence identity to SEQ ID NO. 205.

In one embodiment, the antibody VL domain comprises an amino acid sequence having at least 80%, 85%, 90%, 95%, 98%, 99% sequence identity to SEQ ID NO. 207.

In one embodiment, the scFv VH domain comprises an amino acid sequence having at least 80%, 85%, 90%, 95%, 98%, 99% sequence identity to SEQ ID NO. 209. In one embodiment, the scFv VL domain comprises an amino acid sequence having at least 80%, 85%, 90%, 95%, 98%, 99% sequence identity to SEQ ID NO. 211.

In one embodiment, the antibody VH domain comprises 3 complementary determining regions (CDRs) of SEQ ID NO. 205. In one embodiment, the antibody VL domain comprises 3 CDRs of SEQ ID NO. 207. In one embodiment, the scFv VH domain comprises 3 CDRs of SEQ ID NO. 209. In one embodiment, the scFv VL domain comprises 3 CDRs of SEQ ID NO. 211.

In one embodiment, the biepitopic tetravalent antibody comprises an amino acid sequence having at least 80%, 85%, 90%, 95%, 98%, 99% sequence identity to SEQ ID NO. 5, 7, 15, 45, 47, 49, 51, 53, 55, 57, 59, 61, 63, 65, 67, 69, 71, 73, or 85.

### Dematured Cetuximab Monoclonal Antibodies

In one aspect, the application provides a dematured Cetuximab monoclonal antibody, comprising a light chain having a light chain variable (VL) domain, and a heavy chain having a heavy chain variable (VH) domain, wherein the VL domain and the VH domain forms a Fab region, and wherein the monoclonal antibody comprises a Cetuximab dematuration mutation (sequential numbering) selected from VH-Y101, VH-Y102, VH-D103, VH-Y104, VL-N92, and a combination thereof.

In one embodiment, the Cetuximab dematuration mutation (sequential numbering) comprises VH-Y101A, VH-Y101W, VH-Y102A, VH-D103F, VH-D103W, VH-D103Y, VH-Y104L, VL-N92F, VL-N92K, or a combination thereof.

In one embodiment, the dematured Cetuximab monoclonal antibody is non-humanized.

In one embodiment, the antibody VH domain comprises 3 complementary determining regions (CDRs) having an amino acid sequence having at least 80%, 85%, 90%, 95%, 98%, 99% of sequence identity to SEQ ID NO. 247, 251, 253, 259, 261, 263, 265, or 267. In one embodiment, the antibody VL domain comprises 3 CDRs having an amino acid sequence having at least 80%, 85%, 90%, 95%, 98%, 99% of sequence identity to SEQ ID NO. 243 or 269.

In one embodiment, the heavy chain comprises an amino acid sequence having at least 80%, 85%, 90%, 95%, 98%, 99% of sequence identity to SEQ ID NO. 1, 155, 157, 159, 161, 163, 165, 167, or 169. In one embodiment, the antibody VH domain comprises an amino acid sequence having at least 80%, 85%, 90%, 95%, 98%, 99% of sequence identity to SEQ ID NO. 247, 251, 253, 259, 261, 263, 265, or 267.

In one embodiment, the light chain comprises an amino acid sequence having at least 80%, 85%, 90%, 95%, 98%, 99% of sequence identity to SEQ ID NO. 3, 151, or 153. In one embodiment, the VL domain comprises an amino acid sequence having at least 80%, 85%, 90%, 95%, 98%, 99% of sequence identity to SEQ ID NO. 243 or 269.

In one embodiment, the dematured Cetuximab monoclonal antibody is humanized.

In one embodiment, the antibody VH domain comprises 3 complementary determining regions (CDRs) having an amino acid sequence having at least 80%, 85%, 90%, 95%, 98%, 99% of sequence identity to SEQ ID NO. 245, 249, 255, or 257. In one embodiment, the antibody VL domain comprises 3 complementary determining regions (CDRs) having an amino acid sequence having at least 80%, 85%, 90%, 95%, 98%, 99% of sequence identity to SEQ ID NO. 241.

In one embodiment, the heavy chain comprises an amino acid sequence having at least 80%, 85%, 90%, 95%, 98%, 99% of sequence identity to SEQ ID NO. 123, 127, 129, 131, or 133. In one embodiment, the antibody VH domain comprises an amino acid sequence having at least 80%, 85%, 90%, 95%, 98%, 99% of sequence identity to SEQ ID NO. 245, 249, 255, or 257.

In one embodiment, the light chain comprises an amino acid sequence having at least 80%, 85%, 90%, 95%, 98%, 99% of sequence identity to SEQ ID NO. 99 or 109. In one embodiment, the antibody VL domain comprises an amino acid sequence having at least 80%, 85%, 90%, 95%, 98%, 99% of sequence identity to SEQ ID NO. 241.

In one embodiment, the application provides a multispecific antibody, comprising an antibody backbone, and at least one scFv domain linked to the antibody backbone, wherein the antibody backbone comprises the dematured Cetuximab monoclonal antibody as disclosed herein.

In one embodiment, the application provides a multispecific antibody, comprising an antibody backbone, and at least one scFv domain linked to the antibody backbone, wherein the scFv domain comprise variable regions of dematured Cetuximab monoclonal antibody as disclosed herein.

### Humanized Anti-EGFRvIII Monoclonal Antibodies

In one aspect, the application provides a humanized anti-EGFRvIII monoclonal antibody, comprising a light chain having a light chain variable (VL) domain, and a heavy chain having a heavy chain variable (VH) domain, wherein the VL domain and the VH domain forms a Fab region. In one embodiment, the monoclonal antibody is ABT0806 or humanized ABT-806.

In one embodiment, the heavy chain comprises an amino acid sequence having at least 80%, 85%, 90%, 95%, 98%, 99% of the sequence identity to SEQ ID NO. 171, 149, or 177. In one embodiment, the light chain comprises an amino acid sequence having at least 80%, 85%, 90%, 95%, 98%, 99% of the sequence identity to SEQ ID NO. 111, 173, or 175. In one embodiment, the VH domain comprises an amino acid sequence having at least 80%, 85%, 90%, 95%, 98%, 99% of the sequence identity to SEQ ID NO. 225, 229, or 233. In one embodiment, the VL domain comprises an amino acid sequence having at least 80%, 85%, 90%, 95%, 98%, 99% of the sequence identity to SEQ ID NO. 227, 231, or 235.

In one embodiment, the application provides a multispecific antibody, comprising an antibody backbone, and at least one scFv domain linked to the antibody backbone, wherein the antibody backbone comprises the humanized anti-EGFRvIII monoclonal antibody as disclosed herein.

In one embodiment, the application provides a multispecific antibody, comprising an antibody backbone, and at least one scFv domain linked to the antibody backbone, wherein the scFv comprises variable regions of the humanized anti-EGFRvIII as disclosed herein.

### Tetravalent Antibodies

In one aspect, the application provides a tetravalent antibody having a binding affinity to EGFR, comprising an antibody light chain having an antibody light chain variable (VL) domain, an antibody heavy chain having an antibody heavy chain variable (VH) domain, and a scFv domain having a scFv light chain variable (VL) domain and scFv heavy chain variable (VH) domain, wherein the antibody VL domain and the antibody VH domain forms a Fab region, wherein the scFv domain is linked to at least one end of the antibody light chain or the antibody heavy chain.

In one embodiment, the antibody VH domain comprises an amino acid sequence having a SEQ ID NO. 201. In one embodiment, the antibody VL domain comprises an amino acid sequence having a SEQ ID NO. 203. In one embodiment, the antibody heavy chain comprises an amino acid sequence having a SEQ ID NO. 89. In one embodiment, the antibody light chain comprises an amino acid sequence having a SEQ ID NO. 3. In one embodiment, the scFv domain comprises a light chain variable (VL) domain having an amino acid sequence having a SEQ ID NO. 203. In one embodiment, the scFv domain comprises a heavy chain variable (VH) domain having an amino acid sequence having a SEQ ID NO. 201.

In one embodiment, the antibody heavy chain comprises an amino acid sequence having at least 80%, 85%, 90%, 95%, 98%, 99% of the sequence identity to SEQ ID NO. 89. In one embodiment, the antibody light chain comprises SEQ ID NO. 3. In one embodiment, the antibody VH comprises SEQ ID NO. 201. In one embodiment, the antibody VL comprises SEQ ID NO. 203. In one embodiment, the antibody scFv VH comprises SEQ ID NO. 201. In one embodiment, the antibody scFv VL comprises SEQ ID NO. 203.

In one embodiment, the antibody VH comprises 3 complementary determining regions (CDRs) of SEQ ID NO. 201. In one embodiment, the antibody VL comprises 3 CDRs of SEQ ID NO. 203. In one embodiment, the antibody scFv VH comprises 3 CDRs: SEQ ID NO. 201. In one embodiment, the antibody scFv VL 3 CDRs comprises SEQ ID NO. 203.

In one embodiment, the tetravalent antibody is represented by SI-95X42, as shown in the example below.

In another embodiment, the application provides a tetravalent antibody having a binding affinity to EGFRvIII, comprising an antibody light chain having an antibody light chain variable (VL) domain, an antibody heavy chain having an antibody heavy chain variable (VH) domain, and a scFv domain having a scFv light chain variable (VL) domain and scFv heavy chain variable (VH) domain, wherein the antibody VL domain and the antibody VH domain forms a Fab region, wherein the scFv domain is linked to at least one end of the antibody light chain or the antibody heavy chain.

In one embodiment, the antibody VH domain comprises an amino acid sequence having a SEQ ID NO. 209. In one embodiment, the antibody VL domain comprises an amino acid sequence having a SEQ ID NO. 211.

In one embodiment, the antibody heavy chain comprises an amino acid sequence having a SEQ ID NO. 87. In one embodiment, the antibody light chain comprises an amino acid sequence having a SEQ ID NO. 11. In one embodiment, the scFv domain comprises a light chain variable (VL) domain having an amino acid sequence having a SEQ ID NO. 211. In one embodiment, the scFv domain comprises a heavy chain variable (VH) domain having an amino acid sequence having a SEQ ID NO. 209.

In one embodiment, the antibody heavy chain comprises SEQ ID NO. 87. In one embodiment, the antibody light chain comprises SEQ ID NO. 11. In one embodiment, the antibody VH domain comprises SEQ ID. NO. 209. In one embodiment, the antibody VL domain comprises SEQ ID NO. 211. In one embodiment, the antibody scFv VH domain comprises SEQ ID NO. 209. In one embodiment, the antibody scFv VL domain comprises SEQ ID NO. 211 In one embodiment, the antibody VH domain comprises 3 complementary determining regions (CDRs) of SEQ ID NO. 209. In one embodiment, the antibody VL domain comprises 3 CDRs of SEQ ID NO. 211. In one embodiment, the antibody scFv VH domain comprises 3 CDRs of SEQ ID NO. 209. In one embodiment, the antibody scFv VL domain comprises 3 CDRs of SEQ ID NO. 211.

In one embodiment, the intraspecific antibody is represented by SI-95X41, as shown in the example below.

In another aspect, the application provides isolated nucleic acid sequences encoding the biepitopic tetravalent antibody, monoclonal antibodies, tetraspecific antibodies, or multispecific antibodies as disclosed herein.

In a further aspect, the application provides expression vectors comprising the nucleic acid sequences as disclosed herein, wherein the vector is expressible in a cell.

In a further aspect, the application provides host cells comprising the nucleic acid as disclosed herein. In one embodiment, the host cell comprises the expression vector as disclosed herein. In one embodiment, the host cell is a prokaryotic cell or a eukaryotic cell.

In a further aspect, the application provides methods of producing the antibodies as disclosed herein. In one embodiment, the method includes culturing the host cell disclosed herein so that the disclosed antibody is produced. In one embodiment, the antibody is a biepitopic tetravalent antibody. In one embodiment, the antibody is monoclonal antibodies. In one embodiment, the antibody is tetraspecific antibodies. In one embodiment, the antibody is multispecific antibodies.

In a further aspect, the application provides immunoconjugates comprising the biepitopic tetravalent antibody as disclosed herein and a cytotoxic agent. In one embodiment, the cytotoxic agent comprises a chemotherapeutic agent, a growth inhibitory agent, a toxin, or a radioactive isotope.

In a further aspect, the application provides a pharmaceutical composition, comprising the antibody as disclosed herein and a pharmaceutically acceptable carrier. In one embodiment, the pharmaceutical composition further includes radioisotope, radionuclide, a toxin, a therapeutic agent, a chemotherapeutic agent, or a combination thereof. In one embodiment, the antibody is a biepitopic tetravalent antibody. In one embodiment, the antibody is monoclonal antibodies. In one embodiment, the antibody is tetraspecific antibodies. In one embodiment, the antibody is multispecific antibodies.

In a further aspect, the application provides pharmaceutical composition, comprising the immunoconjugate as disclosed herein and a pharmaceutically acceptable carrier.

In a further aspect, the application provides method of treating a subject with a cancer, comprising administering to the subject an effective amount of the biepitopic tetravalent antibody as disclosed herein. In one embodiment, the cancer comprises cells expressing EGFR, EGFRvIII or both. In one embodiment, the cancer comprises breast cancer, colorectal cancer, pancreatic cancer, head and neck cancer, melanoma, ovarian cancer, prostate cancer, non-small lung cell cancer, small cell lung cancer, glioma, esophageal cancer, nasopharyngeal cancer, kidney cancer, gastric cancer, liver cancer, bladder cancer, cervical cancer, brain cancer, lymphoma, leukemia, myeloma.

In one embodiment, the method of treating further includes co-administering an effective amount of a therapeutic agent. In one embodiment, the therapeutic agent comprises an antibody, a chemotherapy agent, an enzyme, or a combination thereof. In one embodiment, the therapeutic agent comprises capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, a derivative or a combination thereof.

In one embodiment, the subject is a human.

In a further aspect, the application provides a solution comprising an effective concentration of the biepitopic tetravalent antibody as disclosed herein, wherein the solution is blood plasma in a subject.

### BRIEF DESCRIPTION OF THE DRAWINGS

The foregoing and other features of this disclosure may become more fully apparent from the following description and appended claims, taken in conjunction with the accompanying drawings. Understanding that these drawings depict only several embodiments arranged in accordance with the disclosure and are, therefore, not to be considered limiting of its scope, the disclosure may be described with additional specificity and detail through use of the accompanying drawings, in which:

FIG. 1 depicts the configurations of biepitopic tetravalent antibodies capable of targeting two epitopes of EGFR comprising two anti-EGFR Fab domains and two anti-EGFRvIII scFv domains (A) or two anti-EGFRvIII Fab domains and two anti-EGFR scFv domains (B), wherein the scFv domain may be linked to N- or C-terminus of heavy or light chain of the antibody, and the binding domain may be derived from Cetuximab (which may be humanized and/or dematured) or Nimotuzumab in combination with ABT-806 (which may be humanized);

FIG. 2 depicts a structural alignment of Cetuximab (PDB 1YY9), Panitumumab (PDB 5SX4), and Necitumumab (PDB 6B3S) complexes with EGFR or domain III of EGFR, indicating that all three antibodies bind an overlapping epitope (EGFRwt) within domain III of EGFR;

FIG. 3 shows the results of mass analysis of wild-type and humanized versions of ABT806, the wild-type of which contains two purified fractions corresponding to intact mAb and heavy chain dimer contaminant (3A); and two humanized versions of which show a single pure fraction containing the intact mAb of interest but lacking the heavy chain dimer contaminant (3B);

FIG. 4 shows the results of ADCC assay measuring the potency of SI-95m4 (4A) and SI-95m5 (4B) against EGFRwt-expressing U87MG-EGFRwt (GFP) cells and EGFRwt/EGFRvIII-expressing U87MG-EGFRvIII (NR) cells, as either separate or mixed target cells (T), in the presence of NK effector cells (E: T=5:1);

FIG. 5 shows the results of ADCC assay measuring the potency of a panel of anti-EGFRwt monoclonal antibodies (mAbs), including dematured variants SI-95m6, SI-95m7, SI-95m8, SI-95m9, SI-95m10, and controls of anti-EGFRvIII and anti-EGFRwt antibodies against either EGFRwt-expressing U87MG-EGFRwt (GFP) cells (5A) or EGFRwt/EGFRvIII-expressing U87MG-EGFRvIII (NR) cells (5B), in the presence of NK effector cells (E: T=5:1), and the EC50 values for the antibodies are listed;

FIG. 6 shows the results of ADCC assay measuring the potency of a panel of biepitopic tetravalent antibodies (i.e., Bispecifics) targeting EGFRwt and EGFRvIII, including SI-95X44 and its dematured derivatives, including but not limited to SI-95X52, SI-95X53, SI-95X54, SI-95X55, and SI-95X56, against EGFRwt-expressing U87MG-EGFRwt (GFP) cells (6A) and EGFRwt/EGFRvIII-expressing U87MG-EGFRvIII (NR) cells (6B), in the presence of NK effector cells (E: T=5:1), and the EC50 values for the antibodies are listed;

FIG. 7 depicts the effect of dematured Cetuximab mutations VH-Y101A (7A) or VH-Y102A (7B) to SI-95X44, a biepitopic tetravalent antibody (aka, Bispecific) targeting EGFRwt and EGFRvIII, and the effect of having a humanized anti-EGFRvIII antibody ABT-806 backbone in SI-95X53 and SI-95X54 as compared to SI-95X65 and SI-95X66, respectively, as measured by their potency against EGFRwt-expressing U87MG-EGFRwt (GFP) cells, in the presence of NK effector cells (E, ET=5:1), which showing that both SI-95X53 and SI-95X54 have more completed killing against EGFRwt-expressing cells and better EC50 values as listed; and

FIG. 8 depicts the potential increase in therapeutic index obtained by introducing a demature mutation, Y102A, into the mature Cetuximab binding domain of a biepitopic tetravalent antibody or simply (bispecifics) targeting both EGFRwt and EGFRvIII by measuring their ADCC activity against U87MG-EGFRwt-expressing cells and U87MG-EGFRwt/EGFRvIII-expressing cells as either a separate or mixed population, and shows that SI-95X44 with a mature Cetuximab binding mediates overlapping killing curves with similar ED50 values, whereas SI-95X54 with a Y102A mutation differentiates the killing of EGFRwt-expressing cells in a mixed population versus a separate population mimicking glioblastoma versus normal tissues in patients.

### DETAILED DESCRIPTION

Due to the high prevalence of wild-type and mutant EGFR (i.e., the overly expressed EGFRwt protein, and the EGFRvIII mutant protein, respectively) expression in several types of solid tumors, it is an unmet challenge to develop an efficacious therapeutic strategy simultaneously targeting both versions of EGFR. The strategy would involve both a first specificity for EGFRvIII that may allow for preferentially targeting of the most malignant cells and a second specificity to the wild-type receptor that may allow for broad tumor targeting. Such a bispecific antibody would represent a new class of biepitopic tetravalent antibodies characterized by dual binding specificities to two epitopes of the same EGFR target molecule.

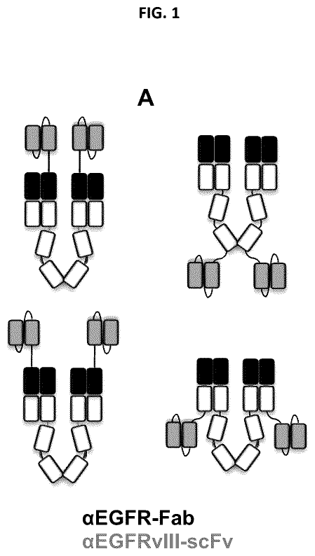

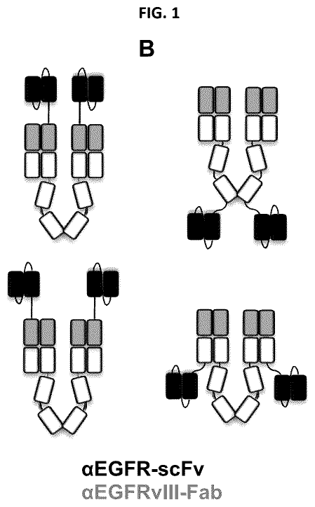

To construct bispecific tetravalent antibodies capable of binding both wild-type and mutant EGFR proteins, with a preference for binding to EGFRvIII, scFv domains were appended to various termini of antibody heavy and light chains. As shown in FIG. 1, an EGFRvIII-targeting scFv was attached to the N- or C-terminus (via the heavy or light chain) of an antibody targeting EGFR (FIG. 1A, 1B, 1C, and 1D). Alternatively, an EGFRwt-targeting scFv was attached to the N- or C-terminus (via the heavy or light chain) of an antibody targeting EGFRvIII. Several parameters, including inter-domain linker length and orientation of scFv VH/VL domains, were optimized for stability and binding affinity (Table 1).

The present application relates to methods of making and using bispecific tetravalent antibodies, specifically, biepitopic tetravalent antibodies.

The term “epitope” defines the region of an antigen that binds to an antibody. The term “antibody” is used in the broadest sense and specifically covers single monoclonal antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, bispecific or bi-effective, human and humanized antibodies as well as active fragments thereof. Examples of active fragments of molecules that bind to known antigens include Fab, F(ab′) 2, scFv and Fv fragments, including the products of a Fab immunoglobulin expression library and epitope-binding fragments of any of the antibodies and fragments mentioned above. In some embodiments, antibody may include immunoglobulin molecules and immunologically active portions of immunoglobulin molecules, i.e., molecules that contain a binding site that immunospecifically bind an antigen. The immunoglobulin can be of any type (IgG, IgM, IgD, IgE, IgA and IgY) or class (IgG1, IgG2, IgG3, IgG4, IgA1 and IgA2) or subclasses of immunoglobulin molecule. In one embodiment, the antibody may be whole antibodies and any antigen-binding fragment derived from the whole antibodies. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against one common antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins.

The modifier “monoclonal” indicates the character of the antibody as being obtained from a substantially homogeneous population of antibodies and is not to be construed as requiring production of the antibody by any particular method. In context of an antibody being directed against more than one common antigenic site, the term “monoclonal antibody” refers to the group of monoclonal monospecific antibodies during the early year of development. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567). In later cases, the modifier “monoclonal” of the term “monoclonal multi-specific antibody” is often understandably omitted.

The monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

Monoclonal antibodies can be produced using various methods including mouse hybridoma or phage display (see Siegel. Transfus. Clin. Biol. 9:15-22 (2002) for a review) or from molecular cloning of antibodies directly from primary B cells (see Tiller. New Biotechnol. 28:453-7 (2011)). The antibody variable genes were isolated using recombinant DNA techniques and the resulting antibodies were expressed recombinantly and further screened for desired features such as ability to inhibit the binding of EGFR in its wild type or mutant forms. This general method of antibody discovery is similar to that described in Seeber et al. PLOS One. 9: e86184 (2014). Once monoclonal antibody genes have been discovered, they may be humanized, engineered, combined, and otherwise modified to generate various types of multispecific antibodies. Cetuximab (anti-EGFR) and ABT-806 (anti-EGFRvIII) were both discovered using mouse hybridoma.^{9,10}

The term “antigen- or epitope-binding portion or fragment” refers to fragments of an antibody that are capable of binding to an antigen such as EGFR in the present application. These fragments may be capable of the antigen-binding function and additional functions of the intact antibody. Examples of binding fragments include but are not limited to a single-chain Fv fragment (scFv) consisting of the VL and VH domains of a single arm of an antibody connected in a single polypeptide chain by a synthetic linker or a Fab fragment which is a monovalent fragment consisting of the VL, constant light (CL), VH and constant heavy 1 (CH1) domain. Antibody fragments can be even smaller sub-fragments and can consist of domains as small as a single CDR domain, in particular the CDR3 regions from either the VL and/or VH domains (for example see Beiboer et al., J. Mol. Biol. 296:833-49 (2000)). Antibody fragments are produced using conventional methods known to those skilled in the art. The antibody fragments can be screened for utility using the same techniques employed with intact antibodies.

The “antigen- or epitope-binding fragments” can be derived from an antibody of the present disclosure by several art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen.

The Fab fragment may contain the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind an antigen, although at a lower affinity than the entire binding site.

The “light chains” of antibodies (immunoglobulins) from any vertebrate species can be assigned to one of two clearly distinct types, called kappa and lambda (A), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called a, delta, epsilon, v, and u, respectively. The subunit structures and three-dimensional configurations of different classes of immunoglobulins are well known.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. (see, e.g., Queen et al., Proc. Natl Acad Sci USA, 86:10029-10032 (1989), Hodgson et al., Bio/Technology, 9:421 (1991)).

The terms “polypeptide”, “peptide”, and “protein”, as used herein, are interchangeable and are defined to mean a biomolecule composed of amino acids linked by a peptide bond.

The terms “a”, “an” and “the” as used herein are defined to mean “one or more” and include the plural unless the context is inappropriate.

By “isolated” is meant a biological molecule free from at least some of the components with which it naturally occurs. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic a binding specificity.

“Recombinant” means the antibodies are generated using recombinant nucleic acid techniques in exogeneous host cells.

The term “antigen” refers to an entity or fragment thereof which can induce an immune response in an organism, particularly an animal, more particularly a mammal including a human. The term includes immunogens and regions thereof responsible for antigenicity or antigenic determinants.

Also, as used herein, the term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated. In case of making a humanized antibody, the term “immunogenicity” refers to an ability of therapeutics, such as a non-humanized antibody, to trigger an undesirable immune response against the therapeutics.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs. The deviations appearing in the comparison between a given sequence and the above-described sequences of the disclosure may be caused for instance by addition, deletion, substitution, insertion, or recombination.

The term “dematuration” refers to a process by which a mature antibody (such as Cetuximab in this application) is engineered to have a mutation in its binding domain (such as a point mutation). The resulting antibody is called a “dematured” antibody, which exhibits an altered but desired binding activity that is characteristically different from that of a “matured” antibody.

The term “ADCC” refers to antibody-dependent cellular cytotoxicity, which is an adaptive immune response where Fc receptor-bearing effector cells recognize and kill antibody-coated target cells that express tumor-associated or pathogen-derived antigens on their surface. ADCC is widely used for characterizing and comparing therapeutic candidates of monoclonal antibodies. Classical ADCC is mediated by natural killer (NK) cells.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

### Examples

### Example 1. Engineering Biepitopic Tetravalent Antibodies Targeting EGFR

Genes encoding antibody heavy and light chains (preceded by Kozak and secretory signal peptide) were cloned into pTT5 vector using standard molecular biology techniques. Antibodies and bispecific antibodies were expressed by transiently transfecting the expression plasmids for heavy and light chains in the ExpiCHO system (Thermo Fisher). Briefly, 10 μg of each expression plasmid was brought to 1 ml with OptiPRO SFM medium. 1 ml of OptiPRO SFM medium containing 80 ul Expifectamine CHO reagent was added to the DNA and incubated at room temperature for 2.5 minutes. The resulting mixture was then added to 25 ml ExpiCHO cells at 6×10⁶ cells/ml in a 125 ml Erlenmeyer flask and incubated at 37° C., 5% CO₂, 150 rpm. Cells were fed with 8.75 ml ExpiCHO feed and 150 μl of CHO enhancer at 24 hours post-transfection and temperature shifted to 32° C., 5% CO₂, 150 rpm. Cells were fed again at 48 hours post-transfection with 8.75 ml ExpiCHO feed. Culture supernatant was harvested 9 days post-transfection, spun for 1 hour at 4500 rpm to pellet the cells and then passed through a 0.2 mm filter. Expression titer was quantitated using biolayer interferometry on an Octet384 system with protein A sensors and a standard curve prepared with purified bispecific antibody protein.

Proteins were purified from the harvested supernatant using a 5-ml MabSelect PrismA protein A column (GE Healthcare) equilibrated in phosphate-buffered saline on the AKTA Avant system. The supernatant was then passed over the column at a flow rate of 5 ml/min and washed with 25 ml PBS (125 mM sodium phosphate, 137 mM sodium chloride, pH 6.8). Protein of interest was then eluted by passing 15 ml of 50 mM sodium acetate, pH 3.5 over the column. The eluted protein was immediately neutralized by addition of 0.5 ml 1M Tris-Cl, pH8.0.

Immediately after first-step protein A purification, proteins were analyzed by analytical SEC using an ACQUITY UPLC® Protein BEH SEC 200 Å, 4.6 mm×150 mm, 1.7 μm column controlled by a Waters Acquity UPLC H-Class. 10 μg of protein was injected into a mobile phase of PBS and flowed at 0.3 ml/min for 10 minutes. Proteins were further purified by preparative SEC using a Superdex Increase 10/300 GL column in mobile phase of 25 mM sodium acetate, 125 mM NaCl, pH 5.5 on the AKTA Pure system, and ultimately buffer-exchanged into 25 mM sodium acetate, 125 mM NaCl, 10% sucrose, pH 5.5. Final samples contained >95% protein of interest as assessed by analytical SEC and were used for subsequent assays.

Biolayer interferometry (Octet) binding assays (affinity) were performed on an Octet384 instrument to quantify binding kinetics of bispecific antibodies to EGFR and EGFRvIII. Antibody was captured to anti-human Fc (AHC) sensor tips by loading for 180 seconds at 5 μg/ml. After a 60-second baseline step, a 180-second association phase with serial dilutions (0-100 nM; 1:2 dilution factor) of His-tagged EGFR or EGFRvIII (expressed and purified in-house) in assay buffer (PBS containing 0.1% BSA, 0.05% Tween20) was performed, followed by a 300-second dissociation phase in assay buffer. Regeneration was achieved using 10 mM glycine, pH 1.5. Binding curves were globally fit to a 1:1 model to extract the dissociation constants, KD, and kinetic association and dissociation rates.

Biolayer interferometry (Octet) binding assays (avidity) were performed on an Octet384 instrument to quantify bivalent binding kinetics of bispecific antibodies to immobilized EGFR and EGFRvIII. Biotinylated EGFR or EGFRvIII was captured to streptavidin (SA) sensor tips by loading for 120 seconds at 1 μg/ml. After a 60-second baseline step, a 180-second association phase with serial dilutions (0-25 nM; 1:2 dilution factor) of antibody in assay buffer was performed, followed by a 300-second dissociation phase in assay buffer. Regeneration was achieved using 10 mM glycine, pH 1.5. Binding curves were globally fit to a 1:1 model to extract the dissociation constants, K_{D}, and kinetic association and dissociation rates.

Biolayer interferometry (Octet) was used for epitope binning studies. Biotinylated EGFR-WT (Acro EGR-H82E3) or EGFRvIII (Acro EGR-H82E0) was immobilized at 1 μg/ml for 180 seconds onto streptavidin sensors in assay buffer (PBS containing 0.1% BSA, 0.05% Tween20). After a 30-second baseline, association with 100 nM of the first antibody was performed for 300 seconds. Finally, association with 100 nM of the second antibody was performed for 300 seconds. Octet Analysis 12.0 software was used to generate epitope binning matrices.

### Example 2: Expression Titer

Protein stability is a key parameter defined by the difference in free energy between the folded and unfolded states. For protein therapeutics, stability may impact immunogenicity, pharmacokinetics, and even efficacy. The reduction of aggregation can help to develop therapeutics that are easier to manufacture and safer for patients. In addition, expression efficiency and protein yield directly determine the cost of protein therapeutics. Manufacturing costs can be reduced significantly if proteins can be efficiently expressed to reach higher titers and increased yield of purified protein.

After transient expression in ExpiCHO cells, titer was quantitated using biolayer interferometry (Table 2). Monoclonal antibodies (EGFR or EGFRvIII) containing simple structures were produced with the highest efficiency, whereas bispecific (EGFR×EGFRvIII, SI-95X1-31, SI-95X35-39) antibodies were produced with variable efficiency depending primarily on the composition of the binding domains. Bispecific antibodies with nimotuzumab scFv (SI-95X4-16) were produced with significantly lower titer, indicative of the instability of the nimotuzumab VH and VL with this scFv format. On the other hand, structures with nimotuzumab in the Fab position of the antibody and ABT-806 scFv (SI-95X2, SI-95X17-31) tended to show higher titer on average.

### Example 3: Stability by Analytical Size-Exclusion Chromatography

Immediately following first-step protein A purification, bispecific EGFR×EGFRvIII antibodies were evaluated for stability and aggregation by analytical size-exclusion chromatography on a Waters UPLC system (Table 2). Antibodies containing nimotuzumab scFv (SI-95X4-16) generally showed low signal due to low expression and tended to have a high abundance of high molecular weight (HMW) or low molecular weight (LMW) contaminants in addition to the main peak corresponding to protein of interest (POI). This suggests that the nimotuzumab scFv is not stable when incorporated into the bispecific antibody. The other bispecific antibodies showed on average more protein of interest, and less HMW and LMW species. The proteins with C-terminal scFvs fused with the heavy chain (SI-95X1-3) showed the highest % POI, which is indicative of good stability. There also appeared to be an effect of scFv orientation on SEC profile, with ABT-806 scFv-containing antibodies showing a complex split profile when in HL orientation (SI-95X17-19, SI-95X23-25, SI-95X29-31), but not corresponding structures when ABT-806 scFv was in LH orientation (SI-95X20-22, SI-95X26-28). This result suggests that LH orientation of this scFv could be more stable and/or conformationally homogeneous. Inter-domain linker length showed negligible effects on aggregation.

### Example 4: Octet Binding to EGFR WT (EGFRwt)

Biolayer interferometry was used to assess the affinity of bispecific antibodies for wild-type EGFR (Table 3). Cetuximab and nimotuzumab antibodies showed strong binding to wild-type EGFR, as demonstrated by response values of 0.75 and 0.32 nm respectively, whereas ABT-806 (an EGFRvIII-specific antibody) showed a much lower response of 0.07 nm. Most EGFR×EGFRvIII bispecific antibodies showed strong binding to wild-type EGFR, with the exception of those containing nimotuzumab scFv and ABT-806 Fab (SI-95X4-16) which showed low binding. This result indicates that nimotuzumab has reduced function when present in scFv format. On the other hand, when nimotuzumab was in the Fab position (SI-95X2, SI-95X17-31), or when Cetuximab was in the Fab (SI-95X1, SI-95X37-39) or scFv (SI-95X3, SI-95X35-36) position, good binding to wild-type EGFR was observed. Affinity data showed that antibodies with the Cetuximab domain had stronger affinity (lower K_{D}) than those with the nimotuzumab domain.

### Example 5: Octet binding to EGFRvIII

Biolayer interferometry was used to assess the affinity of bispecific antibodies for EGFRvIII (Table 4). As expected, all monoclonal and bispecific antibodies targeting wild-type EGFR and/or EGFRvIII showed detectable binding to EGFRvIII, consistent with both binding epitopes being present in the EGFRvIII protein. Cetuximab had stronger binding response and affinity compared to nimotuzumab, whereas binding of ABT-806 was intermediate. For bispecific antibodies containing nimotuzumab scFv and ABT-806 Fab (SI-95X4-16), the observed binding was similar to that of the ABT-806 monoclonal antibody, indicating that nimotuzumab scFv is not contributing extra functionality. In contrast, bispecific antibodies containing nimotuzumab Fab and ABT-806 scFv (SI-95X2, SI-95X17-31) had lower K_{D} values and slower dissociation rates, in some cases showing enhanced affinity compared to the component mAbs. The same affinity enhancement was observed for bispecific antibodies containing Cetuximab and ABT-806 domains (SI-95X1, SI-95X3, SI-95X35-39). This result suggests that the nimotuzumab/Cetuximab and ABT-806 domains could be simultaneously binding to two epitopes of the same EGFRvIII molecule, thereby creating a multivalent avidity effect that enhances apparent affinity.

### Example 6: Epitope Binning (EGFRwt Epitope)

Biolayer interferometry was used for epitope binning of a panel of monoclonal antibodies with wild-type EGFR (Table 5). Antibodies included EGFR-specific Cetuximab (Cetu), Panitumumab (Pani), Nimotuzumab (Nimo), and Necitumumab (Neci), as well as EGFRvIII-specific ABT-806. Larger response values indicate that the second antibody was able to associate with EGFR even after saturation with the first antibody had already occurred (suggesting distinct epitopes). Conversely, smaller numbers indicate that the second antibody was blocked by the first antibody by competitive inhibition (suggesting the same epitope).

As expected, ABT-806 always showed low response as the second antibody, since ABT-806 does not bind appreciably to wild-type EGFR. All EGFR-targeting antibodies showed high response as the second antibody after ABT-806 association, since ABT-806 could not bind and block the second antibody.

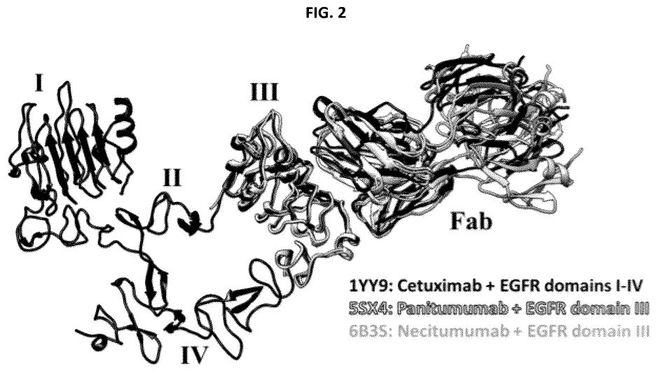

In contrast, each of the four EGFR-targeting antibodies blocked each other to some extent, suggesting the same or overlapping epitopes. Notably, the four EGFR-targeting antibodies had detectable, but decreased binding if Nimotuzumab was used as the first antibody. This is likely because Nimotuzumab has slow association kinetics and was not able to completely saturate the EGFR in the first 300-second step. In conclusion, the data confirm that ABT-806 does not bind significantly to wild-type EGFR and indicate that all four EGFR-targeting antibodies (Cetuximab, Panitumumab, Nimotuzumab, Necitumumab) share a conserved epitope. This latter conclusion is supported by other studies showing that panitumumab, Cetuximab, and Nimotuzumab have overlapping epitopes within EGFR domain III. Crystal structures of EGFR in complex with Cetuximab (1YY9), Panitumumab (5SX4), and Necitumumab (6B3S) Fabs were structurally aligned to compare epitopes of these EGFR-targeting antibodies using the MatchMaker function in Chimera 1.13 (FIG. 2). This alignment demonstrated that all three antibodies bind an overlapping epitope within domain III of EGFR, consistent with the epitope binning data by Octet.¹¹⁻¹²

### Example 7: Epitope Binning (EGFRvIII Epitope)

Biolayer interferometry was used for epitope binning of the same panel of monoclonal antibodies with EGFRvIII (Table 6). Larger response values indicate that the second antibody was able to associate with EGFR even after saturation with the first antibody had already occurred (suggesting distinct epitopes). Conversely, smaller numbers indicate that the second antibody was blocked by the first antibody by competitive inhibition (suggesting the same epitope).

As observed with the wild-type EGFR binning experiment, all four EGFR-targeting antibodies (Cetuximab, Panitumumab, Nimotuzumab, and Necitumumab) showed competitive binding with each other as demonstrated by reduced binding of the second antibody after saturation with the first antibody. This indicates that these four antibodies share an overlapping epitope on EGFRvIII that may not be specific to EGFRvIII, i.e., an EGFRwt epitope. When Nimotuzumab was used as the first (blocking) antibody, slightly higher responses for the second antibody were observed, which could be explained by nimotuzumab not saturating the EGFRvIII protein during the first 300-second blocking step due to its slower association rate.

In contrast, ABT-806 clearly binned into a distinct epitope, i.e., the EGFRvIII-specific epitope, from the four EGFR-targeting antibodies. When ABT-806 was used as the first antibody, each of the other antibodies showed significant association response in the second step. Similarly, when ABT-806 was used as the second antibody, a large additional response was observed. The lack of competitive binding between ABT-806 and the other antibodies is an indication that ABT-806 targets a unique epitope on EGFRvIII, and that ABT-806 and other EGFR-binding antibodies may simultaneously interact with different epitopes on the EGFRvIII protein. That ABT-806 binds a distinct epitope of EGFRvIII from EGFR-targeting antibodies is consistent with ABT-806 binding a conformation of the EGFR protein that is restricted to mutant EGFRvIII and not present on wild-type EGFR.

### Example 8: Generation and Characterization of Humanized ABT-806

To increase the humanness of the ABT-806 variable regions and decrease the potential for immunogenicity, the mouse VH and VK domains were converted to a more human framework. All versions used scFv models generated from the antibody modeling feature of Discovery Studio based on the sequence of ABT-806 variable domains. All humanized versions were designed using the Discovery Studio 2022 suite. VH1, VH2, VK1, and VK2 were designed using the “Predict Humanizing Mutations protocol” with the identity threshold set to 50, the frequent residue substitution tolerance set to 20, the germline substitution tolerance set to 0, and the exclusion of substitutions of Kabat CDR residues, IMGT CDR residues, Vernier zone residues, and human germline residues. This protocol is based exclusively on the amino acid sequence of ABT-806 as the query sequence. VH1 and VK1 were generated based on germline substitutions, whereas versions VH2 and VK2 used frequent residue substitutions. VH3 and VK3 were designed using an input Fv model for ABT806, with “calculate mutation energy” set to true (CHARMm forcefield) in order to generate the “best single mutations” sequences. The query structure was a model for ABT-806 generated by Discovery Studio's Antibody Modeling Cascade. The input sequences were ABT-806 VH and VL. The CDR loop definition was set to Honegger and the maximum templates per loop was set to 3, with the optimization level set to high.

Each humanized chain was combined to generate 9 versions: V1 (VH1, VK1), V2 (VH1, VK2), V3 (VH1, VK3), V4 (VH2, VK1), V5 (VH2, VK2), V6 (VH2, VK3), V7 (VH3, VK1), V8 (VH3, VK2), V9 (VH3, VK3) (Table 7). Heavy chain and light chain genes were cloned and expressed as in Example 1, and then antibodies were evaluated for stability and functional properties.

After transient expression in ExpiCHO cells, titer was quantitated using biolayer interferometry (Table 7). The optimal humanized version was selected (partially) based on overall expression.

Antibodies were purified by protein A and SEC chromatography as described in Example 1. Following protein A purification, many antibodies tended to have a high abundance of high molecular weight (HMW) or low molecular weight (LMW) contaminants in addition to the main peak corresponding to protein of interest (POI). This suggests that these antibodies are less stable, prefer to form other species (HC Dimers), and/or are more prone to aggregation. V3 and V9 seem to be the most stable humanizations based on the above metrics (Table 7). Samples used for subsequent studies were purified to >95% purity by preparative SEC.

Biolayer interferometry (Octet) binding assays were performed on an Octet384 instrument to quantify binding kinetics of humanized antibodies to EGFR and EGFRvIII, as described in Example 1. Table 8 shows that all monoclonal antibodies targeting EGFRvIII displayed detectable binding to EGFRvIII. Although similar binding kinetics were shown across all antibodies, the humanized V3 and V9 antibodies appeared to have higher responses rivaling that of ABT-806 and outperforming competitor Depatuxizumab, AbbVie's humanized ABT-806 (purified in house).

To validate antibody binding on-cell, purified primary antibodies were added to EGFR- or EGFRvIII- or uPAR-transfected CHO cells. A fluorescent secondary antibody against the primary antibody was used to measure antigen binding at the cell surface. All tested antibodies retained their binding properties to cells mimicking a cancer surface (Table 9). Additionally, all antibodies bound to EGFRvIII-expressing CHO cells significantly better than EGFR expressing CHO cells. This demonstrates that these antibodies prefer to bind the ABT-806 epitope as opposed to the Cetuximab epitope.

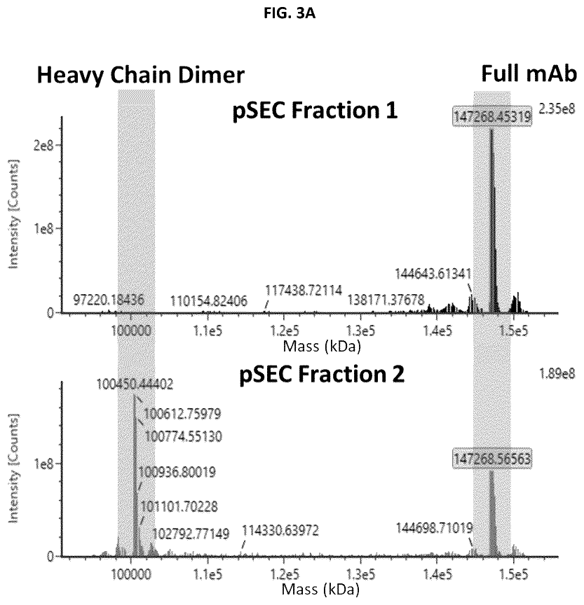

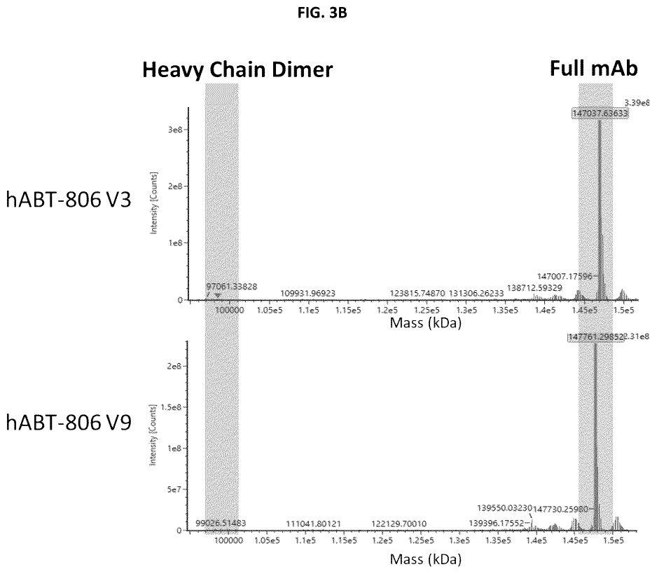

Preparative SEC (pSEC) was used prior to intact mass analysis in order to partially separate full mAb molecules (pSEC fraction 1) from heavy chain dimer molecules (pSEC fraction 2). For mass analysis and confirmation of the pSEC fractions, samples were diluted to 0.5 mg/mL with 50 mM tris pH 7.5. 1.0 μg of prepared sample was injected onto the online analytical SEC column for LC-MS intact mass analysis under denaturing conditions. The data was processed using the MaxEnt 1 algorithm within Unifi (Waters Corp). As shown in FIG. 3A, the pSEC fraction 1 of Depatuzumab contains almost exclusively intact full mAb molecules while the later eluting pSEC fraction 2 contains a mixture of full mAbs and heavy chain dimer. FIG. 3B shows that both purified hABT-806 V3 and V9 antibodies contain mostly intact full mAb. Thus, humanization of ABT806 generated an antibody that is more stable, and that is expressed with fewer problematic contaminants.

The variable domains of the lead heavy and light chains were compared against the LENS Database for sequence similarity. The result indicates that variable region sequences of VH3, VK3, and VH1 are unique (Table 10).

### Example 9. Generation and Characterization of Dematured Cetuximab

Mutations of Cetuximab to demature its binding to EGFR were identified using an in silico-based approach. The contact area between Cetuximab and EGFR from a high-resolution crystal structure of Cetuximab-bound EGFR (1YY9) was calculated using the Analyze Protein Interface Method within Discovery Studio (Biovia). Nine Cetuximab residues with greater than 20 Å² contact surface area were identified as being strongly involved in binding EGFR. The residues of interest are as follows (sequential numbering): from the light chain W94, N91, and N92; from the heavy chain W52, N56, Y101, Y102, D103, Y104. Each residue of interest was individually mutated to one of the 20 naturally occurring amino acids. In total 180 (9 residues*20 amino acids) independent mutations were generated in silico. The effect of a mutation on Cetuximab stability was calculated using the Discovery Studio Calculate Mutation Energy (Stability) function while the effect of the mutation on EGFR binding was calculated using the Calculate Mutation Energy (Binding) function with EGFR set as the ligand molecule. Briefly, the stability function calculates the difference in energy between the WT Cetuximab and the mutant Cetuximab (ΔΔG Stability) whereas the binding function calculates the binding energy between Cetuximab and EGFR (ΔΔG WT), then between the Cetuximab mutant and EGFR (ΔΔG mutant) with the difference between the energies reported as the energy change of binding due to the mutation (ΔΔ Binding=ΔΔG mutant—ΔΔG WT). Energies were calculated using CHARMm in a pH-independent mode. For all the mutations, ΔΔG Stability were plotted against the corresponding ΔΔG Binding to identify mutations with a range of possible binding dematuration that also did not strongly destabilizing Cetuximab. Ten mutations were chosen for further analysis: from the light chain N92K, N92F; from the heavy chain Y101A, Y101W, Y102V, Y102A, D103W, D103Y, D103F, and Y104L (Table 11).

The dematured Cetuximab variants were prepared as follows. Codon-optimized coding regions preceded by Kozak and secretory signal peptide sequences were cloned into the pTT5 vector using standard molecular biology techniques. Antibodies were expressed by transiently transfecting the light chains and heavy chains in the ExpiCHO system (ThermoFischer Scientific) as described in Example 1 but scaling down to a culture volume of 2 ml in 6-well plates. Proteins were purified from the harvested supernatant using a Captureem Protein A 24-Well Plate (TaKaRa). The supernatant was loaded onto the phosphate-buffered saline (PBS, 125 mM sodium phosphate, 137 mM sodium chloride, pH 6.8) equilibrated plate. The plate was washed with 10 mL PBS prior to eluting the bound protein with 0.5 ml of 50 mM sodium acetate, pH 3.5. The eluted protein was immediately neutralized by addition 0.1 mL 1M Tris-Cl, pH 8.0. Immediately after protein A purification, proteins were analyzed by analytical SEC as described in Example 1. Biolayer interferometry (Octet) binding assays were performed as described in Example 1.

To assess the stability of dematured Cetuximab variants, biolayer interferometry and analytical size-exclusion chromatography were used. After transient expression in ExpiCHO cells, titer was quantitated using biolayer interferometry (Table 12). The optimal dematured version selection was informed in part by overall expression. Immediately following first-step protein A purification, antibodies were evaluated for stability and aggregation by analytical size-exclusion chromatography on a Waters UPLC system. Many antibodies tended to have a high molecular weight (HMW) in addition to the main peak corresponding to protein of interest (POI) (Table 12). Higher abundance of HMW species suggests that these antibodies are less stable and are more prone to aggregation.

Biolayer interferometry was used to assess the affinity and avidity of dematured Cetuximab for EGFR and EGFRvIII, respectively. Table 13 shows that most dematured Cetuximab variants have decreased binding affinity and/or decreased binding response to EGFR as compared to that of Cetuximab wild type, indicating that the selected mutations alter the ability of Cetuximab to bind EGFR. However, the antibodies retain good avidity for immobilized EGFR.

Table 14 shows that most dematured Cetuximab variants also have decreased binding affinity and/or decreased binding response to EGFRvIII. Despite the decrease, all selected dematured Cetuximab variants retained their ability to bind to EGFRvIII with similar binding kinetics. This data reinstates that Cetuximab binds to the same epitope on EGFRvIII and EGFR. Although the variants had weaker affinity to EGFR and EGFRvIII, they retained good avidity for EGFR and EGFRvIII due to bivalent binding. Since avidity depends on the level of antigen on the surface, this result suggests that the dematured versions of Cetuximab may be more selective for tumors bearing a high amount of EGFR and/or EGFRvIII.

To validate that dematured Cetuximab variants retain the ability of binding to cell, purified primary antibodies were added to EGFR-, EGFRvIII-, or uPAR-transfected CHO cells. A fluorescent secondary antibody against the primary antibody was used to measure antigen binding at the cell surface. Table 15 shows that all tested antibodies (except HC: D103W) retained their binding properties to cells mimicking a cancer surface. The result indicates that these antibodies retain the binding behavior desirable for making antibody therapeutics.

### Example 10. Generation and Characterization of Biepitopic Tetravalent Antibodies Targeting EGFR

Nimotuzumab has been approved for treatment of solid tumors in several countries after the approval of Cetuximab. At least one of its improvements is reduced skin toxicities, which seems to be correlated with its lower affinity for EGFR, i.e., a K_{D} value of 9.1 nM versus 3.77 nM (Table 3).9 In this context, the most comparable Cetuximab variant kinetically is Y104L (K_{D} 9.2 nM), whereas all other variants displayed even weaker EGFR affinity than Nimotuzumab, including Y101A (K_{D} 67 nM), Y102A (97 nM), and N92F (34 nM). These dematured variants were incorporated into biepitopic tetravalent antibodies to screen for a potential therapeutic candidate for efficaciously targeting EGFRvIII-expressing tumors in the presence of EGFRwt.

Biepitopic tetravalent antibodies targeting EGFR and EGFRvIII, also known as anti-EGFR X EGFRvIII antibodies, were constructed by fusing scFvs onto the N- or C-terminus of antibody heavy or light chains. FIG. 1 depicts four exemplary structures, wherein the EGFR binding domain may be derived from humanized and/or dematured Cetuximab variants (hDC-EGFR) and formatted as either Fab or scFv while ABT-806 or humanized ABT-806 may be reciprocally formatted as either scFv or Fab.

Table 16 lists four groups of configuration formats of anti-hDC-EGFR×hABT-806 antibodies, namely, SI-95X45-49, SI-95X52-56, SI-95X58-62, and SI-95X64-68. Their EGFR binding domain was derived from humanized dematured Cetuximab variants (hDC-EGFR), namely, SI-95m4-10. SI-95X45-49 have a C-terminal fusion of an anti-ABT-806 scFv on the antibody heavy chain with an anti-hDC-EGFR in the Fab position, SI-95X52-56 contain an N-terminal fusion of an anti-hDC-EGFR scFv on the antibody heavy chain with an ABT-806 V9 in the Fab position, SI-95X58-62 contain a C-terminal fusion of ABT-806 V9 scFv on the antibody light chain with an anti-hDC-EGFR in the Fab position, and SI-95X64-68 contain a C-terminal fusion of an anti-hDC-EGFR scFv on the antibody heavy chain with an ABT-806 in the Fab position. The materials and methods used for generating anti-EGFR×EGFRvIII antibodies were the same as or similar to those described in Example 1.

After transient expression in ExpiCHO cells, titer was quantitated using biolayer interferometry (Table 17). As expected, these bispecific antibodies (SI-95X40-68) were produced with variable efficiency depending primarily on the composition and configurations of each binding domain. For example, molecules with an anti-hDC-EGFR scFv fused to the C-terminus of the antibody heavy chain (SI-95X63-68) seemed to express less than those with the with an anti-hDC-EGFR scFv fused to the N-terminus of the heavy chain (SI-95X51-56). Additionally, molecules with the anti-hDC-EGFR Y101A domain (SI-95X47, 53, 58, 65) tended to have high titers whereas those with an anti-hDC-EGFR Y104L domain (SI-95X46, 56, 61, 68) tended to have low titers.

Immediately following first-step protein A purification, anti-EGFR×EGFRvIII bispecific antibodies were evaluated for stability and aggregation by analytical size-exclusion chromatography on a Waters UPLC system (Table 17). The configuration of the molecule seemed to play the largest role in % protein of interest after first step purification. Large amount of high molecular weight aggregate demonstrates instability in the SI-95X57-62 molecules. This trend demonstrates that an ABT-806 V9 scFv fusion on the antibody light chain creates a generally unstable molecule. Conversely, SI-95X51-56 demonstrated a very high % protein of interest after preliminary purification, indicating that an N-terminal fusion of an anti-hDC-EGFR scFv to the antibody heavy chain generally results in a stable molecule.

Biolayer interferometry was used to assess the affinity of bispecific antibodies for EGFR (Table 18). Molecules containing an anti-hDC-EGFR D103Y (SI-95X45, 55, 60, 67) domain demonstrated no binding or very little binding to EGFR. This indicates that the affinity of this domain for EGFR is either very low or ablated. All other molecules containing an anti-hDC-EGFR domain displayed reduction in affinity to EGFR when compared to the control molecule in a “matured” configuration. The decrease in affinity varied depending on both configuration of the molecule and anti-hDC-EGFR domain type.

Biolayer interferometry was used to assess the affinity of bispecific antibodies for EGFRvIII (Table 19). Molecules containing an anti-hDC-EGFR D103Y (SI-95X45, 55, 60, 67) domain demonstrated binding to EGFRvIII, but not EGFR (Table 18, Table 19). Taken together, this indicates that the ABT-806 domains (or domain derivatives) are functional, whereas the anti-hDC-EGFR D103Y is either not functional or able to slightly function depending on configuration of the molecule. All molecules were able to bind EGFRvIII (albeit with slightly less affinity) compared to their control molecules in matured configurations. When compared with the monospecific mAb in Table 14, binding is greatly enhanced in all the bispecific antibodies. Therefore, these molecules can tightly bind EGFR and EGFRvIII even with an anti-hDC-EGFR domain. In fact, the affinity of some bispecific antibodies for EGFRvIII is enhanced compared to wild-type Cetuximab and ABT806 mAbs, replicating the multivalent avidity effect observed in Example 5, and suggesting a mechanism of simultaneous binding of matured Cetuximab and ABT806 domains to the same molecule of EGFRvIII. Furthermore, the binding of some antibodies was tighter to EGFRvIII (Table 19) than to EGFRwt (Table 18), indicating preferential binding to EGFRvIII while still retaining good binding to EGFRwt.

### Example 11: Biepitopic Tetravalent Antibodies Exert ADCC Activity Toward EGFRvIII-Expressing Cells

To analyze any potency change of biepitopic tetravalent antibodies when targeting tumor cells expressing EGFRwt and EGFRvIII, the ADCC assay was performed by using NK cells (1 donor) to co-culture with U87-MG-GFP cells expressing EGFRwt, U87-MG-EGFRvIII-NR (i.e., mKATE2+) cells expressing both EGFRwt and EGFRvIII, or the mixed culture of the two. Mixed cell cultures were seeded and started with 50:50 green/red signals. While the green signals from U87-MG-GFP cells were correlated to the EGFRwt-expressing live cells, the red signals from U87-MG-EGFRvIII-NR cells were correlated to the cells co-expressing EGFRwt and EGFRvIII. Following spheroid formation, NK cells were plated at a 5:1 effector to target ratio, and antibodies were plated on an 8-point log 10 dose response curve. After 60 hours, cellular fluorescence was measured to quantify live cells, and normalized to the timepoint at 0 hours. This ADCC assay was used to screen the monoepitopic bivalent antibodies (i.e., mAb) as well as biepitopic tetravalent antibodies for a therapeutic candidate (Table 16).

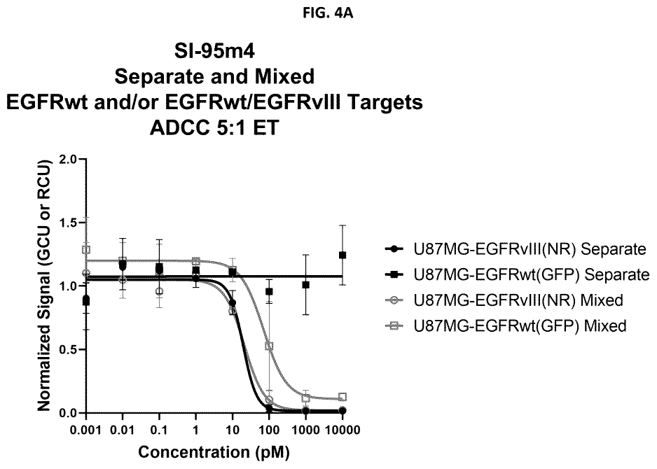

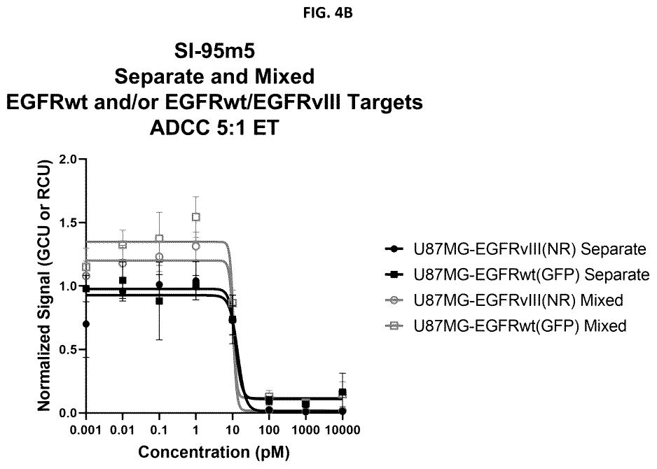

Both SI-95m4 and SI-95m5 antibodies are monoepitopic bivalent antibodies targeting EGFRvIII and EGFRwt, respectively. As shown in FIG. 4A, SI-95m4 failed to kill U87-MG-EGFRwt-GFP cells in the ADCC assay, confirming that this humanized anti-EGFRvIII antibody retained the exclusive binding specificity of the ABT-806 antibody. However, when mixed with U87-MG-EGFRvIII-NR cells, SI-95m4 exerted ADCC activity to U87-MG-EGFRwt-GFP cells (i.e., the EGFRwt target cells, FIG. 4A). This surprising result may be interpreted as a bystander effect of activated NK cells toward EGFRwt-expressing cells. In contrast, SI-95m5 was able to exert killing of both U87-MG-EGFRwt and U87-MG-EGFRvIII cells whether separate or mixed, consistent with this antibody's ability to bind both EGFRwt and EGFRvIII (FIG. 4B).

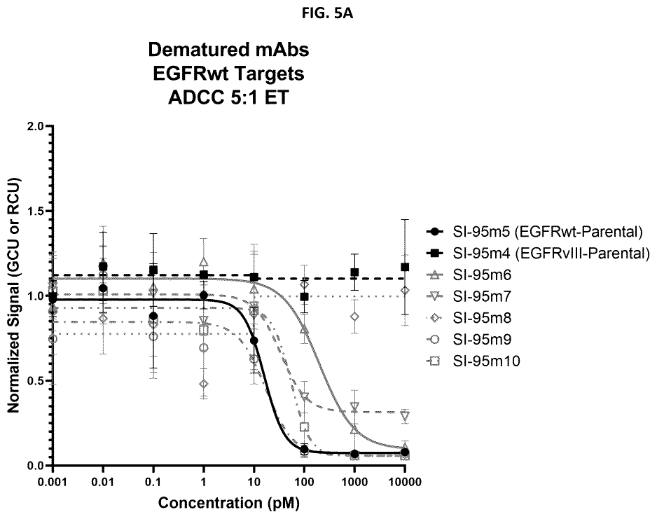

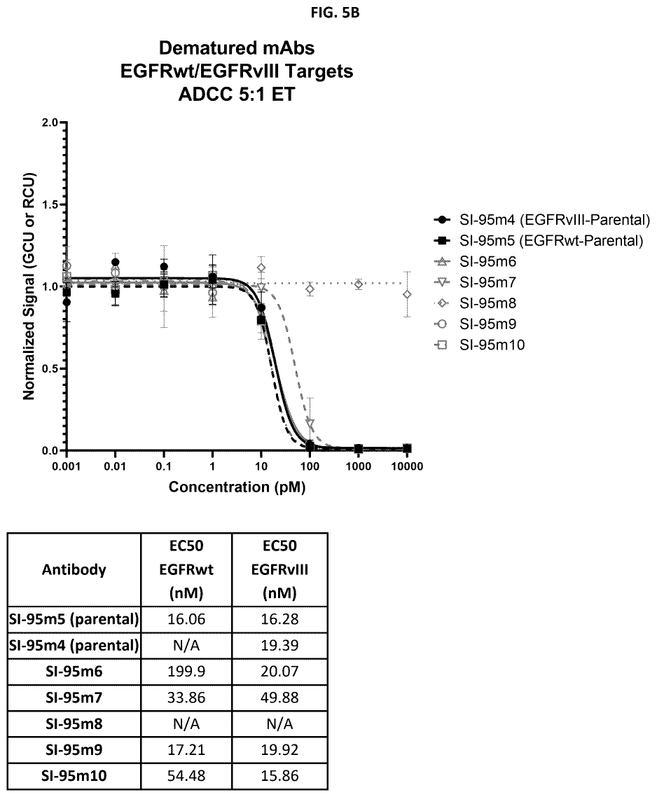

To analyze the effect of dematuration, a panel of monoepitopic bivalent antibodies, with either matured EGFRwt (Cetuximab) or dematured EGFRwt binding domains, were subjected to the ADCC assay using EGFRwt-expressing U87-MG-GFP cells and EGFRwt/EGFRvIII-expressing U87-MG-NR cells, respectively. As shown in FIG. 5, the panel of monoepitopic bivalent antibodies, consisting of SI-95m6, SI-95m7, SI-95m8, SI-95m9, and SI-95m10 (Table 16), displayed a range of reduced ADCC activity toward EGFRwt-expressing cells when compared to the parental anti-hDC-EGFR antibody, SI-95m5 (FIG. 5A). The EC50 value for the variants was increased up to 12-fold (for SI-95m6) compared to that of the parental mAb SI-95m5, indicating weaker potency. As expected, SI-95m4, an anti-EGFRvIII control antibody for the assay, showed no activity. When the EGFRwt/EGFRvIII-expressing cells were used, SI-95m6, SI-95m9, and SI-95m10 exerted their ADCC activity at a level similar to the positive control antibody, SI-95m4 (FIG. 5B). Of this panel of antibodies, SI-95m7 displayed a right-shifted curve with 3-fold increased EC50, indicative of a characteristic reduction in ADCC activity. In the meantime, SI-95m8 no longer showed any activity toward EGFR (FIGS. 5A and 5B), which is likely due to the specific D103Y mutation in its EGFR binding domain. Even with limited data, there seems to be no correlated ADCC reduction between EGFRwt-expressing and EGFRvIII-expressing when comparing the panel of mAb harboring different dematured mutations. This result may be explained by different densities or geometries of EGFRwt/EGFRvIII on the two populations of cells, allowing for differential binding avidity and initiation of ADCC.

To screen for bispecific candidate(s), a panel of biepitopic tetravalent antibodies was constructed based on the panel of monoepitopic bivalent antibodies (Table 16, FIGS. 4 and 5), and was subjected to the same ADCC assay using EGFRwt-expressing cells and EGFRwt/EGFRvIII-expressing cells, respectively.

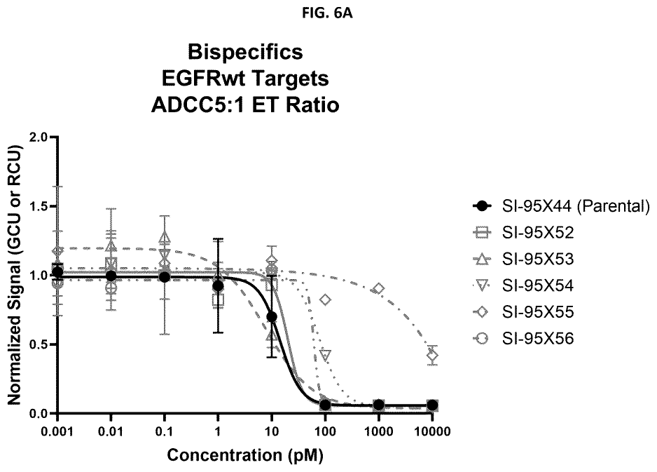

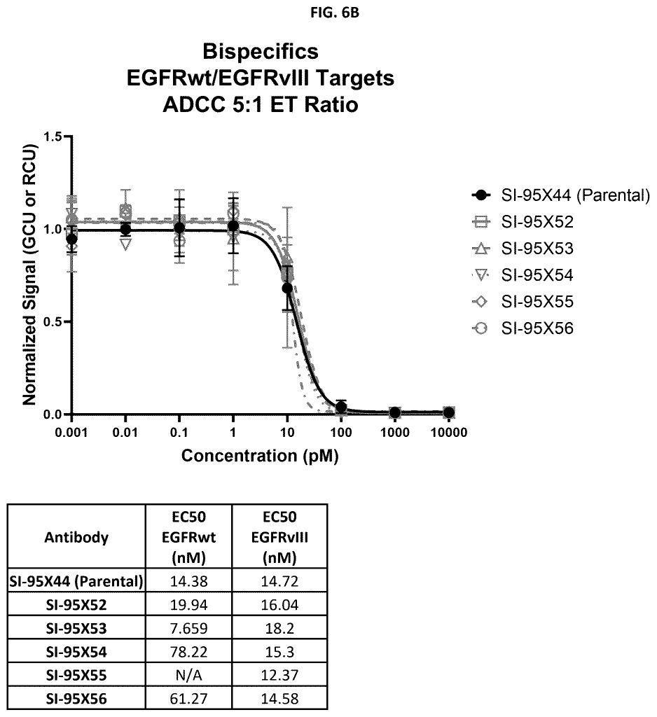

The panel of biepitopic tetravalent antibodies included SI-95X52, SI-95X53, SI-95X54, SI-95X55, and SI-95X56, each of which contains an anti-EGFRvIII Fab binding domain, i.e., a hABT-806 (V9) antibody backbone, and an anti-EGFRwt scFv binding domain. The panel shared a common antibody backbone derived from their parental mAb, SI-94m4. Their EGFRwt binding domains were derived from the anti-hDC-EGFR scFv variants and corresponded to the panel of dematured anti-EGFRwt mAb with SI-95m5 as one of parental antibodies, namely, SI-95m10 (corresponding to SI-95X52), SI-95m6 (SI-95X53), SI-95m7 (SI-95X54), SI-95m8 (SI-95X55), and SI-95m9 (SI-95X56) (Table 16, FIG. 5B). In this regard, SI-95X44 served as a matured biepitopic tetravalent antibody control (Table 16). These biepitopic tetravalent antibodies exhibited a range of ADCC activities toward EGFRwt-expressing cells (FIG. 6A) and seemingly overlapping curves toward EGFRvIII-expressing tumor cells (FIG. 6B). Of note, SI-95X55 had significantly reduced activity toward EGFRwt-expressing cells. The EC50 values for these overlapping curves were calculated and listed in FIG. 6B, revealing lower EC50 numbers than EC50 of 19.3 nM for their parental antibody, SI-95m4. A lower EC50 value is indicative of improved ADCC activity. The observation may imply a synergetic effect for enhanced ADCC activity as a result of simultaneous binding of two EGFR epitopes on EGFRwt/EGFRvIII-expressing cells. Notably, the different configurations of biepitopic antibodies with regard to the geometry and distance of the anti-EGFR and anti-EGFRvIII binding domains may allow for varying capacities for this simultaneous binding mechanism on cells, which could impact the extent of ADCC against the EGFRwt-expressing tumor cells as well as EGFRwt/EGFRvIII-expressing tumor cells. Together with the finding in the mAb format, the synergetic effects of biepitopic tetravalent antibodies are likely influenced by the structural change as a result of humanization, dematuration, and/or configuration. In addition, SI-95X55 exhibited improved potency to EGFRwt/EGFRvIII-expressing cells, as indicated by its EC50 (12.37 nM) as compared to that of either SI-95m4 (19.39 nM) or SI-95X44 (14.72 nM) as listed in FIG. 6B. This observation indicates that even residual affinity/avidity to EGFRwt (Table 15) helped improve its potency of ADCC. It also demonstrates improvement of therapeutic index by decreasing potency against EGFRwt-bearing cells while increasing potency against EGFRwt/EGFRvIII-bearing cells.

### Example 12: Biepitopic Tetravalent Antibodies Targeting Both EGFRwt and EGFRvIII Expressing Tumors

The results from Example 11 indicated that the ADCC activity of a dematured monoepitopic antibody may not predict the same for the biepitopic antibody harboring the same dematuration mutation. For example, SI-95m8, SI-95X55, and SI-95X67 (with a configuration opposite to that of SI-95X55, see Table 16) harbor the same D103Y mutation. The loss of ADCC potency against EGFRwt-expressing cells made SI-95X55 a candidate only for targeting EGFRvIII tumors. In this context, the screen of biepitopic tetravalent antibodies that can efficiently target both EGFRwt and EGFRvIII expressing tumors was focused on those groups of antibodies harboring dematuration mutations, Y101A and Y102A.

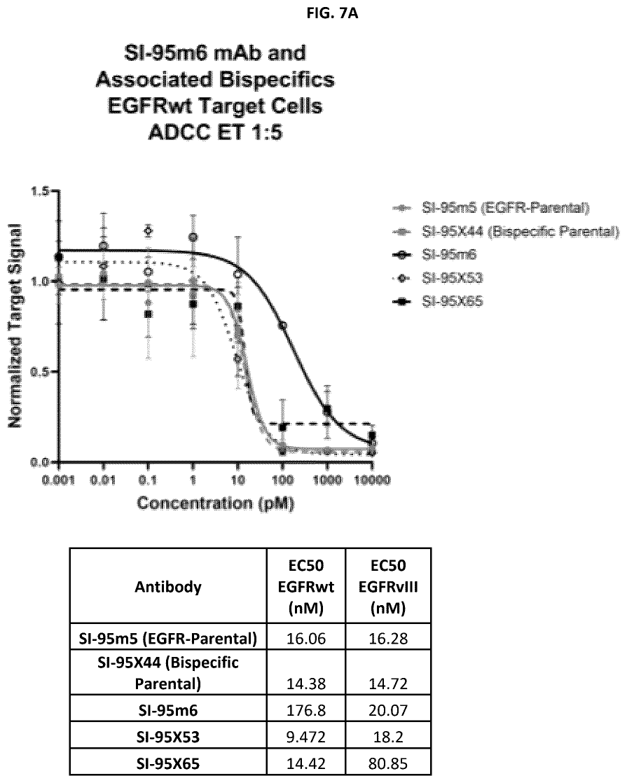

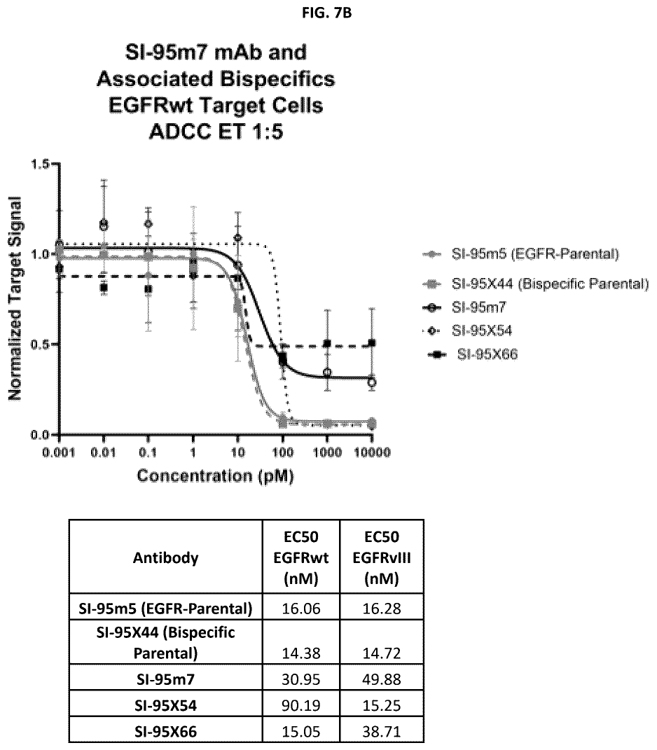

The Y101A mutation is the common feature of dematured antibodies: SI-95m6 and SI-95X53/SI-95X65, of which the antibody backbone of SI-95X65 was not humanized (Table 16). As shown in FIG. 7, SI-95X65, which contains the anti-EGFR scFv at the opposite end of the heavy chain as SI-95X53, displayed an overlapping but incomplete killing curve as compared to SI-95X53 (FIG. 7A), whereas SI-95m6 had a nearly 10-fold higher EC50 against EGFRwt cells (199.9 nM) than SI-95m5 (EC50=16.06 nM), the parental antibody (i.e., with matured binding to EGFRwt) (see table in FIG. 7B). This means that when the dematured mutation, Y101A, was placed into a biepitopic tetravalent antibody, the EC50 (7.659 nM) was similar to that of the parental SI-95m5 antibody as well as the bispecifics containing the parental mAb backbone structure (SI-95X44) (EC50=14.38 nM). In this regard, SI-95X52, which harbored N92F mutation (Table 16), showed similar potency and complete killing (FIG. 6B).

The Y102A mutation is the common feature of dematured antibodies: SI-95m7, SI-95X54, and SI-95X66, of which the antibody backbone of SI-95X66 was not humanized and contains the anti-EGFR scFv domain on the opposite end of the heavy chain when compared to SI-95X54 (Table 16). When compared with the overlapping curves of SI-95m5 and SI-95X44, both SI-95m7 and SI-95X66 displayed right-shifted curves with incomplete killing, whereas SI-95X54 responded with a complete killing curve of EGFRwt-expressing cells in a range between from 10 nM to 100 nM (see table in FIG. 7B). The right shift by SI-95X54 was measured at approximately 5-fold higher than the EC50 values of both SI-95m5 and SI-95X44. SI-95X54 is a biepitopic tetravalent antibody having its scFv domain derived from the anti-hDC-EFGR variant with Y102A. Thus, SI-95X54 unveils its EGFRvIII-biased cell killing while also killing EGFRwt tumors at least 5-fold higher EC50 value.

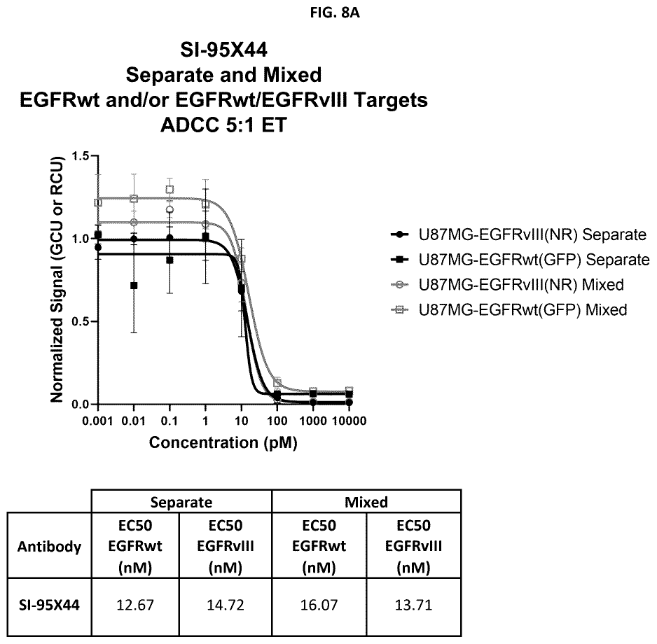

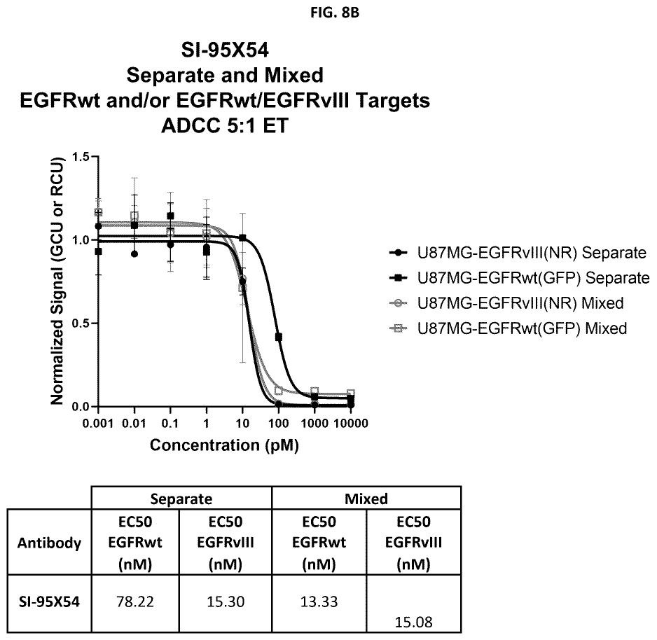

To further compare the changes in ADCC activity, the panel of separate and mixed EGFRwt and EGFRvIII cells was used to evaluate bispecific candidates. As a control, SI-95X44 displayed overlapping curves and similar ED50 values as expected (FIG. 8A). A bispecific candidate such as SI-95X54 displayed a characteristic right-shift curve with a complete killing of mixed cells (FIG. 8B). This result demonstrates that as an exemplary biepitopic tetravalent antibody, SI-95X54 is capable of efficiently killing a mixed EGFRwt and EGFRvIII cell population, which may mimic the heterogeneity of glioblastoma in a human patient, and that a reduction in its sensitivity to kill EGFRwt cells may imply a reduced toxicity to heathy tissues of the patient.

EGFRwt and EGFRvIII play complex and interactive roles in the genesis of brain tumors. EGFR overexpression is widespread among the tumor cells in most cases of glioblastomas, whereas EGFRvIII typically shows patchy tumor positivity of only a minority of the tumor cells, when pan-EGFR antibodies are used for staining. Such tumor heterogeneity remains as an unmet challenge for developing effective and meaningful treatment for brain tumors. Thus, a therapeutic antibody simultaneously targeting both EGFRwt and EGFRvIII cells with EGFRvIII-biased binding may be a desirable solution. In this context, this application provided the proof of concept of a therapeutic strategy by which biepitopic tetravalent antibodies have been generated to effectively eradicate mixed tumor cell populations expressing EGFRwt and/or EGFRvIII with lower toxicity to normal tissues.

### Tables

TABLE 1
Configurations of bispecific tetravalent antibodies

| | αEGFR-WT | αEGFRvIII | scFv | scFv | Interdomain |
|---|---|---|---|---|---|
| Protein | domain | domain | location | orientation | linker length (aa) |
| Cetuximab | Cetuximab (Fab) | n/a | n/a | n/a | n/a |
| Nimotuzumab | Nimotuzumab (Fab) | n/a | n/a | n/a | n/a |
| ABT-806 | n/a | ABT-806 (Fab) | n/a | n/a | n/a |
| SI-95X1 | Cetuximab (Fab) | ABT-806 (scFv) | HC-C-scFv | H-L | 10 |
| SI-95X2 | Nimotuzumab (Fab) | | HC-C-scFv | | |
| SI-95X3 | Cetuximab (scFv) | ABT-806 (Fab) | HC-C-scFv | | |
| SI-95X4 | Nimotuzumab (scFv) | | HC-C-scFv | | |
| SI-95X5 | Nimotuzumab (scFv) | ABT-806 (Fab) | scFv-N-HC | H-L | 20 |
| SI-95X6 | | | | | 25 |
| SI-95X7 | | | | | 30 |
| SI-95X8 | | | | L-H | 20 |
| SI-95X9 | | | | | 25 |
| SI-95X10 | | | | | 30 |
| SI-95X11 | | | scFv-N-LC | H-L | 20 |
| SI-95X12 | | | | | 25 |
| SI-95X13 | | | | | 30 |
| SI-95X14 | | | | L-H | 20 |
| SI-95X15 | | | | | 25 |
| SI-95X16 | | | | | 30 |
| SI-95X17 | Nimotuzumab (Fab) | ABT-806 (scFv) | scFv-N-HC | H-L | 20 |
| SI-95X18 | | | | | 25 |
| SI-95X19 | | | | | 30 |
| SI-95X20 | | | | L-H | 20 |
| SI-95X21 | | | | | 25 |
| SI-95X22 | | | | | 30 |
| SI-95X23 | | | scFv-N-LC | H-L | 20 |
| SI-95X24 | | | | | 25 |
| SI-95X25 | | | | | 30 |
| SI-95X26 | | | | L-H | 20 |
| SI-95X27 | | | | | 25 |
| SI-95X28 | | | | | 30 |
| SI-95X29 | | | LC-C-scFv | H-L | 20 |
| SI-95X30 | | | | | 25 |
| SI-95X31 | | | | | 30 |
| SI-95X35 | Cetuximab (scFv) | ABT-806 (mAb) | HC N-term | LH | 20 |
| SI-95X36 | | | LC N-term | LH | 20 |
| SI-95X37 | Cetuximab (mAb) | ABT-806 | HC N-term | LH | 20 |
| SI-95X38 | | (scFv) | LC N-term | LH | 20 |
| SI-95X39 | | | LC C-term | HL | 20 |

TABLE 2
Stability assessment of bispecific tetravalent antibodies targeting
EGFR-WT × EGFRvIII by expression titer and analytical size-
exclusion chromatography after first step protein A purification.

| | Titer | % | % | % | |
|---|---|---|---|---|---|
| Protein | (ug/ml) | POI | HMW | LMW | Notes |
| Cetuximab | 154 | | | | |
| Nimotuzumab | 39 | | | | |
| ABT-806 | 58 | | | | |
| SI-95X1 | 175 | 83.82 | 16.18 | 0.00 | |
| SI-95X2 | 64 | 89.36 | 10.42 | 0.22 | |
| SI-95X3 | 127 | 92.08 | 7.92 | 0.00 | |
| SI-95X4 | 22 | 38.30 | 60.15 | 1.55 | Low-quality data |
| SI-95X5 | 15 | 55.96 | 42.99 | 1.04 | Low-quality data |
| SI-95X6 | 16 | 61.47 | 37.01 | 1.52 | Low-quality data |
| SI-95X7 | 13 | 72.67 | 21.67 | 5.66 | Low-quality data |
| SI-95X8 | 35 | 73.88 | 14.56 | 11.53 | Low-quality data |
| SI-95X9 | 50 | 24.72 | 72.71 | 2.56 | Low-quality data |
| SI-95X10 | 49 | 22.15 | 76.13 | 1.72 | Low-quality data |
| SI-95X11 | 6 | 70.24 | 28.67 | 1.09 | Low-quality data |
| SI-95X12 | 6 | 20.98 | 64.00 | 15.05 | Low-quality data |
| SI-95X13 | 5 | 71.42 | 1.78 | 26.80 | Low-quality data |
| SI-95X14 | 13 | 4.43 | 86.86 | 8.87 | Low-quality data |
| SI-95X15 | 10 | 3.85 | 85.11 | 11.04 | Low-quality data |
| SI-95X16 | 10 | 7.75 | 82.53 | 9.72 | Low-quality data |
| SI-95X17 | 37 | 63.42 | 36.58 | 0.00 | Split peak |
| SI-95X18 | 34 | 56.42 | 42.94 | 0.64 | Split peak |
| SI-95X19 | 42 | 57.92 | 41.44 | 0.63 | Split peak |
| SI-95X20 | 78 | 73.91 | 25.77 | 0.32 | |
| SI-95X21 | 85 | 71.64 | 21.20 | 7.16 | |
| SI-95X22 | 68 | 77.81 | 18.37 | 3.81 | |
| SI-95X23 | 22 | 54.31 | 15.21 | 30.48 | Split peak |
| SI-95X24 | 21 | 58.03 | 39.50 | 2.47 | Split peak |
| SI-95X25 | 21 | 54.92 | 43.81 | 1.27 | Split peak |
| SI-95X26 | 36 | 63.31 | 35.16 | 1.53 | |
| SI-95X27 | 34 | 66.02 | 29.88 | 4.09 | |
| SI-95X28 | 34 | 69.26 | 27.28 | 3.46 | |
| SI-95X29 | 27 | 61.32 | 35.13 | 3.55 | Broad main peak |
| SI-95X30 | 23 | 52.39 | 43.24 | 4.37 | Broad main peak |
| SI-95X31 | 24 | 54.06 | 42.63 | 3.31 | Broad main peak |
| SI-95X35 | 119 | 68.30 | 31.70 | 0.00 | |
| SI-95X36 | 52 | 72.00 | 28.00 | 0.00 | |
| SI-95X37 | 72 | 75.30 | 25.00 | 0.00 | |
| SI-95X38 | 24 | 67.20 | 32.80 | 0.00 | |
| SI-95X39 | 50 | 60.20 | 39.80 | 0.00 | |

TABLE 3
Binding kinetics of bispecific tetravalent antibodies to EGFRwt

| | Response | KD | kon | koff |
|---|---|---|---|---|
| Protein | (nm) | (M) | (M⁻¹s⁻¹) | (s⁻¹) |
| Cetuximab | 0.75 | 3.77E−09 | 3.41E+05 | 1.29E−03 |
| Nimotuzumab | 0.32 | 9.09E−09 | 1.48E+05 | 1.34E−03 |
| ABT-806 | 0.07 | ND | ND | ND |
| SI-95X1 | 0.62 | 2.36E−09 | 3.89E+05 | 9.18E−04 |
| SI-95X2 | 0.26 | 5.53E−09 | 1.14E+05 | 6.30E−04 |
| SI-95X3 | 0.41 | 6.80E−10 | 4.20E+05 | 2.85E−04 |
| SI-95X4 | 0.04 | ND | ND | ND |
| SI-95X5 | 0.04 | ND | ND | ND |
| SI-95X6 | 0.03 | ND | ND | ND |
| SI-95X7 | 0.02 | ND | ND | ND |
| SI-95X8 | 0.02 | ND | ND | ND |
| SI-95X9 | 0.01 | ND | ND | ND |
| SI-95X10 | 0.00 | ND | ND | ND |
| SI-95X11 | 0.02 | ND | ND | ND |
| SI-95X12 | 0.02 | ND | ND | ND |
| SI-95X13 | 0.01 | ND | ND | ND |
| SI-95X14 | 0.00 | ND | ND | ND |
| SI-95X15 | 0.00 | ND | ND | ND |
| SI-95X16 | 0.00 | ND | ND | ND |
| SI-95X17 | 0.26 | 6.02E−09 | 7.78E+04 | 4.69E−04 |
| SI-95X18 | 0.24 | 4.17E−09 | 6.35E+04 | 2.65E−04 |
| SI-95X19 | 0.23 | 5.01E−09 | 8.78E+04 | 4.40E−04 |
| SI-95X20 | 0.24 | 4.73E−09 | 6.94E+04 | 3.29E−04 |
| SI-95X21 | 0.24 | 5.56E−09 | 7.62E+04 | 4.23E−04 |
| SI-95X22 | 0.21 | 6.10E−09 | 4.67E+04 | 2.85E−04 |
| SI-95X23 | 0.16 | 6.17E−09 | 8.16E+04 | 5.04E−04 |
| SI-95X24 | 0.17 | 7.94E−09 | 5.52E+04 | 4.38E−04 |
| SI-95X25 | 0.13 | 1.34E−08 | 5.56E+04 | 7.44E−04 |
| SI-95X26 | 0.17 | 9.35E−09 | 7.13E+04 | 6.67E−04 |
| SI-95X27 | 0.16 | 1.07E−08 | 4.55E+04 | 4.88E−04 |
| SI-95X28 | 0.15 | 7.56E−09 | 5.52E+04 | 4.17E−04 |
| SI-95X29 | 0.22 | 1.01E−08 | 8.89E+04 | 9.01E−04 |
| SI-95X30 | 0.20 | 8.84E−09 | 9.72E+04 | 8.60E−04 |
| SI-95X31 | 0.19 | 1.05E−08 | 8.77E+04 | 9.23E−04 |
| SI-95X35 | 0.72 | 6.86E−10 | 3.82E+05 | 2.62E−04 |
| SI-95X36 | 0.72 | 1.44E−09 | 5.08E+05 | 7.30E−04 |
| SI-95X37 | 0.76 | 1.55E−09 | 4.61E+05 | 7.12E−04 |
| SI-95X38 | 0.75 | 1.62E−09 | 4.68E+05 | 7.57E−04 |
| SI-95X39 | 0.35 | 5.05E−09 | 1.58E+05 | 7.97E−04 |

TABLE 4
Binding kinetics of bispecific tetravalent antibodies to EGFRvIII

| | Response | KD | kon | koff |
|---|---|---|---|---|
| Protein | (nm) | (M) | (M⁻¹s⁻¹) | (s⁻¹) |
| Cetuximab | 0.58 | 1.98E−09 | 8.09E+05 | 1.60E−03 |
| Nimotuzumab | 0.31 | 6.68E−09 | 2.02E+05 | 1.35E−03 |
| ABT-806 | 0.39 | 4.79E−09 | 3.29E+05 | 1.58E−03 |
| SI-95X1 | 0.55 | 4.45E−10 | 1.13E+06 | 5.03E−04 |
| SI-95X2 | 0.44 | 1.15E−09 | 3.07E+05 | 3.53E−04 |
| SI-95X3 | 0.45 | 3.75E−10 | 8.32E+05 | 3.12E−04 |
| SI-95X4 | 0.27 | 5.52E−09 | 3.61E+05 | 2.00E−03 |
| SI-95X5 | 0.30 | 7.94E−09 | 2.70E+05 | 2.15E−03 |
| SI-95X6 | 0.31 | 6.35E−09 | 3.34E+05 | 2.12E−03 |
| SI-95X7 | 0.31 | 8.41E−09 | 2.87E+05 | 2.41E−03 |
| SI-95X8 | 0.28 | 6.97E−09 | 2.91E+05 | 2.03E−03 |
| SI-95X9 | 0.20 | 6.16E−09 | 3.00E+05 | 1.85E−03 |
| SI-95X10 | 0.14 | 7.00E−09 | 3.27E+05 | 2.29E−03 |
| SI-95X11 | 0.08 | 4.87E−09 | 3.56E+05 | 1.73E−03 |
| SI-95X12 | 0.15 | 7.80E−09 | 2.98E+05 | 2.32E−03 |
| SI-95X13 | 0.22 | 8.77E−09 | 2.73E+05 | 2.40E−03 |
| SI-95X14 | 0.08 | 4.28E−10 | 1.00E+05 | 4.28E−05 |
| SI-95X15 | 0.07 | 1.75E−10 | 1.00E+05 | 1.75E−05 |
| SI-95X16 | 0.08 | 1.94E−08 | 1.50E+05 | 2.91E−03 |
| SI-95X17 | 0.50 | 6.42E−10 | 2.87E+05 | 1.84E−04 |
| SI-95X18 | 0.49 | 5.93E−10 | 2.96E+05 | 1.75E−04 |
| SI-95X19 | 0.47 | 6.76E−10 | 3.04E+05 | 2.06E−04 |
| SI-95X20 | 0.56 | 8.39E−10 | 3.46E+05 | 2.90E−04 |
| SI-95X21 | 0.53 | 6.54E−10 | 3.22E+05 | 2.11E−04 |
| SI-95X22 | 0.52 | 8.34E−10 | 3.45E+05 | 2.88E−04 |
| SI-95X23 | 0.38 | 1.45E−09 | 3.52E+05 | 5.09E−04 |
| SI-95X24 | 0.42 | 1.45E−09 | 3.49E+05 | 5.05E−04 |
| SI-95X25 | 0.32 | 1.21E−09 | 3.50E+05 | 4.23E−04 |
| SI-95X26 | 0.40 | 2.81E−10 | 3.92E+05 | 1.10E−04 |
| SI-95X27 | 0.41 | 5.07E−10 | 3.74E+05 | 1.90E−04 |
| SI-95X28 | 0.41 | 5.79E−10 | 3.70E+05 | 2.14E−04 |
| SI-95X29 | 0.44 | 1.12E−09 | 2.82E+05 | 3.16E−04 |
| SI-95X30 | 0.40 | 1.24E−09 | 2.90E+05 | 3.60E−04 |
| SI-95X31 | 0.40 | 1.08E−09 | 2.84E+05 | 3.07E−04 |
| SI-95X35 | 0.54 | 6.28E−10 | 4.43E+05 | 2.78E−04 |
| SI-95X36 | 0.53 | 4.01E−10 | 5.70E+05 | 2.29E−04 |
| SI-95X37 | 0.59 | 7.30E−10 | 5.34E+05 | 3.90E−04 |
| SI-95X38 | 0.54 | 6.71E−10 | 5.13E+05 | 3.44E−04 |
| SI-95X39 | 0.37 | 1.43E−09 | 2.22E+05 | 3.17E−04 |

TABLE 5
EGFRwt epitope binning of EGFRwt-
and EGFRvIII-targeting antibodies.

| | | 2^{nd} antibody | | | | |
|---|---|---|---|---|---|---|
| | | Cetu | Pani | Nimo | Neci | ABT-806 |
| 1^{st} | Cetu | 0.0198 | 0.0124 | 0.0112 | −0.0007 | −0.0048 |
| antibody | Pani | 0.0156 | 0.0071 | 0.0083 | 0.0031 | −0.0033 |
| | Nimo | 0.1815 | 0.1641 | 0.1045 | 0.1402 | −0.0072 |
| | Neci | 0.0283 | 0.0110 | 0.0153 | 0.0040 | 0.0034 |
| | ABT-806 | 0.5338 | 0.5094 | 0.2723 | 0.4637 | −0.0034 |

TABLE 6
EGFRvIII epitope binning of EGFRwt-
and EGFRvIII-targeting antibodies

| | | 2^{nd} antibody | | | | |
|---|---|---|---|---|---|---|
| | | Cetu | Pani | Nimo | Neci | ABT-806 |
| 1^{st} | Cetu | −0.0009 | 0.0104 | 0.0277 | 0.0000 | 0.6938 |
| antibody | Pani | 0.0108 | 0.0085 | 0.0097 | 0.0058 | 0.7129 |
| | Nimo | 0.1755 | 0.1529 | 0.1015 | 0.1304 | 0.6880 |
| | Neci | 0.0114 | 0.0093 | 0.0303 | 0.0071 | 0.7138 |
| | ABT-806 | 0.9734 | 0.8917 | 0.7272 | 0.8168 | 0.0315 |

TABLE 7
Characterization of humanized ABT-806 variants, such as the
expression titer and purity as assessed by using analytical
size-exclusion chromatography after protein A purification.

| Humanization | Heavy | Light | Titer | % | % | % |
|---|---|---|---|---|---|---|
| Version | Chain | Chain | (ug/mL) | POI | HMW | LMW |
| Version 1 (V1) | VH1 | VK1 | 23.0 | 54.97 | 24.25 | 20.78 |
| Version 2 (V2) | VH1 | VK2 | 36.3 | 50.06 | 31.33 | 18.61 |
| Version 3 (V3) | VH1 | VK3 | 60.0 | 87.49 | 9.8 | 2.71 |
| Version 4 (V4) | VH2 | VK1 | 50.7 | 58.66 | 28.04 | 13.3 |
| Version 5 (V5) | VH2 | VK2 | 25.1 | 43.05 | 40.87 | 16.08 |
| Version 6 (V6) | VH2 | VK3 | 57.8 | 52.69 | 34.97 | 12.34 |
| Version 7 (V7) | VH3 | VK1 | 31.1 | 56.06 | 23.06 | 20.88 |
| Version 8 (V8) | VH3 | VK2 | 32.6 | 63.77 | 16.94 | 19.29 |
| Version 9 (V9) | VH3 | VK3 | 77.5 | 91.90 | 3.38 | 4.01 |

TABLE 8
The binding affinity of humanized
ABT-806 antibodies to EGFRvIII.

| Humanized | Response | KD | kon | koff |
|---|---|---|---|---|
| Version | (nm) | (M) | (M⁻¹s⁻¹) | (s⁻¹) |
| V1 | 0.2074 | 2.95E−08 | 1.26E+05 | 3.70E−03 |
| V2 | 0.2315 | 2.66E−08 | 1.34E+05 | 3.56E−03 |
| V3 | 0.3547 | 1.54E−08 | 1.53E+05 | 2.36E−03 |
| V4 | 0.2738 | 2.32E−08 | 1.27E+05 | 2.95E−03 |
| V5 | 0.2305 | 3.07E−08 | 1.21E+05 | 3.71E−03 |
| V6 | 0.3067 | 1.84E−08 | 1.16E+05 | 2.13E−03 |
| V7 | 0.1691 | 3.78E−08 | 1.09E+05 | 4.13E−03 |
| V8 | 0.198 | 3.19E−08 | 1.09E+05 | 3.46E−03 |
| V9 | 0.3433 | 1.61E−08 | 1.60E+05 | 2.58E−03 |
| ABT-806 | 0.3045 | 1.57E−08 | 1.45E+05 | 2.28E−03 |
| Depatuxizumab | 0.259 | 1.09E−08 | 1.57E+05 | 1.72E−03 |

TABLE 9
On-Cell binding assay validating antibody retained
binding to antigen on-cell. All values reported
are MFI at 1.1 ug/mL of each antibody.

| Humanized Version | EGFR | EGFRvIII | uPAR |
|---|---|---|---|
| V1 | 634 | 10695 | 166 |
| V2 | 661 | 11707 | 199 |
| V3 | 1261 | 14652 | 204 |
| V4 | 620 | 12031 | 195 |
| V5 | 528 | 10224 | 190 |
| V6 | 664 | 11803 | 185 |
| V7 | 749 | 11179 | 196 |
| V8 | 876 | 13080 | 196 |
| V9 | 2713 | 16041 | 114 |
| ABT-806 | 914 | 13986 | 184 |
| Cetuximab | 8896 | 9229 | 177 |

TABLE 10
Similarity of lead humanized ABT-806 sequences to all in LENS Database.

| Humanized | % | |
|---|---|---|
| ABT-806 | Similarity | Sequence |
| VH3 | 89.7 | QVQLQESGPSLVKPSQTLSLTCTVSGYSITSDFAWNWIRQ |
| | | FPGRKLEWMGYISYSGNTRYNPSLKSRFTISRDTSKNQFY |
| | | LKLRSVTIADTAVYYCVTAGRGFPYWGQGTLVTVSS |
| VK3 | 92.5 | DIVMTQSPSSVSLSLGDRVTITCHSSQDINSNIGWLQQKP |
| | | GKAFKGLIYHGTNLDDGVPSRFSGSGSGADYTLTISSLQA |
| | | EDFATYYCVQYAQFPWTFGPGTKLEIK |
| VH1 | 92.2 | QVQLQESGPGLVKPSQSLSLTCTVSGYSITSDFAWNWIRQ |
| | | PPGKGLEWIGYISYSGNTRYNPSLKSRVTISRDTSKNQFS |
| | | LQLNSVTAEDTAVYYCVTAGRGFPYWGQGTLVTVSS |

TABLE 11
Energy calculations of dematured Cetuximab mutations

| | ΔΔG Stability | ΔΔG Binding |
|---|---|---|
| Mutation | (kcal/mol) | (kcal/mol) |
| Cetuximab (HC: D103F) | −0.50 | 1.17 |
| Cetuximab (HC: D103Y) | −1.41 | 3.16 |
| Cetuximab (HC: D103W) | 0.91 | 4.17 |
| Cetuximab (HC: Y101A) | 2.85 | 1.90 |
| Cetuximab (HC: Y101W) | 0.71 | 2.06 |
| Cetuximab (HC: Y102A) | 454 | 3.96 |
| Cetuximab (HC: Y104L) | 1.13 | 1.58 |
| Cetuximab (LC: N92F) | −2.21 | −1.43 |
| Cetuximab (LC: N92K) | −1.06 | 0.05 |

TABLE 12
Characterization of dematured Cetuximab variants, such as the
expression titer and purity as assessed by using analytical
size-exclusion chromatography after protein A purification.

| Dematured Cetuximab | Titer | | | |
|---|---|---|---|---|
| Variants | (ug/mL) | % POI | % HMW | % LMW |
| Cetuximab_WT | 39.0 | 95.19 | 4.81 | 0 |
| Cetuximab (HC: D103F) | 35.6 | 82.37 | 11.68 | 5.95 |
| Cetuximab (HC: D103Y) | 26.5 | 88.77 | 4.79 | 6.44 |
| Cetuximab (HC: D103W) | 22.5 | 69.95 | 30.05 | 0 |
| Cetuximab (HC: Y101A) | 26.0 | 80.74 | 19.26 | 0 |
| Cetuximab (HC: Y101W) | 38.9 | 78.23 | 21.77 | 0 |
| Cetuximab (HC: Y102A) | 31.0 | 90.93 | 9.07 | 0 |
| Cetuximab (HC: Y104L) | 34.1 | 95.31 | 4.69 | 0 |
| Cetuximab (LC: N92F) | 35.5 | 86.06 | 13.94 | 0 |
| Cetuximab (LC: N92K) | 33.8 | 85.15 | 14.85 | 0 |

TABLE 13
The binding affinity and avidity of dematured Cetuximab to EGFR

| | Binding Affinity to EGFR | | | | Binding Avidity to EGFR | | | |
|---|---|---|---|---|---|---|---|---|
| | Response | KD | kon | koff | Response | KD | kon | koff |
| Protein Name | (nm) | (M) | (M⁻¹s⁻¹) | (s⁻¹) | (nm) | (M) | (M⁻¹s⁻¹) | (s⁻¹) |
| Cetuximab_WT | 0.6754 | 5.53E−09 | 4.42E+05 | 2.44E−03 | 0.3947 | 4.80E−11 | 1.89E+06 | 9.08E−05 |
| _HC: D103F | 0.0196 | N/A | N/A | N/A | 0.0018 | N/A | N/A | N/A |
| _HC: D103Y | 0.0130 | N/A | N/A | N/A | −2.50E−03 | N/A | N/A | N/A |
| _HC: D103W | 0.0140 | N/A | N/A | N/A | −2.03E−03 | N/A | N/A | N/A |
| _HC: Y101A | 0.3343 | 6.71E−08 | 5.79E+05 | 3.89E−02 | 0.2809 | 2.01E−10 | 2.02E+06 | 4.05E−04 |
| _HC: Y101W | 0.5531 | 3.48E−09 | 4.71E+05 | 1.64E−03 | 0.2833 | <1.0E−12 | 1.77E+06 | 3.36E−07 |
| _HC: Y102A | 0.2016 | 9.65E−08 | 5.74E+05 | 5.54E−02 | 0.1641 | 5.25E−10 | 2.05E+06 | 1.08E−03 |
| _HC: Y104L | 0.4609 | 9.20E−09 | 5.38E+05 | 4.95E−03 | 0.1486 | 3.35E−11 | 2.40E+06 | 8.05E−05 |
| _LC: N92F | 0.4764 | 3.41E−08 | 5.30E+05 | 1.81E−02 | 0.4202 | 3.58E−11 | 2.12E+06 | 7.56E−05 |
| _LC: N92K | 0.6189 | 3.91E−09 | 3.83E+05 | 1.50E−03 | 0.378 | <1.0E−12 | 1.51E+06 | 5.78E−07 |

TABLE 14
The binding affinity and avidity of dematured Cetuximab to EGFRvIII

| | Binding Affinity to EGFRvIII | | | | Binding Avidity to EGFRvIII | | | |
|---|---|---|---|---|---|---|---|---|
| | Response | KD | kon | koff | Response | KD | kon | koff |
| Protein Name | (nm) | (M) | (M⁻¹s⁻¹) | (s⁻¹) | (nm) | (M) | (M⁻¹s⁻¹) | (s⁻¹) |
| Cetuximab_WT | 0.3236 | 1.97E−09 | 7.13E+05 | 1.40E−03 | 0.7112 | 1.03E−11 | 1.13E+06 | 1.15E−05 |
| _HC: D103F | −7.88E−03 | N/A | N/A | N/A | 0.0087 | N/A | N/A | N/A |
| _HC: D103Y | −2.25E−03 | N/A | N/A | N/A | 0.0034 | N/A | N/A | N/A |
| _HC: D103W | −5.59E−03 | N/A | N/A | N/A | 0.0096 | N/A | N/A | N/A |
| _HC: Y101A | 0.2186 | 3.22E−08 | 9.06E+05 | 2.91E−02 | 0.3358 | 2.44E−10 | 1.98E+06 | 4.83E−04 |
| _HC: Y101W | 0.244 | 1.28E−09 | 7.24E+05 | 9.27E−04 | 0.3164 | <1.0E−12 | 1.55E+06 | 4.90E−07 |
| _HC: Y102A | 0.164 | 3.28E−08 | 7.94E+05 | 2.60E−02 | 0.1879 | 4.21E−10 | 2.00E+06 | 8.44E−04 |
| _HC: Y104L | 0.1875 | 2.52E−09 | 9.77E+05 | 2.46E−03 | 0.1765 | <1.0E−12 | 2.02E+06 | 4.27E−07 |
| _LC: N92F | 0.2444 | 2.13E−08 | 8.99E+05 | 1.92E−02 | 0.5917 | 9.25E−11 | 1.77E+06 | 1.64E−04 |
| _LC: N92K | 0.3062 | 8.13E−10 | 6.44E+05 | 5.23E−04 | 0.4518 | <1.0E−12 | 1.38E+06 | 6.15E−07 |

TABLE 15
On-Cell binding assay validating antibody retained
binding to antigen on-cell. All values reported
are MFI at 10 ug/mL of each antibody.

| Protein Name | EGFR | EGFRvIII | uPAR |
|---|---|---|---|
| Cetuximab_WT | 10844 | 8405 | 43.9 |
| Cetuximab (HC: D103F) | 4655 | 4240 | 93.5 |
| Cetuximab (HC: D103Y) | 4958 | 3920 | 114 |
| Cetuximab (HC: D103W) | 137 | 118 | 96.9 |
| Cetuximab (HC: Y101A) | 9535 | 8660 | 150 |
| Cetuximab (HC: Y101W) | 9158 | 8347 | 113 |
| Cetuximab (HC: Y102A) | 7934 | 7881 | 107 |
| Cetuximab (HC: Y104L) | 10121 | 11074 | 108 |
| Cetuximab (LC: N92F) | 8526 | 8211 | 114 |
| Cetuximab (LC: N92K) | 8057 | 7605 | 112 |
| Anti-uPAR Control mAb | 134 | 98.8 | 26152 |

TABLE 16
Configuration of anti-EGFR × EGFRvIII bispecific antibodies and component mAbs,
comprising humanized dematured EGFR binding domain and humanized EGFRvIII binding domain.

| | αEGFR-WT | αEGFRvIII | scFv | scFv | Interdomain |
|---|---|---|---|---|---|
| Protein | domain | domain | location | orientation | linker length (aa) |
| Cetuximab | Cetuximab (Fab) | n/a | n/a | n/a | n/a |
| Nimotuzumab | Nimotuzumab (Fab) | n/a | n/a | n/a | n/a |
| ABT-806 | n/a | ABT-806 (Fab) | n/a | n/a | n/a |
| SI-95X40 | Nimotuzumab (mAb) | Cetuximab (scFv) | HC C-term | HL | 10 |
| SI-95X41 | ABT-806 (mAb) | ABT-806 (scFv) | HC C-term | HL | 10 |
| SI-95X42 | Cetuximab (mAb) | Cetuximab (scFv) | HC C-term | HL | 10 |
| SI-95X43 | ABT-806 (mAb) | Cetuximab (scFv) | LC C-term | HL | 15 |
| SI-95X44 | Cetuximab (scFv) | ABT-806 (mAb) | HC N-term | LH | 20 |
| SI-95X45 | hCetuximab D103Y (mAb) | ABT-806 (scFv) | HC C-term | HL | 10 |
| SI-95X46 | hCetuximab Y104L (mAb) | ABT-806 (scFv) | HC C-term | HL | 10 |
| SI-95X47 | hCetuximab Y101A (mAb) | ABT-806 (scFv) | HC C-term | HL | 10 |
| SI-95X48 | hCetuximab Y102A (mAb) | ABT-806 (scFv) | HC C-term | HL | 10 |
| SI-95X49 | hCetuximab N92F (mAb) | ABT-806 (scFv) | HC C-term | HL | 10 |
| SI-95X50 | hCetuximab (mAb) | ABT-806 (scFv) | HC C-term | HL | 10 |
| SI-95X51 | hCetuximab (scFv) | ABT-806 V9 (mAb) | HC N-term | LH | 20 |
| SI-95X52 | hCetuximab N92F (scFv) | ABT-806 V9 (mAb) | HC N-term | LH | 20 |
| SI-95X53 | hCetuximab Y101A (scFv) | ABT-806 V9 (mAb) | HC N-term | LH | 20 |
| SI-95X54 | hCetuximab Y102A (scFv) | ABT-806 V9 (mAb) | HC N-term | LH | 20 |
| SI-95X55 | hCetuximab D103Y (scFv) | ABT-806 V9 (mAb) | HC N-term | LH | 20 |
| SI-95X56 | hCetuximab Y104L (scFv) | ABT-806 V9 (mAb) | HC N-term | LH | 20 |
| SI-95X57 | hCetuximab (mAb) | ABT-806 V9 (scFv) | LC C-term | HL | 21 |
| SI-95X58 | hCetuximab Y101A (mAb) | ABT-806 V9 (scFv) | LC C-term | HL | 21 |
| SI-95X59 | hCetuximab Y102A (mAb) | ABT-806 V9 (scFv) | LC C-term | HL | 21 |
| SI-95X60 | hCetuximab D103Y (mAb) | ABT-806 V9 (scFv) | LC C-term | HL | 21 |
| SI-95X61 | hCetuximab Y104L (mAb) | ABT-806 V9 (scFv) | LC C-term | HL | 21 |
| SI-95X62 | hCetuximab N92F (mAb) | ABT-806 V9 (scFv) | LC C-term | HL | 21 |
| SI-95X63 | hCetuximab (scFv) | ABT-806 (mAb) | HC C-term | HL | 10 |
| SI-95X64 | hCetuximab N92F (scFv) | ABT-806 (mAb) | HC C-term | HL | 10 |
| SI-95X65 | hCetuximab Y101A (scFv) | ABT-806 (mAb) | HC C-term | HL | 10 |
| SI-95X66 | hCetuximab Y102A (scFv) | ABT-806 (mAb) | HC C-term | HL | 10 |
| SI-95X67 | hCetuximab D103Y (scFv) | ABT-806 (mAb) | HC C-term | HL | 10 |
| SI-95X68 | hCetuximab Y104L (scFv) | ABT-806 (mAb) | HC C-term | HL | 10 |
| SI-95m4 | n/a | ABT-806 V9 | n/a | n/a | n/a |
| SI-95m5 | hCetuximab (mAb) | n/a | n/a | n/a | n/a |
| SI-95m6 | hCetuximab Y101A (mAb) | n/a | n/a | n/a | n/a |
| SI-95m7 | hCetuximab Y102A (mAb) | n/a | n/a | n/a | n/a |
| SI-95m8 | hCetuximab D103Y (mAb) | n/a | n/a | n/a | n/a |
| SI-95m9 | hCetuximab Y104L (mAb) | n/a | n/a | n/a | n/a |
| SI-95m10 | hCetuximab N92F (mAb) | n/a | n/a | n/a | n/a |
| SI-95m11 | Cetuximab N92F (mAb) | n/a | n/a | n/a | n/a |
| SI-95m12 | Cetuximab N92K (mAb) | n/a | n/a | n/a | n/a |
| SI-95m13 | Cetuximab Y101A (mAb) | n/a | n/a | n/a | n/a |
| SI-95m14 | Cetuximab Y101W (mAb) | n/a | n/a | n/a | n/a |
| SI-95m15 | Cetuximab Y102A (mAb) | n/a | n/a | n/a | n/a |
| SI-95m16 | Cetuximab Y102V (mAb) | n/a | n/a | n/a | n/a |
| SI-95m17 | Cetuximab D103F (mAb) | n/a | n/a | n/a | n/a |
| SI-95m18 | Cetuximab D103W (mAb) | n/a | n/a | n/a | n/a |
| SI-95m19 | Cetuximab D103Y (mAb) | n/a | n/a | n/a | n/a |
| SI-95m20 | Cetuximab Y104L (mAb) | n/a | n/a | n/a | n/a |
| ABT-806 V1 | n/a | ABT-806 VH1 VL1 | n/a | n/a | n/a |
| | | (mAb) | | | |
| ABT-806 V2 | n/a | ABT-806 VH1 VL2 | n/a | n/a | n/a |
| | | (mAb) | | | |
| ABT-806 V3 | n/a | ABT-806 VH1 VL3 | n/a | n/a | n/a |
| | | (mAb) | | | |
| ABT-806 V4 | n/a | ABT-806 VH2 VL1 | n/a | n/a | n/a |
| | | (mAb) | | | |
| ABT-806 V5 | n/a | ABT-806 VH2 VL2 | n/a | n/a | n/a |
| | | (mAb) | | | |
| ABT-806 V6 | n/a | ABT-806 VH2 VL3 | n/a | n/a | n/a |
| | | (mAb) | | | |
| ABT-806 V7 | n/a | ABT-806 VH3 VL1 | n/a | n/a | n/a |
| | | (mAB) | | | |
| ABT-806 V8 | n/a | ABT-806 VH3 VL2 | n/a | n/a | n/a |
| | | (mAb) | | | |
| ABT-806 V9 | n/a | ABT-806 VH3 VL3 | n/a | n/a | n/a |
| | | (mAb) | | | |

TABLE 17
Characterization of anti-EGFR × EGFRvIII bispecific
antibodies comprising humanized dematured EGFR binding domain
and humanized EGFRvIII binding domain, such as the expression
titer and purity as assessed by using analytical size-exclusion
chromatography after protein A purification.

| Protein | Titer (ug/ml) | % POI | % HMW | % LMW |
|---|---|---|---|---|
| SI-95X40 | 16 | 88.00 | 12.00 | 0.00 |
| SI-95X41 | 30 | 72.90 | 27.10 | 0.00 |
| SI-95X42 | 9 | 87.10 | 12.90 | 0.00 |
| SI-95X43 | 32 | 69.77 | 25.81 | 4.43 |
| SI-95X44 | 195 | 92.70 | 7.30 | 0.00 |
| SI-95X45 | 27 | 71.73 | 28.27 | 0.00 |
| SI-95X46 | 16 | 83.34 | 16.66 | 0.00 |
| SI-95X47 | 115 | 85.12 | 14.88 | 0.00 |
| SI-95X48 | 25 | 88.33 | 11.67 | 0.00 |
| SI-95X49 | 66 | 88.98 | 11.02 | 0.00 |
| SI-95X50 | 205 | 84.74 | 15.26 | 0.00 |
| SI-95X51 | 42 | 84.20 | 15.80 | 0.00 |
| SI-95X52 | 83 | 89.10 | 10.90 | 0.00 |
| SI-95X53 | 154 | 77.00 | 23.00 | 0.00 |
| SI-95X54 | 172 | 88.40 | 11.60 | 0.00 |
| SI-95X55 | 89 | 89.00 | 11.00 | 0.00 |
| SI-95X56 | 8 | 95.00 | 5.00 | 0.00 |
| SI-95X57 | 137 | 65.18 | 34.82 | 0.00 |
| SI-95X58 | 87 | 47.74 | 52.26 | 0.00 |
| SI-95X59 | 98 | 47.75 | 52.25 | 0.00 |
| SI-95X60 | 115 | 55.41 | 44.59 | 0.00 |
| SI-95X61 | 91 | 54.69 | 45.31 | 0.00 |
| SI-95X62 | 110 | 74.10 | 25.90 | 0.00 |
| SI-95X63 | 20 | 86.57 | 13.43 | 0.00 |
| SI-95X64 | 46 | 67.52 | 32.48 | 0.00 |
| SI-95X65 | 36 | 69.39 | 30.61 | 0.00 |
| SI-95X66 | 35 | 69.73 | 30.27 | 0.00 |
| SI-95X67 | 48 | 70.32 | 29.68 | 0.00 |
| SI-95X68 | 48 | 84.45 | 15.55 | 0.00 |

TABLE 18
Binding kinetics of anti-EGFR × EGFRvIII biepitopic tetravalent
antibodies comprising humanized dematured EGFR binding domain
and humanized EGFRvIII binding domain to EGFR-WT

| | Response | KD | kon | koff |
|---|---|---|---|---|
| Protein | (nm) | (M) | (M⁻¹s⁻¹) | (s⁻¹) |
| Cetuximab | 0.75 | 3.77E−09 | 3.41E+05 | 1.29E−03 |
| Nimotuzumab | 0.32 | 9.09E−09 | 1.48E+05 | 1.34E−03 |
| ABT-806 | 0.07 | ND | ND | ND |
| SI-95X40 | 0.70 | 9.54E−10 | 3.16E+05 | 3.02E−04 |
| SI-95X41 | 0.09 | NA | NA | NA |
| SI-95X42 | 1.1 | 3.43E−09 | 3.52E+05 | 1.21E−03 |
| SI-95X43 | 0.74 | 1.18E−09 | 3.16E+05 | 3.72E−04 |
| SI-95X44 | 0.47 | 1.81E−09 | 5.61E+05 | 1.26E+03 |
| SI-95X45 | 0.18 | 4.61E−09 | 4.11E+05 | 1.89E−03 |
| SI-95X46 | 0.59 | 7.30E−09 | 4.58E+05 | 3.34E−03 |
| SI-95X47 | 0.48 | 2.06E−08 | 4.29E+05 | 8.85E−03 |
| SI-95X48 | 0.44 | 2.55E−08 | 3.24E+05 | 8.25E−03 |
| SI-95X49 | 0.52 | 1.76E−08 | 3.78E+05 | 6.64E−03 |
| SI-95X50 | 0.59 | 1.38E−09 | 4.50E+05 | 6.23E−04 |
| SI-95X51 | 0.705 | 1.81E−09 | 3.94E+05 | 7.14E−04 |
| SI-95X52 | 0.5267 | 1.93E−08 | 2.75E+05 | 5.29E−03 |
| SI-95X53 | 0.3655 | 1.82E−08 | 2.45E+05 | 4.45E−03 |
| SI-95X54 | 0.3548 | 2.57E−08 | 3.11E+05 | 7.99E−03 |
| SI-95X55 | 0.056 | NA | NA | NA |
| SI-95X56 | 0.4402 | 6.88E−09 | 5.38E+05 | 3.70E−03 |
| SI-95X57 | 0.65 | 7.68E−10 | 4.00E+05 | 3.07E−04 |
| SI-95X58 | 0.48 | 1.60E−08 | 3.98E+05 | 6.36E−03 |
| SI-95X59 | 0.39 | 2.36E−08 | 3.30E+05 | 7.78E−03 |
| SI-95X60 | 0.14 | 4.98E−09 | 3.21E+05 | 1.60E−03 |
| SI-95X61 | 0.57 | 4.89E−09 | 4.13E+05 | 2.02E−03 |
| SI-95X62 | 0.55 | 8.16E−09 | 3.83E+05 | 3.13E−03 |
| SI-95X63 | 0.50 | 4.97E−10 | 4.67E+05 | 2.32E−04 |
| SI-95X64 | 0.43 | 2.31E−09 | 4.85E+05 | 1.12E−03 |
| SI-95X65 | 0.44 | 3.07E−09 | 5.14E+05 | 1.58E−03 |
| SI-95X66 | 0.36 | 4.65E−09 | 4.46E+05 | 2.08E−03 |
| SI-95X67 | 0.11 | 7.06E−09 | 3.23E+05 | 2.28E−03 |
| SI-95X68 | 0.47 | 1.87E−09 | 4.59E+05 | 8.60E−04 |

TABLE 19
Binding kinetics of anti-EGFR × EGFRvIII bispecific
antibodies comprising humanized dematured EGFR binding
domain and humanized EGFRvIII binding domain to EGFRvIII

| | Response | KD | kon | koff |
|---|---|---|---|---|
| Protein | (nm) | (M) | (M⁻¹s⁻¹) | (s⁻¹) |
| Cetuximab | 0.58 | 1.98E−09 | 8.09E+05 | 1.60E−03 |
| Nimotuzumab | 0.31 | 6.68E−09 | 2.02E+05 | 1.35E−03 |
| ABT-806 | 0.39 | 4.79E−09 | 3.29E+05 | 1.58E−03 |
| SI-95X40 | 0.44 | 1.16E−09 | 2.97E+05 | 3.43E−04 |
| SI-95X41 | 0.48 | 1.59E−08 | 1.15E+05 | 1.84E−03 |
| SI-95X42 | 0.73 | 3.60E−09 | 3.52E+05 | 2.03E−03 |
| SI-95X43 | 0.61 | 8.38E−10 | 3.73E+05 | 3.13E−04 |
| SI-95X44 | 0.29 | 4.66E−10 | 6.88E+05 | 1.28E+03 |
| SI-95X45 | 0.36 | 1.11E−09 | 3.84E+05 | 4.27E−04 |
| SI-95X46 | 0.39 | 3.58E−10 | 8.27E+05 | 2.96E−04 |
| SI-95X47 | 0.34 | 2.72E−10 | 7.56E+05 | 2.05E−04 |
| SI-95X48 | 0.42 | 4.04E−10 | 6.03E+05 | 2.43E−04 |
| SI-95X49 | 0.36 | 2.91E−10 | 6.61E+05 | 1.92E−04 |
| SI-95X50 | 0.38 | 2.07E−10 | 7.78E+05 | 1.61E−04 |
| SI-95X51 | 0.40 | 1.16E−09 | 4.89E+05 | 5.66E−04 |
| SI-95X52 | 0.37 | 1.47E−09 | 4.21E+05 | 6.20E−04 |
| SI-95X53 | 0.31 | 1.28E−09 | 4.11E+05 | 5.27E−04 |
| SI-95X54 | 0.32 | 1.37E−09 | 4.68E+05 | 6.40E−04 |
| SI-95X55 | 0.24 | 7.67E−09 | 2.20E+05 | 1.69E−03 |
| SI-95X56 | 0.26 | 1.93E−09 | 7.19E+05 | 1.38E−03 |
| SI-95X57 | 0.40 | 5.49E−10 | 8.74E+05 | 4.80E−04 |
| SI-95X58 | 0.36 | 8.07E−10 | 8.37E+05 | 6.75E−04 |
| SI-95X59 | 0.43 | 1.03E−09 | 6.23E+05 | 6.42E−04 |
| SI-95X60 | 0.37 | 1.95E−09 | 4.16E+05 | 8.14E−04 |
| SI-95X61 | 0.40 | 5.73E−10 | 8.78E+05 | 5.03E−04 |
| SI-95X62 | 0.39 | 1.02E−09 | 7.48E+05 | 7.60E−04 |
| SI-95X63 | 0.34 | 4.93E−10 | 4.57E+05 | 2.25E−04 |
| SI-95X64 | 0.31 | 6.38E−10 | 4.66E+05 | 2.97E−04 |
| SI-95X65 | 0.31 | 8.04E−10 | 4.55E+05 | 3.65E−04 |
| SI-95X66 | 0.29 | 5.76E−10 | 4.33E+05 | 2.49E−04 |
| SI-95X67 | 0.27 | 1.24E−09 | 3.11E+05 | 3.85E−04 |
| SI95X68 | 0.32 | 8.65E−10 | 4.47E+05 | 3.87E−04 |

### REFERENCE

1. Hanif F, Muzaffar K, Perveen K, Malhi S M, Simjee S U. Glioblastoma multiforme: A review of its epidemiology and pathogenesis through clinical presentation and treatment. Asian Pacific J Cancer Prev 2017; 18:3-9.
2. Gan H K, Cvrljevic A N, Johns T G. The epidermal growth factor receptor variant Ill (EGFRvIII): Where wild things are altered. FEBS J 2013; 280:5350-70.
3. Ramakrishnan M S, Eswaraiah A, Crombet T, Piedra P, Saurez G, Iyer H, Arvind A S. Nimotuzumab, a promising therapeutic monoclonal for treatment of tumors of epithelial origin. MAbs [Internet] 2009; 1:41-8. Available from: www.landesbioscience.com
4. AbbVie Provides Update on Depatuxizumab Mafodotin (Depatux-M), an Investigational Medicine for Newly Diagnosed Glioblastoma, an Aggressive Form of Brain Cancer [Internet]. AbbVie Press Release2019 [cited 2022 Mar. 31]; Available from: https://news.abbvie.com/news/press-releases/abbvie-provides-update-on-depatuxizumab-mafodotin-depatux-m-an-investigational-medicine-for-newly-diagnosed-glioblastoma-an-aggressive-form-brain-cancer.htm
5. Ellwanger K, Reusch U, Fucek I, Knackmuss S, Weichel M, Gantke T, Molkenthin V, Zhukovsky E A, Tesar M, Treder M. Highly specific and effective targeting of EGFRvIII-positive tumors with TandAb antibodies. Front Oncol 2017; 7:1-17.
6. Gedeon P C, Schaller T H, Chitneni S K, Choi B D, Kuan C T, Suryadevara C M, Snyder D J, Schmittling R J, Szafranski S E, Cui X, et al. A rationally designed fully human EGFRvIII: CD3-targeted bispecific antibody redirects human T cells to treat patient-derived intracerebral malignant glioma. Clin Cancer Res 2018; 24:3611-31.
7. Li, S.; Schmitz, K. R.; Jeffrey, P. D.; Wiltzius, J. J. W.; Kussie, P.; Ferguson, K. M. Structural basis for inhibition of the epidermal growth factor receptor by Cetuximab. Cancer Cell 2005, 7, 301-311, doi: 10.1016/j.ccr.2005.03.003.
11. Sickmier, E. A.; Kurzeja, R. J. M.; Michelsen, K.; Vazir, M.; Yang, E.; Tasker, A. S. The panitumumab EGFR complex reveals a binding mechanism that overcomes Cetuximab induced resistance. PLoS One 2016, 11, 1-11, doi: 10.1371/journal.pone.0163366.
12. Bagchi, A.; Haidar, J. N.; Eastman, S. W.; Vieth, M.; Topper, M.; lacolina, M. D.; Walker, J. M.; Forest, A.; Shen, Y.; Novosiadly, R. D.; et al. Molecular basis for necitumumab inhibition of EGFR variants associated with acquired Cetuximab resistance. Mol. Cancer Ther. 2018, 17, 521-531, doi: 10.1158/1535-7163.MCT-17-0575.

SEQUENCE LISTING

| | HC amino | HC | LC amino | LC |
|---|---|---|---|---|
| | acid | nucleotide | acid | nucleotide |
| Protein | Seq ID | Seq ID | Seq ID | Seq ID |
| Cetuximab | 1 | 2 | 3 | 4 |
| Nimotuzumab | 5 | 6 | 7 | 8 |
| ABT-806 | 9 | 10 | 11 | 12 |
| SI-95X1 | 13 | 14 | 3 | 4 |
| SI-95X2 | 15 | 16 | 7 | 8 |
| SI-95X3 | 17 | 18 | 11 | 12 |
| SI-95X4 | 19 | 20 | 11 | 12 |
| SI-95X5 | 21 | 22 | 11 | 12 |
| SI-95X6 | 23 | 24 | 11 | 12 |
| SI-95X7 | 25 | 26 | 11 | 12 |
| SI-95X8 | 27 | 28 | 11 | 12 |
| SI-95X9 | 29 | 30 | 11 | 12 |
| SI-95X10 | 31 | 32 | 11 | 12 |
| SI-95X11 | 9 | 10 | 33 | 34 |
| SI-95X12 | 9 | 10 | 35 | 36 |
| SI-95X13 | 9 | 10 | 37 | 38 |
| SI-95X14 | 9 | 10 | 39 | 40 |
| SI-95X15 | 9 | 10 | 41 | 42 |
| SI-95X16 | 9 | 10 | 43 | 44 |
| SI-95X17 | 45 | 46 | 7 | 8 |
| SI-95X18 | 47 | 48 | 7 | 8 |
| SI-95X19 | 49 | 50 | 7 | 8 |
| SI-95X20 | 51 | 52 | 7 | 8 |
| SI-95X21 | 53 | 54 | 7 | 8 |
| SI-95X22 | 55 | 56 | 7 | 8 |
| SI-95X23 | 5 | 6 | 57 | 58 |
| SI-95X24 | 5 | 6 | 59 | 60 |
| SI-95X25 | 5 | 6 | 61 | 62 |
| SI-95X26 | 5 | 6 | 63 | 64 |
| SI-95X27 | 5 | 6 | 65 | 66 |
| SI-95X28 | 5 | 6 | 67 | 68 |
| SI-95X29 | 5 | 6 | 69 | 70 |
| SI-95X30 | 5 | 6 | 71 | 72 |
| SI-95X31 | 5 | 6 | 73 | 74 |
| SI-95X35 | 75 | 76 | 11 | 12 |
| SI-95X36 | 9 | 10 | 77 | 78 |
| SI-95X37 | 79 | 80 | 3 | 4 |
| SI-95X38 | 1 | 2 | 81 | 82 |
| SI-95X39 | 1 | 2 | 83 | 84 |
| SI-95X40 | 85 | 86 | 7 | 8 |
| SI-95X41 | 87 | 88 | 11 | 12 |
| SI-95X42 | 89 | 90 | 3 | 4 |
| SI-95X43 | 9 | 10 | 91 | 92 |
| SI-95X44 | 93 | 94 | 95 | 96 |
| SI-95X45 | 97 | 98 | 99 | 100 |
| SI-95X46 | 101 | 102 | 99 | 100 |
| SI-95X47 | 103 | 104 | 99 | 100 |
| SI-95X48 | 105 | 106 | 99 | 100 |
| SI-95X49 | 107 | 108 | 109 | 110 |
| SI-95X50 | 107 | 108 | 99 | 100 |
| SI-95X51 | 179 | 180 | 111 | 112 |
| SI-95X52 | 113 | 114 | 111 | 112 |
| SI-95X53 | 115 | 116 | 111 | 112 |
| SI-95X54 | 117 | 118 | 111 | 112 |
| SI-95X55 | 119 | 120 | 111 | 112 |
| SI-95X56 | 121 | 122 | 111 | 112 |
| SI-95X57 | 123 | 124 | 125 | 126 |
| SI-95X58 | 127 | 128 | 125 | 126 |
| SI-95X59 | 129 | 130 | 125 | 126 |
| SI-95X60 | 131 | 132 | 125 | 126 |
| SI-95X61 | 133 | 134 | 125 | 126 |
| SI-95X62 | 123 | 124 | 135 | 136 |
| SI-95X63 | 137 | 138 | 11 | 12 |
| SI-95X64 | 139 | 140 | 11 | 12 |
| SI-95X65 | 141 | 142 | 11 | 12 |
| SI-95X66 | 143 | 144 | 11 | 12 |
| SI-95X67 | 145 | 146 | 11 | 12 |
| SI-95X68 | 147 | 148 | 11 | 12 |
| SI-95m4 | 149 | 150 | 111 | 112 |
| SI-95m5 | 123 | 124 | 99 | 100 |
| SI-95m6 | 127 | 128 | 99 | 100 |
| SI-95m7 | 129 | 130 | 99 | 100 |
| SI-95m8 | 131 | 132 | 99 | 100 |
| SI-95m9 | 133 | 134 | 99 | 100 |
| SI-95m10 | 123 | 124 | 109 | 110 |
| SI-95m11 | 1 | 2 | 151 | 152 |
| SI-95m12 | 1 | 2 | 153 | 154 |
| SI-95m13 | 155 | 156 | 3 | 4 |
| SI-95m14 | 157 | 158 | 3 | 4 |
| SI-95m15 | 159 | 160 | 3 | 4 |
| SI-95m16 | 161 | 162 | 3 | 4 |
| SI-95m17 | 163 | 164 | 3 | 4 |
| SI-95m18 | 165 | 166 | 3 | 4 |
| SI-95m19 | 167 | 168 | 3 | 4 |
| SI-95m20 | 169 | 170 | 3 | 4 |
| ABT-806 V1 | 171 | 172 | 173 | 174 |
| ABT-806 V2 | 171 | 172 | 175 | 176 |
| ABT-806 V3 | 171 | 172 | 111 | 112 |
| ABT-806 V4 | 177 | 178 | 173 | 174 |
| ABT-806 V5 | 177 | 178 | 175 | 176 |
| ABT-806 V6 | 177 | 178 | 111 | 112 |
| ABT-806 V7 | 149 | 150 | 173 | 174 |
| ABT-806 V8 | 149 | 150 | 175 | 176 |
| ABT-806 V9 | 149 | 150 | 111 | 112 |

| Domain | Amino acid Seq ID | Nucleotide Seq ID |
|---|---|---|
| Cetuximab VH | 201 | 202 |
| Cetuximab VL | 203 | 204 |
| Nimotuzumab VH | 205 | 206 |
| Nimotuzumab VL | 207 | 208 |
| ABT-806 VH | 209 | 210 |
| ABT-806 VL | 211 | 212 |
| (G4S)2 linker | 213 | 214 |
| (G4S)4 linker | 215 | 216 |
| (G4S)5 linker | 217 | 218 |
| (G4S)6 linker | 219 | 220 |
| Human IgG1 | 221 | 222 |
| Human CK | 223 | 224 |
| ABT-806 V1 VH | 225 | 226 |
| ABT-806 V1 VL | 227 | 228 |
| ABT-806 V2 VH | 229 | 230 |
| ABT-806 V2 VL | 231 | 232 |
| ABT-806 V3 VH | 233 | 234 |
| ABT-806 V3 VL | 235 | 236 |
| SI-95m5 (hCetux) VH | 237 | 238 |
| SI-95m5 (hCetux) VL | 239 | 240 |
| hCetuximab N92F VL | 241 | 242 |
| Cetuximab N92K VL | 243 | 244 |
| hCetuximab Y101A VH | 245 | 246 |
| Cetuximab Y101W VH | 247 | 248 |
| hCetuximab Y102A VH | 249 | 250 |
| Cetuximab D103F VH | 251 | 252 |
| Cetuximab D103W VH | 253 | 254 |
| hCetuximab D103Y VH | 255 | 256 |
| hCetuximab Y104L VH | 257 | 258 |
| Cetuximab Y101A VH | 259 | 260 |
| Cetuximab Y102A VH | 261 | 262 |
| Cetuximab Y102V VH | 263 | 264 |
| Cetuximab D103Y VH | 265 | 266 |
| Cetuximab Y104L VH | 267 | 268 |
| Cetuximab N92F VL | 269 | 270 |

>Seq ID 1: Cetuximab heavy chain amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSPG
>Seq ID 2: Cetuximab heavy chain nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTACTATGA
TTACGAGTTTGCTTACTGGGGCCAAGGGACTCTGGTCACTGTCTCTAGCGCTAGCACCAAGGGCCCATCGGTCTTCC
CCCTGGCACCCTCCTCCAAGAGCACCTCTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACCCAGACCTACATCTGCAACGTGAATC
ACAAGCCCAGCAACACCAAGGTGGACAAGAGAGTTGAGCCCAAATCTTGTGACAAAACTCACACATGCCCACCGTGC
CCAGCACCTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGTGGTCAGCGTCCTC
ACCGTCCTGCACCAGGACTGGCTGAATGGCAAGGAGTACAAGTGCAAGGTCTCCAACAAAGCCCTCCCAGCCCCCAT
CGAGAAAACCATCTCCAAAGCCAAAGGGCAGCCCCGAGAACCACAGGTGTACACCCTGCCCCCATCCCGGGATGAGC
TGACCAAGAACCAGGTCAGCCTGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGGCTCCTTCTTCCTCTATAGCAA
GCTCACCGTGGACAAGAGCAGGTGGCAGCAGGGGAACGTCTTCTCATGCTCCGTGATGCATGAGGCTCTGCACAACC
ACTACACGCAGAAGAGCCTCTCCCTGTCTCCGGGTTAA
>Seq ID 3: Cetuximab light chain amino acid sequence
DILLTQSPVILSVSPGERVSFSCRASQSIGTNIHWYQQRTNGSPRLLIKYASESISGIPSRFSGSGSGTDFTLSINS
VESEDIADYYCQQNNNWPTTFGAGTKLELKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
>Seq ID 4: Cetuximab light chain nucleotide sequence
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATAATAACTGGCCAACCACGTTCGGTGCTGGGACCAA
GCTGGAGCTGAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTTAG
>Seq ID 5: Nimotuzumab heavy chain amino acid sequence
QVQLQQSGAEVKKPGSSVKVSCKASGYTFTNYYIYWVRQAPGQGLEWIGGINPTSGGSNFNEKFKTRVTITADESST
TAYMELSSLRSEDTAFYFCTRQGLWFDSDGRGFDFWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPG
>Seq ID 6: Nimotuzumab heavy chain nucleotide sequence
CAGGTGCAGCTGCAGCAGAGCGGCGCCGAGGTGAAGAAGCCCGGCAGCAGCGTGAAGGTGAGCTGCAAGGCCAGCGG
CTACACCTTCACCAACTACTACATCTACTGGGTGCGGCAGGCCCCCGGCCAGGGCCTGGAGTGGATCGGCGGCATCA
ACCCCACCAGCGGCGGCAGCAACTTCAACGAGAAGTTCAAGACCCGGGTGACCATCACCGCCGACGAGAGCAGCACC
ACCGCCTACATGGAGCTGAGCAGCCTGCGGAGCGAGGACACCGCCTTCTACTTCTGCACCCGGCAGGGCCTGTGGTT
CGACAGCGACGGCCGGGGCTTCGACTTCTGGGGCCAGGGCACCACCGTGACCGTGAGCAGCGCTAGCACCAAGGGCC
CATCGGTCTTCCCCCTGGCACCCTCCTCCAAGAGCACCTCTGGGGGCACAGCGGCCCTGGGCTGCCTGGTCAAGGAC
TACTTCCCCGAACCGGTGACGGTGTCGTGGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCCAGCAGCTTGGGCACCCAGACCTACATCT
GCAACGTGAATCACAAGCCCAGCAACACCAAGGTGGACAAGAGAGTTGAGCCCAAATCTTGTGACAAAACTCACACA
TGCCCACCGTGCCCAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGTACACCCTGCCCCCAT
CCCGGGATGAGCTGACCAAGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTGCTGGACTCCGACGGCTCCTTCTT
CCTCTATAGCAAGCTCACCGTGGACAAGAGCAGGTGGCAGCAGGGGAACGTCTTCTCATGCTCCGTGATGCATGAGG
CTCTGCACAACCACTACACGCAGAAGAGCCTCTCCCTGTCTCCGGGTTAA
>Seq ID 7: Nimotuzumab light chain amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
>Seq ID 8: Nimotuzumab light chain nucleotide sequence
GATATTCAAATGACTCAATCTCCTTCTTCTCTTTCTGCTTCTGTTGGTGATCGTGTTACTATTACTTGTCGTTCTTC
TCAAAATATTGTTCATTCTAATGGTAATACTTATCTTGATTGGTATCAACAAACTCCTGGTAAAGCTCCTAAACTTC
TTATTTATAAAGTTTCTAATCGTTTTTCTGGTGTTCCTTCTCGTTTTTCTGGTTCTGGTTCTGGTACTGATTTTACT
TTTACTATTTCTTCTCTTCAACCTGAAGATATTGCTACTTATTATTGTTTTCAATATTCTCATGTTCCTTGGACTTT
TGGTCAAGGTACTAAACTTCAAATTACTCGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTAG
>Seq ID 9: ABT-806 heavy chain amino acid sequence
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSAASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>Seq ID 10: ABT-806 heavy chain nucleotide sequence
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCAGCTAGCACCAAGGGCCCATCGGTCTTCCCCCTGGCAC
CCTCCTCCAAGAGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGACTCTACTC
CCTCAGCAGCGTGGTGACCGTGCCCTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCACACATGCCCACCGTGCCCAGCACCT
GAACTCCTGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGCGTGGAGG
TGCATAATGCCAAGACAAAGCCGCGGGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAGCCCTCCCAGCCCCCATCGAGAAAAC
CATCTCCAAAGCCAAAGGGCAGCCCCGAGAACCACAGGTGTACACCCTGCCCCCATCCCGGGAGGAGATGACCAAGA
ACCAGGTCAGCCTGACCTGCCTGGTCAAAGGCTTCTATCCCAGCGACATCGCCGTGGAGTGGGAGAGCAATGGGCAG
CCGGAGAACAACTACAAGACCACGCCTCCCGTGCTGGACTCCGACGGCTCCTTCTTCCTCTATAGCAAGCTCACCGT
GGACAAGAGCAGGTGGCAGCAGGGGAACGTCTTCTCATGCTCCGTGATGCATGAGGCTCTGCACAACCACTACACGC
AGAAGAGCCTCTCCCTGTCTCCGGGTAAATGA
>Seq ID 11: ABT-806 light chain amino acid sequence
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
>Seq ID 12: ABT-806 light chain nucleotide sequence
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTTAG
>Seq ID 13: SI-95X1 heavy chain amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTYYDYEFAYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSPGGGGGSGGGGSDVQL
QESGPSLVKPSQSLSLTCTVTGYSITSDFAWNWIRQFPGNKLEWMGYISYSGNTRYNPSLKSRISITRDTSKNQFFL
QLNSVTIEDTATYYCVTAGRGFPYWGQGTLVTVSAGGGGSGGGGSGGGGSGGGGSDILMTQSPSSMSVSLGDTVSIT
CHSSQDINSNIGWLQQRPGKSFKGLIYHGTNLDDEVPSRFSGSGSGADYSLTISSLESEDFADYYCVQYAQFPWTFG
GGTKLEIK
>Seq ID 14: SI-95X1 heavy chain nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTACTATGA
TTACGAGTTTGCTTACTGGGGCCAAGGGACTCTGGTCACTGTCTCTAGCGCTAGCACCAAGGGCCCATCGGTCTTCC
CCCTGGCACCCTCCTCCAAGAGCACCTCTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACCCAGACCTACATCTGCAACGTGAATC
ACAAGCCCAGCAACACCAAGGTGGACAAGAGAGTTGAGCCCAAATCTTGTGACAAAACTCACACATGCCCACCGTGC
CCAGCACCTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGTGGTCAGCGTCCTC
ACCGTCCTGCACCAGGACTGGCTGAATGGCAAGGAGTACAAGTGCAAGGTCTCCAACAAAGCCCTCCCAGCCCCCAT
CGAGAAAACCATCTCCAAAGCCAAAGGGCAGCCCCGAGAACCACAGGTGTACACCCTGCCCCCATCCCGGGATGAGC
TGACCAAGAACCAGGTCAGCCTGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGGCTCCTTCTTCCTCTATAGCAA
GCTCACCGTGGACAAGAGCAGGTGGCAGCAGGGGAACGTCTTCTCATGCTCCGTGATGCATGAGGCTCTGCACAACC
ACTACACGCAGAAGAGCCTCTCCCTGTCTCCGGGTGGCGGTGGAGGGTCCGGCGGTGGTGGATCCGATGTGCAGCTT
CAGGAGTCGGGACCTAGCCTGGTGAAACCTTCTCAGTCTCTGTCCCTCACCTGCACTGTCACTGGCTACTCAATCAC
CAGTGATTTTGCCTGGAACTGGATTCGGCAGTTTCCAGGAAACAAGCTGGAGTGGATGGGCTACATAAGTTATAGTG
GTAACACTAGGTACAACCCATCTCTCAAAAGTCGAATCTCTATCACTCGCGACACATCCAAGAACCAATTCTTCCTG
CAGTTGAACTCTGTGACTATTGAGGACACAGCCACATATTACTGTGTAACGGCGGGACGCGGGTTTCCTTATTGGGG
CCAAGGGACTCTGGTCACTGTCTCTGCAGGCGGTGGCGGTAGTGGGGGAGGCGGTTCTGGCGGCGGAGGGTCCGGCG
GTGGAGGATCAGACATCCTGATGACCCAATCTCCATCCTCCATGTCTGTATCTCTGGGAGACACAGTCAGCATCACT
TGCCATTCAAGTCAGGACATTAACAGTAATATAGGGTGGTTGCAGCAGAGACCAGGGAAATCATTTAAGGGCCTGAT
CTATCATGGAACCAACTTGGACGATGAAGTTCCATCAAGGTTCAGTGGCAGTGGATCTGGAGCCGATTATTCTCTCA
CCATCAGCAGCCTGGAATCTGAAGATTTTGCAGACTATTACTGTGTACAGTATGCTCAGTTTCCGTGGACGTTCGGT
GGAGGCACCAAGCTGGAAATCAAATGA
>Seq ID 15: SI-95X2 heavy chain amino acid sequence
QVQLQQSGAEVKKPGSSVKVSCKASGYTFTNYYIYWVRQAPGQGLEWIGGINPTSGGSNFNEKFKTRVTITADESST
TAYMELSSLRSEDTAFYFCTRQGLWFDSDGRGFDFWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPGGGGGSGGGGS
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSAGGGGSGGGGSGGGGSGGGGSDILMTQSPSSMSVSLGDT
VSITCHSSQDINSNIGWLQQRPGKSFKGLIYHGTNLDDEVPSRFSGSGSGADYSLTISSLESEDFADYYCVQYAQFP
WTFGGGTKLEIK
>Seq ID 16: SI-95X2 heavy chain nucleotide sequence
CAGGTGCAGCTGCAGCAGAGCGGCGCCGAGGTGAAGAAGCCCGGCAGCAGCGTGAAGGTGAGCTGCAAGGCCAGCGG
CTACACCTTCACCAACTACTACATCTACTGGGTGCGGCAGGCCCCCGGCCAGGGCCTGGAGTGGATCGGCGGCATCA
ACCCCACCAGCGGCGGCAGCAACTTCAACGAGAAGTTCAAGACCCGGGTGACCATCACCGCCGACGAGAGCAGCACC
ACCGCCTACATGGAGCTGAGCAGCCTGCGGAGCGAGGACACCGCCTTCTACTTCTGCACCCGGCAGGGCCTGTGGTT
CGACAGCGACGGCCGGGGCTTCGACTTCTGGGGCCAGGGCACCACCGTGACCGTGAGCAGCGCTAGCACCAAGGGCC
CATCGGTCTTCCCCCTGGCACCCTCCTCCAAGAGCACCTCTGGGGGCACAGCGGCCCTGGGCTGCCTGGTCAAGGAC
TACTTCCCCGAACCGGTGACGGTGTCGTGGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCCAGCAGCTTGGGCACCCAGACCTACATCT
GCAACGTGAATCACAAGCCCAGCAACACCAAGGTGGACAAGAGAGTTGAGCCCAAATCTTGTGACAAAACTCACACA
TGCCCACCGTGCCCAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGTACACCCTGCCCCCAT
CCCGGGATGAGCTGACCAAGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTGCTGGACTCCGACGGCTCCTTCTT
CCTCTATAGCAAGCTCACCGTGGACAAGAGCAGGTGGCAGCAGGGGAACGTCTTCTCATGCTCCGTGATGCATGAGG
CTCTGCACAACCACTACACGCAGAAGAGCCTCTCCCTGTCTCCGGGTGGCGGTGGAGGGTCCGGCGGTGGTGGATCC
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCAGGCGGTGGCGGTAGTGGGGGAGGCGGTTCTGGCGGCG
GAGGGTCCGGCGGTGGAGGATCAGACATCCTGATGACCCAATCTCCATCCTCCATGTCTGTATCTCTGGGAGACACA
GTCAGCATCACTTGCCATTCAAGTCAGGACATTAACAGTAATATAGGGTGGTTGCAGCAGAGACCAGGGAAATCATT
TAAGGGCCTGATCTATCATGGAACCAACTTGGACGATGAAGTTCCATCAAGGTTCAGTGGCAGTGGATCTGGAGCCG
ATTATTCTCTCACCATCAGCAGCCTGGAATCTGAAGATTTTGCAGACTATTACTGTGTACAGTATGCTCAGTTTCCG
TGGACGTTCGGTGGAGGCACCAAGCTGGAAATCAAATGA
>Seq ID 17: SI-95X3 heavy chain amino acid sequence
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSAASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGGGGGSGGGGSQVQLKQS
GPGLVQPSQSLSITCTVSGFSLTNYGVHWVRQSPGKGLEWLGVIWSGGNTDYNTPFTSRLSINKDNSKSQVFFKMNS
LQSNDTAIYYCARALTYYDYEFAYWGQGTLVTVSSGGGGSGGGGSGGGGSGGGGSDILLTQSPVILSVSPGERVSFS
CRASQSIGTNIHWYQQRTNGSPRLLIKYASESISGIPSRFSGSGSGTDFTLSINSVESEDIADYYCQQNNNWPTTFG
AGTKLELK
>Seq ID 18: SI-95X3 heavy chain nucleotide sequence
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCAGCTAGCACCAAGGGCCCATCGGTCTTCCCCCTGGCAC
CCTCCTCCAAGAGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGACTCTACTC
CCTCAGCAGCGTGGTGACCGTGCCCTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCACACATGCCCACCGTGCCCAGCACCT
GAACTCCTGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGCGTGGAGG
TGCATAATGCCAAGACAAAGCCGCGGGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAGCCCTCCCAGCCCCCATCGAGAAAAC
CATCTCCAAAGCCAAAGGGCAGCCCCGAGAACCACAGGTGTACACCCTGCCCCCATCCCGGGATGAGCTGACCAAGA
ACCAGGTCAGCCTGACCTGCCTGGTCAAAGGCTTCTATCCCAGCGACATCGCCGTGGAGTGGGAGAGCAATGGGCAG
CCGGAGAACAACTACAAGACCACGCCTCCCGTGCTGGACTCCGACGGCTCCTTCTTCCTCTATAGCAAGCTCACCGT
GGACAAGAGCAGGTGGCAGCAGGGGAACGTCTTCTCATGCTCCGTGATGCATGAGGCTCTGCACAACCACTACACGC
AGAAGAGCCTCTCCCTGTCTCCGGGTGGCGGTGGAGGGTCCGGCGGTGGTGGATCCCAGGTGCAGCTGAAGCAGTCA
GGACCTGGCCTAGTGCAGCCCTCACAGAGCCTGTCCATCACCTGCACAGTCTCTGGTTTCTCATTAACTAACTATGG
TGTACACTGGGTTCGCCAGTCTCCAGGAAAGGGTCTGGAGTGGCTGGGAGTGATATGGAGTGGTGGAAACACAGACT
ATAATACACCTTTCACATCCAGACTGAGCATCAACAAGGACAATTCCAAGAGCCAAGTTTTCTTTAAAATGAACAGT
CTGCAATCTAATGACACAGCCATATATTACTGTGCCAGAGCCCTCACCTACTATGATTACGAGTTTGCTTACTGGGG
CCAAGGGACTCTGGTCACTGTCTCTAGCGGCGGTGGCGGTAGTGGGGGAGGCGGTTCTGGCGGCGGAGGGTCCGGCG
GTGGAGGATCAGACATCTTGCTGACTCAGTCTCCAGTCATCCTGTCTGTGAGTCCAGGAGAAAGAGTCAGTTTCTCC
TGCAGGGCCAGTCAGAGTATTGGCACAAACATACACTGGTATCAGCAAAGAACAAATGGTTCTCCAAGGCTTCTCAT
AAAGTATGCTTCTGAGTCTATCTCTGGGATTCCTTCCAGGTTTAGTGGCAGTGGATCAGGGACAGATTTTACTCTTA
GCATCAACAGTGTGGAGTCTGAAGATATTGCAGATTATTACTGTCAACAAAATAATAACTGGCCAACCACGTTCGGT
GCTGGGACCAAGCTGGAGCTGAAATGA
>Seq ID 19: SI-95x4 heavy chain amino acid sequence
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSAASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGGGGGSGGGGSQVQLQQS
GAEVKKPGSSVKVSCKASGYTFTNYYIYWVRQAPGQGLEWIGGINPTSGGSNFNEKFKTRVTITADESSTTAYMELS
SLRSEDTAFYFCTRQGLWFDSDGRGFDFWGQGTTVTVSSGGGGSGGGGSGGGGSGGGGSDIQMTQSPSSLSASVGDR
VTITCRSSQNIVHSNGNTYLDWYQQTPGKAPKLLIYKVSNRFSGVPSRFSGSGSGTDFTFTISSLQPEDIATYYCFQ
YSHVPWTFGQGTKLQIT
>Seq ID 20: SI-95x4 heavy chain nucleotide sequence
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCAGCTAGCACCAAGGGCCCATCGGTCTTCCCCCTGGCAC
CCTCCTCCAAGAGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGACTCTACTC
CCTCAGCAGCGTGGTGACCGTGCCCTCCAGCAGCTTGGGCACCCAGACCTACATCTGCAACGTGAATCACAAGCCCA
GCAACACCAAGGTGGACAAGAGAGTTGAGCCCAAATCTTGTGACAAAACTCACACATGCCCACCGTGCCCAGCACCT
GAACTCCTGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGCGTGGAGG
TGCATAATGCCAAGACAAAGCCGCGGGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAGCCCTCCCAGCCCCCATCGAGAAAAC
CATCTCCAAAGCCAAAGGGCAGCCCCGAGAACCACAGGTGTACACCCTGCCCCCATCCCGGGATGAGCTGACCAAGA
ACCAGGTCAGCCTGACCTGCCTGGTCAAAGGCTTCTATCCCAGCGACATCGCCGTGGAGTGGGAGAGCAATGGGCAG
CCGGAGAACAACTACAAGACCACGCCTCCCGTGCTGGACTCCGACGGCTCCTTCTTCCTCTATAGCAAGCTCACCGT
GGACAAGAGCAGGTGGCAGCAGGGGAACGTCTTCTCATGCTCCGTGATGCATGAGGCTCTGCACAACCACTACACGC
AGAAGAGCCTCTCCCTGTCTCCGGGTGGCGGTGGAGGGTCCGGCGGTGGTGGATCCCAAGTGCAGCTGCAGCAGAGC
GGCGCCGAGGTGAAGAAGCCTGGCAGCAGCGTGAAGGTGAGCTGCAAGGCTAGCGGCTACACCTTCACCAACTACTA
CATCTACTGGGTGAGACAAGCCCCTGGCCAAGGCCTGGAGTGGATCGGCGGCATCAACCCTACAAGCGGCGGCAGCA
ACTTCAACGAGAAGTTCAAGACAAGAGTTACCATAACTGCCGACGAGAGCAGCACCACCGCCTACATGGAGCTGAGC
AGCCTGAGAAGCGAGGACACCGCCTTCTACTTCTGCACAAGACAAGGCCTGTGGTTCGACAGCGACGGCAGAGGCTT
CGACTTTTGGGGCCAAGGAACCACAGTTACTGTGTCTTCCGGTGGTGGAGGATCCGGCGGCGGTGGTAGCGGCGGCG
GTGGGAGCGGCGGTGGTGGCAGCGACATTCAGATGACACAGAGCCCTAGCAGCCTGAGCGCTAGCGTGGGCGACAGA
GTGACCATCACCTGTAGAAGCTCTCAGAACATCGTGCACAGCAACGGCAACACCTACCTGGACTGGTATCAGCAGAC
CCCTGGCAAGGCCCCTAAGCTGCTGATCTACAAGGTGAGCAACAGATTTAGTGGTGTCCCTAGCAGATTCAGCGGCA
GCGGCAGCGGCACCGACTTCACCTTCACCATCAGCAGCCTGCAGCCTGAGGACATCGCCACCTACTACTGCTTTCAG
TACAGCCACGTGCCTTGGACCTTCGGACAAGGCACGAAACTGCAGATCACCTGA
>Seq ID 21: SI-95x5 heavy chain amino acid sequence
QVQLQQSGAEVKKPGSSVKVSCKASGYTFTNYYIYWVRQAPGQGLEWIGGINPTSGGSNENEKFKTRVTITADESST
TAYMELSSLRSEDTAFYFCTRQGLWFDSDGRGFDFWGQGTTVTVSSGGGGSGGGGSGGGGSDIQMTQSPSSLSASVG
DRVTITCRSSQNIVHSNGNTYLDWYQQTPGKAPKLLIYKVSNRFSGVPSRFSGSGSGTDFTFTISSLQPEDIATYYC
FQYSHVPWTFGQGTKLQITSGSGSGGGGGSGGGGSGGGGSDVQLQESGPSLVKPSQSLSLTCTVTGYSITSDFAWNW
IRQFPGNKLEWMGYISYSGNTRYNPSLKSRISITRDTSKNQFFLQLNSVTIEDTATYYCVTAGRGFPYWGQGTLVTV
SAASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVF
SCSVMHEALHNHYTQKSLSLSPG
>Seq ID 22: SI-95X5 heavy chain nucleotide sequence
CAAGTGCAGCTGCAGCAGAGCGGCGCCGAGGTGAAGAAGCCTGGCAGCAGCGTGAAGGTGAGCTGCAAGGCTAGCGG
CTACACCTTCACCAACTACTACATCTACTGGGTGAGACAAGCCCCTGGCCAAGGCCTGGAGTGGATCGGCGGCATCA
ACCCTACAAGCGGCGGCAGCAACTTCAACGAGAAGTTCAAGACAAGAGTTACCATAACTGCCGACGAGAGCAGCACC
ACCGCCTACATGGAGCTGAGCAGCCTGAGAAGCGAGGACACCGCCTTCTACTTCTGCACAAGACAAGGCCTGTGGTT
CGACAGCGACGGCAGAGGCTTCGACTTTTGGGGCCAAGGAACCACAGTTACTGTGTCTTCCGGTGGAGGAGGCAGTG
GTGGTGGAGGAAGCGGAGGTGGTGGCAGCGACATTCAGATGACACAGAGCCCTAGCAGCCTGAGCGCTAGCGTGGGC
GACAGAGTGACCATCACCTGTAGAAGCTCTCAGAACATCGTGCACAGCAACGGCAACACCTACCTGGACTGGTATCA
GCAGACCCCTGGCAAGGCCCCTAAGCTGCTGATCTACAAGGTGAGCAACAGATTTAGTGGTGTCCCTAGCAGATTCA
GCGGCAGCGGCAGCGGCACCGACTTCACCTTCACCATCAGCAGCCTGCAGCCTGAGGACATCGCCACCTACTACTGC
TTTCAGTACAGCCACGTGCCTTGGACCTTCGGACAAGGCACGAAACTGCAGATCACCTCCGGATCCGGTTCTGGAGG
AGGCGGTGGAAGCGGAGGCGGTGGCTCTGGAGGAGGCGGTTCGGATGTGCAGCTTCAGGAGTCGGGACCTAGCCTGG
TGAAACCTTCTCAGTCTCTGTCCCTCACCTGCACTGTCACTGGCTACTCAATCACCAGTGATTTTGCCTGGAACTGG
ATTCGGCAGTTTCCAGGAAACAAGCTGGAGTGGATGGGCTACATAAGTTATAGTGGTAACACTAGGTACAACCCATC
TCTCAAAAGTCGAATCTCTATCACTCGCGACACATCCAAGAACCAATTCTTCCTGCAGTTGAACTCTGTGACTATTG
AGGACACAGCCACATATTACTGTGTAACGGCGGGACGCGGGTTTCCTTATTGGGGCCAAGGGACTCTGGTCACTGTC
TCTGCAGCTAGCACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCAGCGGCG
TGCACACCTTCCCGGCTGTCCTACAGTCCTCAGGACTCTACTCCCTCAGCAGCGTGGTGACCGTGCCCTCCAGCAGC
TTGGGCACCCAGACCTACATCTGCAACGTGAATCACAAGCCCAGCAACACCAAGGTGGACAAGAGAGTTGAGCCCAA
ATCTTGTGACAAAACTCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAAGCCGCGGGAGGAGCA
GTACAACAGCACGTACCGTGTGGTCAGCGTCCTCACCGTCCTGCACCAGGACTGGCTGAATGGCAAGGAGTACAAGT
GCAAGGTCTCCAACAAAGCCCTCCCAGCCCCCATCGAGAAAACCATCTCCAAAGCCAAAGGGCAGCCCCGAGAACCA
CAGGTGTACACCCTGCCCCCATCCCGGGATGAGCTGACCAAGAACCAGGTCAGCCTGACCTGCCTGGTCAAAGGCTT
CTATCCCAGCGACATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCTCCGGGTTAG
>Seq ID 23: SI-95X6 heavy chain amino acid sequence
QVQLQQSGAEVKKPGSSVKVSCKASGYTFTNYYIYWVRQAPGQGLEWIGGINPTSGGSNFNEKFKTRVTITADESST
TAYMELSSLRSEDTAFYFCTRQGLWFDSDGRGFDFWGQGTTVTVSSGGGGSGGGGSGGGGSDIQMTQSPSSLSASVG
DRVTITCRSSQNIVHSNGNTYLDWYQQTPGKAPKLLIYKVSNRFSGVPSRFSGSGSGTDFTFTISSLQPEDIATYYC
FQYSHVPWTFGQGTKLQITGGGGSGSGSGGGGGSGGGGSGGGGSDVQLQESGPSLVKPSQSLSLTCTVTGYSITSDF
AWNWIRQFPGNKLEWMGYISYSGNTRYNPSLKSRISITRDTSKNQFFLQLNSVTIEDTATYYCVTAGRGFPYWGQGT
LVTVSAASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPG
>Seq ID 24: SI-95X6 heavy chain nucleotide sequence
CAAGTGCAGCTGCAGCAGAGCGGCGCCGAGGTGAAGAAGCCTGGCAGCAGCGTGAAGGTGAGCTGCAAGGCTAGCGG
CTACACCTTCACCAACTACTACATCTACTGGGTGAGACAAGCCCCTGGCCAAGGCCTGGAGTGGATCGGCGGCATCA
ACCCTACAAGCGGCGGCAGCAACTTCAACGAGAAGTTCAAGACAAGAGTTACCATAACTGCCGACGAGAGCAGCACC
ACCGCCTACATGGAGCTGAGCAGCCTGAGAAGCGAGGACACCGCCTTCTACTTCTGCACAAGACAAGGCCTGTGGTT
CGACAGCGACGGCAGAGGCTTCGACTTTTGGGGCCAAGGAACCACAGTTACTGTGTCTTCCGGTGGAGGAGGCAGTG
GTGGTGGAGGAAGCGGAGGTGGTGGCAGCGACATTCAGATGACACAGAGCCCTAGCAGCCTGAGCGCTAGCGTGGGC
GACAGAGTGACCATCACCTGTAGAAGCTCTCAGAACATCGTGCACAGCAACGGCAACACCTACCTGGACTGGTATCA
GCAGACCCCTGGCAAGGCCCCTAAGCTGCTGATCTACAAGGTGAGCAACAGATTTAGTGGTGTCCCTAGCAGATTCA
GCGGCAGCGGCAGCGGCACCGACTTCACCTTCACCATCAGCAGCCTGCAGCCTGAGGACATCGCCACCTACTACTGC
TTTCAGTACAGCCACGTGCCTTGGACCTTCGGACAAGGCACGAAACTGCAGATCACCGGTGGCGGTGGCTCCGGATC
CGGTTCTGGAGGAGGCGGTGGAAGCGGAGGCGGTGGCTCTGGAGGAGGCGGTTCGGATGTGCAGCTTCAGGAGTCGG
GACCTAGCCTGGTGAAACCTTCTCAGTCTCTGTCCCTCACCTGCACTGTCACTGGCTACTCAATCACCAGTGATTTT
GCCTGGAACTGGATTCGGCAGTTTCCAGGAAACAAGCTGGAGTGGATGGGCTACATAAGTTATAGTGGTAACACTAG
GTACAACCCATCTCTCAAAAGTCGAATCTCTATCACTCGCGACACATCCAAGAACCAATTCTTCCTGCAGTTGAACT
CTGTGACTATTGAGGACACAGCCACATATTACTGTGTAACGGCGGGACGCGGGTTTCCTTATTGGGGCCAAGGGACT
CTGGTCACTGTCTCTGCAGCTAGCACCAAGGGCCCATCGGTCTTCCCCCTGGCACCCTCCTCCAAGAGCACCTCTGG
GGGCACAGCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCCCAGCAACACCAAGGTGGACAAGAG
AGTTGAGCCCAAATCTTGTGACAAAACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCTGAGGTCACATGCGTGGTGGTGGAC
GTGAGCCACGAAGACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGACTGGCTGAATGGCA
AGGAGTACAAGTGCAAGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAACCAGGTCAGCCTGACCTGCCT
GGTCAAAGGCTTCTATCCCAGCGACATCGCCGTGGAGTGGGAGAGCAATGGGCAGCCGGAGAACAACTACAAGACCA
CGCCTCCCGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCCTGTCTCC
GGGTTAG
>Seq ID 25: SI-95X7 heavy chain amino acid sequence
QVQLQQSGAEVKKPGSSVKVSCKASGYTFTNYYIYWVRQAPGQGLEWIGGINPTSGGSNFNEKFKTRVTITADESST
TAYMELSSLRSEDTAFYFCTRQGLWFDSDGRGFDFWGQGTTVTVSSGGGGSGGGGSGGGGSDIQMTQSPSSLSASVG
DRVTITCRSSQNIVHSNGNTYLDWYQQTPGKAPKLLIYKVSNRFSGVPSRFSGSGSGTDFTFTISSLQPEDIATYYC
FQYSHVPWTFGQGTKLQITGGGGSGGGGSGSGSGGGGGSGGGGSGGGGSDVQLQESGPSLVKPSQSLSLTCTVTGYS
ITSDFAWNWIRQFPGNKLEWMGYISYSGNTRYNPSLKSRISITRDTSKNQFFLQLNSVTIEDTATYYCVTAGRGFPY
WGQGTLVTVSAASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPG
>Seq ID 26: SI-95X7 heavy chain nucleotide sequence
CAAGTGCAGCTGCAGCAGAGCGGCGCCGAGGTGAAGAAGCCTGGCAGCAGCGTGAAGGTGAGCTGCAAGGCTAGCGG
CTACACCTTCACCAACTACTACATCTACTGGGTGAGACAAGCCCCTGGCCAAGGCCTGGAGTGGATCGGCGGCATCA
ACCCTACAAGCGGCGGCAGCAACTTCAACGAGAAGTTCAAGACAAGAGTTACCATAACTGCCGACGAGAGCAGCACC
ACCGCCTACATGGAGCTGAGCAGCCTGAGAAGCGAGGACACCGCCTTCTACTTCTGCACAAGACAAGGCCTGTGGTT
CGACAGCGACGGCAGAGGCTTCGACTTTTGGGGCCAAGGAACCACAGTTACTGTGTCTTCCGGTGGAGGAGGCAGTG
GTGGTGGAGGAAGCGGAGGTGGTGGCAGCGACATTCAGATGACACAGAGCCCTAGCAGCCTGAGCGCTAGCGTGGGC
GACAGAGTGACCATCACCTGTAGAAGCTCTCAGAACATCGTGCACAGCAACGGCAACACCTACCTGGACTGGTATCA
GCAGACCCCTGGCAAGGCCCCTAAGCTGCTGATCTACAAGGTGAGCAACAGATTTAGTGGTGTCCCTAGCAGATTCA
GCGGCAGCGGCAGCGGCACCGACTTCACCTTCACCATCAGCAGCCTGCAGCCTGAGGACATCGCCACCTACTACTGC
TTTCAGTACAGCCACGTGCCTTGGACCTTCGGACAAGGCACGAAACTGCAGATCACCGGAGGCGGTGGTAGCGGTGG
CGGTGGCTCCGGATCCGGTTCTGGAGGAGGCGGTGGAAGCGGAGGCGGTGGCTCTGGAGGAGGCGGTTCGGATGTGC
AGCTTCAGGAGTCGGGACCTAGCCTGGTGAAACCTTCTCAGTCTCTGTCCCTCACCTGCACTGTCACTGGCTACTCA
ATCACCAGTGATTTTGCCTGGAACTGGATTCGGCAGTTTCCAGGAAACAAGCTGGAGTGGATGGGCTACATAAGTTA
TAGTGGTAACACTAGGTACAACCCATCTCTCAAAAGTCGAATCTCTATCACTCGCGACACATCCAAGAACCAATTCT
TCCTGCAGTTGAACTCTGTGACTATTGAGGACACAGCCACATATTACTGTGTAACGGCGGGACGCGGGTTTCCTTAT
TGGGGCCAAGGGACTCTGGTCACTGTCTCTGCAGCTAGCACCAAGGGCCCATCGGTCTTCCCCCTGGCACCCTCCTC
CAAGAGCACCTCTGGGGGCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGGACTCTACTCCCTCAGC
AGCGTGGTGACCGTGCCCTCCAGCAGCTTGGGCACCCAGACCTACATCTGCAACGTGAATCACAAGCCCAGCAACAC
CAAGGTGGACAAGAGAGTTGAGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACCGTCCTGCACCAGG
ACTGGCTGAATGGCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGATGAGCTGACCAAGAACCAGGT
CAGCCTGACCTGCCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTATAGCAAGCTCACCGTGGACAAG
AGCAGGTGGCAGCAGGGGAACGTCTTCTCATGCTCCGTGATGCATGAGGCTCTGCACAACCACTACACGCAGAAGAG
CCTCTCCCTGTCTCCGGGTTAG
>Seq ID 27: SI-95X8 heavy chain amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITGGGGSGGGGSGGGGSQVQLQQSGAEVKKPGSSVKVSCKASGY
TFTNYYIYWVRQAPGQGLEWIGGINPTSGGSNFNEKFKTRVTITADESSTTAYMELSSLRSEDTAFYFCTRQGLWED
SDGRGFDFWGQGTTVTVSSSGSGSGGGGGSGGGGSGGGGSDVQLQESGPSLVKPSQSLSLTCTVTGYSITSDFAWNW
IRQFPGNKLEWMGYISYSGNTRYNPSLKSRISITRDTSKNQFFLQLNSVTIEDTATYYCVTAGRGFPYWGQGTLVTV
SAASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVF
SCSVMHEALHNHYTQKSLSLSPG
>Seq ID 28: SI-95X8 heavy chain nucleotide sequence
GACATTCAGATGACACAGAGCCCTAGCAGCCTGAGCGCTAGCGTGGGCGACAGAGTGACCATCACCTGTAGAAGCTC
TCAGAACATCGTGCACAGCAACGGCAACACCTACCTGGACTGGTATCAGCAGACCCCTGGCAAGGCCCCTAAGCTGC
TGATCTACAAGGTGAGCAACAGATTTAGTGGTGTCCCTAGCAGATTCAGCGGCAGCGGCAGCGGCACCGACTTCACC
TTCACCATCAGCAGCCTGCAGCCTGAGGACATCGCCACCTACTACTGCTTTCAGTACAGCCACGTGCCTTGGACCTT
CGGACAAGGCACGAAACTGCAGATCACCGGTGGAGGAGGCAGTGGTGGTGGAGGAAGCGGAGGTGGTGGCAGCCAAG
TGCAGCTGCAGCAGAGCGGCGCCGAGGTGAAGAAGCCTGGCAGCAGCGTGAAGGTGAGCTGCAAGGCTAGCGGCTAC
ACCTTCACCAACTACTACATCTACTGGGTGAGACAAGCCCCTGGCCAAGGCCTGGAGTGGATCGGCGGCATCAACCC
TACAAGCGGCGGCAGCAACTTCAACGAGAAGTTCAAGACAAGAGTTACCATAACTGCCGACGAGAGCAGCACCACCG
CCTACATGGAGCTGAGCAGCCTGAGAAGCGAGGACACCGCCTTCTACTTCTGCACAAGACAAGGCCTGTGGTTCGAC
AGCGACGGCAGAGGCTTCGACTTTTGGGGCCAAGGAACCACAGTTACTGTGTCTTCCTCCGGATCCGGTTCTGGAGG
AGGCGGTGGAAGCGGAGGCGGTGGCTCTGGAGGAGGCGGTTCGGATGTGCAGCTTCAGGAGTCGGGACCTAGCCTGG
TGAAACCTTCTCAGTCTCTGTCCCTCACCTGCACTGTCACTGGCTACTCAATCACCAGTGATTTTGCCTGGAACTGG
ATTCGGCAGTTTCCAGGAAACAAGCTGGAGTGGATGGGCTACATAAGTTATAGTGGTAACACTAGGTACAACCCATC
TCTCAAAAGTCGAATCTCTATCACTCGCGACACATCCAAGAACCAATTCTTCCTGCAGTTGAACTCTGTGACTATTG
AGGACACAGCCACATATTACTGTGTAACGGCGGGACGCGGGTTTCCTTATTGGGGCCAAGGGACTCTGGTCACTGTC
TCTGCAGCTAGCACCAAGGGCCCATCGGTCTTCCCCCTGGCACCCTCCTCCAAGAGCACCTCTGGGGGCACAGCGGC
CCTGGGCTGCCTGGTCAAGGACTACTTCCCCGAACCGGTGACGGTGTCGTGGAACTCAGGCGCCCTGACCAGCGGCG
TGCACACCTTCCCGGCTGTCCTACAGTCCTCAGGACTCTACTCCCTCAGCAGCGTGGTGACCGTGCCCTCCAGCAGC
TTGGGCACCCAGACCTACATCTGCAACGTGAATCACAAGCCCAGCAACACCAAGGTGGACAAGAGAGTTGAGCCCAA
ATCTTGTGACAAAACTCACACATGCCCACCGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAAGCCGCGGGAGGAGCA
GTACAACAGCACGTACCGTGTGGTCAGCGTCCTCACCGTCCTGCACCAGGACTGGCTGAATGGCAAGGAGTACAAGT
GCAAGGTCTCCAACAAAGCCCTCCCAGCCCCCATCGAGAAAACCATCTCCAAAGCCAAAGGGCAGCCCCGAGAACCA
CAGGTGTACACCCTGCCCCCATCCCGGGATGAGCTGACCAAGAACCAGGTCAGCCTGACCTGCCTGGTCAAAGGCTT
CTATCCCAGCGACATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCTCCGGGTTAG
>Seq ID 29: SI-95X9 heavy chain amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITGGGGSGGGGSGGGGSQVQLQQSGAEVKKPGSSVKVSCKASGY
TFTNYYIYWVRQAPGQGLEWIGGINPTSGGSNFNEKFKTRVTITADESSTTAYMELSSLRSEDTAFYFCTRQGLWFD
SDGRGFDFWGQGTTVTVSSGGGGSGSGSGGGGGSGGGGSGGGGSDVQLQESGPSLVKPSQSLSLTCTVTGYSITSDF
AWNWIRQFPGNKLEWMGYISYSGNTRYNPSLKSRISITRDTSKNQFFLQLNSVTIEDTATYYCVTAGRGFPYWGQGT
LVTVSAASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPG
>Seq ID 30: SI-95X9 heavy chain nucleotide sequence
GACATTCAGATGACACAGAGCCCTAGCAGCCTGAGCGCTAGCGTGGGCGACAGAGTGACCATCACCTGTAGAAGCTC
TCAGAACATCGTGCACAGCAACGGCAACACCTACCTGGACTGGTATCAGCAGACCCCTGGCAAGGCCCCTAAGCTGC
TGATCTACAAGGTGAGCAACAGATTTAGTGGTGTCCCTAGCAGATTCAGCGGCAGCGGCAGCGGCACCGACTTCACC
TTCACCATCAGCAGCCTGCAGCCTGAGGACATCGCCACCTACTACTGCTTTCAGTACAGCCACGTGCCTTGGACCTT
CGGACAAGGCACGAAACTGCAGATCACCGGTGGAGGAGGCAGTGGTGGTGGAGGAAGCGGAGGTGGTGGCAGCCAAG
TGCAGCTGCAGCAGAGCGGCGCCGAGGTGAAGAAGCCTGGCAGCAGCGTGAAGGTGAGCTGCAAGGCTAGCGGCTAC
ACCTTCACCAACTACTACATCTACTGGGTGAGACAAGCCCCTGGCCAAGGCCTGGAGTGGATCGGCGGCATCAACCC
TACAAGCGGCGGCAGCAACTTCAACGAGAAGTTCAAGACAAGAGTTACCATAACTGCCGACGAGAGCAGCACCACCG
CCTACATGGAGCTGAGCAGCCTGAGAAGCGAGGACACCGCCTTCTACTTCTGCACAAGACAAGGCCTGTGGTTCGAC
AGCGACGGCAGAGGCTTCGACTTTTGGGGCCAAGGAACCACAGTTACTGTGTCTTCCGGTGGCGGTGGCTCCGGATC
CGGTTCTGGAGGAGGCGGTGGAAGCGGAGGCGGTGGCTCTGGAGGAGGCGGTTCGGATGTGCAGCTTCAGGAGTCGG
GACCTAGCCTGGTGAAACCTTCTCAGTCTCTGTCCCTCACCTGCACTGTCACTGGCTACTCAATCACCAGTGATTTT
GCCTGGAACTGGATTCGGCAGTTTCCAGGAAACAAGCTGGAGTGGATGGGCTACATAAGTTATAGTGGTAACACTAG
GTACAACCCATCTCTCAAAAGTCGAATCTCTATCACTCGCGACACATCCAAGAACCAATTCTTCCTGCAGTTGAACT
CTGTGACTATTGAGGACACAGCCACATATTACTGTGTAACGGCGGGACGCGGGTTTCCTTATTGGGGCCAAGGGACT
CTGGTCACTGTCTCTGCAGCTAGCACCAAGGGCCCATCGGTCTTCCCCCTGGCACCCTCCTCCAAGAGCACCTCTGG
GGGCACAGCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCCCAGCAACACCAAGGTGGACAAGAG
AGTTGAGCCCAAATCTTGTGACAAAACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCTGAGGTCACATGCGTGGTGGTGGAC
GTGAGCCACGAAGACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGACTGGCTGAATGGCA
AGGAGTACAAGTGCAAGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAACCAGGTCAGCCTGACCTGCCT
GGTCAAAGGCTTCTATCCCAGCGACATCGCCGTGGAGTGGGAGAGCAATGGGCAGCCGGAGAACAACTACAAGACCA
CGCCTCCCGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCCTGTCTCC
GGGTTAG
>Seq ID 31: SI-95X10 heavy chain amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITGGGGSGGGGSGGGGSQVQLQQSGAEVKKPGSSVKVSCKASGY
TFTNYYIYWVRQAPGQGLEWIGGINPTSGGSNFNEKFKTRVTITADESSTTAYMELSSLRSEDTAFYFCTRQGLWFD
SDGRGFDFWGQGTTVTVSSGGGGSGGGGSGSGSGGGGGSGGGGSGGGGSDVQLQESGPSLVKPSQSLSLTCTVTGYS
ITSDFAWNWIRQFPGNKLEWMGYISYSGNTRYNPSLKSRISITRDTSKNQFFLQLNSVTIEDTATYYCVTAGRGFPY
WGQGTLVTVSAASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPG
>Seq ID 32: SI-95X10 heavy chain nucleotide sequence
GACATTCAGATGACACAGAGCCCTAGCAGCCTGAGCGCTAGCGTGGGCGACAGAGTGACCATCACCTGTAGAAGCTC
TCAGAACATCGTGCACAGCAACGGCAACACCTACCTGGACTGGTATCAGCAGACCCCTGGCAAGGCCCCTAAGCTGC
TGATCTACAAGGTGAGCAACAGATTTAGTGGTGTCCCTAGCAGATTCAGCGGCAGCGGCAGCGGCACCGACTTCACC
TTCACCATCAGCAGCCTGCAGCCTGAGGACATCGCCACCTACTACTGCTTTCAGTACAGCCACGTGCCTTGGACCTT
CGGACAAGGCACGAAACTGCAGATCACCGGTGGAGGAGGCAGTGGTGGTGGAGGAAGCGGAGGTGGTGGCAGCCAAG
TGCAGCTGCAGCAGAGCGGCGCCGAGGTGAAGAAGCCTGGCAGCAGCGTGAAGGTGAGCTGCAAGGCTAGCGGCTAC
ACCTTCACCAACTACTACATCTACTGGGTGAGACAAGCCCCTGGCCAAGGCCTGGAGTGGATCGGCGGCATCAACCC
TACAAGCGGCGGCAGCAACTTCAACGAGAAGTTCAAGACAAGAGTTACCATAACTGCCGACGAGAGCAGCACCACCG
CCTACATGGAGCTGAGCAGCCTGAGAAGCGAGGACACCGCCTTCTACTTCTGCACAAGACAAGGCCTGTGGTTCGAC
AGCGACGGCAGAGGCTTCGACTTTTGGGGCCAAGGAACCACAGTTACTGTGTCTTCCGGAGGCGGTGGTAGCGGTGG
CGGTGGCTCCGGATCCGGTTCTGGAGGAGGCGGTGGAAGCGGAGGCGGTGGCTCTGGAGGAGGCGGTTCGGATGTGC
AGCTTCAGGAGTCGGGACCTAGCCTGGTGAAACCTTCTCAGTCTCTGTCCCTCACCTGCACTGTCACTGGCTACTCA
ATCACCAGTGATTTTGCCTGGAACTGGATTCGGCAGTTTCCAGGAAACAAGCTGGAGTGGATGGGCTACATAAGTTA
TAGTGGTAACACTAGGTACAACCCATCTCTCAAAAGTCGAATCTCTATCACTCGCGACACATCCAAGAACCAATTCT
TCCTGCAGTTGAACTCTGTGACTATTGAGGACACAGCCACATATTACTGTGTAACGGCGGGACGCGGGTTTCCTTAT
TGGGGCCAAGGGACTCTGGTCACTGTCTCTGCAGCTAGCACCAAGGGCCCATCGGTCTTCCCCCTGGCACCCTCCTC
CAAGAGCACCTCTGGGGGCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGGACTCTACTCCCTCAGC
AGCGTGGTGACCGTGCCCTCCAGCAGCTTGGGCACCCAGACCTACATCTGCAACGTGAATCACAAGCCCAGCAACAC
CAAGGTGGACAAGAGAGTTGAGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACCGTCCTGCACCAGG
ACTGGCTGAATGGCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGATGAGCTGACCAAGAACCAGGT
CAGCCTGACCTGCCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTATAGCAAGCTCACCGTGGACAAG
AGCAGGTGGCAGCAGGGGAACGTCTTCTCATGCTCCGTGATGCATGAGGCTCTGCACAACCACTACACGCAGAAGAG
CCTCTCCCTGTCTCCGGGTTAG
>Seq ID 33: SI-95X11 light chain amino acid sequence
QVQLQQSGAEVKKPGSSVKVSCKASGYTFTNYYIYWVRQAPGQGLEWIGGINPTSGGSNFNEKFKTRVTITADESST
TAYMELSSLRSEDTAFYFCTRQGLWFDSDGRGFDFWGQGTTVTVSSGGGGSGGGGSGGGGSDIQMTQSPSSLSASVG
DRVTITCRSSQNIVHSNGNTYLDWYQQTPGKAPKLLIYKVSNRFSGVPSRFSGSGSGTDFTFTISSLQPEDIATYYC
FQYSHVPWTFGQGTKLQITSGSGSGGGGGSGGGGSGGGGSDILMTQSPSSMSVSLGDTVSITCHSSQDINSNIGWLQ
QRPGKSFKGLIYHGTNLDDEVPSRFSGSGSGADYSLTISSLESEDFADYYCVQYAQFPWTFGGGTKLEIKRTVAAPS
VFIFPPSDEQLKSGTASVVCLLNNFYPREAKVQWKVDNALQSGNSQESVTEQDSKDSTYSLSSTLTLSKADYEKHKV
YACEVTHQGLSSPVTKSFNRGEC
>Seq ID 34: SI-95X11 light chain nucleotide sequence
CAAGTGCAGCTGCAGCAGAGCGGCGCCGAGGTGAAGAAGCCTGGCAGCAGCGTGAAGGTGAGCTGCAAGGCTAGCGG
CTACACCTTCACCAACTACTACATCTACTGGGTGAGACAAGCCCCTGGCCAAGGCCTGGAGTGGATCGGCGGCATCA
ACCCTACAAGCGGCGGCAGCAACTTCAACGAGAAGTTCAAGACAAGAGTTACCATAACTGCCGACGAGAGCAGCACC
ACCGCCTACATGGAGCTGAGCAGCCTGAGAAGCGAGGACACCGCCTTCTACTTCTGCACAAGACAAGGCCTGTGGTT
CGACAGCGACGGCAGAGGCTTCGACTTTTGGGGCCAAGGAACCACAGTTACTGTGTCTTCCGGTGGAGGAGGCAGTG
GTGGTGGAGGAAGCGGAGGTGGTGGCAGCGACATTCAGATGACACAGAGCCCTAGCAGCCTGAGCGCTAGCGTGGGC
GACAGAGTGACCATCACCTGTAGAAGCTCTCAGAACATCGTGCACAGCAACGGCAACACCTACCTGGACTGGTATCA
GCAGACCCCTGGCAAGGCCCCTAAGCTGCTGATCTACAAGGTGAGCAACAGATTTAGTGGTGTCCCTAGCAGATTCA
GCGGCAGCGGCAGCGGCACCGACTTCACCTTCACCATCAGCAGCCTGCAGCCTGAGGACATCGCCACCTACTACTGC
TTTCAGTACAGCCACGTGCCTTGGACCTTCGGACAAGGCACGAAACTGCAGATCACCTCCGGATCCGGTTCTGGAGG
AGGCGGTGGAAGCGGAGGCGGTGGCTCTGGAGGAGGCGGTTCGGACATCCTGATGACCCAATCTCCATCCTCCATGT
CTGTATCTCTGGGAGACACAGTCAGCATCACTTGCCATTCAAGTCAGGACATTAACAGTAATATAGGGTGGTTGCAG
CAGAGACCAGGGAAATCATTTAAGGGCCTGATCTATCATGGAACCAACTTGGACGATGAAGTTCCATCAAGGTTCAG
TGGCAGTGGATCTGGAGCCGATTATTCTCTCACCATCAGCAGCCTGGAATCTGAAGATTTTGCAGACTATTACTGTG
TACAGTATGCTCAGTTTCCGTGGACGTTCGGTGGAGGCACCAAGCTGGAAATCAAACGTACGGTGGCTGCACCATCT
GTCTTCATCTTCCCGCCATCTGATGAGCAGTTGAAATCTGGAACTGCCTCTGTTGTGTGCCTGCTGAATAACTTCTA
TCCCAGAGAGGCCAAAGTACAGTGGAAGGTGGATAACGCCCTCCAATCGGGTAACTCCCAGGAGAGTGTCACAGAGC
AGGACAGCAAGGACAGCACCTACAGCCTCAGCAGCACCCTGACGCTGAGCAAAGCAGACTACGAGAAACACAAAGTC
TACGCCTGCGAAGTCACCCATCAGGGCCTGAGCTCGCCCGTCACAAAGAGCTTCAACAGGGGAGAGTGTTAG
>Seq ID 35: SI-95X12 light chain amino acid sequence
QVQLQQSGAEVKKPGSSVKVSCKASGYTFTNYYIYWVRQAPGQGLEWIGGINPTSGGSNFNEKFKTRVTITADESST
TAYMELSSLRSEDTAFYFCTRQGLWFDSDGRGFDFWGQGTTVTVSSGGGGSGGGGSGGGGSDIQMTQSPSSLSASVG
DRVTITCRSSQNIVHSNGNTYLDWYQQTPGKAPKLLIYKVSNRFSGVPSRFSGSGSGTDFTFTISSLQPEDIATYYC
FQYSHVPWTFGQGTKLQITGGGGSGSGSGGGGGSGGGGSGGGGSDILMTQSPSSMSVSLGDTVSITCHSSQDINSNI
GWLQQRPGKSFKGLIYHGTNLDDEVPSRFSGSGSGADYSLTISSLESEDFADYYCVQYAQFPWTFGGGTKLEIKRTV
AAPSVFIFPPSDEQLKSGTASVVCLLNNFYPREAKVQWKVDNALQSGNSQESVTEQDSKDSTYSLSSTLTLSKADYE
KHKVYACEVTHQGLSSPVTKSFNRGEC
>Seq ID 36: SI-95X12 light chain nucleotide sequence
CAAGTGCAGCTGCAGCAGAGCGGCGCCGAGGTGAAGAAGCCTGGCAGCAGCGTGAAGGTGAGCTGCAAGGCTAGCGG
CTACACCTTCACCAACTACTACATCTACTGGGTGAGACAAGCCCCTGGCCAAGGCCTGGAGTGGATCGGCGGCATCA
ACCCTACAAGCGGCGGCAGCAACTTCAACGAGAAGTTCAAGACAAGAGTTACCATAACTGCCGACGAGAGCAGCACC
ACCGCCTACATGGAGCTGAGCAGCCTGAGAAGCGAGGACACCGCCTTCTACTTCTGCACAAGACAAGGCCTGTGGTT
CGACAGCGACGGCAGAGGCTTCGACTTTTGGGGCCAAGGAACCACAGTTACTGTGTCTTCCGGTGGAGGAGGCAGTG
GTGGTGGAGGAAGCGGAGGTGGTGGCAGCGACATTCAGATGACACAGAGCCCTAGCAGCCTGAGCGCTAGCGTGGGC
GACAGAGTGACCATCACCTGTAGAAGCTCTCAGAACATCGTGCACAGCAACGGCAACACCTACCTGGACTGGTATCA
GCAGACCCCTGGCAAGGCCCCTAAGCTGCTGATCTACAAGGTGAGCAACAGATTTAGTGGTGTCCCTAGCAGATTCA
GCGGCAGCGGCAGCGGCACCGACTTCACCTTCACCATCAGCAGCCTGCAGCCTGAGGACATCGCCACCTACTACTGC
TTTCAGTACAGCCACGTGCCTTGGACCTTCGGACAAGGCACGAAACTGCAGATCACCGGTGGCGGTGGCTCCGGATC
CGGTTCTGGAGGAGGCGGTGGAAGCGGAGGCGGTGGCTCTGGAGGAGGCGGTTCGGACATCCTGATGACCCAATCTC
CATCCTCCATGTCTGTATCTCTGGGAGACACAGTCAGCATCACTTGCCATTCAAGTCAGGACATTAACAGTAATATA
GGGTGGTTGCAGCAGAGACCAGGGAAATCATTTAAGGGCCTGATCTATCATGGAACCAACTTGGACGATGAAGTTCC
ATCAAGGTTCAGTGGCAGTGGATCTGGAGCCGATTATTCTCTCACCATCAGCAGCCTGGAATCTGAAGATTTTGCAG
ACTATTACTGTGTACAGTATGCTCAGTTTCCGTGGACGTTCGGTGGAGGCACCAAGCTGGAAATCAAACGTACGGTG
GCTGCACCATCTGTCTTCATCTTCCCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCCCTCCAATCGGGTAACTCCCAGGAGA
GTGTCACAGAGCAGGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCTTCAACAGGGGAGA
GTGTTAG
>Seq ID 37: SI-95X13 light chain amino acid sequence
QVQLQQSGAEVKKPGSSVKVSCKASGYTFTNYYIYWVRQAPGQGLEWIGGINPTSGGSNFNEKFKTRVTITADESST
TAYMELSSLRSEDTAFYFCTRQGLWFDSDGRGFDFWGQGTTVTVSSGGGGSGGGGSGGGGSDIQMTQSPSSLSASVG
DRVTITCRSSQNIVHSNGNTYLDWYQQTPGKAPKLLIYKVSNRFSGVPSRFSGSGSGTDFTFTISSLQPEDIATYYC
FQYSHVPWTFGQGTKLQITGGGGSGGGGSGSGSGGGGGSGGGGSGGGGSDILMTQSPSSMSVSLGDTVSITCHSSQD
INSNIGWLQQRPGKSFKGLIYHGTNLDDEVPSRFSGSGSGADYSLTISSLESEDFADYYCVQYAQFPWTFGGGTKLE
IKRTVAAPSVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
>Seq ID 38: SI-95X13 light chain nucleotide sequence
CAAGTGCAGCTGCAGCAGAGCGGCGCCGAGGTGAAGAAGCCTGGCAGCAGCGTGAAGGTGAGCTGCAAGGCTAGCGG
CTACACCTTCACCAACTACTACATCTACTGGGTGAGACAAGCCCCTGGCCAAGGCCTGGAGTGGATCGGCGGCATCA
ACCCTACAAGCGGCGGCAGCAACTTCAACGAGAAGTTCAAGACAAGAGTTACCATAACTGCCGACGAGAGCAGCACC
ACCGCCTACATGGAGCTGAGCAGCCTGAGAAGCGAGGACACCGCCTTCTACTTCTGCACAAGACAAGGCCTGTGGTT
CGACAGCGACGGCAGAGGCTTCGACTTTTGGGGCCAAGGAACCACAGTTACTGTGTCTTCCGGTGGAGGAGGCAGTG
GTGGTGGAGGAAGCGGAGGTGGTGGCAGCGACATTCAGATGACACAGAGCCCTAGCAGCCTGAGCGCTAGCGTGGGC
GACAGAGTGACCATCACCTGTAGAAGCTCTCAGAACATCGTGCACAGCAACGGCAACACCTACCTGGACTGGTATCA
GCAGACCCCTGGCAAGGCCCCTAAGCTGCTGATCTACAAGGTGAGCAACAGATTTAGTGGTGTCCCTAGCAGATTCA
GCGGCAGCGGCAGCGGCACCGACTTCACCTTCACCATCAGCAGCCTGCAGCCTGAGGACATCGCCACCTACTACTGC
TTTCAGTACAGCCACGTGCCTTGGACCTTCGGACAAGGCACGAAACTGCAGATCACCGGAGGCGGTGGTAGCGGTGG
CGGTGGCTCCGGATCCGGTTCTGGAGGAGGCGGTGGAAGCGGAGGCGGTGGCTCTGGAGGAGGCGGTTCGGACATCC
TGATGACCCAATCTCCATCCTCCATGTCTGTATCTCTGGGAGACACAGTCAGCATCACTTGCCATTCAAGTCAGGAC
ATTAACAGTAATATAGGGTGGTTGCAGCAGAGACCAGGGAAATCATTTAAGGGCCTGATCTATCATGGAACCAACTT
GGACGATGAAGTTCCATCAAGGTTCAGTGGCAGTGGATCTGGAGCCGATTATTCTCTCACCATCAGCAGCCTGGAAT
CTGAAGATTTTGCAGACTATTACTGTGTACAGTATGCTCAGTTTCCGTGGACGTTCGGTGGAGGCACCAAGCTGGAA
ATCAAACGTACGGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCCAATCGG
GTAACTCCCAGGAGAGTGTCACAGAGCAGGACAGCAAGGACAGCACCTACAGCCTCAGCAGCACCCTGACGCTGAGC
AAAGCAGACTACGAGAAACACAAAGTCTACGCCTGCGAAGTCACCCATCAGGGCCTGAGCTCGCCCGTCACAAAGAG
CTTCAACAGGGGAGAGTGTTAG
>Seq ID 39: SI-95X14 light chain amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITGGGGSGGGGSGGGGSQVQLQQSGAEVKKPGSSVKVSCKASGY
TFTNYYIYWVRQAPGQGLEWIGGINPTSGGSNFNEKFKTRVTITADESSTTAYMELSSLRSEDTAFYFCTRQGLWFD
SDGRGFDFWGQGTTVTVSSSGSGSGGGGGSGGGGSGGGGSDILMTQSPSSMSVSLGDTVSITCHSSQDINSNIGWLQ
QRPGKSFKGLIYHGTNLDDEVPSRFSGSGSGADYSLTISSLESEDFADYYCVQYAQFPWTFGGGTKLEIKRTVAAPS
VFIFPPSDEQLKSGTASVVCLLNNFYPREAKVQWKVDNALQSGNSQESVTEQDSKDSTYSLSSTLTLSKADYEKHKV
YACEVTHQGLSSPVTKSFNRGEC
>Seq ID 40: SI-95X14 light chain nucleotide sequence
GACATTCAGATGACACAGAGCCCTAGCAGCCTGAGCGCTAGCGTGGGCGACAGAGTGACCATCACCTGTAGAAGCTC
TCAGAACATCGTGCACAGCAACGGCAACACCTACCTGGACTGGTATCAGCAGACCCCTGGCAAGGCCCCTAAGCTGC
TGATCTACAAGGTGAGCAACAGATTTAGTGGTGTCCCTAGCAGATTCAGCGGCAGCGGCAGCGGCACCGACTTCACC
TTCACCATCAGCAGCCTGCAGCCTGAGGACATCGCCACCTACTACTGCTTTCAGTACAGCCACGTGCCTTGGACCTT
CGGACAAGGCACGAAACTGCAGATCACCGGTGGAGGAGGCAGTGGTGGTGGAGGAAGCGGAGGTGGTGGCAGCCAAG
TGCAGCTGCAGCAGAGCGGCGCCGAGGTGAAGAAGCCTGGCAGCAGCGTGAAGGTGAGCTGCAAGGCTAGCGGCTAC
ACCTTCACCAACTACTACATCTACTGGGTGAGACAAGCCCCTGGCCAAGGCCTGGAGTGGATCGGCGGCATCAACCC
TACAAGCGGCGGCAGCAACTTCAACGAGAAGTTCAAGACAAGAGTTACCATAACTGCCGACGAGAGCAGCACCACCG
CCTACATGGAGCTGAGCAGCCTGAGAAGCGAGGACACCGCCTTCTACTTCTGCACAAGACAAGGCCTGTGGTTCGAC
AGCGACGGCAGAGGCTTCGACTTTTGGGGCCAAGGAACCACAGTTACTGTGTCTTCCTCCGGATCCGGTTCTGGAGG
AGGCGGTGGAAGCGGAGGCGGTGGCTCTGGAGGAGGCGGTTCGGACATCCTGATGACCCAATCTCCATCCTCCATGT
CTGTATCTCTGGGAGACACAGTCAGCATCACTTGCCATTCAAGTCAGGACATTAACAGTAATATAGGGTGGTTGCAG
CAGAGACCAGGGAAATCATTTAAGGGCCTGATCTATCATGGAACCAACTTGGACGATGAAGTTCCATCAAGGTTCAG
TGGCAGTGGATCTGGAGCCGATTATTCTCTCACCATCAGCAGCCTGGAATCTGAAGATTTTGCAGACTATTACTGTG
TACAGTATGCTCAGTTTCCGTGGACGTTCGGTGGAGGCACCAAGCTGGAAATCAAACGTACGGTGGCTGCACCATCT
GTCTTCATCTTCCCGCCATCTGATGAGCAGTTGAAATCTGGAACTGCCTCTGTTGTGTGCCTGCTGAATAACTTCTA
TCCCAGAGAGGCCAAAGTACAGTGGAAGGTGGATAACGCCCTCCAATCGGGTAACTCCCAGGAGAGTGTCACAGAGC
AGGACAGCAAGGACAGCACCTACAGCCTCAGCAGCACCCTGACGCTGAGCAAAGCAGACTACGAGAAACACAAAGTC
TACGCCTGCGAAGTCACCCATCAGGGCCTGAGCTCGCCCGTCACAAAGAGCTTCAACAGGGGAGAGTGTTAG
>Seq ID 41: SI-95X15 light chain amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITGGGGSGGGGSGGGGSQVQLQQSGAEVKKPGSSVKVSCKASGY
TFTNYYIYWVRQAPGQGLEWIGGINPTSGGSNFNEKFKTRVTITADESSTTAYMELSSLRSEDTAFYFCTRQGLWFD
SDGRGFDFWGQGTTVTVSSGGGGSGSGSGGGGGSGGGGSGGGGSDILMTQSPSSMSVSLGDTVSITCHSSQDINSNI
GWLQQRPGKSFKGLIYHGTNLDDEVPSRFSGSGSGADYSLTISSLESEDFADYYCVQYAQFPWTFGGGTKLEIKRTV
AAPSVFIFPPSDEQLKSGTASVVCLLNNFYPREAKVQWKVDNALQSGNSQESVTEQDSKDSTYSLSSTLTLSKADYE
KHKVYACEVTHQGLSSPVTKSFNRGEC
>Seq ID 42: SI-95X15 light chain nucleotide sequence
GACATTCAGATGACACAGAGCCCTAGCAGCCTGAGCGCTAGCGTGGGCGACAGAGTGACCATCACCTGTAGAAGCTC
TCAGAACATCGTGCACAGCAACGGCAACACCTACCTGGACTGGTATCAGCAGACCCCTGGCAAGGCCCCTAAGCTGC
TGATCTACAAGGTGAGCAACAGATTTAGTGGTGTCCCTAGCAGATTCAGCGGCAGCGGCAGCGGCACCGACTTCACC
TTCACCATCAGCAGCCTGCAGCCTGAGGACATCGCCACCTACTACTGCTTTCAGTACAGCCACGTGCCTTGGACCTT
CGGACAAGGCACGAAACTGCAGATCACCGGTGGAGGAGGCAGTGGTGGTGGAGGAAGCGGAGGTGGTGGCAGCCAAG
TGCAGCTGCAGCAGAGCGGCGCCGAGGTGAAGAAGCCTGGCAGCAGCGTGAAGGTGAGCTGCAAGGCTAGCGGCTAC
ACCTTCACCAACTACTACATCTACTGGGTGAGACAAGCCCCTGGCCAAGGCCTGGAGTGGATCGGCGGCATCAACCC
TACAAGCGGCGGCAGCAACTTCAACGAGAAGTTCAAGACAAGAGTTACCATAACTGCCGACGAGAGCAGCACCACCG
CCTACATGGAGCTGAGCAGCCTGAGAAGCGAGGACACCGCCTTCTACTTCTGCACAAGACAAGGCCTGTGGTTCGAC
AGCGACGGCAGAGGCTTCGACTTTTGGGGCCAAGGAACCACAGTTACTGTGTCTTCCGGTGGCGGTGGCTCCGGATC
CGGTTCTGGAGGAGGCGGTGGAAGCGGAGGCGGTGGCTCTGGAGGAGGCGGTTCGGACATCCTGATGACCCAATCTC
CATCCTCCATGTCTGTATCTCTGGGAGACACAGTCAGCATCACTTGCCATTCAAGTCAGGACATTAACAGTAATATA
GGGTGGTTGCAGCAGAGACCAGGGAAATCATTTAAGGGCCTGATCTATCATGGAACCAACTTGGACGATGAAGTTCC
ATCAAGGTTCAGTGGCAGTGGATCTGGAGCCGATTATTCTCTCACCATCAGCAGCCTGGAATCTGAAGATTTTGCAG
ACTATTACTGTGTACAGTATGCTCAGTTTCCGTGGACGTTCGGTGGAGGCACCAAGCTGGAAATCAAACGTACGGTG
GCTGCACCATCTGTCTTCATCTTCCCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCCCTCCAATCGGGTAACTCCCAGGAGA
GTGTCACAGAGCAGGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCTTCAACAGGGGAGA
GTGTTAG
>Seq ID 43: SI-95X16 light chain amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITGGGGSGGGGSGGGGSQVQLQQSGAEVKKPGSSVKVSCKASGY
TFTNYYIYWVRQAPGQGLEWIGGINPTSGGSNFNEKFKTRVTITADESSTTAYMELSSLRSEDTAFYFCTRQGLWFD
SDGRGFDFWGQGTTVTVSSGGGGSGGGGSGSGSGGGGGSGGGGSGGGGSDILMTQSPSSMSVSLGDTVSITCHSSQD
INSNIGWLQQRPGKSFKGLIYHGTNLDDEVPSRFSGSGSGADYSLTISSLESEDFADYYCVQYAQFPWTFGGGTKLE
IKRTVAAPSVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
>Seq ID 44: SI-95X16 light chain nucleotide sequence
GACATTCAGATGACACAGAGCCCTAGCAGCCTGAGCGCTAGCGTGGGCGACAGAGTGACCATCACCTGTAGAAGCTC
TCAGAACATCGTGCACAGCAACGGCAACACCTACCTGGACTGGTATCAGCAGACCCCTGGCAAGGCCCCTAAGCTGC
TGATCTACAAGGTGAGCAACAGATTTAGTGGTGTCCCTAGCAGATTCAGCGGCAGCGGCAGCGGCACCGACTTCACC
TTCACCATCAGCAGCCTGCAGCCTGAGGACATCGCCACCTACTACTGCTTTCAGTACAGCCACGTGCCTTGGACCTT
CGGACAAGGCACGAAACTGCAGATCACCGGTGGAGGAGGCAGTGGTGGTGGAGGAAGCGGAGGTGGTGGCAGCCAAG
TGCAGCTGCAGCAGAGCGGCGCCGAGGTGAAGAAGCCTGGCAGCAGCGTGAAGGTGAGCTGCAAGGCTAGCGGCTAC
ACCTTCACCAACTACTACATCTACTGGGTGAGACAAGCCCCTGGCCAAGGCCTGGAGTGGATCGGCGGCATCAACCC
TACAAGCGGCGGCAGCAACTTCAACGAGAAGTTCAAGACAAGAGTTACCATAACTGCCGACGAGAGCAGCACCACCG
CCTACATGGAGCTGAGCAGCCTGAGAAGCGAGGACACCGCCTTCTACTTCTGCACAAGACAAGGCCTGTGGTTCGAC
AGCGACGGCAGAGGCTTCGACTTTTGGGGCCAAGGAACCACAGTTACTGTGTCTTCCGGAGGCGGTGGTAGCGGTGG
CGGTGGCTCCGGATCCGGTTCTGGAGGAGGCGGTGGAAGCGGAGGCGGTGGCTCTGGAGGAGGCGGTTCGGACATCC
TGATGACCCAATCTCCATCCTCCATGTCTGTATCTCTGGGAGACACAGTCAGCATCACTTGCCATTCAAGTCAGGAC
ATTAACAGTAATATAGGGTGGTTGCAGCAGAGACCAGGGAAATCATTTAAGGGCCTGATCTATCATGGAACCAACTT
GGACGATGAAGTTCCATCAAGGTTCAGTGGCAGTGGATCTGGAGCCGATTATTCTCTCACCATCAGCAGCCTGGAAT
CTGAAGATTTTGCAGACTATTACTGTGTACAGTATGCTCAGTTTCCGTGGACGTTCGGTGGAGGCACCAAGCTGGAA
ATCAAACGTACGGTGGCTGCACCATCTGTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGTGGAAGGTGGATAACGCCCTCCAATCGG
GTAACTCCCAGGAGAGTGTCACAGAGCAGGACAGCAAGGACAGCACCTACAGCCTCAGCAGCACCCTGACGCTGAGC
AAAGCAGACTACGAGAAACACAAAGTCTACGCCTGCGAAGTCACCCATCAGGGCCTGAGCTCGCCCGTCACAAAGAG
CTTCAACAGGGGAGAGTGTTAG
>Seq ID 45: SI-95X17 heavy chain amino acid sequence
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSAGGGGSGGGGSGGGGSDILMTQSPSSMSVSLGDTVSITC
HSSQDINSNIGWLQQRPGKSFKGLIYHGTNLDDEVPSRFSGSGSGADYSLTISSLESEDFADYYCVQYAQFPWTFGG
GTKLEIKSGSGSGGGGGSGGGGSGGGGSQVQLQQSGAEVKKPGSSVKVSCKASGYTFTNYYIYWVRQAPGQGLEWIG
GINPTSGGSNFNEKFKTRVTITADESSTTAYMELSSLRSEDTAFYFCTRQGLWFDSDGRGFDFWGQGTTVTVSSAST
KGPSVFPLAPSSKSTSGGTAALGCLVKDYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGNVFSCSVM
HEALHNHYTQKSLSLSPG
>Seq ID 46: SI-95X17 heavy chain nucleotide sequence
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCAGGTGGAGGAGGCAGTGGTGGTGGAGGAAGCGGAGGTG
GTGGCAGCGACATCCTGATGACCCAATCTCCATCCTCCATGTCTGTATCTCTGGGAGACACAGTCAGCATCACTTGC
CATTCAAGTCAGGACATTAACAGTAATATAGGGTGGTTGCAGCAGAGACCAGGGAAATCATTTAAGGGCCTGATCTA
TCATGGAACCAACTTGGACGATGAAGTTCCATCAAGGTTCAGTGGCAGTGGATCTGGAGCCGATTATTCTCTCACCA
TCAGCAGCCTGGAATCTGAAGATTTTGCAGACTATTACTGTGTACAGTATGCTCAGTTTCCGTGGACGTTCGGTGGA
GGCACCAAGCTGGAAATCAAATCCGGATCCGGTTCTGGAGGAGGCGGTGGAAGCGGAGGCGGTGGCTCTGGAGGAGG
CGGTTCGCAGGTGCAGCTGCAGCAGAGCGGCGCCGAGGTGAAGAAGCCCGGCAGCAGCGTGAAGGTGAGCTGCAAGG
CCAGCGGCTACACCTTCACCAACTACTACATCTACTGGGTGCGGCAGGCCCCCGGCCAGGGCCTGGAGTGGATCGGC
GGCATCAACCCCACCAGCGGCGGCAGCAACTTCAACGAGAAGTTCAAGACCCGGGTGACCATCACCGCCGACGAGAG
CAGCACCACCGCCTACATGGAGCTGAGCAGCCTGCGGAGCGAGGACACCGCCTTCTACTTCTGCACCCGGCAGGGCC
TGTGGTTCGACAGCGACGGCCGGGGCTTCGACTTCTGGGGCCAGGGCACCACCGTGACCGTGAGCAGCGCTAGCACC
AAGGGCCCATCGGTCTTCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAGAGTTGAGCCCAAATCTTGTGACAAAAC
TCACACATGCCCACCGTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGAGGTCAAG
TTCAACTGGTACGTGGACGGCGTGGAGGTGCATAATGCCAAGACAAAGCCGCGGGAGGAGCAGTACAACAGCACGTA
CCGTGTGGTCAGCGTCCTCACCGTCCTGCACCAGGACTGGCTGAATGGCAAGGAGTACAAGTGCAAGGTCTCCAACA
AAGCCCTCCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTGGACTCCGACGGCT
CCTTCTTCCTCTATAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTTAG
>Seq ID 47: SI-95X18 heavy chain amino acid sequence
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSAGGGGSGGGGSGGGGSDILMTQSPSSMSVSLGDTVSITC
HSSQDINSNIGWLQQRPGKSFKGLIYHGTNLDDEVPSRFSGSGSGADYSLTISSLESEDFADYYCVQYAQFPWTFGG
GTKLEIKGGGGSGSGSGGGGGSGGGGSGGGGSQVQLQQSGAEVKKPGSSVKVSCKASGYTFTNYYIYWVRQAPGQGL
EWIGGINPTSGGSNFNEKFKTRVTITADESSTTAYMELSSLRSEDTAFYFCTRQGLWFDSDGRGFDFWGQGTTVTVS
SASTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQQGNVES
CSVMHEALHNHYTQKSLSLSPG
>Seq ID 48: SI-95X18 heavy chain nucleotide sequence
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCAGGTGGAGGAGGCAGTGGTGGTGGAGGAAGCGGAGGTG
GTGGCAGCGACATCCTGATGACCCAATCTCCATCCTCCATGTCTGTATCTCTGGGAGACACAGTCAGCATCACTTGC
CATTCAAGTCAGGACATTAACAGTAATATAGGGTGGTTGCAGCAGAGACCAGGGAAATCATTTAAGGGCCTGATCTA
TCATGGAACCAACTTGGACGATGAAGTTCCATCAAGGTTCAGTGGCAGTGGATCTGGAGCCGATTATTCTCTCACCA
TCAGCAGCCTGGAATCTGAAGATTTTGCAGACTATTACTGTGTACAGTATGCTCAGTTTCCGTGGACGTTCGGTGGA
GGCACCAAGCTGGAAATCAAAGGTGGCGGTGGCTCCGGATCCGGTTCTGGAGGAGGCGGTGGAAGCGGAGGCGGTGG
CTCTGGAGGAGGCGGTTCGCAGGTGCAGCTGCAGCAGAGCGGCGCCGAGGTGAAGAAGCCCGGCAGCAGCGTGAAGG
TGAGCTGCAAGGCCAGCGGCTACACCTTCACCAACTACTACATCTACTGGGTGCGGCAGGCCCCCGGCCAGGGCCTG
GAGTGGATCGGCGGCATCAACCCCACCAGCGGCGGCAGCAACTTCAACGAGAAGTTCAAGACCCGGGTGACCATCAC
CGCCGACGAGAGCAGCACCACCGCCTACATGGAGCTGAGCAGCCTGCGGAGCGAGGACACCGCCTTCTACTTCTGCA
CCCGGCAGGGCCTGTGGTTCGACAGCGACGGCCGGGGCTTCGACTTCTGGGGCCAGGGCACCACCGTGACCGTGAGC
AGCGCTAGCACCAAGGGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAGTTGAGCCCAAATC
TTGTGACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGAAGAC
CCTGAGGTCAAGTTCAACTGGTACGTGGACGGCGTGGAGGTGCATAATGCCAAGACAAAGCCGCGGGAGGAGCAGTA
CAACAGCACGTACCGTGTGGTCAGCGTCCTCACCGTCCTGCACCAGGACTGGCTGAATGGCAAGGAGTACAAGTGCA
AGGTCTCCAACAAAGCCCTCCCAGCCCCCATCGAGAAAACCATCTCCAAAGCCAAAGGGCAGCCCCGAGAACCACAG
GTGTACACCCTGCCCCCATCCCGGGATGAGCTGACCAAGAACCAGGTCAGCCTGACCTGCCTGGTCAAAGGCTTCTA
TCCCAGCGACATCGCCGTGGAGTGGGAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTTAG
>Seq ID 49: SI-95X19 heavy chain amino acid sequence
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSAGGGGSGGGGSGGGGSDILMTQSPSSMSVSLGDTVSITC
HSSQDINSNIGWLQQRPGKSFKGLIYHGTNLDDEVPSRFSGSGSGADYSLTISSLESEDFADYYCVQYAQFPWTFGG
GTKLEIKGGGGSGGGGSGSGSGGGGGSGGGGSGGGGSQVQLQQSGAEVKKPGSSVKVSCKASGYTFTNYYIYWVRQA
PGQGLEWIGGINPTSGGSNFNEKFKTRVTITADESSTTAYMELSSLRSEDTAFYFCTRQGLWFDSDGRGFDFWGQGT
TVTVSSASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPG
>Seq ID 50: SI-95X19 heavy chain nucleotide sequence
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCAGGTGGAGGAGGCAGTGGTGGTGGAGGAAGCGGAGGTG
GTGGCAGCGACATCCTGATGACCCAATCTCCATCCTCCATGTCTGTATCTCTGGGAGACACAGTCAGCATCACTTGC
CATTCAAGTCAGGACATTAACAGTAATATAGGGTGGTTGCAGCAGAGACCAGGGAAATCATTTAAGGGCCTGATCTA
TCATGGAACCAACTTGGACGATGAAGTTCCATCAAGGTTCAGTGGCAGTGGATCTGGAGCCGATTATTCTCTCACCA
TCAGCAGCCTGGAATCTGAAGATTTTGCAGACTATTACTGTGTACAGTATGCTCAGTTTCCGTGGACGTTCGGTGGA
GGCACCAAGCTGGAAATCAAAGGAGGCGGTGGTAGCGGTGGCGGTGGCTCCGGATCCGGTTCTGGAGGAGGCGGTGG
AAGCGGAGGCGGTGGCTCTGGAGGAGGCGGTTCGCAGGTGCAGCTGCAGCAGAGCGGCGCCGAGGTGAAGAAGCCCG
GCAGCAGCGTGAAGGTGAGCTGCAAGGCCAGCGGCTACACCTTCACCAACTACTACATCTACTGGGTGCGGCAGGCC
CCCGGCCAGGGCCTGGAGTGGATCGGCGGCATCAACCCCACCAGCGGCGGCAGCAACTTCAACGAGAAGTTCAAGAC
CCGGGTGACCATCACCGCCGACGAGAGCAGCACCACCGCCTACATGGAGCTGAGCAGCCTGCGGAGCGAGGACACCG
CCTTCTACTTCTGCACCCGGCAGGGCCTGTGGTTCGACAGCGACGGCCGGGGCTTCGACTTCTGGGGCCAGGGCACC
ACCGTGACCGTGAGCAGCGCTAGCACCAAGGGCCCATCGGTCTTCCCCCTGGCACCCTCCTCCAAGAGCACCTCTGG
GGGCACAGCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCCCAGCAACACCAAGGTGGACAAGAG
AGTTGAGCCCAAATCTTGTGACAAAACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCTGAGGTCACATGCGTGGTGGTGGAC
GTGAGCCACGAAGACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGACTGGCTGAATGGCA
AGGAGTACAAGTGCAAGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAACCAGGTCAGCCTGACCTGCCT
GGTCAAAGGCTTCTATCCCAGCGACATCGCCGTGGAGTGGGAGAGCAATGGGCAGCCGGAGAACAACTACAAGACCA
CGCCTCCCGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCCTGTCTCC
GGGTTAG
>Seq ID 51: SI-95X20 heavy chain amino acid sequence
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKGGGGSGGGGSGGGGSDVQLQESGPSLVKPSQSLSLTCTVTGYSITSD
FAWNWIRQFPGNKLEWMGYISYSGNTRYNPSLKSRISITRDTSKNQFFLQLNSVTIEDTATYYCVTAGRGFPYWGQG
TLVTVSASGSGSGGGGGSGGGGSGGGGSQVQLQQSGAEVKKPGSSVKVSCKASGYTFTNYYIYWVRQAPGQGLEWIG
GINPTSGGSNFNEKFKTRVTITADESSTTAYMELSSLRSEDTAFYFCTRQGLWFDSDGRGFDFWGQGTTVTVSSAST
KGPSVFPLAPSSKSTSGGTAALGCLVKDYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGNVFSCSVM
HEALHNHYTQKSLSLSPG
>Seq ID 52: SI-95X20 heavy chain nucleotide sequence
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAAGGTGGAGGAGGCAGTGGTGGTGGAGGAAGCGGAGGTGGTGGCAGCGATGTGCAGCTTCAGGAGT
CGGGACCTAGCCTGGTGAAACCTTCTCAGTCTCTGTCCCTCACCTGCACTGTCACTGGCTACTCAATCACCAGTGAT
TTTGCCTGGAACTGGATTCGGCAGTTTCCAGGAAACAAGCTGGAGTGGATGGGCTACATAAGTTATAGTGGTAACAC
TAGGTACAACCCATCTCTCAAAAGTCGAATCTCTATCACTCGCGACACATCCAAGAACCAATTCTTCCTGCAGTTGA
ACTCTGTGACTATTGAGGACACAGCCACATATTACTGTGTAACGGCGGGACGCGGGTTTCCTTATTGGGGCCAAGGG
ACTCTGGTCACTGTCTCTGCATCCGGATCCGGTTCTGGAGGAGGCGGTGGAAGCGGAGGCGGTGGCTCTGGAGGAGG
CGGTTCGCAGGTGCAGCTGCAGCAGAGCGGCGCCGAGGTGAAGAAGCCCGGCAGCAGCGTGAAGGTGAGCTGCAAGG
CCAGCGGCTACACCTTCACCAACTACTACATCTACTGGGTGCGGCAGGCCCCCGGCCAGGGCCTGGAGTGGATCGGC
GGCATCAACCCCACCAGCGGCGGCAGCAACTTCAACGAGAAGTTCAAGACCCGGGTGACCATCACCGCCGACGAGAG
CAGCACCACCGCCTACATGGAGCTGAGCAGCCTGCGGAGCGAGGACACCGCCTTCTACTTCTGCACCCGGCAGGGCC
TGTGGTTCGACAGCGACGGCCGGGGCTTCGACTTCTGGGGCCAGGGCACCACCGTGACCGTGAGCAGCGCTAGCACC
AAGGGCCCATCGGTCTTCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAGAGTTGAGCCCAAATCTTGTGACAAAAC
TCACACATGCCCACCGTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGAGGTCAAG
TTCAACTGGTACGTGGACGGCGTGGAGGTGCATAATGCCAAGACAAAGCCGCGGGAGGAGCAGTACAACAGCACGTA
CCGTGTGGTCAGCGTCCTCACCGTCCTGCACCAGGACTGGCTGAATGGCAAGGAGTACAAGTGCAAGGTCTCCAACA
AAGCCCTCCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTGGACTCCGACGGCT
CCTTCTTCCTCTATAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTTAG
>Seq ID 53: SI-95X21 heavy chain amino acid sequence
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKGGGGSGGGGSGGGGSDVQLQESGPSLVKPSQSLSLTCTVTGYSITSD
FAWNWIRQFPGNKLEWMGYISYSGNTRYNPSLKSRISITRDTSKNQFFLQLNSVTIEDTATYYCVTAGRGFPYWGQG
TLVTVSAGGGGSGSGSGGGGGSGGGGSGGGGSQVQLQQSGAEVKKPGSSVKVSCKASGYTFTNYYIYWVRQAPGQGL
EWIGGINPTSGGSNFNEKFKTRVTITADESSTTAYMELSSLRSEDTAFYFCTRQGLWFDSDGRGFDFWGQGTTVTVS
SASTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQQGNVFS
CSVMHEALHNHYTQKSLSLSPG
>Seq ID 54: SI-95X21 heavy chain nucleotide sequence
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAAGGTGGAGGAGGCAGTGGTGGTGGAGGAAGCGGAGGTGGTGGCAGCGATGTGCAGCTTCAGGAGT
CGGGACCTAGCCTGGTGAAACCTTCTCAGTCTCTGTCCCTCACCTGCACTGTCACTGGCTACTCAATCACCAGTGAT
TTTGCCTGGAACTGGATTCGGCAGTTTCCAGGAAACAAGCTGGAGTGGATGGGCTACATAAGTTATAGTGGTAACAC
TAGGTACAACCCATCTCTCAAAAGTCGAATCTCTATCACTCGCGACACATCCAAGAACCAATTCTTCCTGCAGTTGA
ACTCTGTGACTATTGAGGACACAGCCACATATTACTGTGTAACGGCGGGACGCGGGTTTCCTTATTGGGGCCAAGGG
ACTCTGGTCACTGTCTCTGCAGGTGGCGGTGGCTCCGGATCCGGTTCTGGAGGAGGCGGTGGAAGCGGAGGCGGTGG
CTCTGGAGGAGGCGGTTCGCAGGTGCAGCTGCAGCAGAGCGGCGCCGAGGTGAAGAAGCCCGGCAGCAGCGTGAAGG
TGAGCTGCAAGGCCAGCGGCTACACCTTCACCAACTACTACATCTACTGGGTGCGGCAGGCCCCCGGCCAGGGCCTG
GAGTGGATCGGCGGCATCAACCCCACCAGCGGCGGCAGCAACTTCAACGAGAAGTTCAAGACCCGGGTGACCATCAC
CGCCGACGAGAGCAGCACCACCGCCTACATGGAGCTGAGCAGCCTGCGGAGCGAGGACACCGCCTTCTACTTCTGCA
CCCGGCAGGGCCTGTGGTTCGACAGCGACGGCCGGGGCTTCGACTTCTGGGGCCAGGGCACCACCGTGACCGTGAGC
AGCGCTAGCACCAAGGGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAGTTGAGCCCAAATC
TTGTGACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGAAGAC
CCTGAGGTCAAGTTCAACTGGTACGTGGACGGCGTGGAGGTGCATAATGCCAAGACAAAGCCGCGGGAGGAGCAGTA
CAACAGCACGTACCGTGTGGTCAGCGTCCTCACCGTCCTGCACCAGGACTGGCTGAATGGCAAGGAGTACAAGTGCA
AGGTCTCCAACAAAGCCCTCCCAGCCCCCATCGAGAAAACCATCTCCAAAGCCAAAGGGCAGCCCCGAGAACCACAG
GTGTACACCCTGCCCCCATCCCGGGATGAGCTGACCAAGAACCAGGTCAGCCTGACCTGCCTGGTCAAAGGCTTCTA
TCCCAGCGACATCGCCGTGGAGTGGGAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTTAG
>Seq ID 55: SI-95X22 heavy chain amino acid sequence
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKGGGGSGGGGSGGGGSDVQLQESGPSLVKPSQSLSLTCTVTGYSITSD
FAWNWIRQFPGNKLEWMGYISYSGNTRYNPSLKSRISITRDTSKNQFFLQLNSVTIEDTATYYCVTAGRGFPYWGQG
TLVTVSAGGGGSGGGGSGSGSGGGGGSGGGGSGGGGSQVQLQQSGAEVKKPGSSVKVSCKASGYTFTNYYIYWVRQA
PGQGLEWIGGINPTSGGSNFNEKFKTRVTITADESSTTAYMELSSLRSEDTAFYFCTRQGLWFDSDGRGFDFWGQGT
TVTVSSASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPG
>Seq ID 56: SI-95X22 heavy chain nucleotide sequence
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAAGGTGGAGGAGGCAGTGGTGGTGGAGGAAGCGGAGGTGGTGGCAGCGATGTGCAGCTTCAGGAGT
CGGGACCTAGCCTGGTGAAACCTTCTCAGTCTCTGTCCCTCACCTGCACTGTCACTGGCTACTCAATCACCAGTGAT
TTTGCCTGGAACTGGATTCGGCAGTTTCCAGGAAACAAGCTGGAGTGGATGGGCTACATAAGTTATAGTGGTAACAC
TAGGTACAACCCATCTCTCAAAAGTCGAATCTCTATCACTCGCGACACATCCAAGAACCAATTCTTCCTGCAGTTGA
ACTCTGTGACTATTGAGGACACAGCCACATATTACTGTGTAACGGCGGGACGCGGGTTTCCTTATTGGGGCCAAGGG
ACTCTGGTCACTGTCTCTGCAGGAGGCGGTGGTAGCGGTGGCGGTGGCTCCGGATCCGGTTCTGGAGGAGGCGGTGG
AAGCGGAGGCGGTGGCTCTGGAGGAGGCGGTTCGCAGGTGCAGCTGCAGCAGAGCGGCGCCGAGGTGAAGAAGCCCG
GCAGCAGCGTGAAGGTGAGCTGCAAGGCCAGCGGCTACACCTTCACCAACTACTACATCTACTGGGTGCGGCAGGCC
CCCGGCCAGGGCCTGGAGTGGATCGGCGGCATCAACCCCACCAGCGGCGGCAGCAACTTCAACGAGAAGTTCAAGAC
CCGGGTGACCATCACCGCCGACGAGAGCAGCACCACCGCCTACATGGAGCTGAGCAGCCTGCGGAGCGAGGACACCG
CCTTCTACTTCTGCACCCGGCAGGGCCTGTGGTTCGACAGCGACGGCCGGGGCTTCGACTTCTGGGGCCAGGGCACC
ACCGTGACCGTGAGCAGCGCTAGCACCAAGGGCCCATCGGTCTTCCCCCTGGCACCCTCCTCCAAGAGCACCTCTGG
GGGCACAGCGGCCCTGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCCCAGCAACACCAAGGTGGACAAGAG
AGTTGAGCCCAAATCTTGTGACAAAACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCTGAGGTCACATGCGTGGTGGTGGAC
GTGAGCCACGAAGACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGACTGGCTGAATGGCA
AGGAGTACAAGTGCAAGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAACCAGGTCAGCCTGACCTGCCT
GGTCAAAGGCTTCTATCCCAGCGACATCGCCGTGGAGTGGGAGAGCAATGGGCAGCCGGAGAACAACTACAAGACCA
CGCCTCCCGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCCTGTCTCC
GGGTTAG
>Seq ID 57: SI-95X23 light chain amino acid sequence
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSAGGGGSGGGGSGGGGSDILMTQSPSSMSVSLGDTVSITC
HSSQDINSNIGWLQQRPGKSFKGLIYHGTNLDDEVPSRFSGSGSGADYSLTISSLESEDFADYYCVQYAQFPWTFGG
GTKLEIKSGSGSGGGGGSGGGGSGGGGSDIQMTQSPSSLSASVGDRVTITCRSSQNIVHSNGNTYLDWYQQTPGKAP
KLLIYKVSNRFSGVPSRFSGSGSGTDFTFTISSLQPEDIATYYCFQYSHVPWTFGQGTKLQITRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGEC
>Seq ID 58: SI-95X23 light chain nucleotide sequence
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCAGGTGGAGGAGGCAGTGGTGGTGGAGGAAGCGGAGGTG
GTGGCAGCGACATCCTGATGACCCAATCTCCATCCTCCATGTCTGTATCTCTGGGAGACACAGTCAGCATCACTTGC
CATTCAAGTCAGGACATTAACAGTAATATAGGGTGGTTGCAGCAGAGACCAGGGAAATCATTTAAGGGCCTGATCTA
TCATGGAACCAACTTGGACGATGAAGTTCCATCAAGGTTCAGTGGCAGTGGATCTGGAGCCGATTATTCTCTCACCA
TCAGCAGCCTGGAATCTGAAGATTTTGCAGACTATTACTGTGTACAGTATGCTCAGTTTCCGTGGACGTTCGGTGGA
GGCACCAAGCTGGAAATCAAATCCGGATCCGGTTCTGGAGGAGGCGGTGGAAGCGGAGGCGGTGGCTCTGGAGGAGG
CGGTTCGGACATTCAGATGACACAGAGCCCTAGCAGCCTGAGCGCTAGCGTGGGCGACAGAGTGACCATCACCTGTA
GAAGCTCTCAGAACATCGTGCACAGCAACGGCAACACCTACCTGGACTGGTATCAGCAGACCCCTGGCAAGGCCCCT
AAGCTGCTGATCTACAAGGTGAGCAACAGATTTAGTGGTGTCCCTAGCAGATTCAGCGGCAGCGGCAGCGGCACCGA
CTTCACCTTCACCATCAGCAGCCTGCAGCCTGAGGACATCGCCACCTACTACTGCTTTCAGTACAGCCACGTGCCTT
GGACCTTCGGACAAGGCACGAAACTGCAGATCACCCGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTTAG
>Seq ID 59: SI-95x24 light chain amino acid sequence
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSAGGGGSGGGGSGGGGSDILMTQSPSSMSVSLGDTVSITC
HSSQDINSNIGWLQQRPGKSFKGLIYHGTNLDDEVPSRFSGSGSGADYSLTISSLESEDFADYYCVQYAQFPWTFGG
GTKLEIKGGGGSGSGSGGGGGSGGGGSGGGGSDIQMTQSPSSLSASVGDRVTITCRSSQNIVHSNGNTYLDWYQQTP
GKAPKLLIYKVSNRFSGVPSRFSGSGSGTDFTFTISSLQPEDIATYYCFQYSHVPWTFGQGTKLQITRTVAAPSVFI
FPPSDEQLKSGTASVVCLLNNFYPREAKVQWKVDNALQSGNSQESVTEQDSKDSTYSLSSTLTLSKADYEKHKVYAC
EVTHQGLSSPVTKSFNRGEC
>Seq ID 60: SI-95X24 light chain nucleotide sequence
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCAGGTGGAGGAGGCAGTGGTGGTGGAGGAAGCGGAGGTG
GTGGCAGCGACATCCTGATGACCCAATCTCCATCCTCCATGTCTGTATCTCTGGGAGACACAGTCAGCATCACTTGC
CATTCAAGTCAGGACATTAACAGTAATATAGGGTGGTTGCAGCAGAGACCAGGGAAATCATTTAAGGGCCTGATCTA
TCATGGAACCAACTTGGACGATGAAGTTCCATCAAGGTTCAGTGGCAGTGGATCTGGAGCCGATTATTCTCTCACCA
TCAGCAGCCTGGAATCTGAAGATTTTGCAGACTATTACTGTGTACAGTATGCTCAGTTTCCGTGGACGTTCGGTGGA
GGCACCAAGCTGGAAATCAAAGGTGGCGGTGGCTCCGGATCCGGTTCTGGAGGAGGCGGTGGAAGCGGAGGCGGTGG
CTCTGGAGGAGGCGGTTCGGACATTCAGATGACACAGAGCCCTAGCAGCCTGAGCGCTAGCGTGGGCGACAGAGTGA
CCATCACCTGTAGAAGCTCTCAGAACATCGTGCACAGCAACGGCAACACCTACCTGGACTGGTATCAGCAGACCCCT
GGCAAGGCCCCTAAGCTGCTGATCTACAAGGTGAGCAACAGATTTAGTGGTGTCCCTAGCAGATTCAGCGGCAGCGG
CAGCGGCACCGACTTCACCTTCACCATCAGCAGCCTGCAGCCTGAGGACATCGCCACCTACTACTGCTTTCAGTACA
GCCACGTGCCTTGGACCTTCGGACAAGGCACGAAACTGCAGATCACCCGTACGGTGGCTGCACCATCTGTCTTCATC
TTCCCGCCATCTGATGAGCAGTTGAAATCTGGAACTGCCTCTGTTGTGTGCCTGCTGAATAACTTCTATCCCAGAGA
GGCCAAAGTACAGTGGAAGGTGGATAACGCCCTCCAATCGGGTAACTCCCAGGAGAGTGTCACAGAGCAGGACAGCA
AGGACAGCACCTACAGCCTCAGCAGCACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGGAGAGTGTTAG
>Seq ID 61: SI-95X25 light chain amino acid sequence
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSAGGGGSGGGGSGGGGSDILMTQSPSSMSVSLGDTVSITC
HSSQDINSNIGWLQQRPGKSFKGLIYHGTNLDDEVPSRFSGSGSGADYSLTISSLESEDFADYYCVQYAQFPWTFGG
GTKLEIKGGGGSGGGGSGSGSGGGGGSGGGGSGGGGSDIQMTQSPSSLSASVGDRVTITCRSSQNIVHSNGNTYLDW
YQQTPGKAPKLLIYKVSNRFSGVPSRFSGSGSGTDFTFTISSLQPEDIATYYCFQYSHVPWTFGQGTKLQITRTVAA
PSVFIFPPSDEQLKSGTASVVCLLNNFYPREAKVQWKVDNALQSGNSQESVTEQDSKDSTYSLSSTLTLSKADYEKH
KVYACEVTHQGLSSPVTKSFNRGEC
>Seq ID 62: SI-95X25 light chain nucleotide sequence
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCAGGTGGAGGAGGCAGTGGTGGTGGAGGAAGCGGAGGTG
GTGGCAGCGACATCCTGATGACCCAATCTCCATCCTCCATGTCTGTATCTCTGGGAGACACAGTCAGCATCACTTGC
CATTCAAGTCAGGACATTAACAGTAATATAGGGTGGTTGCAGCAGAGACCAGGGAAATCATTTAAGGGCCTGATCTA
TCATGGAACCAACTTGGACGATGAAGTTCCATCAAGGTTCAGTGGCAGTGGATCTGGAGCCGATTATTCTCTCACCA
TCAGCAGCCTGGAATCTGAAGATTTTGCAGACTATTACTGTGTACAGTATGCTCAGTTTCCGTGGACGTTCGGTGGA
GGCACCAAGCTGGAAATCAAAGGAGGCGGTGGTAGCGGTGGCGGTGGCTCCGGATCCGGTTCTGGAGGAGGCGGTGG
AAGCGGAGGCGGTGGCTCTGGAGGAGGCGGTTCGGACATTCAGATGACACAGAGCCCTAGCAGCCTGAGCGCTAGCG
TGGGCGACAGAGTGACCATCACCTGTAGAAGCTCTCAGAACATCGTGCACAGCAACGGCAACACCTACCTGGACTGG
TATCAGCAGACCCCTGGCAAGGCCCCTAAGCTGCTGATCTACAAGGTGAGCAACAGATTTAGTGGTGTCCCTAGCAG
ATTCAGCGGCAGCGGCAGCGGCACCGACTTCACCTTCACCATCAGCAGCCTGCAGCCTGAGGACATCGCCACCTACT
ACTGCTTTCAGTACAGCCACGTGCCTTGGACCTTCGGACAAGGCACGAAACTGCAGATCACCCGTACGGTGGCTGCA
CCATCTGTCTTCATCTTCCCGCCATCTGATGAGCAGTTGAAATCTGGAACTGCCTCTGTTGTGTGCCTGCTGAATAA
CTTCTATCCCAGAGAGGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAAGAGCTTCAACAGGGGAGAGTGTTA
G
>Seq ID 63: SI-95X26 light chain amino acid sequence
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKGGGGSGGGGSGGGGSDVQLQESGPSLVKPSQSLSLTCTVTGYSITSD
FAWNWIRQFPGNKLEWMGYISYSGNTRYNPSLKSRISITRDTSKNQFFLQLNSVTIEDTATYYCVTAGRGFPYWGQG
TLVTVSASGSGSGGGGGSGGGGSGGGGSDIQMTQSPSSLSASVGDRVTITCRSSQNIVHSNGNTYLDWYQQTPGKAP
KLLIYKVSNRFSGVPSRFSGSGSGTDFTFTISSLQPEDIATYYCFQYSHVPWTFGQGTKLQITRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGEC
>Seq ID 64: SI-95X26 light chain nucleotide sequence
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAAGGTGGAGGAGGCAGTGGTGGTGGAGGAAGCGGAGGTGGTGGCAGCGATGTGCAGCTTCAGGAGT
CGGGACCTAGCCTGGTGAAACCTTCTCAGTCTCTGTCCCTCACCTGCACTGTCACTGGCTACTCAATCACCAGTGAT
TTTGCCTGGAACTGGATTCGGCAGTTTCCAGGAAACAAGCTGGAGTGGATGGGCTACATAAGTTATAGTGGTAACAC
TAGGTACAACCCATCTCTCAAAAGTCGAATCTCTATCACTCGCGACACATCCAAGAACCAATTCTTCCTGCAGTTGA
ACTCTGTGACTATTGAGGACACAGCCACATATTACTGTGTAACGGCGGGACGCGGGTTTCCTTATTGGGGCCAAGGG
ACTCTGGTCACTGTCTCTGCATCCGGATCCGGTTCTGGAGGAGGCGGTGGAAGCGGAGGCGGTGGCTCTGGAGGAGG
CGGTTCGGACATTCAGATGACACAGAGCCCTAGCAGCCTGAGCGCTAGCGTGGGCGACAGAGTGACCATCACCTGTA
GAAGCTCTCAGAACATCGTGCACAGCAACGGCAACACCTACCTGGACTGGTATCAGCAGACCCCTGGCAAGGCCCCT
AAGCTGCTGATCTACAAGGTGAGCAACAGATTTAGTGGTGTCCCTAGCAGATTCAGCGGCAGCGGCAGCGGCACCGA
CTTCACCTTCACCATCAGCAGCCTGCAGCCTGAGGACATCGCCACCTACTACTGCTTTCAGTACAGCCACGTGCCTT
GGACCTTCGGACAAGGCACGAAACTGCAGATCACCCGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTTAG
>Seq ID 65: SI-95X27 light chain amino acid sequence
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKGGGGSGGGGSGGGGSDVQLQESGPSLVKPSQSLSLTCTVTGYSITSD
FAWNWIRQFPGNKLEWMGYISYSGNTRYNPSLKSRISITRDTSKNQFFLQLNSVTIEDTATYYCVTAGRGFPYWGQG
TLVTVSAGGGGSGSGSGGGGGSGGGGSGGGGSDIQMTQSPSSLSASVGDRVTITCRSSQNIVHSNGNTYLDWYQQTP
GKAPKLLIYKVSNRFSGVPSRFSGSGSGTDFTFTISSLQPEDIATYYCFQYSHVPWTFGQGTKLQITRTVAAPSVFI
FPPSDEQLKSGTASVVCLLNNFYPREAKVQWKVDNALQSGNSQESVTEQDSKDSTYSLSSTLTLSKADYEKHKVYAC
EVTHQGLSSPVTKSFNRGEC
>Seq ID 66: SI-95X27 light chain nucleotide sequence
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAAGGTGGAGGAGGCAGTGGTGGTGGAGGAAGCGGAGGTGGTGGCAGCGATGTGCAGCTTCAGGAGT
CGGGACCTAGCCTGGTGAAACCTTCTCAGTCTCTGTCCCTCACCTGCACTGTCACTGGCTACTCAATCACCAGTGAT
TTTGCCTGGAACTGGATTCGGCAGTTTCCAGGAAACAAGCTGGAGTGGATGGGCTACATAAGTTATAGTGGTAACAC
TAGGTACAACCCATCTCTCAAAAGTCGAATCTCTATCACTCGCGACACATCCAAGAACCAATTCTTCCTGCAGTTGA
ACTCTGTGACTATTGAGGACACAGCCACATATTACTGTGTAACGGCGGGACGCGGGTTTCCTTATTGGGGCCAAGGG
ACTCTGGTCACTGTCTCTGCAGGTGGCGGTGGCTCCGGATCCGGTTCTGGAGGAGGCGGTGGAAGCGGAGGCGGTGG
CTCTGGAGGAGGCGGTTCGGACATTCAGATGACACAGAGCCCTAGCAGCCTGAGCGCTAGCGTGGGCGACAGAGTGA
CCATCACCTGTAGAAGCTCTCAGAACATCGTGCACAGCAACGGCAACACCTACCTGGACTGGTATCAGCAGACCCCT
GGCAAGGCCCCTAAGCTGCTGATCTACAAGGTGAGCAACAGATTTAGTGGTGTCCCTAGCAGATTCAGCGGCAGCGG
CAGCGGCACCGACTTCACCTTCACCATCAGCAGCCTGCAGCCTGAGGACATCGCCACCTACTACTGCTTTCAGTACA
GCCACGTGCCTTGGACCTTCGGACAAGGCACGAAACTGCAGATCACCCGTACGGTGGCTGCACCATCTGTCTTCATC
TTCCCGCCATCTGATGAGCAGTTGAAATCTGGAACTGCCTCTGTTGTGTGCCTGCTGAATAACTTCTATCCCAGAGA
GGCCAAAGTACAGTGGAAGGTGGATAACGCCCTCCAATCGGGTAACTCCCAGGAGAGTGTCACAGAGCAGGACAGCA
AGGACAGCACCTACAGCCTCAGCAGCACCCTGACGCTGAGCAAAGCAGACTACGAGAAACACAAAGTCTACGCCTGC
GAAGTCACCCATCAGGGCCTGAGCTCGCCCGTCACAAAGAGCTTCAACAGGGGAGAGTGTTAG
>Seq ID 67: SI-95X28 light chain amino acid sequence
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKGGGGSGGGGSGGGGSDVQLQESGPSLVKPSQSLSLTCTVTGYSITSD
FAWNWIRQFPGNKLEWMGYISYSGNTRYNPSLKSRISITRDTSKNQFFLQLNSVTIEDTATYYCVTAGRGFPYWGQG
TLVTVSAGGGGSGGGGSGSGSGGGGGSGGGGSGGGGSDIQMTQSPSSLSASVGDRVTITCRSSQNIVHSNGNTYLDW
YQQTPGKAPKLLIYKVSNRFSGVPSRFSGSGSGTDFTFTISSLQPEDIATYYCFQYSHVPWTFGQGTKLQITRTVAA
PSVFIFPPSDEQLKSGTASVVCLLNNFYPREAKVQWKVDNALQSGNSQESVTEQDSKDSTYSLSSTLTLSKADYEKH
KVYACEVTHQGLSSPVTKSFNRGEC
>Seq ID 68: SI-95X28 light chain nucleotide sequence
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAAGGTGGAGGAGGCAGTGGTGGTGGAGGAAGCGGAGGTGGTGGCAGCGATGTGCAGCTTCAGGAGT
CGGGACCTAGCCTGGTGAAACCTTCTCAGTCTCTGTCCCTCACCTGCACTGTCACTGGCTACTCAATCACCAGTGAT
TTTGCCTGGAACTGGATTCGGCAGTTTCCAGGAAACAAGCTGGAGTGGATGGGCTACATAAGTTATAGTGGTAACAC
TAGGTACAACCCATCTCTCAAAAGTCGAATCTCTATCACTCGCGACACATCCAAGAACCAATTCTTCCTGCAGTTGA
ACTCTGTGACTATTGAGGACACAGCCACATATTACTGTGTAACGGCGGGACGCGGGTTTCCTTATTGGGGCCAAGGG
ACTCTGGTCACTGTCTCTGCAGGAGGCGGTGGTAGCGGTGGCGGTGGCTCCGGATCCGGTTCTGGAGGAGGCGGTGG
AAGCGGAGGCGGTGGCTCTGGAGGAGGCGGTTCGGACATTCAGATGACACAGAGCCCTAGCAGCCTGAGCGCTAGCG
TGGGCGACAGAGTGACCATCACCTGTAGAAGCTCTCAGAACATCGTGCACAGCAACGGCAACACCTACCTGGACTGG
TATCAGCAGACCCCTGGCAAGGCCCCTAAGCTGCTGATCTACAAGGTGAGCAACAGATTTAGTGGTGTCCCTAGCAG
ATTCAGCGGCAGCGGCAGCGGCACCGACTTCACCTTCACCATCAGCAGCCTGCAGCCTGAGGACATCGCCACCTACT
ACTGCTTTCAGTACAGCCACGTGCCTTGGACCTTCGGACAAGGCACGAAACTGCAGATCACCCGTACGGTGGCTGCA
CCATCTGTCTTCATCTTCCCGCCATCTGATGAGCAGTTGAAATCTGGAACTGCCTCTGTTGTGTGCCTGCTGAATAA
CTTCTATCCCAGAGAGGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAAGAGCTTCAACAGGGGAGAGTGTTA
G
>Seq ID 69: SI-95X29 light chain amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGECSGSGSGGGGGSG
GGGSGGGGSDVQLQESGPSLVKPSQSLSLTCTVTGYSITSDFAWNWIRQFPGNKLEWMGYISYSGNTRYNPSLKSRI
SITRDTSKNQFFLQLNSVTIEDTATYYCVTAGRGFPYWGQGTLVTVSAGGGGSGGGGSGGGGSDILMTQSPSSMSVS
LGDTVSITCHSSQDINSNIGWLQQRPGKSFKGLIYHGTNLDDEVPSRFSGSGSGADYSLTISSLESEDFADYYCVQY
AQFPWTFGGGTKLEIK
>Seq ID 70: SI-95X29 light chain nucleotide sequence
GATATTCAAATGACTCAATCTCCTTCTTCTCTTTCTGCTTCTGTTGGTGATCGTGTTACTATTACTTGTCGTTCTTC
TCAAAATATTGTTCATTCTAATGGTAATACTTATCTTGATTGGTATCAACAAACTCCTGGTAAAGCTCCTAAACTTC
TTATTTATAAAGTTTCTAATCGTTTTTCTGGTGTTCCTTCTCGTTTTTCTGGTTCTGGTTCTGGTACTGATTTTACT
TTTACTATTTCTTCTCTTCAACCTGAAGATATTGCTACTTATTATTGTTTTCAATATTCTCATGTTCCTTGGACTTT
TGGTCAAGGTACTAAACTTCAAATTACTCGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTCCGGATCCGGTTCTGGAGGAGGCGGTGGAAGCGGA
GGCGGTGGCTCTGGAGGAGGCGGTTCGGATGTGCAGCTTCAGGAGTCGGGACCTAGCCTGGTGAAACCTTCTCAGTC
TCTGTCCCTCACCTGCACTGTCACTGGCTACTCAATCACCAGTGATTTTGCCTGGAACTGGATTCGGCAGTTTCCAG
GAAACAAGCTGGAGTGGATGGGCTACATAAGTTATAGTGGTAACACTAGGTACAACCCATCTCTCAAAAGTCGAATC
TCTATCACTCGCGACACATCCAAGAACCAATTCTTCCTGCAGTTGAACTCTGTGACTATTGAGGACACAGCCACATA
TTACTGTGTAACGGCGGGACGCGGGTTTCCTTATTGGGGCCAAGGGACTCTGGTCACTGTCTCTGCAGGTGGAGGAG
GCAGTGGTGGTGGAGGAAGCGGAGGTGGTGGCAGCGACATCCTGATGACCCAATCTCCATCCTCCATGTCTGTATCT
CTGGGAGACACAGTCAGCATCACTTGCCATTCAAGTCAGGACATTAACAGTAATATAGGGTGGTTGCAGCAGAGACC
AGGGAAATCATTTAAGGGCCTGATCTATCATGGAACCAACTTGGACGATGAAGTTCCATCAAGGTTCAGTGGCAGTG
GATCTGGAGCCGATTATTCTCTCACCATCAGCAGCCTGGAATCTGAAGATTTTGCAGACTATTACTGTGTACAGTAT
GCTCAGTTTCCGTGGACGTTCGGTGGAGGCACCAAGCTGGAAATCAAATAG
>Seq ID 71: SI-95X30 light chain amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGECGGGGSGSGSGGG
GGSGGGGSGGGGSDVQLQESGPSLVKPSQSLSLTCTVTGYSITSDFAWNWIRQFPGNKLEWMGYISYSGNTRYNPSL
KSRISITRDTSKNQFFLQLNSVTIEDTATYYCVTAGRGFPYWGQGTLVTVSAGGGGSGGGGSGGGGSDILMTQSPSS
MSVSLGDTVSITCHSSQDINSNIGWLQQRPGKSFKGLIYHGTNLDDEVPSRFSGSGSGADYSLTISSLESEDFADYY
CVQYAQFPWTFGGGTKLEIK
>Seq ID 72: SI-95X30 light chain nucleotide sequence
GATATTCAAATGACTCAATCTCCTTCTTCTCTTTCTGCTTCTGTTGGTGATCGTGTTACTATTACTTGTCGTTCTTC
TCAAAATATTGTTCATTCTAATGGTAATACTTATCTTGATTGGTATCAACAAACTCCTGGTAAAGCTCCTAAACTTC
TTATTTATAAAGTTTCTAATCGTTTTTCTGGTGTTCCTTCTCGTTTTTCTGGTTCTGGTTCTGGTACTGATTTTACT
TTTACTATTTCTTCTCTTCAACCTGAAGATATTGCTACTTATTATTGTTTTCAATATTCTCATGTTCCTTGGACTTT
TGGTCAAGGTACTAAACTTCAAATTACTCGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTGGTGGCGGTGGCTCCGGATCCGGTTCTGGAGGAGGC
GGTGGAAGCGGAGGCGGTGGCTCTGGAGGAGGCGGTTCGGATGTGCAGCTTCAGGAGTCGGGACCTAGCCTGGTGAA
ACCTTCTCAGTCTCTGTCCCTCACCTGCACTGTCACTGGCTACTCAATCACCAGTGATTTTGCCTGGAACTGGATTC
GGCAGTTTCCAGGAAACAAGCTGGAGTGGATGGGCTACATAAGTTATAGTGGTAACACTAGGTACAACCCATCTCTC
AAAAGTCGAATCTCTATCACTCGCGACACATCCAAGAACCAATTCTTCCTGCAGTTGAACTCTGTGACTATTGAGGA
CACAGCCACATATTACTGTGTAACGGCGGGACGCGGGTTTCCTTATTGGGGCCAAGGGACTCTGGTCACTGTCTCTG
CAGGTGGAGGAGGCAGTGGTGGTGGAGGAAGCGGAGGTGGTGGCAGCGACATCCTGATGACCCAATCTCCATCCTCC
ATGTCTGTATCTCTGGGAGACACAGTCAGCATCACTTGCCATTCAAGTCAGGACATTAACAGTAATATAGGGTGGTT
GCAGCAGAGACCAGGGAAATCATTTAAGGGCCTGATCTATCATGGAACCAACTTGGACGATGAAGTTCCATCAAGGT
TCAGTGGCAGTGGATCTGGAGCCGATTATTCTCTCACCATCAGCAGCCTGGAATCTGAAGATTTTGCAGACTATTAC
TGTGTACAGTATGCTCAGTTTCCGTGGACGTTCGGTGGAGGCACCAAGCTGGAAATCAAATAG
>Seq ID 73: SI-95X31 light chain amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGECGGGGSGGGGSGS
GSGGGGGSGGGGSGGGGSDVQLQESGPSLVKPSQSLSLTCTVTGYSITSDFAWNWIRQFPGNKLEWMGYISYSGNTR
YNPSLKSRISITRDTSKNQFFLQLNSVTIEDTATYYCVTAGRGFPYWGQGTLVTVSAGGGGSGGGGSGGGGSDILMT
QSPSSMSVSLGDTVSITCHSSQDINSNIGWLQQRPGKSFKGLIYHGTNLDDEVPSRFSGSGSGADYSLTISSLESED
FADYYCVQYAQFPWTFGGGTKLEIK
>Seq ID 74: SI-95X31 light chain nucleotide sequence
GATATTCAAATGACTCAATCTCCTTCTTCTCTTTCTGCTTCTGTTGGTGATCGTGTTACTATTACTTGTCGTTCTTC
TCAAAATATTGTTCATTCTAATGGTAATACTTATCTTGATTGGTATCAACAAACTCCTGGTAAAGCTCCTAAACTTC
TTATTTATAAAGTTTCTAATCGTTTTTCTGGTGTTCCTTCTCGTTTTTCTGGTTCTGGTTCTGGTACTGATTTTACT
TTTACTATTTCTTCTCTTCAACCTGAAGATATTGCTACTTATTATTGTTTTCAATATTCTCATGTTCCTTGGACTTT
TGGTCAAGGTACTAAACTTCAAATTACTCGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTGGAGGCGGTGGTAGCGGTGGCGGTGGCTCCGGATCC
GGTTCTGGAGGAGGCGGTGGAAGCGGAGGCGGTGGCTCTGGAGGAGGCGGTTCGGATGTGCAGCTTCAGGAGTCGGG
ACCTAGCCTGGTGAAACCTTCTCAGTCTCTGTCCCTCACCTGCACTGTCACTGGCTACTCAATCACCAGTGATTTTG
CCTGGAACTGGATTCGGCAGTTTCCAGGAAACAAGCTGGAGTGGATGGGCTACATAAGTTATAGTGGTAACACTAGG
TACAACCCATCTCTCAAAAGTCGAATCTCTATCACTCGCGACACATCCAAGAACCAATTCTTCCTGCAGTTGAACTC
TGTGACTATTGAGGACACAGCCACATATTACTGTGTAACGGCGGGACGCGGGTTTCCTTATTGGGGCCAAGGGACTC
TGGTCACTGTCTCTGCAGGTGGAGGAGGCAGTGGTGGTGGAGGAAGCGGAGGTGGTGGCAGCGACATCCTGATGACC
CAATCTCCATCCTCCATGTCTGTATCTCTGGGAGACACAGTCAGCATCACTTGCCATTCAAGTCAGGACATTAACAG
TAATATAGGGTGGTTGCAGCAGAGACCAGGGAAATCATTTAAGGGCCTGATCTATCATGGAACCAACTTGGACGATG
AAGTTCCATCAAGGTTCAGTGGCAGTGGATCTGGAGCCGATTATTCTCTCACCATCAGCAGCCTGGAATCTGAAGAT
TTTGCAGACTATTACTGTGTACAGTATGCTCAGTTTCCGTGGACGTTCGGTGGAGGCACCAAGCTGGAAATCAAATA
G
>Seq ID 75: SI-95X35 heavy chain amino acid sequence
DILLTQSPVILSVSPGERVSFSCRASQSIGTNIHWYQQRTNGSPRLLIKYASESISGIPSRFSGSGSGTDFTLSINS
VESEDIADYYCQQNNNWPTTFGAGTKLELKGGGGSGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQVFFKMNSLQSNDTAIYYCARALTYYDY
EFAYWGQGTLVTVSSSGSGSGGGGGSGGGGSGGGGSDVQLQESGPSLVKPSQSLSLTCTVTGYSITSDFAWNWIRQF
PGNKLEWMGYISYSGNTRYNPSLKSRISITRDTSKNQFFLQLNSVTIEDTATYYCVTAGRGFPYWGQGTLVTVSAAS
TKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVESCSV
MHEALHNHYTQKSLSLSPG
>Seq ID 76: SI-95X35 heavy chain nucleotide sequence
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATAATAACTGGCCAACCACGTTCGGTGCTGGGACCAA
GCTGGAGCTGAAAGGCGGTGGCGGTAGTGGGGGAGGCGGTTCTGGCGGCGGAGGGTCCGGCGGTGGAGGATCACAGG
TGCAGCTGAAGCAGTCAGGACCTGGCCTAGTGCAGCCCTCACAGAGCCTGTCCATCACCTGCACAGTCTCTGGTTTC
TCATTAACTAACTATGGTGTACACTGGGTTCGCCAGTCTCCAGGAAAGGGTCTGGAGTGGCTGGGAGTGATATGGAG
TGGTGGAAACACAGACTATAATACACCTTTCACATCCAGACTGAGCATCAACAAGGACAATTCCAAGAGCCAAGTTT
TCTTTAAAATGAACAGTCTGCAATCTAATGACACAGCCATATATTACTGTGCCAGAGCCCTCACCTACTATGATTAC
GAGTTTGCTTACTGGGGCCAAGGGACTCTGGTCACTGTCTCGAGCTCCGGATCCGGTTCTGGAGGAGGCGGTGGAAG
CGGAGGCGGTGGCTCTGGAGGAGGCGGTTCGGATGTGCAGCTTCAGGAGTCGGGACCTAGCCTGGTGAAACCTTCTC
AGTCTCTGTCCCTCACCTGCACTGTCACTGGCTACTCAATCACCAGTGATTTTGCCTGGAACTGGATTCGGCAGTTT
CCAGGAAACAAGCTGGAGTGGATGGGCTACATAAGTTATAGTGGTAACACTAGGTACAACCCATCTCTCAAAAGTCG
AATCTCTATCACTCGCGACACATCCAAGAACCAATTCTTCCTGCAGTTGAACTCTGTGACTATTGAGGACACAGCCA
CATATTACTGTGTAACGGCGGGACGCGGGTTTCCTTATTGGGGCCAAGGGACTCTGGTCACTGTCTCTGCAGCTAGC
ACCAAGGGCCCATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCAAATCTTGTGACAA
AACTCACACATGCCCACCGTGCCCAGCACCTGAACTCCTGGGGGGACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCAAGGTCTCCA
ACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGT
>Seq ID 77: SI-95X36 light chain amino acid sequence
DILLTQSPVILSVSPGERVSFSCRASQSIGTNIHWYQQRTNGSPRLLIKYASESISGIPSRFSGSGSGTDFTLSINS
VESEDIADYYCQQNNNWPTTFGAGTKLELKGGGGSGGGGSGGGGSGGGGSQVQLKQSGPGLVQPSQSLSITCTVSGF
SLTNYGVHWVRQSPGKGLEWLGVIWSGGNTDYNTPFTSRLSINKDNSKSQVFFKMNSLQSNDTAIYYCARALTYYDY
EFAYWGQGTLVTVSSSGSGSGGGGGSGGGGSGGGGSDILMTQSPSSMSVSLGDTVSITCHSSQDINSNIGWLQQRPG
KSFKGLIYHGTNLDDEVPSRFSGSGSGADYSLTISSLESEDFADYYCVQYAQFPWTFGGGTKLEIKRTVAAPSVFIF
PPSDEQLKSGTASVVCLLNNFYPREAKVQWKVDNALQSGNSQESVTEQDSKDSTYSLSSTLTLSKADYEKHKVYACE
VTHQGLSSPVTKSFNRGEC
>Seq ID 78: SI-95X36 light chain nucleotide sequence
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATAATAACTGGCCAACCACGTTCGGTGCTGGGACCAA
GCTGGAGCTGAAAGGCGGTGGCGGTAGTGGGGGAGGCGGTTCTGGCGGCGGAGGGTCCGGCGGTGGAGGATCACAGG
TGCAGCTGAAGCAGTCAGGACCTGGCCTAGTGCAGCCCTCACAGAGCCTGTCCATCACCTGCACAGTCTCTGGTTTC
TCATTAACTAACTATGGTGTACACTGGGTTCGCCAGTCTCCAGGAAAGGGTCTGGAGTGGCTGGGAGTGATATGGAG
TGGTGGAAACACAGACTATAATACACCTTTCACATCCAGACTGAGCATCAACAAGGACAATTCCAAGAGCCAAGTTT
TCTTTAAAATGAACAGTCTGCAATCTAATGACACAGCCATATATTACTGTGCCAGAGCCCTCACCTACTATGATTAC
GAGTTTGCTTACTGGGGCCAAGGGACTCTGGTCACTGTCTCGAGCTCCGGATCCGGTTCTGGAGGAGGCGGTGGAAG
CGGAGGCGGTGGCTCTGGAGGAGGCGGTTCGGACATCCTGATGACCCAATCTCCATCCTCCATGTCTGTATCTCTGG
GAGACACAGTCAGCATCACTTGCCATTCAAGTCAGGACATTAACAGTAATATAGGGTGGTTGCAGCAGAGACCAGGG
AAATCATTTAAGGGCCTGATCTATCATGGAACCAACTTGGACGATGAAGTTCCATCAAGGTTCAGTGGCAGTGGATC
TGGAGCCGATTATTCTCTCACCATCAGCAGCCTGGAATCTGAAGATTTTGCAGACTATTACTGTGTACAGTATGCTC
AGTTTCCGTGGACGTTCGGTGGAGGCACCAAGCTGGAAATCAAACGTACGGTGGCTGCACCATCTGTCTTCATCTTC
CCGCCATCTGATGAGCAGTTGAAATCTGGAACTGCCTCTGTTGTGTGCCTGCTGAATAACTTCTATCCCAGAGAGGC
CAAAGTACAGTGGAAGGTGGATAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAACACAAAGTCTACGCCTGCGAA
GTCACCCATCAGGGCCTGAGCTCGCCCGTCACAAAGAGCTTCAACAGGGGAGAGTGT
>Seq ID 79: SI-95X37 heavy chain amino acid sequence
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKGGGGSGGGGSGGGGSDVQLQESGPSLVKPSQSLSLTCTVTGYSITSD
FAWNWIRQFPGNKLEWMGYISYSGNTRYNPSLKSRISITRDTSKNQFFLQLNSVTIEDTATYYCVTAGRGFPYWGQG
TLVTVSASGSGSGGGGGSGGGGSGGGGSQVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARALTYYDYEFAYWGQGTLVTVSSASTKGPS
VFPLAPSSKSTSGGTAALGCLVKDYFPEPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVMHEAL
HNHYTQKSLSLSPG
>Seq ID 80: SI-95X37 heavy chain nucleotide sequence
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAAGGTGGAGGAGGCAGTGGTGGTGGAGGAAGCGGAGGTGGTGGCAGCGATGTGCAGCTTCAGGAGT
CGGGACCTAGCCTGGTGAAACCTTCTCAGTCTCTGTCCCTCACCTGCACTGTCACTGGCTACTCAATCACCAGTGAT
TTTGCCTGGAACTGGATTCGGCAGTTTCCAGGAAACAAGCTGGAGTGGATGGGCTACATAAGTTATAGTGGTAACAC
TAGGTACAACCCATCTCTCAAAAGTCGAATCTCTATCACTCGCGACACATCCAAGAACCAATTCTTCCTGCAGTTGA
ACTCTGTGACTATTGAGGACACAGCCACATATTACTGTGTAACGGCGGGACGCGGGTTTCCTTATTGGGGCCAAGGG
ACTCTGGTCACTGTCTCTGCATCCGGATCCGGTTCTGGAGGAGGCGGTGGAAGCGGAGGCGGTGGCTCTGGAGGAGG
CGGTTCGCAGGTGCAGCTGAAGCAGTCAGGACCTGGCCTAGTGCAGCCCTCACAGAGCCTGTCCATCACCTGCACAG
TCTCTGGTTTCTCATTAACTAACTATGGTGTACACTGGGTTCGCCAGTCTCCAGGAAAGGGTCTGGAGTGGCTGGGA
GTGATATGGAGTGGTGGAAACACAGACTATAATACACCTTTCACATCCAGACTGAGCATCAACAAGGACAATTCCAA
GAGCCAAGTTTTCTTTAAAATGAACAGTCTGCAATCTAATGACACAGCCATATATTACTGTGCCAGAGCCCTCACCT
ACTATGATTACGAGTTTGCTTACTGGGGCCAAGGGACTCTGGTCACTGTCTCTAGCGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGGCTGTCCTACAGT
CCTCAGGACTCTACTCCCTCAGCAGCGTGGTGACCGTGCCCTCCAGCAGCTTGGGCACCCAGACCTACATCTGCAAC
GTGAATCACAAGCCCAGCAACACCAAGGTGGACAAGAGAGTTGAGCCCAAATCTTGTGACAAAACTCACACATGCCC
ACCGTGCCCAGCACCTGAACTCCTGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTGTGGTCAG
CGTCCTCACCGTCCTGCACCAGGACTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACAGGTGTACACCCTGCCCCCATCCCGG
GATGAGCTGACCAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTCTTCCTCT
ATAGCAAGCTCACCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTTAA
>Seq ID 81: SI-95X38 light chain amino acid sequence
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKGGGGSGGGGSGGGGSDVQLQESGPSLVKPSQSLSLTCTVTGYSITSD
FAWNWIRQFPGNKLEWMGYISYSGNTRYNPSLKSRISITRDTSKNQFFLQLNSVTIEDTATYYCVTAGRGFPYWGQG
TLVTVSASGSGSGGGGGSGGGGSGGGGSDILLTQSPVILSVSPGERVSFSCRASQSIGTNIHWYQQRTNGSPRLLIK
YASESISGIPSRFSGSGSGTDFTLSINSVESEDIADYYCQQNNNWPTTFGAGTKLELKRTVAAPSVFIFPPSDEQLK
SGTASVVCLLNNFYPREAKVQWKVDNALQSGNSQESVTEQDSKDSTYSLSSTLTLSKADYEKHKVYACEVTHQGLSS
PVTKSFNRGEC
>Seq ID 82: SI-95X38 light chain nucleotide sequence
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAAGGTGGAGGAGGCAGTGGTGGTGGAGGAAGCGGAGGTGGTGGCAGCGATGTGCAGCTTCAGGAGT
CGGGACCTAGCCTGGTGAAACCTTCTCAGTCTCTGTCCCTCACCTGCACTGTCACTGGCTACTCAATCACCAGTGAT
TTTGCCTGGAACTGGATTCGGCAGTTTCCAGGAAACAAGCTGGAGTGGATGGGCTACATAAGTTATAGTGGTAACAC
TAGGTACAACCCATCTCTCAAAAGTCGAATCTCTATCACTCGCGACACATCCAAGAACCAATTCTTCCTGCAGTTGA
ACTCTGTGACTATTGAGGACACAGCCACATATTACTGTGTAACGGCGGGACGCGGGTTTCCTTATTGGGGCCAAGGG
ACTCTGGTCACTGTCTCTGCATCCGGATCCGGTTCTGGAGGAGGCGGTGGAAGCGGAGGCGGTGGCTCTGGAGGAGG
CGGTTCGGACATCTTGCTGACTCAGTCTCCAGTCATCCTGTCTGTGAGTCCAGGAGAAAGAGTCAGTTTCTCCTGCA
GGGCCAGTCAGAGTATTGGCACAAACATACACTGGTATCAGCAAAGAACAAATGGTTCTCCAAGGCTTCTCATAAAG
TATGCTTCTGAGTCTATCTCTGGGATTCCTTCCAGGTTTAGTGGCAGTGGATCAGGGACAGATTTTACTCTTAGCAT
CAACAGTGTGGAGTCTGAAGATATTGCAGATTATTACTGTCAACAAAATAATAACTGGCCAACCACGTTCGGTGCTG
GGACCAAGCTGGAGCTGAAACGTACGGTGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCAAGGACAGCACCTACAGCCTCAGCAGCA
CCCTGACGCTGAGCAAAGCAGACTACGAGAAACACAAAGTCTACGCCTGCGAAGTCACCCATCAGGGCCTGAGCTCG
CCCGTCACAAAGAGCTTCAACAGGGGAGAGTGTTAG
>Seq ID 83: SI-95X39 light chain amino acid sequence
DILLTQSPVILSVSPGERVSFSCRASQSIGTNIHWYQQRINGSPRLLIKYASESISGIPSRFSGSGSGTDFTLSINS
VESEDIADYYCQQNNNWPTTFGAGTKLELKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECSGSGSGGGGGSGGGGSG
GGGSDVQLQESGPSLVKPSQSLSLTCTVTGYSITSDFAWNWIRQFPGNKLEWMGYISYSGNTRYNPSLKSRISITRD
TSKNQFFLQLNSVTIEDTATYYCVTAGRGFPYWGQGTLVTVSAGGGGSGGGGSGGGGSDILMTQSPSSMSVSLGDTV
SITCHSSQDINSNIGWLQQRPGKSFKGLIYHGTNLDDEVPSRFSGSGSGADYSLTISSLESEDFADYYCVQYAQFPW
TFGGGTKLEIK
>Seq ID 84: SI-95X39 light chain nucleotide sequence
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATAATAACTGGCCAACCACGTTCGGTGCTGGGACCAA
GCTGGAGCTGAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTTCCGGATCCGGTTCTGGAGGAGGCGGTGGAAGCGGAGGCGGTGGCTCTGGA
GGAGGCGGTTCGGATGTGCAGCTTCAGGAGTCGGGACCTAGCCTGGTGAAACCTTCTCAGTCTCTGTCCCTCACCTG
CACTGTCACTGGCTACTCAATCACCAGTGATTTTGCCTGGAACTGGATTCGGCAGTTTCCAGGAAACAAGCTGGAGT
GGATGGGCTACATAAGTTATAGTGGTAACACTAGGTACAACCCATCTCTCAAAAGTCGAATCTCTATCACTCGCGAC
ACATCCAAGAACCAATTCTTCCTGCAGTTGAACTCTGTGACTATTGAGGACACAGCCACATATTACTGTGTAACGGC
GGGACGCGGGTTTCCTTATTGGGGCCAAGGGACTCTGGTCACTGTCTCTGCAGGTGGAGGAGGCAGTGGTGGTGGAG
GAAGCGGAGGTGGTGGCAGCGACATCCTGATGACCCAATCTCCATCCTCCATGTCTGTATCTCTGGGAGACACAGTC
AGCATCACTTGCCATTCAAGTCAGGACATTAACAGTAATATAGGGTGGTTGCAGCAGAGACCAGGGAAATCATTTAA
GGGCCTGATCTATCATGGAACCAACTTGGACGATGAAGTTCCATCAAGGTTCAGTGGCAGTGGATCTGGAGCCGATT
ATTCTCTCACCATCAGCAGCCTGGAATCTGAAGATTTTGCAGACTATTACTGTGTACAGTATGCTCAGTTTCCGTGG
ACGTTCGGTGGAGGCACCAAGCTGGAAATCAAA
>Seq ID 85: SI-95x40 heavy chain amino acid sequence
QVQLQQSGAEVKKPGSSVKVSCKASGYTFTNYYIYWVRQAPGQGLEWIGGINPTSGGSNFNEKFKTRVTI
TADESSTTAYMELSSLRSEDTAFYFCTRQGLWFDSDGRGFDFWGQGTTVTVSSASTKGPSVFPLAPSSKS
TSGGTAALGCLVKDYFPEPVTVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGGGGGSGGGGSQVQLKQSGPGLVQPSQSLSITCTVSGFS
LTNYGVHWVRQSPGKGLEWLGVIWSGGNTDYNTPFTSRLSINKDNSKSQVFFKMNSLQSNDTAIYYCARA
LTYYDYEFAYWGQGTLVTVSSGGGGSGGGGSGGGGSGGGGSDILLTQSPVILSVSPGERVSFSCRASQSI
GTNIHWYQQRTNGSPRLLIKYASESISGIPSRFSGSGSGTDFTLSINSVESEDIADYYCQQNNNWPTTFG
AGTKLELK
>Seq ID 86: SI-95x40 heavy chain nucleotide sequence
CAGGTGCAGCTGCAGCAGAGCGGCGCCGAGGTGAAGAAGCCCGGCAGCAGCGTGAAGGTGAGCTGCAAGG
CCAGCGGCTACACCTTCACCAACTACTACATCTACTGGGTGCGGCAGGCCCCCGGCCAGGGCCTGGAGTG
GATCGGCGGCATCAACCCCACCAGCGGCGGCAGCAACTTCAACGAGAAGTTCAAGACCCGGGTGACCATC
ACCGCCGACGAGAGCAGCACCACCGCCTACATGGAGCTGAGCAGCCTGCGGAGCGAGGACACCGCCTTCT
ACTTCTGCACCCGGCAGGGCCTGTGGTTCGACAGCGACGGCCGGGGCTTCGACTTCTGGGGCCAGGGCAC
CACCGTGACCGTGAGCAGCGCTAGCACCAAGGGCCCATCGGTCTTCCCCCTGGCACCCTCCTCCAAGAGC
ACCTCTGGGGGCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGGACTCTACTC
CCTCAGCAGCGTGGTGACCGTGCCCTCCAGCAGCTTGGGCACCCAGACCTACATCTGCAACGTGAATCAC
AAGCCCAGCAACACCAAGGTGGACAAGAGAGTTGAGCCCAAATCTTGTGACAAAACTCACACATGCCCAC
CGTGCCCAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAGGTCAAG
TTCAACTGGTACGTGGACGGCGTGGAGGTGCATAATGCCAAGACAAAGCCGCGGGAGGAGCAGTACAACA
GCACGTACCGTGTGGTCAGCGTCCTCACCGTCCTGCACCAGGACTGGCTGAATGGCAAGGAGTACAAGTG
CAAGGTCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGATGAGCTGACCAAGAACCAGGTCAGCCTGACCTGCC
TGGTCAAAGGCTTCTATCCCAGCGACATCGCCGTGGAGTGGGAGAGCAATGGGCAGCCGGAGAACAACTA
CAAGACCACGCCTCCCGTGCTGGACTCCGACGGCTCCTTCTTCCTCTATAGCAAGCTCACCGTGGACAAG
AGCAGGTGGCAGCAGGGGAACGTCTTCTCATGCTCCGTGATGCATGAGGCTCTGCACAACCACTACACGC
AGAAGAGCCTCTCCCTGTCTCCGGGTGGCGGTGGAGGGTCCGGCGGTGGTGGATCCCAGGTGCAGCTGAA
GCAGTCAGGACCTGGCCTAGTGCAGCCCTCACAGAGCCTGTCCATCACCTGCACAGTCTCTGGTTTCTCA
TTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAA
GAGCCAAGTTTTCTTTAAAATGAACAGTCTGCAATCTAATGACACAGCCATATATTACTGTGCCAGAGCC
CTCACCTACTATGATTACGAGTTTGCTTACTGGGGCCAAGGGACTCTGGTCACTGTCTCTAGCGGCGGTG
GCGGTAGTGGGGGAGGCGGTTCTGGCGGCGGAGGGTCCGGCGGTGGAGGATCAGACATCTTGCTGACTCA
GTCTCCAGTCATCCTGTCTGTGAGTCCAGGAGAAAGAGTCAGTTTCTCCTGCAGGGCCAGTCAGAGTATT
GGCACAAACATACACTGGTATCAGCAAAGAACAAATGGTTCTCCAAGGCTTCTCATAAAGTATGCTTCTG
AGTCTATCTCTGGGATTCCTTCCAGGTTTAGTGGCAGTGGATCAGGGACAGATTTTACTCTTAGCATCAA
CAGTGTGGAGTCTGAAGATATTGCAGATTATTACTGTCAACAAAATAATAACTGGCCAACCACGTTCGGT
GCTGGGACCAAGCTGGAGCTGAAA
>Seq ID 87: SI-95x41 heavy chain amino acid sequence
DVQLQESGPSLVKPSQSLSLTCTVTGYSITSDFAWNWIRQFPGNKLEWMGYISYSGNTRYNPSLKSRISI
TRDTSKNQFFLQLNSVTIEDTATYYCVTAGRGFPYWGQGTLVTVSAASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGGGGGSGGGGSDVQLQESGPSLVKPSQSLSLTCTVTGYSITSDFAW
NWIRQFPGNKLEWMGYISYSGNTRYNPSLKSRISITRDTSKNQFFLQLNSVTIEDTATYYCVTAGRGFPY
WGQGTLVTVSAGGGGSGGGGSGGGGSGGGGSDILMTQSPSSMSVSLGDTVSITCHSSQDINSNIGWLQQR
PGKSFKGLIYHGTNLDDEVPSRFSGSGSGADYSLTISSLESEDFADYYCVQYAQFPWTFGGGTKLEIK
>Seq ID 88: SI-95x41 heavy chain nucleotide sequence
GATGTGCAGCTTCAGGAGTCGGGACCTAGCCTGGTGAAACCTTCTCAGTCTCTGTCCCTCACCTGCACTG
TCACTGGCTACTCAATCACCAGTGATTTTGCCTGGAACTGGATTCGGCAGTTTCCAGGAAACAAGCTGGA
GTGGATGGGCTACATAAGTTATAGTGGTAACACTAGGTACAACCCATCTCTCAAAAGTCGAATCTCTATC
ACTCGCGACACATCCAAGAACCAATTCTTCCTGCAGTTGAACTCTGTGACTATTGAGGACACAGCCACAT
ATTACTGTGTAACGGCGGGACGCGGGTTTCCTTATTGGGGCCAAGGGACTCTGGTCACTGTCTCTGCAGC
TAGCACCAAGGGCCCATCGGTCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCAGCGTGGTGACCGT
GCCCTCCAGCAGCTTGGGCACCCAGACCTACATCTGCAACGTGAATCACAAGCCCAGCAACACCAAGGTG
GACAAGAGAGTTGAGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCT
GGACTCCGACGGCTCCTTCTTCCTCTATAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTCCCTGTCTC
CGGGTGGCGGTGGAGGGTCCGGCGGTGGTGGATCCGATGTGCAGCTTCAGGAGTCGGGACCTAGCCTGGT
GAAACCTTCTCAGTCTCTGTCCCTCACCTGCACTGTCACTGGCTACTCAATCACCAGTGATTTTGCCTGG
AACTGGATTCGGCAGTTTCCAGGAAACAAGCTGGAGTGGATGGGCTACATAAGTTATAGTGGTAACACTA
GGTACAACCCATCTCTCAAAAGTCGAATCTCTATCACTCGCGACACATCCAAGAACCAATTCTTCCTGCA
GTTGAACTCTGTGACTATTGAGGACACAGCCACATATTACTGTGTAACGGCGGGACGCGGGTTTCCTTAT
TGGGGCCAAGGGACTCTGGTCACTGTCTCTGCAGGCGGTGGCGGTAGTGGGGGAGGCGGTTCTGGCGGCG
GAGGGTCCGGCGGTGGAGGATCAGACATCCTGATGACCCAATCTCCATCCTCCATGTCTGTATCTCTGGG
AGACACAGTCAGCATCACTTGCCATTCAAGTCAGGACATTAACAGTAATATAGGGTGGTTGCAGCAGAGA
CCAGGGAAATCATTTAAGGGCCTGATCTATCATGGAACCAACTTGGACGATGAAGTTCCATCAAGGTTCA
GTGGCAGTGGATCTGGAGCCGATTATTCTCTCACCATCAGCAGCCTGGAATCTGAAGATTTTGCAGACTA
TTACTGTGTACAGTATGCTCAGTTTCCGTGGACGTTCGGTGGAGGCACCAAGCTGGAAATCAAA
>Seq ID 89: SI-95X42 heavy chain amino acid sequence
QVQLKQSGPGLVQPSQSLSITCTVSGFSLTNYGVHWVRQSPGKGLEWLGVIWSGGNTDYNTPFTSRLSIN
KDNSKSQVFFKMNSLQSNDTAIYYCARALTYYDYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGGGGGSGGGGSQVQLKQSGPGLVQPSQSLSITCTVSGFSLTNY
GVHWVRQSPGKGLEWLGVIWSGGNTDYNTPFTSRLSINKDNSKSQVFFKMNSLQSNDTAIYYCARALTYY
DYEFAYWGQGTLVTVSSGGGGSGGGGSGGGGSGGGGSDILLTQSPVILSVSPGERVSFSCRASQSIGTNI
HWYQQRTNGSPRLLIKYASESISGIPSRFSGSGSGTDFTLSINSVESEDIADYYCQQNNNWPTTFGAGTK
LELK
>Seq ID 90: SI-95x42 heavy chain nucleotide sequence
CAGGTGCAGCTGAAGCAGTCAGGACCTGGCCTAGTGCAGCCCTCACAGAGCCTGTCCATCACCTGCACAG
TCTCTGGTTTCTCATTAACTAACTATGGTGTACACTGGGTTCGCCAGTCTCCAGGAAAGGGTCTGGAGTG
GCTGGGAGTGATATGGAGTGGTGGAAACACAGACTATAATACACCTTTCACATCCAGACTGAGCATCAAC
AAGGACAATTCCAAGAGCCAAGTTTTCTTTAAAATGAACAGTCTGCAATCTAATGACACAGCCATATATT
ACTGTGCCAGAGCCCTCACCTACTATGATTACGAGTTTGCTTACTGGGGCCAAGGGACTCTGGTCACTGT
CTCTAGCGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTGGCGGTGGAGGGTCCGGCGGTGGTGGATCCCAGGTGCAGCTGAAGCAGTCAGGACC
TGGCCTAGTGCAGCCCTCACAGAGCCTGTCCATCACCTGCACAGTCTCTGGTTTCTCATTAACTAACTAT
GGTGTACACTGGGTTCGCCAGTCTCCAGGAAAGGGTCTGGAGTGGCTGGGAGTGATATGGAGTGGTGGAA
ACACAGACTATAATACACCTTTCACATCCAGACTGAGCATCAACAAGGACAATTCCAAGAGCCAAGTTTT
CTTTAAAATGAACAGTCTGCAATCTAATGACACAGCCATATATTACTGTGCCAGAGCCCTCACCTACTAT
GATTACGAGTTTGCTTACTGGGGCCAAGGGACTCTGGTCACTGTCTCTAGCGGCGGTGGCGGTAGTGGGG
GAGGCGGTTCTGGCGGCGGAGGGTCCGGCGGTGGAGGATCAGACATCTTGCTGACTCAGTCTCCAGTCAT
CCTGTCTGTGAGTCCAGGAGAAAGAGTCAGTTTCTCCTGCAGGGCCAGTCAGAGTATTGGCACAAACATA
CACTGGTATCAGCAAAGAACAAATGGTTCTCCAAGGCTTCTCATAAAGTATGCTTCTGAGTCTATCTCTG
GGATTCCTTCCAGGTTTAGTGGCAGTGGATCAGGGACAGATTTTACTCTTAGCATCAACAGTGTGGAGTC
TGAAGATATTGCAGATTATTACTGTCAACAAAATAATAACTGGCCAACCACGTTCGGTGCTGGGACCAAG
CTGGAGCTGAAA
>Seq ID 91: SI-95x43 light chain amino acid sequence
DILMTQSPSSMSVSLGDTVSITCHSSQDINSNIGWLQQRPGKSFKGLIYHGTNLDDEVPSRFSGSGSGAD
YSLTISSLESEDFADYYCVQYAQFPWTFGGGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSEN
RGECGGGGSGGGGSGGGGSQVQLKQSGPGLVQPSQSLSITCTVSGFSLTNYGVHWVRQSPGKGLEWLGVI
WSGGNTDYNTPFTSRLSINKDNSKSQVFFKMNSLQSNDTAIYYCARALTYYDYEFAYWGQGTLVTVSSGG
GGSGGGGSGGGGSGGGGSDILLTQSPVILSVSPGERVSFSCRASQSIGTNIHWYQQRTNGSPRLLIKYAS
ESISGIPSRFSGSGSGTDFTLSINSVESEDIADYYCQQNNNWPTTFGAGTKLELK
>Seq ID 92: SI-95x43 light chain nucleotide sequence
GACATCCTGATGACCCAATCTCCATCCTCCATGTCTGTATCTCTGGGAGACACAGTCAGCATCACTTGCC
ATTCAAGTCAGGACATTAACAGTAATATAGGGTGGTTGCAGCAGAGACCAGGGAAATCATTTAAGGGCCT
GATCTATCATGGAACCAACTTGGACGATGAAGTTCCATCAAGGTTCAGTGGCAGTGGATCTGGAGCCGAT
TATTCTCTCACCATCAGCAGCCTGGAATCTGAAGATTTTGCAGACTATTACTGTGTACAGTATGCTCAGT
TTCCGTGGACGTTCGGTGGAGGCACCAAGCTGGAAATCAAACGTACGGTGGCTGCACCATCTGTCTTCAT
CTTCCCGCCATCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAAC
AGGGGAGAGTGTGGCGGTGGCGGTAGCGGTGGCGGCGGAAGTGGTGGCGGAGGATCCCAGGTGCAGCTGA
AGCAGTCAGGACCTGGCCTAGTGCAGCCCTCACAGAGCCTGTCCATCACCTGCACAGTCTCTGGTTTCTC
ATTAACTAACTATGGTGTACACTGGGTTCGCCAGTCTCCAGGAAAGGGTCTGGAGTGGCTGGGAGTGATA
TGGAGTGGTGGAAACACAGACTATAATACACCTTTCACATCCAGACTGAGCATCAACAAGGACAATTCCA
AGAGCCAAGTTTTCTTTAAAATGAACAGTCTGCAATCTAATGACACAGCCATATATTACTGTGCCAGAGC
CCTCACCTACTATGATTACGAGTTTGCTTACTGGGGCCAAGGGACTCTGGTCACTGTCTCTAGCGGCGGT
GGCGGTAGTGGGGGAGGCGGTTCTGGCGGCGGAGGGTCCGGCGGTGGAGGATCAGACATCTTGCTGACTC
AGTCTCCAGTCATCCTGTCTGTGAGTCCAGGAGAAAGAGTCAGTTTCTCCTGCAGGGCCAGTCAGAGTAT
TGGCACAAACATACACTGGTATCAGCAAAGAACAAATGGTTCTCCAAGGCTTCTCATAAAGTATGCTTCT
GAGTCTATCTCTGGGATTCCTTCCAGGTTTAGTGGCAGTGGATCAGGGACAGATTTTACTCTTAGCATCA
ACAGTGTGGAGTCTGAAGATATTGCAGATTATTACTGTCAACAAAATAATAACTGGCCAACCACGTTCGG
TGCTGGGACCAAGCTGGAGCTGAAA
>Seq ID 93: SI-95x44 heavy chain amino acid sequence
DILLTQSPVILSVSPGERVSFSCRASQSIGTNIHWYQQRTNGSPRLLIKYASESISGIPSRES
GSGSGTDFTLSINSVESEDIADYYCQQNNNWPTTFGAGTKLELKGGGGSGGGGSGGGGSGGGG
SQVQLKQSGPGLVQPSQSLSITCTVSGFSLTNYGVHWVRQSPGKGLEWLGVIWSGGNTDYNTP
FTSRLSINKDNSKSQVFFKMNSLQSNDTAIYYCARALTYYDYEFAYWGQGTLVTVSSGGGGSG
GGGSGGGGSGGGGSDVQLQESGPSLVKPSQSLSLTCTVTGYSITSDFAWNWIRQFPGNKLEWM
GYISYSGNTRYNPSLKSRISITRDTSKNQFFLQLNSVTIEDTATYYCVTAGRGFPYWGQGTLV
TVSAASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTCPPCPAPELLGGPS
VFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVM
HEALHNHYTQKSLSLSPG
>Seq ID 94: SI-95x44 heavy chain nucleotide sequence
GACATCTTGCTGACTCAGTCTCCAGTCATCCTGTCTGTGAGTCCAGGAGAAAGAGTCAGTTTCTCCTGCA
GGGCCAGTCAGAGTATTGGCACAAACATACACTGGTATCAGCAAAGAACAAATGGTTCTCCAAGGCTTCT
CATAAAGTATGCTTCTGAGTCTATCTCTGGGATTCCTTCCAGGTTTAGTGGCAGTGGATCAGGGACAGAT
TTTACTCTTAGCATCAACAGTGTGGAGTCTGAAGATATTGCAGATTATTACTGTCAACAAAATAATAACT
GGCCAACCACGTTCGGTGCTGGGACCAAGCTGGAGCTGAAAGGCGGTGGCGGTAGTGGGGGAGGCGGTTC
TGGCGGCGGAGGGTCCGGCGGTGGAGGATCACAGGTGCAGCTGAAGCAGTCAGGACCTGGCCTAGTGCAG
CCCTCACAGAGCCTGTCCATCACCTGCACAGTCTCTGGTTTCTCATTAACTAACTATGGTGTACACTGGG
TTCGCCAGTCTCCAGGAAAGGGTCTGGAGTGGCTGGGAGTGATATGGAGTGGTGGAAACACAGACTATAA
TACACCTTTCACATCCAGACTGAGCATCAACAAGGACAATTCCAAGAGCCAAGTTTTCTTTAAAATGAAC
AGTCTGCAATCTAATGACACAGCCATATATTACTGTGCCAGAGCCCTCACCTACTATGATTACGAGTTTG
CTTACTGGGGCCAAGGGACTCTGGTCACTGTCTCGAGCGGCGGTGGCGGTAGTGGGGGAGGCGGTTCTGG
CGGCGGAGGGTCCGGCGGTGGAGGATCAGATGTGCAGCTTCAGGAGTCGGGACCTAGCCTGGTGAAACCT
TCTCAGTCTCTGTCCCTCACCTGCACTGTCACTGGCTACTCAATCACCAGTGATTTTGCCTGGAACTGGA
TTCGGCAGTTTCCAGGAAACAAGCTGGAGTGGATGGGCTACATAAGTTATAGTGGTAACACTAGGTACAA
CCCATCTCTCAAAAGTCGAATCTCTATCACTCGCGACACATCCAAGAACCAATTCTTCCTGCAGTTGAAC
TCTGTGACTATTGAGGACACAGCCACATATTACTGTGTAACGGCGGGACGCGGGTTTCCTTATTGGGGCC
AAGGGACTCTGGTCACTGTCTCTGCAGCTAGCACCAAGGGCCCATCGGTCTTCCCCCTGGCACCCTCCTC
CAAGAGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGAC
TCTACTCCCTCAGCAGCGTGGTGACCGTGCCCTCCAGCAGCTTGGGCACCCAGACCTACATCTGCAACGT
GAATCACAAGCCCAGCAACACCAAGGTGGACAAGAGAGTTGAGCCCAAATCTTGTGACAAAACTCACACA
TGCCCACCGTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTATAGCAAGCTCACCGT
GGACAAGAGCAGGTGGCAGCAGGGGAACGTCTTCTCATGCTCCGTGATGCATGAGGCTCTGCACAACCAC
TACACGCAGAAGAGCCTCTCCCTGTCTCCGGGT
>Seq ID 95: SI-95x44 light chain amino acid sequence
DILMTQSPSSMSVSLGDTVSITCHSSQDINSNIGWLQQRPGKSFKGLIYHGTNLDDEVPSRFSGSGS
GADYSLTISSLESEDFADYYCVQYAQFPWTFGGGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGL
SSPVTKSFNRGEC
>Seq ID 96: SI-95x44 light chain nucleotide sequence
GACATCCTGATGACCCAATCTCCATCCTCCATGTCTGTATCTCTGGGAGACACAGTCAGCATCACTT
GCCATTCAAGTCAGGACATTAACAGTAATATAGGGTGGTTGCAGCAGAGACCAGGGAAATCATTTAA
GGGCCTGATCTATCATGGAACCAACTTGGACGATGAAGTTCCATCAAGGTTCAGTGGCAGTGGATCT
GGAGCCGATTATTCTCTCACCATCAGCAGCCTGGAATCTGAAGATTTTGCAGACTATTACTGTGTAC
AGTATGCTCAGTTTCCGTGGACGTTCGGTGGAGGCACCAAGCTGGAAATCAAACGTACGGTGGCTGC
ACCATCTGTCTTCATCTTCCCGCCATCTGATGAGCAGTTGAAATCTGGAACTGCCTCTGTTGTGTGC
CTGCTGAATAACTTCTATCCCAGAGAGGCCAAAGTACAGTGGAAGGTGGATAACGCCCTCCAATCGG
GTAACTCCCAGGAGAGTGTCACAGAGCAGGACAGCAAGGACAGCACCTACAGCCTCAGCAGCACCCT
GACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGT
>Seq ID 97:
SI-95x45 heavy chain amino acid sequence
QVQLQQSGPGLVKPSETLSITCTVSGFSLTNYGVHWIRQAPGKGLEWLGVIWSGGNTDYNTPFTSRFTIT
KDNSKNQVYFKLRSVRADDTAIYYCARALTYYYYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGGGGGGSGGGGSDVQLQESGPSLVKPSQSLSLTCTVTGYSITS
DFAWNWIRQFPGNKLEWMGYISYSGNTRYNPSLKSRISITRDTSKNQFFLQLNSVTIEDTATYYCVTAGR
GFPYWGQGTLVTVSAGGGGSGGGGSGGGGSGGGGSDILMTQSPSSMSVSLGDTVSITCHSSQDINSNIGW
LQQRPGKSFKGLIYHGTNLDDEVPSRFSGSGSGADYSLTISSLESEDFADYYCVQYAQFPWTFGGGTKLE
IK
>Seq ID 98: SI-95x45 heavy chain nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCTACTACTACTACGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTGGTGGCGGTGGAGGGTCCGGCGGTGGTGGATCCGATGTGCAGCTTCAGGAGTCGGG
ACCTAGCCTGGTGAAACCTTCTCAGTCTCTGTCCCTCACCTGCACTGTCACTGGCTACTCAATCACCAGT
GATTTTGCCTGGAACTGGATTCGGCAGTTTCCAGGAAACAAGCTGGAGTGGATGGGCTACATAAGTTATA
GTGGTAACACTAGGTACAACCCATCTCTCAAAAGTCGAATCTCTATCACTCGCGACACATCCAAGAACCA
ATTCTTCCTGCAGTTGAACTCTGTGACTATTGAGGACACAGCCACATATTACTGTGTAACGGCGGGACGC
GGGTTTCCTTATTGGGGCCAAGGGACTCTGGTCACTGTCTCTGCAGGCGGTGGCGGTAGTGGGGGAGGCG
GTTCTGGCGGCGGAGGGTCCGGCGGTGGAGGATCAGACATCCTGATGACCCAATCTCCATCCTCCATGTC
TGTATCTCTGGGAGACACAGTCAGCATCACTTGCCATTCAAGTCAGGACATTAACAGTAATATAGGGTGG
TTGCAGCAGAGACCAGGGAAATCATTTAAGGGCCTGATCTATCATGGAACCAACTTGGACGATGAAGTTC
CATCAAGGTTCAGTGGCAGTGGATCTGGAGCCGATTATTCTCTCACCATCAGCAGCCTGGAATCTGAAGA
TTTTGCAGACTATTACTGTGTACAGTATGCTCAGTTTCCGTGGACGTTCGGTGGAGGCACCAAGCTGGAA
ATCAAA
>Seq ID 99: SI-95m5 light chain amino acid sequence
EIVLTQSPSTLSVSPGERATFSCRASQSIGTNIHWYQQKPGKPPRLLIKYASESISGIPDRFSGSGSGTE
FTLTISSVQSEDFAVYYCQQNNNWPTTFGPGTKLTVLRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSEN
RGEC
>Seq ID 100: SI-95m5 light chain nucleotide sequence
GAGATCGTGCTGACCCAGTCTCCTTCCACACTGTCTGTGTCTCCCGGCGAGAGAGCCACCTTCAGCTGTA
GAGCCTCTCAGTCCATCGGCACCAACATCCACTGGTATCAGCAGAAGCCCGGCAAGCCTCCTCGGCTGCT
GATTAAGTACGCCTCCGAGTCCATCAGCGGCATCCCTGACAGATTCTCCGGCTCTGGCTCTGGCACCGAG
TTTACCCTGACCATCTCCTCCGTGCAGTCCGAGGATTTCGCCGTGTACTACTGCCAGCAGAACAACAACT
GGCCCACCACCTTTGGACCCGGCACCAAGCTGACCGTGCTGCGTACGGTGGCTGCACCATCTGTCTTCAT
CTTCCCGCCATCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAAC
AGGGGAGAGTGT
>Seq ID 101: SI-95x46 heavy chain amino acid sequence
QVQLQQSGPGLVKPSETLSITCTVSGFSLTNYGVHWIRQAPGKGLEWLGVIWSGGNTDYNTPFTSRFTIT
KDNSKNQVYFKLRSVRADDTAIYYCARALTYYDLEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGGGGGGSGGGGSDVQLQESGPSLVKPSQSLSLTCTVTGYSITS
DFAWNWIRQFPGNKLEWMGYISYSGNTRYNPSLKSRISITRDTSKNQFFLQLNSVTIEDTATYYCVTAGR
GFPYWGQGTLVTVSAGGGGSGGGGSGGGGSGGGGSDILMTQSPSSMSVSLGDTVSITCHSSQDINSNIGW
LQQRPGKSFKGLIYHGTNLDDEVPSRFSGSGSGADYSLTISSLESEDFADYYCVQYAQFPWTFGGGTKLE
IK
>Seq ID 102: SI-95x46 heavy chain nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCTACTACGACCTCGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTGGTGGCGGTGGAGGGTCCGGCGGTGGTGGATCCGATGTGCAGCTTCAGGAGTCGGG
ACCTAGCCTGGTGAAACCTTCTCAGTCTCTGTCCCTCACCTGCACTGTCACTGGCTACTCAATCACCAGT
GATTTTGCCTGGAACTGGATTCGGCAGTTTCCAGGAAACAAGCTGGAGTGGATGGGCTACATAAGTTATA
GTGGTAACACTAGGTACAACCCATCTCTCAAAAGTCGAATCTCTATCACTCGCGACACATCCAAGAACCA
ATTCTTCCTGCAGTTGAACTCTGTGACTATTGAGGACACAGCCACATATTACTGTGTAACGGCGGGACGC
GGGTTTCCTTATTGGGGCCAAGGGACTCTGGTCACTGTCTCTGCAGGCGGTGGCGGTAGTGGGGGAGGCG
GTTCTGGCGGCGGAGGGTCCGGCGGTGGAGGATCAGACATCCTGATGACCCAATCTCCATCCTCCATGTC
TGTATCTCTGGGAGACACAGTCAGCATCACTTGCCATTCAAGTCAGGACATTAACAGTAATATAGGGTGG
TTGCAGCAGAGACCAGGGAAATCATTTAAGGGCCTGATCTATCATGGAACCAACTTGGACGATGAAGTTC
CATCAAGGTTCAGTGGCAGTGGATCTGGAGCCGATTATTCTCTCACCATCAGCAGCCTGGAATCTGAAGA
TTTTGCAGACTATTACTGTGTACAGTATGCTCAGTTTCCGTGGACGTTCGGTGGAGGCACCAAGCTGGAA
ATCAAA
>Seq ID 103: SI-95x47 heavy chain amino acid sequence
QVQLQQSGPGLVKPSETLSITCTVSGFSLTNYGVHWIRQAPGKGLEWLGVIWSGGNTDYNTPFTSRFTIT
KDNSKNQVYFKLRSVRADDTAIYYCARALTAYDYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGGGGGGSGGGGSDVQLQESGPSLVKPSQSLSLTCTVTGYSITS
DFAWNWIRQFPGNKLEWMGYISYSGNTRYNPSLKSRISITRDTSKNQFFLQLNSVTIEDTATYYCVTAGR
GFPYWGQGTLVTVSAGGGGSGGGGSGGGGSGGGGSDILMTQSPSSMSVSLGDTVSITCHSSQDINSNIGW
LQQRPGKSFKGLIYHGTNLDDEVPSRFSGSGSGADYSLTISSLESEDFADYYCVQYAQFPWTFGGGTKLE
IK
>Seq ID 104: SI-95x47 heavy chain nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCGCCTACGACTACGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTGGTGGCGGTGGAGGGTCCGGCGGTGGTGGATCCGATGTGCAGCTTCAGGAGTCGGG
ACCTAGCCTGGTGAAACCTTCTCAGTCTCTGTCCCTCACCTGCACTGTCACTGGCTACTCAATCACCAGT
GATTTTGCCTGGAACTGGATTCGGCAGTTTCCAGGAAACAAGCTGGAGTGGATGGGCTACATAAGTTATA
GTGGTAACACTAGGTACAACCCATCTCTCAAAAGTCGAATCTCTATCACTCGCGACACATCCAAGAACCA
ATTCTTCCTGCAGTTGAACTCTGTGACTATTGAGGACACAGCCACATATTACTGTGTAACGGCGGGACGC
GGGTTTCCTTATTGGGGCCAAGGGACTCTGGTCACTGTCTCTGCAGGCGGTGGCGGTAGTGGGGGAGGCG
GTTCTGGCGGCGGAGGGTCCGGCGGTGGAGGATCAGACATCCTGATGACCCAATCTCCATCCTCCATGTC
TGTATCTCTGGGAGACACAGTCAGCATCACTTGCCATTCAAGTCAGGACATTAACAGTAATATAGGGTGG
TTGCAGCAGAGACCAGGGAAATCATTTAAGGGCCTGATCTATCATGGAACCAACTTGGACGATGAAGTTC
CATCAAGGTTCAGTGGCAGTGGATCTGGAGCCGATTATTCTCTCACCATCAGCAGCCTGGAATCTGAAGA
TTTTGCAGACTATTACTGTGTACAGTATGCTCAGTTTCCGTGGACGTTCGGTGGAGGCACCAAGCTGGAA
ATCAAA
>Seq ID 105: SI-95x48 heavy chain amino acid sequence
QVQLQQSGPGLVKPSETLSITCTVSGFSLTNYGVHWIRQAPGKGLEWLGVIWSGGNTDYNTPFTSRFTIT
KDNSKNQVYFKLRSVRADDTAIYYCARALTYADYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGGGGGGSGGGGSDVQLQESGPSLVKPSQSLSLTCTVTGYSITS
DFAWNWIRQFPGNKLEWMGYISYSGNTRYNPSLKSRISITRDTSKNQFFLQLNSVTIEDTATYYCVTAGR
GFPYWGQGTLVTVSAGGGGSGGGGSGGGGSGGGGSDILMTQSPSSMSVSLGDTVSITCHSSQDINSNIGW
LQQRPGKSFKGLIYHGTNLDDEVPSRFSGSGSGADYSLTISSLESEDFADYYCVQYAQFPWTFGGGTKLE
IK
>Seq ID 106: SI-95x48 heavy chain nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCTACGCCGACTACGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTGGTGGCGGTGGAGGGTCCGGCGGTGGTGGATCCGATGTGCAGCTTCAGGAGTCGGG
ACCTAGCCTGGTGAAACCTTCTCAGTCTCTGTCCCTCACCTGCACTGTCACTGGCTACTCAATCACCAGT
GATTTTGCCTGGAACTGGATTCGGCAGTTTCCAGGAAACAAGCTGGAGTGGATGGGCTACATAAGTTATA
GTGGTAACACTAGGTACAACCCATCTCTCAAAAGTCGAATCTCTATCACTCGCGACACATCCAAGAACCA
ATTCTTCCTGCAGTTGAACTCTGTGACTATTGAGGACACAGCCACATATTACTGTGTAACGGCGGGACGC
GGGTTTCCTTATTGGGGCCAAGGGACTCTGGTCACTGTCTCTGCAGGCGGTGGCGGTAGTGGGGGAGGCG
GTTCTGGCGGCGGAGGGTCCGGCGGTGGAGGATCAGACATCCTGATGACCCAATCTCCATCCTCCATGTC
TGTATCTCTGGGAGACACAGTCAGCATCACTTGCCATTCAAGTCAGGACATTAACAGTAATATAGGGTGG
TTGCAGCAGAGACCAGGGAAATCATTTAAGGGCCTGATCTATCATGGAACCAACTTGGACGATGAAGTTC
CATCAAGGTTCAGTGGCAGTGGATCTGGAGCCGATTATTCTCTCACCATCAGCAGCCTGGAATCTGAAGA
TTTTGCAGACTATTACTGTGTACAGTATGCTCAGTTTCCGTGGACGTTCGGTGGAGGCACCAAGCTGGAA
ATCAAA
>Seq ID 107: SI-95x49 heavy chain amino acid sequence
QVQLQQSGPGLVKPSETLSITCTVSGFSLTNYGVHWIRQAPGKGLEWLGVIWSGGNTDYNTPFTSRFTIT
KDNSKNQVYFKLRSVRADDTAIYYCARALTYYDYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGGGGGSGGGGSDVQLQESGPSLVKPSQSLSLTCTVTGYSITSD
FAWNWIRQFPGNKLEWMGYISYSGNTRYNPSLKSRISITRDTSKNQFFLQLNSVTIEDTATYYCVTAGRG
FPYWGQGTLVTVSAGGGGSGGGGSGGGGSGGGGSDILMTQSPSSMSVSLGDTVSITCHSSQDINSNIGWL
QQRPGKSFKGLIYHGTNLDDEVPSRFSGSGSGADYSLTISSLESEDFADYYCVQYAQFPWTFGGGTKLEI
K
>Seq ID 108: SI-95x49 heavy chain nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCTACTACGACTACGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTGGCGGTGGAGGGTCCGGCGGTGGTGGATCCGATGTGCAGCTTCAGGAGTCGGGACC
TAGCCTGGTGAAACCTTCTCAGTCTCTGTCCCTCACCTGCACTGTCACTGGCTACTCAATCACCAGTGAT
TTTGCCTGGAACTGGATTCGGCAGTTTCCAGGAAACAAGCTGGAGTGGATGGGCTACATAAGTTATAGTG
GTAACACTAGGTACAACCCATCTCTCAAAAGTCGAATCTCTATCACTCGCGACACATCCAAGAACCAATT
CTTCCTGCAGTTGAACTCTGTGACTATTGAGGACACAGCCACATATTACTGTGTAACGGCGGGACGCGGG
TTTCCTTATTGGGGCCAAGGGACTCTGGTCACTGTCTCTGCAGGCGGTGGCGGTAGTGGGGGAGGCGGTT
CTGGCGGCGGAGGGTCCGGCGGTGGAGGATCAGACATCCTGATGACCCAATCTCCATCCTCCATGTCTGT
ATCTCTGGGAGACACAGTCAGCATCACTTGCCATTCAAGTCAGGACATTAACAGTAATATAGGGTGGTTG
CAGCAGAGACCAGGGAAATCATTTAAGGGCCTGATCTATCATGGAACCAACTTGGACGATGAAGTTCCAT
CAAGGTTCAGTGGCAGTGGATCTGGAGCCGATTATTCTCTCACCATCAGCAGCCTGGAATCTGAAGATTT
TGCAGACTATTACTGTGTACAGTATGCTCAGTTTCCGTGGACGTTCGGTGGAGGCACCAAGCTGGAAATC
AAA
>Seq ID 109: SI-95x49 light chain amino acid sequence
EIVLTQSPSTLSVSPGERATFSCRASQSIGTNIHWYQQKPGKPPRLLIKYASESISGIPDRESGSGSGTE
FTLTISSVQSEDFAVYYCQQNFNWPTTFGPGTKLTVLRTVAAPSVFI FPPSDEQLKSGTASVVCLLNNFY
PREAKVQWKVDNALQSGNSQESVTEQDSKDSTYSLSSTLTLSKADYEKHKVYACEVTHQGLSSPVTKSEN
RGEC
>Seq ID 110: SI-95x49 light chain nucleotide sequence
GAGATCGTGCTGACCCAGTCTCCTTCCACACTGTCTGTGTCTCCCGGCGAGAGAGCCACCTTCAGCTGTA
GAGCCTCTCAGTCCATCGGCACCAACATCCACTGGTATCAGCAGAAGCCCGGCAAGCCTCCTCGGCTGCT
GATTAAGTACGCCTCCGAGTCCATCAGCGGCATCCCTGACAGATTCTCCGGCTCTGGCTCTGGCACCGAG
TTTACCCTGACCATCTCCTCCGTGCAGTCCGAGGATTTCGCCGTGTACTACTGCCAGCAGAACTTCAACT
GGCCCACCACCTTTGGACCCGGCACCAAGCTGACCGTGCTGCGTACGGTGGCTGCACCATCTGTCTTCAT
CTTCCCGCCATCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAAC
AGGGGAGAGTGT
>Seq ID 111: SI-95m4 light chain amino acid sequence
DIVMTQSPSSVSLSLGDRVTITCHSSQDINSNIGWLQQKPGKAFKGLIYHGTNLDDGVPSRFSGSGSGAD
YTLTISSLQAEDFATYYCVQYAQFPWTFGPGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGEC
>Seq ID 112: SI-95m4 light chain nucleotide sequence
GACATCGTGATGACCCAATCTCCAAGCTCAGTGAGTCTGTCACTAGGCGACCGTGTCACCATTACATGTC
ATAGTTCCCAGGATATCAACAGTAATATTGGGTGGCTTCAGCAAAAGCCTGGTAAAGCCTTTAAAGGACT
GATTTATCACGGGACCAACTTGGACGATGGAGTTCCCTCCCGGTTTTCTGGCTCCGGGTCCGGTGCAGAC
TATACTTTGACAATTAGCTCTCTCCAGGCAGAGGACTTCGCTACATATTACTGCGTCCAGTATGCCCAGT
TCCCTTGGACCTTTGGACCCGGCACTAAACTGGAAATTAAACGTACGGTGGCTGCACCATCTGTCTTCAT
CTTCCCGCCATCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAAC
AGGGGAGAGTGT
>Seq ID 113: SI-95X52 heavy chain amino acid sequence
EIVLTQSPSTLSVSPGERATFSCRASQSIGTNIHWYQQKPGKPPRLLIKYASESISGIPDRFSGSGSGTE
FTLTISSVQSEDFAVYYCQQNFNWPTTFGPGTKLTVLGGGGSGGGGSGGGGSGGGGSQVQLQQSGPGLVK
PSETLSITCTVSGFSLTNYGVHWIRQAPGKGLEWLGVIWSGGNTDYNTPFTSRFTITKDNSKNQVYFKLR
SVRADDTAIYYCARALTYYDYEFAYWGQGTLVTVSSGGGGSGGGGSGGGGSGGGGSQVQLQESGPSLVKP
SQTLSLTCTVSGYSITSDFAWNWIRQFPGRKLEWMGYISYSGNTRYNPSLKSRFTISRDTSKNQFYLKLR
SVTIADTAVYYCVTAGRGFPYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHEALHNH
YTQKSLSLSPG
>Seq ID 114: SI-95X52 heavy chain nucleotide sequence
GAGATCGTGCTGACCCAGTCTCCTTCCACACTGTCTGTGTCTCCCGGCGAGAGAGCCACCTTCAGCTGTA
GAGCCTCTCAGTCCATCGGCACCAACATCCACTGGTATCAGCAGAAGCCCGGCAAGCCTCCTCGGCTGCT
GATTAAGTACGCCTCCGAGTCCATCAGCGGCATCCCTGACAGATTCTCCGGCTCTGGCTCTGGCACCGAG
TTTACCCTGACCATCTCCTCCGTGCAGTCCGAGGATTTCGCCGTGTACTACTGCCAGCAGAACTTCAACT
GGCCCACCACCTTTGGACCCGGCACCAAGCTGACAGTTCTTGGCGGTGGCGGTAGTGGGGGAGGCGGTTC
TGGCGGCGGAGGGTCCGGCGGTGGAGGATCACAAGTTCAGTTGCAGCAGTCTGGCCCTGGCCTGGTCAAG
CCTTCTGAGACACTGTCCATCACCTGTACCGTGTCCGGCTTCTCCCTGACCAATTACGGCGTGCACTGGA
TCAGACAGGCCCCTGGCAAAGGACTGGAATGGCTGGGAGTGATTTGGAGCGGCGGCAACACCGACTACAA
CACCCCTTTCACCAGCCGGTTCACCATCACCAAGGACAACTCCAAGAACCAGGTGTACTTCAAGCTGCGG
AGCGTGCGGGCTGATGACACCGCCATCTACTACTGTGCTCGGGCCCTGACCTACTACGACTACGAGTTTG
CTTACTGGGGCCAGGGCACCCTGGTCACAGTTTCTTCTGGCGGTGGCGGTAGTGGGGGAGGCGGTTCTGG
CGGCGGAGGGTCCGGCGGTGGAGGATCACAAGTGCAGCTCCAGGAGTCAGGCCCTAGCCTCGTGAAGCCC
TCCCAGACCCTGAGCCTGACCTGCACCGTATCTGGTTATAGTATTACAAGTGACTTCGCCTGGAACTGGA
TTAGGCAGTTTCCAGGTCGCAAGCTGGAGTGGATGGGGTATATCTCCTACTCCGGTAACACTCGATACAA
CCCTTCCCTCAAAAGCAGATTTACCATTTCCAGAGACACATCTAAGAACCAATTTTATCTGAAGTTGAGA
TCTGTTACCATCGCTGATACTGCTGTGTACTATTGCGTTACCGCCGGACGCGGCTTCCCCTATTGGGGGC
AGGGAACCCTGGTGACTGTATCATCCGCTAGCACCAAGGGCCCATCGGTCTTCCCCCTGGCACCCTCCTC
CAAGAGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGAC
TCTACTCCCTCAGCAGCGTGGTGACCGTGCCCTCCAGCAGCTTGGGCACCCAGACCTACATCTGCAACGT
GAATCACAAGCCCAGCAACACCAAGGTGGACAAGAGAGTTGAGCCCAAATCTTGTGACAAAACTCACACA
TGCCCACCGTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTATAGCAAGCTCACCGT
GGACAAGAGCAGGTGGCAGCAGGGGAACGTCTTCTCATGCTCCGTGATGCATGAGGCTCTGCACAACCAC
TACACGCAGAAGAGCCTCTCCCTGTCTCCGGGT
>Seq ID 115: SI-95X53 heavy chain amino acid sequence
EIVLTQSPSTLSVSPGERATFSCRASQSIGTNIHWYQQKPGKPPRLLIKYASESISGIPDRESGSGSGTE
FTLTISSVQSEDFAVYYCQQNNNWPTTFGPGTKLTVLGGGGSGGGGSGGGGSGGGGSQVQLQQSGPGLVK
PSETLSITCTVSGFSLTNYGVHWIRQAPGKGLEWLGVIWSGGNTDYNTPFTSRFTITKDNSKNQVYFKLR
SVRADDTAIYYCARALTAYDYEFAYWGQGTLVTVSSGGGGSGGGGSGGGGSGGGGSQVQLQESGPSLVKP
SQTLSLTCTVSGYSITSDFAWNWIRQFPGRKLEWMGYISYSGNTRYNPSLKSRFTISRDTSKNQFYLKLR
SVTIADTAVYYCVTAGRGFPYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHEALHNH
YTQKSLSLSPG
>Seq ID 116: SI-95X53 heavy chain nucleotide sequence
GAGATCGTGCTGACCCAGTCTCCTTCCACACTGTCTGTGTCTCCCGGCGAGAGAGCCACCTTCAGCTGTA
GAGCCTCTCAGTCCATCGGCACCAACATCCACTGGTATCAGCAGAAGCCCGGCAAGCCTCCTCGGCTGCT
GATTAAGTACGCCTCCGAGTCCATCAGCGGCATCCCTGACAGATTCTCCGGCTCTGGCTCTGGCACCGAG
TTTACCCTGACCATCTCCTCCGTGCAGTCCGAGGATTTCGCCGTGTACTACTGCCAGCAGAACAACAACT
GGCCCACCACCTTTGGACCCGGCACCAAGCTGACAGTTCTTGGCGGTGGCGGTAGTGGGGGAGGCGGTTC
TGGCGGCGGAGGGTCCGGCGGTGGAGGATCACAAGTTCAGTTGCAGCAGTCTGGCCCTGGCCTGGTCAAG
CCTTCTGAGACACTGTCCATCACCTGTACCGTGTCCGGCTTCTCCCTGACCAATTACGGCGTGCACTGGA
TCAGACAGGCCCCTGGCAAAGGACTGGAATGGCTGGGAGTGATTTGGAGCGGCGGCAACACCGACTACAA
CACCCCTTTCACCAGCCGGTTCACCATCACCAAGGACAACTCCAAGAACCAGGTGTACTTCAAGCTGCGG
AGCGTGCGGGCTGATGACACCGCCATCTACTACTGTGCTCGGGCCCTGACCGCCTACGACTACGAGTTTG
CTTACTGGGGCCAGGGCACCCTGGTCACAGTTTCTTCTGGCGGTGGCGGTAGTGGGGGAGGCGGTTCTGG
CGGCGGAGGGTCCGGCGGTGGAGGATCACAAGTGCAGCTCCAGGAGTCAGGCCCTAGCCTCGTGAAGCCC
TCCCAGACCCTGAGCCTGACCTGCACCGTATCTGGTTATAGTATTACAAGTGACTTCGCCTGGAACTGGA
TTAGGCAGTTTCCAGGTCGCAAGCTGGAGTGGATGGGGTATATCTCCTACTCCGGTAACACTCGATACAA
CCCTTCCCTCAAAAGCAGATTTACCATTTCCAGAGACACATCTAAGAACCAATTTTATCTGAAGTTGAGA
TCTGTTACCATCGCTGATACTGCTGTGTACTATTGCGTTACCGCCGGACGCGGCTTCCCCTATTGGGGGC
AGGGAACCCTGGTGACTGTATCATCCGCTAGCACCAAGGGCCCATCGGTCTTCCCCCTGGCACCCTCCTC
CAAGAGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGAC
TCTACTCCCTCAGCAGCGTGGTGACCGTGCCCTCCAGCAGCTTGGGCACCCAGACCTACATCTGCAACGT
GAATCACAAGCCCAGCAACACCAAGGTGGACAAGAGAGTTGAGCCCAAATCTTGTGACAAAACTCACACA
TGCCCACCGTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTATAGCAAGCTCACCGT
GGACAAGAGCAGGTGGCAGCAGGGGAACGTCTTCTCATGCTCCGTGATGCATGAGGCTCTGCACAACCAC
TACACGCAGAAGAGCCTCTCCCTGTCTCCGGGT
>Seq ID 117: SI-95X54 heavy chain amino acid sequence
EIVLTQSPSTLSVSPGERATFSCRASQSIGTNIHWYQQKPGKPPRLLIKYASESISGIPDRESGSGSGTE
FTLTISSVQSEDFAVYYCQQNNNWPTTFGPGTKLTVLGGGGSGGGGSGGGGSGGGGSQVQLQQSGPGLVK
PSETLSITCTVSGFSLTNYGVHWIRQAPGKGLEWLGVIWSGGNTDYNTPFTSRFTITKDNSKNQVYFKLR
SVRADDTAIYYCARALTYADYEFAYWGQGTLVTVSSGGGGSGGGGSGGGGSGGGGSQVQLQESGPSLVKP
SQTLSLTCTVSGYSITSDFAWNWIRQFPGRKLEWMGYISYSGNTRYNPSLKSRFTISRDTSKNQFYLKLR
SVTIADTAVYYCVTAGRGFPYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHEALHNH
YTQKSLSLSPG
>Seq ID 118: SI-95X54 heavy chain nucleotide sequence
GAGATCGTGCTGACCCAGTCTCCTTCCACACTGTCTGTGTCTCCCGGCGAGAGAGCCACCTTCAGCTGTA
GAGCCTCTCAGTCCATCGGCACCAACATCCACTGGTATCAGCAGAAGCCCGGCAAGCCTCCTCGGCTGCT
GATTAAGTACGCCTCCGAGTCCATCAGCGGCATCCCTGACAGATTCTCCGGCTCTGGCTCTGGCACCGAG
TTTACCCTGACCATCTCCTCCGTGCAGTCCGAGGATTTCGCCGTGTACTACTGCCAGCAGAACAACAACT
GGCCCACCACCTTTGGACCCGGCACCAAGCTGACAGTTCTTGGCGGTGGCGGTAGTGGGGGAGGCGGTTC
TGGCGGCGGAGGGTCCGGCGGTGGAGGATCACAAGTTCAGTTGCAGCAGTCTGGCCCTGGCCTGGTCAAG
CCTTCTGAGACACTGTCCATCACCTGTACCGTGTCCGGCTTCTCCCTGACCAATTACGGCGTGCACTGGA
TCAGACAGGCCCCTGGCAAAGGACTGGAATGGCTGGGAGTGATTTGGAGCGGCGGCAACACCGACTACAA
CACCCCTTTCACCAGCCGGTTCACCATCACCAAGGACAACTCCAAGAACCAGGTGTACTTCAAGCTGCGG
AGCGTGCGGGCTGATGACACCGCCATCTACTACTGTGCTCGGGCCCTGACCTACGCCGACTACGAGTTTG
CTTACTGGGGCCAGGGCACCCTGGTCACAGTTTCTTCTGGCGGTGGCGGTAGTGGGGGAGGCGGTTCTGG
CGGCGGAGGGTCCGGCGGTGGAGGATCACAAGTGCAGCTCCAGGAGTCAGGCCCTAGCCTCGTGAAGCCC
TCCCAGACCCTGAGCCTGACCTGCACCGTATCTGGTTATAGTATTACAAGTGACTTCGCCTGGAACTGGA
TTAGGCAGTTTCCAGGTCGCAAGCTGGAGTGGATGGGGTATATCTCCTACTCCGGTAACACTCGATACAA
CCCTTCCCTCAAAAGCAGATTTACCATTTCCAGAGACACATCTAAGAACCAATTTTATCTGAAGTTGAGA
TCTGTTACCATCGCTGATACTGCTGTGTACTATTGCGTTACCGCCGGACGCGGCTTCCCCTATTGGGGGC
AGGGAACCCTGGTGACTGTATCATCCGCTAGCACCAAGGGCCCATCGGTCTTCCCCCTGGCACCCTCCTC
CAAGAGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGAC
TCTACTCCCTCAGCAGCGTGGTGACCGTGCCCTCCAGCAGCTTGGGCACCCAGACCTACATCTGCAACGT
GAATCACAAGCCCAGCAACACCAAGGTGGACAAGAGAGTTGAGCCCAAATCTTGTGACAAAACTCACACA
TGCCCACCGTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTATAGCAAGCTCACCGT
GGACAAGAGCAGGTGGCAGCAGGGGAACGTCTTCTCATGCTCCGTGATGCATGAGGCTCTGCACAACCAC
TACACGCAGAAGAGCCTCTCCCTGTCTCCGGGT
>Seq ID 119: SI-95X55 heavy chain amino acid sequence
EIVLTQSPSTLSVSPGERATFSCRASQSIGTNIHWYQQKPGKPPRLLIKYASESISGIPDRFSGSGSGTE
FTLTISSVQSEDFAVYYCQQNNNWPTTFGPGTKLTVLGGGGSGGGGSGGGGSGGGGSQVQLQQSGPGLVK
PSETLSITCTVSGFSLTNYGVHWIRQAPGKGLEWLGVIWSGGNTDYNTPFTSRFTITKDNSKNQVYFKLR
SVRADDTAIYYCARALTYYYYEFAYWGQGTLVTVSSGGGGSGGGGSGGGGSGGGGSQVQLQESGPSLVKP
SQTLSLTCTVSGYSITSDFAWNWIRQFPGRKLEWMGYISYSGNTRYNPSLKSRFTISRDTSKNQFYLKLR
SVTIADTAVYYCVTAGRGFPYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHEALHNH
YTQKSLSLSPG
>Seq ID 120: SI-95X55 heavy chain nucleotide sequence
GAGATCGTGCTGACCCAGTCTCCTTCCACACTGTCTGTGTCTCCCGGCGAGAGAGCCACCTTCAGCTGTA
GAGCCTCTCAGTCCATCGGCACCAACATCCACTGGTATCAGCAGAAGCCCGGCAAGCCTCCTCGGCTGCT
GATTAAGTACGCCTCCGAGTCCATCAGCGGCATCCCTGACAGATTCTCCGGCTCTGGCTCTGGCACCGAG
TTTACCCTGACCATCTCCTCCGTGCAGTCCGAGGATTTCGCCGTGTACTACTGCCAGCAGAACAACAACT
GGCCCACCACCTTTGGACCCGGCACCAAGCTGACAGTTCTTGGCGGTGGCGGTAGTGGGGGAGGCGGTTC
TGGCGGCGGAGGGTCCGGCGGTGGAGGATCACAAGTTCAGTTGCAGCAGTCTGGCCCTGGCCTGGTCAAG
CCTTCTGAGACACTGTCCATCACCTGTACCGTGTCCGGCTTCTCCCTGACCAATTACGGCGTGCACTGGA
TCAGACAGGCCCCTGGCAAAGGACTGGAATGGCTGGGAGTGATTTGGAGCGGCGGCAACACCGACTACAA
CACCCCTTTCACCAGCCGGTTCACCATCACCAAGGACAACTCCAAGAACCAGGTGTACTTCAAGCTGCGG
AGCGTGCGGGCTGATGACACCGCCATCTACTACTGTGCTCGGGCCCTGACCTACTACTACTACGAGTTTG
CTTACTGGGGCCAGGGCACCCTGGTCACAGTTTCTTCTGGCGGTGGCGGTAGTGGGGGAGGCGGTTCTGG
CGGCGGAGGGTCCGGCGGTGGAGGATCACAAGTGCAGCTCCAGGAGTCAGGCCCTAGCCTCGTGAAGCCC
TCCCAGACCCTGAGCCTGACCTGCACCGTATCTGGTTATAGTATTACAAGTGACTTCGCCTGGAACTGGA
TTAGGCAGTTTCCAGGTCGCAAGCTGGAGTGGATGGGGTATATCTCCTACTCCGGTAACACTCGATACAA
CCCTTCCCTCAAAAGCAGATTTACCATTTCCAGAGACACATCTAAGAACCAATTTTATCTGAAGTTGAGA
TCTGTTACCATCGCTGATACTGCTGTGTACTATTGCGTTACCGCCGGACGCGGCTTCCCCTATTGGGGGC
AGGGAACCCTGGTGACTGTATCATCCGCTAGCACCAAGGGCCCATCGGTCTTCCCCCTGGCACCCTCCTC
CAAGAGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGAC
TCTACTCCCTCAGCAGCGTGGTGACCGTGCCCTCCAGCAGCTTGGGCACCCAGACCTACATCTGCAACGT
GAATCACAAGCCCAGCAACACCAAGGTGGACAAGAGAGTTGAGCCCAAATCTTGTGACAAAACTCACACA
TGCCCACCGTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTATAGCAAGCTCACCGT
GGACAAGAGCAGGTGGCAGCAGGGGAACGTCTTCTCATGCTCCGTGATGCATGAGGCTCTGCACAACCAC
TACACGCAGAAGAGCCTCTCCCTGTCTCCGGGT
>Seq ID 121: SI-95X56 heavy chain amino acid sequence
EIVLTQSPSTLSVSPGERATFSCRASQSIGTNIHWYQQKPGKPPRLLIKYASESISGIPDRFSGSGSGTE
FTLTISSVQSEDFAVYYCQQNNNWPTTFGPGTKLTVLGGGGSGGGGSGGGGSGGGGSQVQLQQSGPGLVK
PSETLSITCTVSGFSLTNYGVHWIRQAPGKGLEWLGVIWSGGNTDYNTPFTSRFTITKDNSKNQVYFKLR
SVRADDTAIYYCARALTYYDLEFAYWGQGTLVTVSSGGGGSGGGGSGGGGSGGGGSQVQLQESGPSLVKP
SQTLSLTCTVSGYSITSDFAWNWIRQFPGRKLEWMGYISYSGNTRYNPSLKSRFTISRDTSKNQFYLKLR
SVTIADTAVYYCVTAGRGFPYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHEALHNH
YTQKSLSLSPG
>Seq ID 122: SI-95X56 heavy chain nucleotide sequence
GAGATCGTGCTGACCCAGTCTCCTTCCACACTGTCTGTGTCTCCCGGCGAGAGAGCCACCTTCAGCTGTA
GAGCCTCTCAGTCCATCGGCACCAACATCCACTGGTATCAGCAGAAGCCCGGCAAGCCTCCTCGGCTGCT
GATTAAGTACGCCTCCGAGTCCATCAGCGGCATCCCTGACAGATTCTCCGGCTCTGGCTCTGGCACCGAG
TTTACCCTGACCATCTCCTCCGTGCAGTCCGAGGATTTCGCCGTGTACTACTGCCAGCAGAACAACAACT
GGCCCACCACCTTTGGACCCGGCACCAAGCTGACAGTTCTTGGCGGTGGCGGTAGTGGGGGAGGCGGTTC
TGGCGGCGGAGGGTCCGGCGGTGGAGGATCACAAGTTCAGTTGCAGCAGTCTGGCCCTGGCCTGGTCAAG
CCTTCTGAGACACTGTCCATCACCTGTACCGTGTCCGGCTTCTCCCTGACCAATTACGGCGTGCACTGGA
TCAGACAGGCCCCTGGCAAAGGACTGGAATGGCTGGGAGTGATTTGGAGCGGCGGCAACACCGACTACAA
CACCCCTTTCACCAGCCGGTTCACCATCACCAAGGACAACTCCAAGAACCAGGTGTACTTCAAGCTGCGG
AGCGTGCGGGCTGATGACACCGCCATCTACTACTGTGCTCGGGCCCTGACCTACTACGACCTCGAGTTTG
CTTACTGGGGCCAGGGCACCCTGGTCACAGTTTCTTCTGGCGGTGGCGGTAGTGGGGGAGGCGGTTCTGG
CGGCGGAGGGTCCGGCGGTGGAGGATCACAAGTGCAGCTCCAGGAGTCAGGCCCTAGCCTCGTGAAGCCC
TCCCAGACCCTGAGCCTGACCTGCACCGTATCTGGTTATAGTATTACAAGTGACTTCGCCTGGAACTGGA
TTAGGCAGTTTCCAGGTCGCAAGCTGGAGTGGATGGGGTATATCTCCTACTCCGGTAACACTCGATACAA
CCCTTCCCTCAAAAGCAGATTTACCATTTCCAGAGACACATCTAAGAACCAATTTTATCTGAAGTTGAGA
TCTGTTACCATCGCTGATACTGCTGTGTACTATTGCGTTACCGCCGGACGCGGCTTCCCCTATTGGGGGC
AGGGAACCCTGGTGACTGTATCATCCGCTAGCACCAAGGGCCCATCGGTCTTCCCCCTGGCACCCTCCTC
CAAGAGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGAC
TCTACTCCCTCAGCAGCGTGGTGACCGTGCCCTCCAGCAGCTTGGGCACCCAGACCTACATCTGCAACGT
GAATCACAAGCCCAGCAACACCAAGGTGGACAAGAGAGTTGAGCCCAAATCTTGTGACAAAACTCACACA
TGCCCACCGTGCCCAGCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTATAGCAAGCTCACCGT
GGACAAGAGCAGGTGGCAGCAGGGGAACGTCTTCTCATGCTCCGTGATGCATGAGGCTCTGCACAACCAC
TACACGCAGAAGAGCCTCTCCCTGTCTCCGGGT
>Seq ID 123: SI-95m5 heavy chain amino acid sequence
QVQLQQSGPGLVKPSETLSITCTVSGFSLTNYGVHWIRQAPGKGLEWLGVIWSGGNTDYNTPFTSRFTIT
KDNSKNQVYFKLRSVRADDTAIYYCARALTYYDYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 124: SI-95m5 heavy chain nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCTACTACGACTACGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTTAG
>Seq ID 125: SI-95X57 light chain amino acid sequence
EIVLTQSPSTLSVSPGERATFSCRASQSIGTNIHWYQQKPGKPPRLLIKYASESISGIPDRFSGSGSGTE
FTLTISSVQSEDFAVYYCQQNNNWPTTFGPGTKLTVLRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGECGGGGSGGGGGSGGGGSGGGGSQVQLQESGPSLVKPSQTLSLTCTVSGYSITSDFAWNWIRQFPGRK
LEWMGYISYSGNTRYNPSLKSRFTISRDTSKNQFYLKLRSVTIADTAVYYCVTAGRGFPYWGQGTLVTVS
SGGGGSGGGGSGGGGSDIVMTQSPSSVSLSLGDRVTITCHSSQDINSNIGWLQQKPGKAFKGLIYHGTNL
DDGVPSRFSGSGSGADYTLTISSLQAEDFATYYCVQYAQFPWTFGPGTKLEIK
>Seq ID 126: SI-95X50 light chain nucleotide sequence
GAGATCGTGCTGACCCAGTCTCCTTCCACACTGTCTGTGTCTCCCGGCGAGAGAGCCACCTTCAGCTGTA
GAGCCTCTCAGTCCATCGGCACCAACATCCACTGGTATCAGCAGAAGCCCGGCAAGCCTCCTCGGCTGCT
GATTAAGTACGCCTCCGAGTCCATCAGCGGCATCCCTGACAGATTCTCCGGCTCTGGCTCTGGCACCGAG
TTTACCCTGACCATCTCCTCCGTGCAGTCCGAGGATTTCGCCGTGTACTACTGCCAGCAGAACAACAACT
GGCCCACCACCTTTGGACCCGGCACCAAGCTGACAGTTCTTCGTACGGTGGCTGCACCATCTGTCTTCAT
CTTCCCGCCATCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAAC
AGGGGAGAGTGTGGCGGTGGCGGATCCGGAGGAGGCGGTGGAAGCGGAGGCGGTGGCTCTGGAGGAGGCG
GTTCGCAAGTGCAGCTCCAGGAGTCAGGCCCTAGCCTCGTGAAGCCCTCCCAGACCCTGAGCCTGACCTG
CACCGTATCTGGTTATAGTATTACAAGTGACTTCGCCTGGAACTGGATTAGGCAGTTTCCAGGTCGCAAG
CTGGAGTGGATGGGGTATATCTCCTACTCCGGTAACACTCGATACAACCCTTCCCTCAAAAGCAGATTTA
CCATTTCCAGAGACACATCTAAGAACCAATTTTATCTGAAGTTGAGATCTGTTACCATCGCTGATACTGC
TGTGTACTATTGCGTTACCGCCGGACGCGGCTTCCCCTATTGGGGGCAGGGAACCCTGGTGACTGTATCA
TCCGGTGGAGGAGGCAGTGGTGGTGGAGGAAGCGGAGGTGGTGGCAGCGACATCGTGATGACCCAATCTC
CAAGCTCAGTGAGTCTGTCACTAGGCGACCGTGTCACCATTACATGTCATAGTTCCCAGGATATCAACAG
TAATATTGGGTGGCTTCAGCAAAAGCCTGGTAAAGCCTTTAAAGGACTGATTTATCACGGGACCAACTTG
GACGATGGAGTTCCCTCCCGGTTTTCTGGCTCCGGGTCCGGTGCAGACTATACTTTGACAATTAGCTCTC
TCCAGGCAGAGGACTTCGCTACATATTACTGCGTCCAGTATGCCCAGTTCCCTTGGACCTTTGGACCCGG
CACTAAACTGGAAATTAAA
>Seq ID 127: SI-95X58 heavy chain amino acid sequence
QVQLQQSGPGLVKPSETLSITCTVSGFSLTNYGVHWIRQAPGKGLEWLGVIWSGGNTDYNTPFTSRFTIT
KDNSKNQVYFKLRSVRADDTAIYYCARALTAYDYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 128: SI-95X58 heavy chain nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCGCCTACGACTACGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTTAG
>Seq ID 129: SI-95X59 heavy chain amino acid sequence
QVQLQQSGPGLVKPSETLSITCTVSGFSLTNYGVHWIRQAPGKGLEWLGVIWSGGNTDYNTPFTSRFTIT
KDNSKNQVYFKLRSVRADDTAIYYCARALTYADYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 130: SI-95X59 heavy chain nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCTACGCCGACTACGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTTAG
>Seq ID 131: SI-95X60 heavy chain amino acid sequence
QVQLQQSGPGLVKPSETLSITCTVSGFSLTNYGVHWIRQAPGKGLEWLGVIWSGGNTDYNTPFTSRFTIT
KDNSKNQVYFKLRSVRADDTAIYYCARALTYYYYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 132: SI-95X60 heavy chain nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCTACTACTACTACGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTTAG
>Seq ID 133: SI-95X61 heavy chain amino acid sequence
QVQLQQSGPGLVKPSETLSITCTVSGFSLTNYGVHWIRQAPGKGLEWLGVIWSGGNTDYNTPFTSRFTIT
KDNSKNQVYFKLRSVRADDTAIYYCARALTYYDLEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 134: SI-95X61 heavy chain nucleotide sequence
CAAGTTCAGTTGCAGCAGTCTGGCCCTGGCCTGGTCAAGCCTTCTGAGACACTGTCCATCACCTGTACCG
TGTCCGGCTTCTCCCTGACCAATTACGGCGTGCACTGGATCAGACAGGCCCCTGGCAAAGGACTGGAATG
GCTGGGAGTGATTTGGAGCGGCGGCAACACCGACTACAACACCCCTTTCACCAGCCGGTTCACCATCACC
AAGGACAACTCCAAGAACCAGGTGTACTTCAAGCTGCGGAGCGTGCGGGCTGATGACACCGCCATCTACT
ACTGTGCTCGGGCCCTGACCTACTACGACCTCGAGTTTGCTTACTGGGGCCAGGGCACCCTGGTCACAGT
TTCTTCTGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGTTAG
>Seq ID 135: SI-95X62 light chain amino acid sequence
EIVLTQSPSTLSVSPGERATFSCRASQSIGTNIHWYQQKPGKPPRLLIKYASESISGIPDRFSGSGSGTE
FTLTISSVQSEDFAVYYCQQNFNWPTTFGPGTKLTVLRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGECGGGGSGGGGGSGGGGSGGGGSQVQLQESGPSLVKPSQTLSLTCTVSGYSITSDFAWNWIRQFPGRK
LEWMGYISYSGNTRYNPSLKSRFTISRDTSKNQFYLKLRSVTIADTAVYYCVTAGRGFPYWGQGTLVTVS
SGGGGSGGGGSGGGGSDIVMTQSPSSVSLSLGDRVTITCHSSQDINSNIGWLQQKPGKAFKGLIYHGTNL
DDGVPSRFSGSGSGADYTLTISSLQAEDFATYYCVQYAQFPWTFGPGTKLEIK
>Seq ID 136: SI-95X62 light chain nucleotide sequence
GAGATCGTGCTGACCCAGTCTCCTTCCACACTGTCTGTGTCTCCCGGCGAGAGAGCCACCTTCAGCTGTA
GAGCCTCTCAGTCCATCGGCACCAACATCCACTGGTATCAGCAGAAGCCCGGCAAGCCTCCTCGGCTGCT
GATTAAGTACGCCTCCGAGTCCATCAGCGGCATCCCTGACAGATTCTCCGGCTCTGGCTCTGGCACCGAG
TTTACCCTGACCATCTCCTCCGTGCAGTCCGAGGATTTCGCCGTGTACTACTGCCAGCAGAACTTCAACT
GGCCCACCACCTTTGGACCCGGCACCAAGCTGACCGTGCTGCGTACGGTGGCTGCACCATCTGTCTTCAT
CTTCCCGCCATCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAAC
AGGGGAGAGTGTGGCGGTGGCGGATCCGGAGGAGGCGGTGGAAGCGGAGGCGGTGGCTCTGGAGGAGGCG
GTTCGCAAGTGCAGCTCCAGGAGTCAGGCCCTAGCCTCGTGAAGCCCTCCCAGACCCTGAGCCTGACCTG
CACCGTATCTGGTTATAGTATTACAAGTGACTTCGCCTGGAACTGGATTAGGCAGTTTCCAGGTCGCAAG
CTGGAGTGGATGGGGTATATCTCCTACTCCGGTAACACTCGATACAACCCTTCCCTCAAAAGCAGATTTA
CCATTTCCAGAGACACATCTAAGAACCAATTTTATCTGAAGTTGAGATCTGTTACCATCGCTGATACTGC
TGTGTACTATTGCGTTACCGCCGGACGCGGCTTCCCCTATTGGGGGCAGGGAACCCTGGTGACTGTATCA
TCCGGTGGAGGAGGCAGTGGTGGTGGAGGAAGCGGAGGTGGTGGCAGCGACATCGTGATGACCCAATCTC
CAAGCTCAGTGAGTCTGTCACTAGGCGACCGTGTCACCATTACATGTCATAGTTCCCAGGATATCAACAG
TAATATTGGGTGGCTTCAGCAAAAGCCTGGTAAAGCCTTTAAAGGACTGATTTATCACGGGACCAACTTG
GACGATGGAGTTCCCTCCCGGTTTTCTGGCTCCGGGTCCGGTGCAGACTATACTTTGACAATTAGCTCTC
TCCAGGCAGAGGACTTCGCTACATATTACTGCGTCCAGTATGCCCAGTTCCCTTGGACCTTTGGACCCGG
CACTAAACTGGAAATTAAA
>Seq ID 137: SI-95X63 heavy chain amino acid sequence
DVQLQESGPSLVKPSQSLSLTCTVTGYSITSDFAWNWIRQFPGNKLEWMGYISYSGNTRYNPSLKSRISI
TRDTSKNQFFLQLNSVTIEDTATYYCVTAGRGFPYWGQGTLVTVSAASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGGGGGSGGGGSQVQLQQSGPGLVKPSETLSITCTVSGFSLTNYGVH
WIRQAPGKGLEWLGVIWSGGNTDYNTPFTSRFTITKDNSKNQVYFKLRSVRADDTAIYYCARALTYYDYE
FAYWGQGTLVTVSSGGGGSGGGGSGGGGSGGGGSEIVLTQSPSTLSVSPGERATFSCRASQSIGTNIHWY
QQKPGKPPRLLIKYASESISGIPDRFSGSGSGTEFTLTISSVQSEDFAVYYCQQNNNWPTTFGPGTKLTV
L
>Seq ID 138: SI-95X63 heavy chain nucleotide sequence
GATGTGCAGCTTCAGGAGTCGGGACCTAGCCTGGTGAAACCTTCTCAGTCTCTGTCCCTCACCTGCACTG
TCACTGGCTACTCAATCACCAGTGATTTTGCCTGGAACTGGATTCGGCAGTTTCCAGGAAACAAGCTGGA
GTGGATGGGCTACATAAGTTATAGTGGTAACACTAGGTACAACCCATCTCTCAAAAGTCGAATCTCTATC
ACTCGCGACACATCCAAGAACCAATTCTTCCTGCAGTTGAACTCTGTGACTATTGAGGACACAGCCACAT
ATTACTGTGTAACGGCGGGACGCGGGTTTCCTTATTGGGGCCAAGGGACTCTGGTCACTGTCTCTGCAGC
TAGCACCAAGGGCCCATCGGTCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCAGCGTGGTGACCGT
GCCCTCCAGCAGCTTGGGCACCCAGACCTACATCTGCAACGTGAATCACAAGCCCAGCAACACCAAGGTG
GACAAGAGAGTTGAGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCT
GGACTCCGACGGCTCCTTCTTCCTCTATAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTCCCTGTCTC
CGGGTGGCGGTGGAGGGTCCGGCGGTGGTGGATCCCAAGTTCAGTTGCAGCAGTCTGGCCCTGGCCTGGT
CAAGCCTTCTGAGACACTGTCCATCACCTGTACCGTGTCCGGCTTCTCCCTGACCAATTACGGCGTGCAC
TGGATCAGACAGGCCCCTGGCAAAGGACTGGAATGGCTGGGAGTGATTTGGAGCGGCGGCAACACCGACT
ACAACACCCCTTTCACCAGCCGGTTCACCATCACCAAGGACAACTCCAAGAACCAGGTGTACTTCAAGCT
GCGGAGCGTGCGGGCTGATGACACCGCCATCTACTACTGTGCTCGGGCCCTGACCTACTACGACTACGAG
TTTGCTTACTGGGGCCAGGGCACCCTGGTCACAGTTTCTTCTGGCGGTGGCGGTAGTGGGGGAGGCGGTT
CTGGCGGCGGAGGGTCCGGCGGTGGAGGATCAGAGATCGTGCTGACCCAGTCTCCTTCCACACTGTCTGT
GTCTCCCGGCGAGAGAGCCACCTTCAGCTGTAGAGCCTCTCAGTCCATCGGCACCAACATCCACTGGTAT
CAGCAGAAGCCCGGCAAGCCTCCTCGGCTGCTGATTAAGTACGCCTCCGAGTCCATCAGCGGCATCCCTG
ACAGATTCTCCGGCTCTGGCTCTGGCACCGAGTTTACCCTGACCATCTCCTCCGTGCAGTCCGAGGATTT
CGCCGTGTACTACTGCCAGCAGAACAACAACTGGCCCACCACCTTTGGACCCGGCACCAAGCTGACAGTT
CTTTGA
>Seq ID 139: SI-95X64 heavy chain amino acid sequence
DVQLQESGPSLVKPSQSLSLTCTVTGYSITSDFAWNWIRQFPGNKLEWMGYISYSGNTRYNPSLKSRISI
TRDTSKNQFFLQLNSVTIEDTATYYCVTAGRGFPYWGQGTLVTVSAASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGGGGGSGGGGSQVQLQQSGPGLVKPSETLSITCTVSGFSLTNYGVH
WIRQAPGKGLEWLGVIWSGGNTDYNTPFTSRFTITKDNSKNQVYFKLRSVRADDTAIYYCARALTYYDYE
FAYWGQGTLVTVSSGGGGSGGGGSGGGGSGGGGSEIVLTQSPSTLSVSPGERATFSCRASQSIGTNIHWY
QQKPGKPPRLLIKYASESISGIPDRFSGSGSGTEFTLTISSVQSEDFAVYYCQQNFNWPTTFGPGTKLTV
L
>Seq ID 140: SI-95X64 heavy chain nucleotide sequence
GATGTGCAGCTTCAGGAGTCGGGACCTAGCCTGGTGAAACCTTCTCAGTCTCTGTCCCTCACCTGCACTG
TCACTGGCTACTCAATCACCAGTGATTTTGCCTGGAACTGGATTCGGCAGTTTCCAGGAAACAAGCTGGA
GTGGATGGGCTACATAAGTTATAGTGGTAACACTAGGTACAACCCATCTCTCAAAAGTCGAATCTCTATC
ACTCGCGACACATCCAAGAACCAATTCTTCCTGCAGTTGAACTCTGTGACTATTGAGGACACAGCCACAT
ATTACTGTGTAACGGCGGGACGCGGGTTTCCTTATTGGGGCCAAGGGACTCTGGTCACTGTCTCTGCAGC
TAGCACCAAGGGCCCATCGGTCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCAGCGTGGTGACCGT
GCCCTCCAGCAGCTTGGGCACCCAGACCTACATCTGCAACGTGAATCACAAGCCCAGCAACACCAAGGTG
GACAAGAGAGTTGAGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCT
GGACTCCGACGGCTCCTTCTTCCTCTATAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTCCCTGTCTC
CGGGTGGCGGTGGAGGGTCCGGCGGTGGTGGATCCCAAGTTCAGTTGCAGCAGTCTGGCCCTGGCCTGGT
CAAGCCTTCTGAGACACTGTCCATCACCTGTACCGTGTCCGGCTTCTCCCTGACCAATTACGGCGTGCAC
TGGATCAGACAGGCCCCTGGCAAAGGACTGGAATGGCTGGGAGTGATTTGGAGCGGCGGCAACACCGACT
ACAACACCCCTTTCACCAGCCGGTTCACCATCACCAAGGACAACTCCAAGAACCAGGTGTACTTCAAGCT
GCGGAGCGTGCGGGCTGATGACACCGCCATCTACTACTGTGCTCGGGCCCTGACCTACTACGACTACGAG
TTTGCTTACTGGGGCCAGGGCACCCTGGTCACAGTTTCTTCTGGCGGTGGCGGTAGTGGGGGAGGCGGTT
CTGGCGGCGGAGGGTCCGGCGGTGGAGGATCAGAGATCGTGCTGACCCAGTCTCCTTCCACACTGTCTGT
GTCTCCCGGCGAGAGAGCCACCTTCAGCTGTAGAGCCTCTCAGTCCATCGGCACCAACATCCACTGGTAT
CAGCAGAAGCCCGGCAAGCCTCCTCGGCTGCTGATTAAGTACGCCTCCGAGTCCATCAGCGGCATCCCTG
ACAGATTCTCCGGCTCTGGCTCTGGCACCGAGTTTACCCTGACCATCTCCTCCGTGCAGTCCGAGGATTT
CGCCGTGTACTACTGCCAGCAGAACTTCAACTGGCCCACCACCTTTGGACCCGGCACCAAGCTGACAGTT
CTT
>Seq ID 141: SI-95X65 heavy chain amino acid sequence
DVQLQESGPSLVKPSQSLSLTCTVTGYSITSDFAWNWIRQFPGNKLEWMGYISYSGNTRYNPSLKSRISI
TRDTSKNQFFLQLNSVTIEDTATYYCVTAGRGFPYWGQGTLVTVSAASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGGGGGSGGGGSQVQLQQSGPGLVKPSETLSITCTVSGFSLTNYGVH
WIRQAPGKGLEWLGVIWSGGNTDYNTPFTSRFTITKDNSKNQVYFKLRSVRADDTAIYYCARALTAYDYE
FAYWGQGTLVTVSSGGGGSGGGGSGGGGSGGGGSEIVLTQSPSTLSVSPGERATFSCRASQSIGTNIHWY
QQKPGKPPRLLIKYASESISGIPDRFSGSGSGTEFTLTISSVQSEDFAVYYCQQNNNWPTTFGPGTKLTV
L
>Seq ID 142: SI-95X65 heavy chain nucleotide sequence
GATGTGCAGCTTCAGGAGTCGGGACCTAGCCTGGTGAAACCTTCTCAGTCTCTGTCCCTCACCTGCACTG
TCACTGGCTACTCAATCACCAGTGATTTTGCCTGGAACTGGATTCGGCAGTTTCCAGGAAACAAGCTGGA
GTGGATGGGCTACATAAGTTATAGTGGTAACACTAGGTACAACCCATCTCTCAAAAGTCGAATCTCTATC
ACTCGCGACACATCCAAGAACCAATTCTTCCTGCAGTTGAACTCTGTGACTATTGAGGACACAGCCACAT
ATTACTGTGTAACGGCGGGACGCGGGTTTCCTTATTGGGGCCAAGGGACTCTGGTCACTGTCTCTGCAGC
TAGCACCAAGGGCCCATCGGTCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCAGCGTGGTGACCGT
GCCCTCCAGCAGCTTGGGCACCCAGACCTACATCTGCAACGTGAATCACAAGCCCAGCAACACCAAGGTG
GACAAGAGAGTTGAGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCT
GGACTCCGACGGCTCCTTCTTCCTCTATAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTCCCTGTCTC
CGGGTGGCGGTGGAGGGTCCGGCGGTGGTGGATCCCAAGTTCAGTTGCAGCAGTCTGGCCCTGGCCTGGT
CAAGCCTTCTGAGACACTGTCCATCACCTGTACCGTGTCCGGCTTCTCCCTGACCAATTACGGCGTGCAC
TGGATCAGACAGGCCCCTGGCAAAGGACTGGAATGGCTGGGAGTGATTTGGAGCGGCGGCAACACCGACT
ACAACACCCCTTTCACCAGCCGGTTCACCATCACCAAGGACAACTCCAAGAACCAGGTGTACTTCAAGCT
GCGGAGCGTGCGGGCTGATGACACCGCCATCTACTACTGTGCTCGGGCCCTGACCGCCTACGACTACGAG
TTTGCTTACTGGGGCCAGGGCACCCTGGTCACAGTTTCTTCTGGCGGTGGCGGTAGTGGGGGAGGCGGTT
CTGGCGGCGGAGGGTCCGGCGGTGGAGGATCAGAGATCGTGCTGACCCAGTCTCCTTCCACACTGTCTGT
GTCTCCCGGCGAGAGAGCCACCTTCAGCTGTAGAGCCTCTCAGTCCATCGGCACCAACATCCACTGGTAT
CAGCAGAAGCCCGGCAAGCCTCCTCGGCTGCTGATTAAGTACGCCTCCGAGTCCATCAGCGGCATCCCTG
ACAGATTCTCCGGCTCTGGCTCTGGCACCGAGTTTACCCTGACCATCTCCTCCGTGCAGTCCGAGGATTT
CGCCGTGTACTACTGCCAGCAGAACAACAACTGGCCCACCACCTTTGGACCCGGCACCAAGCTGACAGTT
CTT
>Seq ID 143: SI-95X66 heavy chain amino acid sequence
DVQLQESGPSLVKPSQSLSLTCTVTGYSITSDFAWNWIRQFPGNKLEWMGYISYSGNTRYNPSLKSRISI
TRDTSKNQFFLQLNSVTIEDTATYYCVTAGRGFPYWGQGTLVTVSAASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGGGGGSGGGGSQVQLQQSGPGLVKPSETLSITCTVSGFSLTNYGVH
WIRQAPGKGLEWLGVIWSGGNTDYNTPFTSRFTITKDNSKNQVYFKLRSVRADDTAIYYCARALTYADYE
FAYWGQGTLVTVSSGGGGSGGGGSGGGGSGGGGSEIVLTQSPSTLSVSPGERATFSCRASQSIGTNIHWY
QQKPGKPPRLLIKYASESISGIPDRFSGSGSGTEFTLTISSVQSEDFAVYYCQQNNNWPTTFGPGTKLTV
L
>Seq ID 144: SI-95X66 heavy chain nucleotide sequence
GATGTGCAGCTTCAGGAGTCGGGACCTAGCCTGGTGAAACCTTCTCAGTCTCTGTCCCTCACCTGCACTG
TCACTGGCTACTCAATCACCAGTGATTTTGCCTGGAACTGGATTCGGCAGTTTCCAGGAAACAAGCTGGA
GTGGATGGGCTACATAAGTTATAGTGGTAACACTAGGTACAACCCATCTCTCAAAAGTCGAATCTCTATC
ACTCGCGACACATCCAAGAACCAATTCTTCCTGCAGTTGAACTCTGTGACTATTGAGGACACAGCCACAT
ATTACTGTGTAACGGCGGGACGCGGGTTTCCTTATTGGGGCCAAGGGACTCTGGTCACTGTCTCTGCAGC
TAGCACCAAGGGCCCATCGGTCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCAGCGTGGTGACCGT
GCCCTCCAGCAGCTTGGGCACCCAGACCTACATCTGCAACGTGAATCACAAGCCCAGCAACACCAAGGTG
GACAAGAGAGTTGAGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCT
GGACTCCGACGGCTCCTTCTTCCTCTATAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTCCCTGTCTC
CGGGTGGCGGTGGAGGGTCCGGCGGTGGTGGATCCCAAGTTCAGTTGCAGCAGTCTGGCCCTGGCCTGGT
CAAGCCTTCTGAGACACTGTCCATCACCTGTACCGTGTCCGGCTTCTCCCTGACCAATTACGGCGTGCAC
TGGATCAGACAGGCCCCTGGCAAAGGACTGGAATGGCTGGGAGTGATTTGGAGCGGCGGCAACACCGACT
ACAACACCCCTTTCACCAGCCGGTTCACCATCACCAAGGACAACTCCAAGAACCAGGTGTACTTCAAGCT
GCGGAGCGTGCGGGCTGATGACACCGCCATCTACTACTGTGCTCGGGCCCTGACCTACGCCGACTACGAG
TTTGCTTACTGGGGCCAGGGCACCCTGGTCACAGTTTCTTCTGGCGGTGGCGGTAGTGGGGGAGGCGGTT
CTGGCGGCGGAGGGTCCGGCGGTGGAGGATCAGAGATCGTGCTGACCCAGTCTCCTTCCACACTGTCTGT
GTCTCCCGGCGAGAGAGCCACCTTCAGCTGTAGAGCCTCTCAGTCCATCGGCACCAACATCCACTGGTAT
CAGCAGAAGCCCGGCAAGCCTCCTCGGCTGCTGATTAAGTACGCCTCCGAGTCCATCAGCGGCATCCCTG
ACAGATTCTCCGGCTCTGGCTCTGGCACCGAGTTTACCCTGACCATCTCCTCCGTGCAGTCCGAGGATTT
CGCCGTGTACTACTGCCAGCAGAACAACAACTGGCCCACCACCTTTGGACCCGGCACCAAGCTGACAGTT
CTT
>Seq ID 145: SI-95X67 heavy chain amino acid sequence
DVQLQESGPSLVKPSQSLSLTCTVTGYSITSDFAWNWIRQFPGNKLEWMGYISYSGNTRYNPSLKSRISI
TRDTSKNQFFLQLNSVTIEDTATYYCVTAGRGFPYWGQGTLVTVSAASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGGGGGSGGGGSQVQLQQSGPGLVKPSETLSITCTVSGFSLTNYGVH
WIRQAPGKGLEWLGVIWSGGNTDYNTPFTSRFTITKDNSKNQVYFKLRSVRADDTAIYYCARALTYYYYE
FAYWGQGTLVTVSSGGGGSGGGGSGGGGSGGGGSEIVLTQSPSTLSVSPGERATFSCRASQSIGTNIHWY
QQKPGKPPRLLIKYASESISGIPDRFSGSGSGTEFTLTISSVQSEDFAVYYCQQNNNWPTTFGPGTKLTV
L
>Seq ID 146: SI-95X67 heavy chain nucleotide sequence
GATGTGCAGCTTCAGGAGTCGGGACCTAGCCTGGTGAAACCTTCTCAGTCTCTGTCCCTCACCTGCACTG
TCACTGGCTACTCAATCACCAGTGATTTTGCCTGGAACTGGATTCGGCAGTTTCCAGGAAACAAGCTGGA
GTGGATGGGCTACATAAGTTATAGTGGTAACACTAGGTACAACCCATCTCTCAAAAGTCGAATCTCTATC
ACTCGCGACACATCCAAGAACCAATTCTTCCTGCAGTTGAACTCTGTGACTATTGAGGACACAGCCACAT
ATTACTGTGTAACGGCGGGACGCGGGTTTCCTTATTGGGGCCAAGGGACTCTGGTCACTGTCTCTGCAGC
TAGCACCAAGGGCCCATCGGTCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCAGCGTGGTGACCGT
GCCCTCCAGCAGCTTGGGCACCCAGACCTACATCTGCAACGTGAATCACAAGCCCAGCAACACCAAGGTG
GACAAGAGAGTTGAGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCT
GGACTCCGACGGCTCCTTCTTCCTCTATAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTCCCTGTCTC
CGGGTGGCGGTGGAGGGTCCGGCGGTGGTGGATCCCAAGTTCAGTTGCAGCAGTCTGGCCCTGGCCTGGT
CAAGCCTTCTGAGACACTGTCCATCACCTGTACCGTGTCCGGCTTCTCCCTGACCAATTACGGCGTGCAC
TGGATCAGACAGGCCCCTGGCAAAGGACTGGAATGGCTGGGAGTGATTTGGAGCGGCGGCAACACCGACT
ACAACACCCCTTTCACCAGCCGGTTCACCATCACCAAGGACAACTCCAAGAACCAGGTGTACTTCAAGCT
GCGGAGCGTGCGGGCTGATGACACCGCCATCTACTACTGTGCTCGGGCCCTGACCTACTACTACTACGAG
TTTGCTTACTGGGGCCAGGGCACCCTGGTCACAGTTTCTTCTGGCGGTGGCGGTAGTGGGGGAGGCGGTT
CTGGCGGCGGAGGGTCCGGCGGTGGAGGATCAGAGATCGTGCTGACCCAGTCTCCTTCCACACTGTCTGT
GTCTCCCGGCGAGAGAGCCACCTTCAGCTGTAGAGCCTCTCAGTCCATCGGCACCAACATCCACTGGTAT
CAGCAGAAGCCCGGCAAGCCTCCTCGGCTGCTGATTAAGTACGCCTCCGAGTCCATCAGCGGCATCCCTG
ACAGATTCTCCGGCTCTGGCTCTGGCACCGAGTTTACCCTGACCATCTCCTCCGTGCAGTCCGAGGATTT
CGCCGTGTACTACTGCCAGCAGAACAACAACTGGCCCACCACCTTTGGACCCGGCACCAAGCTGACAGTT
CTT
>Seq ID 147: SI-95X68 heavy chain amino acid sequence
DVQLQESGPSLVKPSQSLSLTCTVTGYSITSDFAWNWIRQFPGNKLEWMGYISYSGNTRYNPSLKSRISI
TRDTSKNQFFLQLNSVTIEDTATYYCVTAGRGFPYWGQGTLVTVSAASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGGGGGSGGGGSQVQLQQSGPGLVKPSETLSITCTVSGFSLTNYGVH
WIRQAPGKGLEWLGVIWSGGNTDYNTPFTSRFTITKDNSKNQVYFKLRSVRADDTAIYYCARALTYYDLE
FAYWGQGTLVTVSSGGGGSGGGGSGGGGSGGGGSEIVLTQSPSTLSVSPGERATFSCRASQSIGTNIHWY
QQKPGKPPRLLIKYASESISGIPDRFSGSGSGTEFTLTISSVQSEDFAVYYCQQNNNWPTTFGPGTKLTV
L
>Seq ID 148: SI-95X68 heavy chain nucleotide sequence
GATGTGCAGCTTCAGGAGTCGGGACCTAGCCTGGTGAAACCTTCTCAGTCTCTGTCCCTCACCTGCACTG
TCACTGGCTACTCAATCACCAGTGATTTTGCCTGGAACTGGATTCGGCAGTTTCCAGGAAACAAGCTGGA
GTGGATGGGCTACATAAGTTATAGTGGTAACACTAGGTACAACCCATCTCTCAAAAGTCGAATCTCTATC
ACTCGCGACACATCCAAGAACCAATTCTTCCTGCAGTTGAACTCTGTGACTATTGAGGACACAGCCACAT
ATTACTGTGTAACGGCGGGACGCGGGTTTCCTTATTGGGGCCAAGGGACTCTGGTCACTGTCTCTGCAGC
TAGCACCAAGGGCCCATCGGTCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCAGCGTGGTGACCGT
GCCCTCCAGCAGCTTGGGCACCCAGACCTACATCTGCAACGTGAATCACAAGCCCAGCAACACCAAGGTG
GACAAGAGAGTTGAGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCT
GGACTCCGACGGCTCCTTCTTCCTCTATAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTCCCTGTCTC
CGGGTGGCGGTGGAGGGTCCGGCGGTGGTGGATCCCAAGTTCAGTTGCAGCAGTCTGGCCCTGGCCTGGT
CAAGCCTTCTGAGACACTGTCCATCACCTGTACCGTGTCCGGCTTCTCCCTGACCAATTACGGCGTGCAC
TGGATCAGACAGGCCCCTGGCAAAGGACTGGAATGGCTGGGAGTGATTTGGAGCGGCGGCAACACCGACT
ACAACACCCCTTTCACCAGCCGGTTCACCATCACCAAGGACAACTCCAAGAACCAGGTGTACTTCAAGCT
GCGGAGCGTGCGGGCTGATGACACCGCCATCTACTACTGTGCTCGGGCCCTGACCTACTACGACCTCGAG
TTTGCTTACTGGGGCCAGGGCACCCTGGTCACAGTTTCTTCTGGCGGTGGCGGTAGTGGGGGAGGCGGTT
CTGGCGGCGGAGGGTCCGGCGGTGGAGGATCAGAGATCGTGCTGACCCAGTCTCCTTCCACACTGTCTGT
GTCTCCCGGCGAGAGAGCCACCTTCAGCTGTAGAGCCTCTCAGTCCATCGGCACCAACATCCACTGGTAT
CAGCAGAAGCCCGGCAAGCCTCCTCGGCTGCTGATTAAGTACGCCTCCGAGTCCATCAGCGGCATCCCTG
ACAGATTCTCCGGCTCTGGCTCTGGCACCGAGTTTACCCTGACCATCTCCTCCGTGCAGTCCGAGGATTT
CGCCGTGTACTACTGCCAGCAGAACAACAACTGGCCCACCACCTTTGGACCCGGCACCAAGCTGACAGTT
CTT
>Seq ID 149: SI-95m4 heavy chain amino acid sequence
QVQLQESGPSLVKPSQTLSLTCTVSGYSITSDFAWNWIRQFPGRKLEWMGYISYSGNTRYNPSLKSRFTI
SRDTSKNQFYLKLRSVTIADTAVYYCVTAGRGFPYWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
>Seq ID 150: SI-95m4 heavy chain nucleotide sequence
CAAGTGCAGCTCCAGGAGTCAGGCCCTAGCCTCGTGAAGCCCTCCCAGACCCTGAGCCTGACCTGCACCG
TATCTGGTTATAGTATTACAAGTGACTTCGCCTGGAACTGGATTAGGCAGTTTCCAGGTCGCAAGCTGGA
GTGGATGGGGTATATCTCCTACTCCGGTAACACTCGATACAACCCTTCCCTCAAAAGCAGATTTACCATT
TCCAGAGACACATCTAAGAACCAATTTTATCTGAAGTTGAGATCTGTTACCATCGCTGATACTGCTGTGT
ACTATTGCGTTACCGCCGGACGCGGCTTCCCCTATTGGGGGCAGGGAACCCTGGTGACTGTATCATCCGC
TAGCACCAAGGGCCCATCGGTCTTCCCCCTGGCACCCTCCTCCAAGAGCACCTCTGGGGGCACAGCGGCC
CTGGGCTGCCTGGTCAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCAGCGTGGTGACCGT
GCCCTCCAGCAGCTTGGGCACCCAGACCTACATCTGCAACGTGAATCACAAGCCCAGCAACACCAAGGTG
GACAAGAGAGTTGAGCCCAAATCTTGTGACAAAACTCACACATGCCCACCGTGCCCAGCACCTGAACTCC
TGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGTACACCCTG
CCCCCATCCCGGGAGGAGATGACCAAGAACCAGGTCAGCCTGACCTGCCTGGTCAAAGGCTTCTATCCCA
GCGACATCGCCGTGGAGTGGGAGAGCAATGGGCAGCCGGAGAACAACTACAAGACCACGCCTCCCGTGCT
GGACTCCGACGGCTCCTTCTTCCTCTATAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTCCCTGTCTC
CGGGTAAA
>Seq ID 151: SI-95ml1 light chain amino acid sequence
DILLTQSPVILSVSPGERVSFSCRASQSIGTNIHWYQQRTNGSPRLLIKYASESISGIPSRFSGSGSGTD
FTLSINSVESEDIADYYCQQNFNWPTTFGAGTKLELKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGEC
>Seq ID 152: SI-95m11 light chain nucleotide sequence
GACATCTTGCTGACTCAGTCTCCAGTCATCCTGTCTGTGAGTCCAGGAGAAAGAGTCAGTTTCTCCTGCA
GGGCCAGTCAGAGTATTGGCACAAACATACACTGGTATCAGCAAAGAACAAATGGTTCTCCAAGGCTTCT
CATAAAGTATGCTTCTGAGTCTATCTCTGGGATTCCTTCCAGGTTTAGTGGCAGTGGATCAGGGACAGAT
TTTACTCTTAGCATCAACAGTGTGGAGTCTGAAGATATTGCAGATTATTACTGTCAACAAAATTTCAACT
GGCCAACCACGTTCGGTGCTGGGACCAAGCTGGAGCTGAAACGTACGGTGGCTGCACCATCTGTCTTCAT
CTTCCCGCCATCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAAC
AGGGGAGAGTGT
>Seq ID 153: SI-95m12 light chain amino acid sequence
DILLTQSPVILSVSPGERVSFSCRASQSIGTNIHWYQQRTNGSPRLLIKYASESISGIPSRFSGSGSGTD
FTLSINSVESEDIADYYCQQNKNWPTTFGAGTKLELKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGEC
>Seq ID 154: SI-95m12 light chain nucleotide sequence
GACATCTTGCTGACTCAGTCTCCAGTCATCCTGTCTGTGAGTCCAGGAGAAAGAGTCAGTTTCTCCTGCA
GGGCCAGTCAGAGTATTGGCACAAACATACACTGGTATCAGCAAAGAACAAATGGTTCTCCAAGGCTTCT
CATAAAGTATGCTTCTGAGTCTATCTCTGGGATTCCTTCCAGGTTTAGTGGCAGTGGATCAGGGACAGAT
TTTACTCTTAGCATCAACAGTGTGGAGTCTGAAGATATTGCAGATTATTACTGTCAACAAAATAAGAACT
GGCCAACCACGTTCGGTGCTGGGACCAAGCTGGAGCTGAAACGTACGGTGGCTGCACCATCTGTCTTCAT
CTTCCCGCCATCTGATGAGCAGTTGAAATCTGGAACTGCCTCTGTTGTGTGCCTGCTGAATAACTTCTAT
CCCAGAGAGGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAAC
AGGGGAGAGTGT
>Seq ID 155: SI-95m13 heavy chain amino acid sequence
QVQLKQSGPGLVQPSQSLSITCTVSGFSLTNYGVHWVRQSPGKGLEWLGVIWSGGNTDYNTPFTSRLSIN
KDNSKSQVFFKMNSLQSNDTAIYYCARALTAYDYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 156: SI-95m13 heavy chain nucleotide sequence
CAGGTGCAGCTGAAGCAGTCAGGACCTGGCCTAGTGCAGCCCTCACAGAGCCTGTCCATCACCTGCACAG
TCTCTGGTTTCTCATTAACTAACTATGGTGTACACTGGGTTCGCCAGTCTCCAGGAAAGGGTCTGGAGTG
GCTGGGAGTGATATGGAGTGGTGGAAACACAGACTATAATACACCTTTCACATCCAGACTGAGCATCAAC
AAGGACAATTCCAAGAGCCAAGTTTTCTTTAAAATGAACAGTCTGCAATCTAATGACACAGCCATATATT
ACTGTGCCAGAGCCCTCACCGCCTATGATTACGAGTTTGCTTACTGGGGCCAAGGGACTCTGGTCACTGT
CTCTAGCGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGT
>Seq ID 157: SI-95m14 heavy chain amino acid sequence
QVQLKQSGPGLVQPSQSLSITCTVSGFSLTNYGVHWVRQSPGKGLEWLGVIWSGGNTDYNTPFTSRLSIN
KDNSKSQVFFKMNSLQSNDTAIYYCARALTWYDYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 158: SI-95m14 heavy chain nucleotide sequence
CAGGTGCAGCTGAAGCAGTCAGGACCTGGCCTAGTGCAGCCCTCACAGAGCCTGTCCATCACCTGCACAG
TCTCTGGTTTCTCATTAACTAACTATGGTGTACACTGGGTTCGCCAGTCTCCAGGAAAGGGTCTGGAGTG
GCTGGGAGTGATATGGAGTGGTGGAAACACAGACTATAATACACCTTTCACATCCAGACTGAGCATCAAC
AAGGACAATTCCAAGAGCCAAGTTTTCTTTAAAATGAACAGTCTGCAATCTAATGACACAGCCATATATT
ACTGTGCCAGAGCCCTCACCTGGTATGATTACGAGTTTGCTTACTGGGGCCAAGGGACTCTGGTCACTGT
CTCTAGCGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGT
>Seq ID 159: SI-95m15 heavy chain amino acid sequence
QVQLKQSGPGLVQPSQSLSITCTVSGFSLTNYGVHWVRQSPGKGLEWLGVIWSGGNTDYNTPFTSRLSIN
KDNSKSQVFFKMNSLQSNDTAIYYCARALTYADYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 160: SI-95m15 heavy chain nucleotide sequence
CAGGTGCAGCTGAAGCAGTCAGGACCTGGCCTAGTGCAGCCCTCACAGAGCCTGTCCATCACCTGCACAG
TCTCTGGTTTCTCATTAACTAACTATGGTGTACACTGGGTTCGCCAGTCTCCAGGAAAGGGTCTGGAGTG
GCTGGGAGTGATATGGAGTGGTGGAAACACAGACTATAATACACCTTTCACATCCAGACTGAGCATCAAC
AAGGACAATTCCAAGAGCCAAGTTTTCTTTAAAATGAACAGTCTGCAATCTAATGACACAGCCATATATT
ACTGTGCCAGAGCCCTCACCTACGCCGATTACGAGTTTGCTTACTGGGGCCAAGGGACTCTGGTCACTGT
CTCTAGCGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGT
>Seq ID 161: SI-95m16 heavy chain amino acid sequence
QVQLKQSGPGLVQPSQSLSITCTVSGFSLTNYGVHWVRQSPGKGLEWLGVIWSGGNTDYNTPFTSRLSIN
KDNSKSQVFFKMNSLQSNDTAIYYCARALTYVDYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 162: SI-95m16 heavy chain nucleotide sequence
CAGGTGCAGCTGAAGCAGTCAGGACCTGGCCTAGTGCAGCCCTCACAGAGCCTGTCCATCACCTGCACAG
TCTCTGGTTTCTCATTAACTAACTATGGTGTACACTGGGTTCGCCAGTCTCCAGGAAAGGGTCTGGAGTG
GCTGGGAGTGATATGGAGTGGTGGAAACACAGACTATAATACACCTTTCACATCCAGACTGAGCATCAAC
AAGGACAATTCCAAGAGCCAAGTTTTCTTTAAAATGAACAGTCTGCAATCTAATGACACAGCCATATATT
ACTGTGCCAGAGCCCTCACCTACGTGGATTACGAGTTTGCTTACTGGGGCCAAGGGACTCTGGTCACTGT
CTCTAGCGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGT
>Seq ID 163: SI-95m17 heavy chain amino acid sequence
QVQLKQSGPGLVQPSQSLSITCTVSGFSLTNYGVHWVRQSPGKGLEWLGVIWSGGNTDYNTPFTSRLSIN
KDNSKSQVFFKMNSLQSNDTAIYYCARALTYYFYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 164: SI-95m17 heavy chain nucleotide sequence
CAGGTGCAGCTGAAGCAGTCAGGACCTGGCCTAGTGCAGCCCTCACAGAGCCTGTCCATCACCTGCACAG
TCTCTGGTTTCTCATTAACTAACTATGGTGTACACTGGGTTCGCCAGTCTCCAGGAAAGGGTCTGGAGTG
GCTGGGAGTGATATGGAGTGGTGGAAACACAGACTATAATACACCTTTCACATCCAGACTGAGCATCAAC
AAGGACAATTCCAAGAGCCAAGTTTTCTTTAAAATGAACAGTCTGCAATCTAATGACACAGCCATATATT
ACTGTGCCAGAGCCCTCACCTACTATTTCTACGAGTTTGCTTACTGGGGCCAAGGGACTCTGGTCACTGT
CTCTAGCGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGT
>Seq ID 165: SI-95m18 heavy chain amino acid sequence
QVQLKQSGPGLVQPSQSLSITCTVSGFSLTNYGVHWVRQSPGKGLEWLGVIWSGGNTDYNTPFTSRLSIN
KDNSKSQVFFKMNSLQSNDTAIYYCARALTYYWYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 166: SI-95m18 heavy chain nucleotide sequence
CAGGTGCAGCTGAAGCAGTCAGGACCTGGCCTAGTGCAGCCCTCACAGAGCCTGTCCATCACCTGCACAG
TCTCTGGTTTCTCATTAACTAACTATGGTGTACACTGGGTTCGCCAGTCTCCAGGAAAGGGTCTGGAGTG
GCTGGGAGTGATATGGAGTGGTGGAAACACAGACTATAATACACCTTTCACATCCAGACTGAGCATCAAC
AAGGACAATTCCAAGAGCCAAGTTTTCTTTAAAATGAACAGTCTGCAATCTAATGACACAGCCATATATT
ACTGTGCCAGAGCCCTCACCTACTATTGGTACGAGTTTGCTTACTGGGGCCAAGGGACTCTGGTCACTGT
CTCTAGCGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGT
>Seq ID 167: SI-95m19 heavy chain amino acid sequence
QVQLKQSGPGLVQPSQSLSITCTVSGFSLTNYGVHWVRQSPGKGLEWLGVIWSGGNTDYNTPFTSRLSIN
KDNSKSQVFFKMNSLQSNDTAIYYCARALTYYYYEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 168: SI-95m19 heavy chain nucleotide sequence
CAGGTGCAGCTGAAGCAGTCAGGACCTGGCCTAGTGCAGCCCTCACAGAGCCTGTCCATCACCTGCACAG
TCTCTGGTTTCTCATTAACTAACTATGGTGTACACTGGGTTCGCCAGTCTCCAGGAAAGGGTCTGGAGTG
GCTGGGAGTGATATGGAGTGGTGGAAACACAGACTATAATACACCTTTCACATCCAGACTGAGCATCAAC
AAGGACAATTCCAAGAGCCAAGTTTTCTTTAAAATGAACAGTCTGCAATCTAATGACACAGCCATATATT
ACTGTGCCAGAGCCCTCACCTACTATTACTACGAGTTTGCTTACTGGGGCCAAGGGACTCTGGTCACTGT
CTCTAGCGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGT
>Seq ID 169: SI-95m20 heavy chain amino acid sequence
QVQLKQSGPGLVQPSQSLSITCTVSGFSLTNYGVHWVRQSPGKGLEWLGVIWSGGNTDYNTPFTSRLSIN
KDNSKSQVFFKMNSLQSNDTAIYYCARALTYYDLEFAYWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPG
>Seq ID 170: SI-95m20 heavy chain nucleotide sequence
CAGGTGCAGCTGAAGCAGTCAGGACCTGGCCTAGTGCAGCCCTCACAGAGCCTGTCCATCACCTGCACAG
TCTCTGGTTTCTCATTAACTAACTATGGTGTACACTGGGTTCGCCAGTCTCCAGGAAAGGGTCTGGAGTG
GCTGGGAGTGATATGGAGTGGTGGAAACACAGACTATAATACACCTTTCACATCCAGACTGAGCATCAAC
AAGGACAATTCCAAGAGCCAAGTTTTCTTTAAAATGAACAGTCTGCAATCTAATGACACAGCCATATATT
ACTGTGCCAGAGCCCTCACCTACTATGATCTCGAGTTTGCTTACTGGGGCCAAGGGACTCTGGTCACTGT
CTCTAGCGCTAGCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTCAGGCG
CCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACCCAGACCTACATCTGCAACGTGAATCACAAGCCCAGCAAC
ACCAAGGTGGACAAGAGAGTTGAGCCCAAATCTTGTGACAAAACTCACACATGCCCACCGTGCCCAGCAC
CTGAACTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTG
TGGTCAGCGTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCT
TCTATCCCAGCGACATCGCCGTGGAGTGGGAGAGCAATGGGCAGCCGGAGAACAACTACAAGACCACGCC
TCCCGTGCTGGACTCCGACGGCTCCTTCTTCCTCTATAGCAAGCTCACCGTGGACAAGAGCAGGTGGCAG
CAGGGGAACGTCTTCTCATGCTCCGTGATGCATGAGGCTCTGCACAACCACTACACGCAGAAGAGCCTCT
CCCTGTCTCCGGGT
>Seq ID 171: ABT-806 V1 heavy chain amino acid sequence
QVQLQESGPGLVKPSQSLSLTCTVSGYSITSDFAWNWIRQPPGKGLEWIGYISYSGNTRYNPSLKSRVTISRDT
SKNQFSLQLNSVTAEDTAVYYCVTAGRGFPYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALHNHYTQKSLSLSP
GK
>Seq ID 172: ABT-806 V1 heavy chain nucleotide sequence
CAAGTGCAGTTGCAGGAGAGTGGTCCGGGCCTGGTGAAGCCTTCTCAGTCACTGAGCCTCACGTGCACCGTAAG
CGGTTATAGTATCACCAGTGATTTTGCCTGGAACTGGATTCGGCAGCCGCCCGGGAAGGGACTGGAGTGGATCG
GATACATTTCCTACAGTGGAAACACTCGATATAATCCTAGTCTTAAAAGTCGGGTAACCATCTCTAGGGATACC
AGCAAGAATCAGTTCTCATTGCAACTCAATTCAGTGACCGCAGAAGACACCGCAGTCTACTATTGCGTCACAGC
CGGACGCGGTTTCCCGTACTGGGGCCAAGGAACACTAGTCACCGTGTCTTCCGCTAGCACCAAGGGCCCATCGG
TCTTCCCCCTGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCCAGCAGCTTGGGCACCCAGACCTACA
TCTGCAACGTGAATCACAAGCCCAGCAACACCAAGGTGGACAAGAGAGTTGAGCCCAAATCTTGTGACAAAACT
CACACATGCCCACCGTGCCCAGCACCTGAACTCCTGGGGGGACCGTCAGTCTTCCTCTTCCCCCCAAAACCCAA
GGACACCCTCATGATCTCCCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCTGAATGGCAAGGAGTACAAGTGCAA
GGTCTCCAACAAAGCCCTCCCAGCCCCCATCGAGAAAACCATCTCCAAAGCCAAAGGGCAGCCCCGAGAACCAC
AGGTGTACACCCTGCCCCCATCCCGGGAGGAGATGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCCCTGTCTCCG
GGTAAA
>Seq ID 173: ABT-806 V1 light chain amino acid sequence
DIQMTQSPSSVSASVGDRVTITCHSSQDINSNIGWLQQKPGKAPKGLIYHGTNLDDGVPSRFSGSGSGADYTLT
ISSLQPEDFATYYCVQYAQFPWTFGQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
>Seq ID 174: ABT-806 V1 light chain nucleotide sequence
GACATTCAGATGACACAGAGCCCAAGTAGCGTTTCTGCCAGCGTGGGGGACCGAGTCACCATTACTTGTCACAG
TTCACAAGATATTAACTCTAACATCGGCTGGCTGCAGCAAAAACCAGGTAAAGCACCAAAAGGGCTCATTTATC
ATGGGACCAACCTGGATGACGGAGTCCCGAGTCGATTCAGCGGGAGTGGAAGCGGAGCTGATTATACCCTGACG
ATCTCAAGCTTGCAGCCAGAGGATTTCGCCACATACTATTGTGTCCAGTATGCCCAGTTCCCCTGGACATTTGG
CCAGGGTACGAAGGTGGAGATTAAGCGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAGTGT
>Seq ID 175: ABT-806 V2 light chain amino acid sequence
DIVMTQSPSSLSVSPGDRVTITCHSSQDINSNIGWLQQKPGKAPKGLIYHGTNLDDGVPSRFSGSGSGADYTLT
ISSLEPEDFAVYYCVQYAQFPWTFGQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
>Seq ID 176: ABT-806 V2 light chain nucleotide sequence
GACATAGTGATGACACAGTCTCCAAGTTCTTTGAGCGTATCCCCAGGCGACCGCGTAACAATTACCTGTCACAG
CTCCCAGGATATTAATAGTAATATTGGCTGGCTTCAACAGAAGCCAGGGAAGGCACCAAAAGGACTGATCTACC
ACGGAACTAATCTGGACGATGGGGTTCCTTCACGGTTCTCCGGGTCCGGCTCTGGTGCCGACTACACTCTGACA
ATTAGCTCCCTGGAGCCCGAGGACTTCGCAGTGTACTATTGCGTGCAGTACGCACAATTTCCCTGGACATTTGG
TCAAGGCACCAAAGTCGAAATTAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAGTGT
>Seq ID 177: ABT-806 V4 heavy chain amino acid sequence
QVQLQESGPGLVKPSQSLSLTCTVSGYSITSDFAWNWIRQAPGKGLEWVGYISYSGNTRYNPSLKSRFTISRDT
SKNQFYLQLNSVTAEDTAVYYCVTAGRGFPYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALHNHYTQKSLSLSP
GK
>Seq ID 178: ABT-806 V4 heavy chain nucleotide sequence
CAAGTCCAGCTGCAGGAAAGTGGCCCTGGACTCGTTAAGCCTAGTCAGAGCTTGTCATTGACTTGCACAGTCAG
TGGTTATTCCATCACAAGTGACTTTGCCTGGAATTGGATTCGGCAGGCTCCCGGGAAGGGGCTGGAATGGGTCG
GGTATATTTCCTACAGCGGAAATACTCGATATAACCCATCCCTTAAAAGTCGCTTCACCATATCTCGTGACACC
AGCAAAAATCAATTTTATCTTCAACTCAATTCAGTGACAGCCGAGGATACAGCTGTTTACTATTGTGTGACAGC
TGGCAGAGGATTTCCCTATTGGGGACAGGGAACCCTTGTAACGGTCTCCAGCGCTAGCACCAAGGGCCCATCGG
TCTTCCCCCTGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCCAGCAGCTTGGGCACCCAGACCTACA
TCTGCAACGTGAATCACAAGCCCAGCAACACCAAGGTGGACAAGAGAGTTGAGCCCAAATCTTGTGACAAAACT
CACACATGCCCACCGTGCCCAGCACCTGAACTCCTGGGGGGACCGTCAGTCTTCCTCTTCCCCCCAAAACCCAA
GGACACCCTCATGATCTCCCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCTGAATGGCAAGGAGTACAAGTGCAA
GGTCTCCAACAAAGCCCTCCCAGCCCCCATCGAGAAAACCATCTCCAAAGCCAAAGGGCAGCCCCGAGAACCAC
AGGTGTACACCCTGCCCCCATCCCGGGAGGAGATGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCCCTGTCTCCG
GGTAAA
>Seq ID 179: SI-95X51 heavy chain amino acid sequence
EIVLTQSPSTLSVSPGERATFSCRASQSIGTNIHWYQQKPGKPPRLLIKYASESISGIPDRESGSGSGTE
FTLTISSVQSEDFAVYYCQQNNNWPTTFGPGTKLTVLGGGGSGGGGSGGGGSGGGGSQVQLQQSGPGLVK
PSETLSITCTVSGFSLTNYGVHWIRQAPGKGLEWLGVIWSGGNTDYNTPFTSRFTITKDNSKNQVYFKLR
SVRADDTAIYYCARALTYYDYEFAYWGQGTLVTVSSGGGGSGGGGSGGGGSGGGGSQVQLQESGPSLVKP
SQTLSLTCTVSGYSITSDFAWNWIRQFPGRKLEWMGYISYSGNTRYNPSLKSRFTISRDTSKNQFYLKLR
SVTIADTAVYYCVTAGRGFPYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHEALHNH
YTQKSLSLSPG
>Seq ID 180: SI-95X51 heavy chain nucleotide sequence
GAGATCGTGCTGACCCAGTCTCCTTCCACACTGTCTGTGTCTCCCGGCGAGAGAGCCACCTTCAGCTGTAGA
GCCTCTCAGTCCATCGGCACCAACATCCACTGGTATCAGCAGAAGCCCGGCAAGCCTCCTCGGCTGCTGATT
AAGTACGCCTCCGAGTCCATCAGCGGCATCCCTGACAGATTCTCCGGCTCTGGCTCTGGCACCGAGTTTACC
CTGACCATCTCCTCCGTGCAGTCCGAGGATTTCGCCGTGTACTACTGCCAGCAGAACAACAACTGGCCCACC
ACCTTTGGACCCGGCACCAAGCTGACAGTTCTTGGCGGTGGCGGTAGTGGGGGAGGCGGTTCTGGCGGCGGA
GGGTCCGGCGGTGGAGGATCACAAGTTCAGTTGCAGCAGTCTGGCCCTGGCCTGGTCAAGCCTTCTGAGACA
CTGTCCATCACCTGTACCGTGTCCGGCTTCTCCCTGACCAATTACGGCGTGCACTGGATCAGACAGGCCCCT
GGCAAAGGACTGGAATGGCTGGGAGTGATTTGGAGCGGCGGCAACACCGACTACAACACCCCTTTCACCAGC
CGGTTCACCATCACCAAGGACAACTCCAAGAACCAGGTGTACTTCAAGCTGCGGAGCGTGCGGGCTGATGAC
ACCGCCATCTACTACTGTGCTCGGGCCCTGACCTACTACGACTACGAGTTTGCTTACTGGGGCCAGGGCACC
CTGGTCACAGTTTCTTCTGGCGGTGGCGGTAGTGGGGGAGGCGGTTCTGGCGGCGGAGGGTCCGGCGGTGGA
GGATCACAAGTGCAGCTCCAGGAGTCAGGCCCTAGCCTCGTGAAGCCCTCCCAGACCCTGAGCCTGACCTGC
ACCGTATCTGGTTATAGTATTACAAGTGACTTCGCCTGGAACTGGATTAGGCAGTTTCCAGGTCGCAAGCTG
GAGTGGATGGGGTATATCTCCTACTCCGGTAACACTCGATACAACCCTTCCCTCAAAAGCAGATTTACCATT
TCCAGAGACACATCTAAGAACCAATTTTATCTGAAGTTGAGATCTGTTACCATCGCTGATACTGCTGTGTAC
TATTGCGTTACCGCCGGACGCGGCTTCCCCTATTGGGGGCAGGGAACCCTGGTGACTGTATCATCCGCTAGC
ACCAAGGGCCCATCGGTCTTCCCCCTGGCACCCTCCTCCAAGAGCACCTCTGGGGGCACAGCGGCCCTGGGC
TGCCTGGTCAAGGACTACTTCCCCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAGCGTGGTGACCGTGCCCTCCAGC
AGCTTGGGCACCCAGACCTACATCTGCAACGTGAATCACAAGCCCAGCAACACCAAGGTGGACAAGAGAGTT
GAGCCCAAATCTTGTGACAAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTGAGGTCACATGCGTGGTG
GTGGACGTGAGCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGTCCTGCACCAG
GACTGGCTGAATGGCAAGGAGTACAAGTGCAAGGTCTCCAACAAAGCCCTCCCAGCCCCCATCGAGAAAACC
ATCTCCAAAGCCAAAGGGCAGCCCCGAGAACCACAGGTGTACACCCTGCCCCCATCCCGGGATGAGCTGACC
AAGAACCAGGTCAGCCTGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGGCTCCTTCTTCCTCTAT
AGCAAGCTCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGT
>Seq ID 201: Cetuximab VH amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTYYDYEFAYWGQGTLVTVSS
>Seq ID 202: Cetuximab VH nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTACTATGA
TTACGAGTTTGCTTACTGGGGCCAAGGGACTCTGGTCACTGTCTCTAGC
>Seq ID 203: Cetuximab VL amino acid sequence
DILLTQSPVILSVSPGERVSFSCRASQSIGTNIHWYQQRTNGSPRLLIKYASESISGIPSRFSGSGSGTDFTLSINS
VESEDIADYYCQQNNNWPTTFGAGTKLELK
>Seq ID 204: Cetuximab VL nucleotide sequence
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATAATAACTGGCCAACCACGTTCGGTGCTGGGACCAA
GCTGGAGCTGAAA
>Seq ID 205: Nimotuzumab VH amino acid sequence
QVQLQQSGAEVKKPGSSVKVSCKASGYTFTNYYIYWVRQAPGQGLEWIGGINPTSGGSNFNEKFKTRVTITADESST
TAYMELSSLRSEDTAFYFCTRQGLWEDSDGRGEDFWGQGTTVTVSS
>Seq ID 206: Nimotuzumab VH nucleotide sequence
CAGGTGCAGCTGCAGCAGAGCGGCGCCGAGGTGAAGAAGCCCGGCAGCAGCGTGAAGGTGAGCTGCAAGGCCAGCGG
CTACACCTTCACCAACTACTACATCTACTGGGTGCGGCAGGCCCCCGGCCAGGGCCTGGAGTGGATCGGCGGCATCA
ACCCCACCAGCGGCGGCAGCAACTTCAACGAGAAGTTCAAGACCCGGGTGACCATCACCGCCGACGAGAGCAGCACC
ACCGCCTACATGGAGCTGAGCAGCCTGCGGAGCGAGGACACCGCCTTCTACTTCTGCACCCGGCAGGGCCTGTGGTT
CGACAGCGACGGCCGGGGCTTCGACTTCTGGGGCCAGGGCACCACCGTGACCGTGAGCAGC
>Seq ID 207: Nimotuzumab VL amino acid sequence
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQIT
>Seq ID 208: Nimotuzumab VL nucleotide sequence
GATATTCAAATGACTCAATCTCCTTCTTCTCTTTCTGCTTCTGTTGGTGATCGTGTTACTATTACTTGTCGTTCTTC
TCAAAATATTGTTCATTCTAATGGTAATACTTATCTTGATTGGTATCAACAAACTCCTGGTAAAGCTCCTAAACTTC
TTATTTATAAAGTTTCTAATCGTTTTTCTGGTGTTCCTTCTCGTTTTTCTGGTTCTGGTTCTGGTACTGATTTTACT
TTTACTATTTCTTCTCTTCAACCTGAAGATATTGCTACTTATTATTGTTTTCAATATTCTCATGTTCCTTGGACTTT
TGGTCAAGGTACTAAACTTCAAATTACT
>Seq ID 209: ABT-806 VH amino acid sequence
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSA
>Seq ID 210: ABT-806 VH nucleotide sequence
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCA
>Seq ID 211: ABT-806 VL amino acid sequence
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIK
>Seq ID 212: ABT-806 VL nucleotide sequence
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAA
>Seq ID 213: (G4S)2 linker amino acid sequence
GGGGSGGGGS
>Seq ID 214: (G4S)2 linker nucleotide sequence
GGCGGTGGAGGGTCCGGCGGTGGTGGATCC
>Seq ID 215: (G4S)4 linker amino acid sequence
GGGGSGGGGSGGGGS
>Seq ID 216: (G4S)4 linker nucleotide sequence
GGTGGAGGAGGCAGTGGTGGTGGAGGAAGCGGAGGTGGTGGCAGC
>Seq ID 217: (G4S)5 linker amino acid sequence
GGGGSGSGSGGGGGSGGGGSGGGGS
>Seq ID 218: (G4S)5 linker nucleotide sequence
GGTGGCGGTGGCTCCGGATCCGGTTCTGGAGGAGGCGGTGGAAGCGGAGGCGGTGGCTCTGGAGGAGGCGGTTCG
>Seq ID 219: (G4S)6 linker amino acid sequence
GGGGSGGGGSGSGSGGGGGSGGGGSGGGGS
>Seq ID 220: (G4S)6 linker nucleotide sequence
GGAGGCGGTGGTAGCGGTGGCGGTGGCTCCGGATCCGGTTCTGGAGGAGGCGGTGGAAGCGGAGGCGGTGGCTCTGG
AGGAGGCGGTTCG
>Seq ID 221: Human IgG1 amino acid sequence
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQQGNVFSC
SVMHEALHNHYTQKSLSLSPG
>Seq ID 222: Human IgG1 nucleotide sequence
GCTAGCACCAAGGGCCCATCGGTCTTCCCCCTGGCACCCTCCTCCAAGAGCACCTCTGGGGGCACAGCGGCCCTGGG
CTGCCTGGTCAAGGACTACTTCCCCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAGAGTTGAGCCCAAATCTTG
TGACAAAACTCACACATGCCCACCGTGCCCAGCACCTGAACTCCTGGGGGGACCGTCAGTCTTCCTCTTCCCCCCAA
AACCCAAGGACACCCTCATGATCTCCCGGACCCCTGAGGTCACATGCGTGGTGGTGGACGTGAGCCACGAAGACCCT
GAGGTCAAGTTCAACTGGTACGTGGACGGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGCTGAATGGCAAGGAGTACAAGTGCAAGG
TCTCCAACAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCTTCTATCC
CAGCGACATCGCCGTGGAGTGGGAGAGCAATGGGCAGCCGGAGAACAACTACAAGACCACGCCTCCCGTGCTGGACT
CCGACGGCTCCTTCTTCCTCTATAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGT
>Seq ID 223: Human CK amino acid sequence
RTVAAPSVFIFPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGEC
>Seq ID 224: Human CK nucleotide sequence
CGTACGGTGGCTGCACCATCTGTCTTCATCTTCCCGCCATCTGATGAGCAGTTGAAATCTGGAACTGCCTCTGTTGT
GTGCCTGCTGAATAACTTCTATCCCAGAGAGGCCAAAGTACAGTGGAAGGTGGATAACGCCCTCCAATCGGGTAACT
CCCAGGAGAGTGTCACAGAGCAGGACAGCAAGGACAGCACCTACAGCCTCAGCAGCACCCTGACGCTGAGCAAAGCA
GACTACGAGAAACACAAAGTCTACGCCTGCGAAGTCACCCATCAGGGCCTGAGCTCGCCCGTCACAAAGAGCTTCAA
CAGGGGAGAGTGT
>Seq ID 225: ABT-806 V1 VH amino acid sequence
QVQLQESGPGLVKPSQSLSLTCTVSGYSITSDFAWNWIRQPPGKGLEWIGYISYSGNTRYNPSLKSRVTISRDTSKN
QFSLQLNSVTAEDTAVYYCVTAGRGFPYWGQGTLVTVSS
>Seq ID 226: ABT-806 V1 VH nucleotide sequence
CAAGTGCAGTTGCAGGAGAGTGGTCCGGGCCTGGTGAAGCCTTCTCAGTCACTGAGCCTCACGTGCACCGTAAGCGG
TTATAGTATCACCAGTGATTTTGCCTGGAACTGGATTCGGCAGCCGCCCGGGAAGGGACTGGAGTGGATCGGATACA
TTTCCTACAGTGGAAACACTCGATATAATCCTAGTCTTAAAAGTCGGGTAACCATCTCTAGGGATACCAGCAAGAAT
CAGTTCTCATTGCAACTCAATTCAGTGACCGCAGAAGACACCGCAGTCTACTATTGCGTCACAGCCGGACGCGGTTT
CCCGTACTGGGGCCAAGGAACACTAGTCACCGTGTCTTCC
>Seq ID 227: ABT-806 V1 VL amino acid sequence
DIQMTQSPSSVSASVGDRVTITCHSSQDINSNIGWLQQKPGKAPKGLIYHGTNLDDGVPSRFSGSGSGADYTLTISS
LQPEDFATYYCVQYAQFPWTFGQGTKVEIK
>Seq ID 228: ABT-806 V1 VL nucleotide sequence
GACATTCAGATGACACAGAGCCCAAGTAGCGTTTCTGCCAGCGTGGGGGACCGAGTCACCATTACTTGTCACAGTTC
ACAAGATATTAACTCTAACATCGGCTGGCTGCAGCAAAAACCAGGTAAAGCACCAAAAGGGCTCATTTATCATGGGA
CCAACCTGGATGACGGAGTCCCGAGTCGATTCAGCGGGAGTGGAAGCGGAGCTGATTATACCCTGACGATCTCAAGC
TTGCAGCCAGAGGATTTCGCCACATACTATTGTGTCCAGTATGCCCAGTTCCCCTGGACATTTGGCCAGGGTACGAA
GGTGGAGATTAAG
>Seq ID 229: ABT-806 V2 VH amino acid sequence
QVQLQESGPGLVKPSQSLSLTCTVSGYSITSDFAWNWIRQAPGKGLEWVGYISYSGNTRYNPSLKSRFTISRDTSKN
QFYLQLNSVTAEDTAVYYCVTAGRGFPYWGQGTLVTVSS
>Seq ID 230: ABT-806 V2 VH nucleotide sequence
CAAGTCCAGCTGCAGGAAAGTGGCCCTGGACTCGTTAAGCCTAGTCAGAGCTTGTCATTGACTTGCACAGTCAGTGG
TTATTCCATCACAAGTGACTTTGCCTGGAATTGGATTCGGCAGGCTCCCGGGAAGGGGCTGGAATGGGTCGGGTATA
TTTCCTACAGCGGAAATACTCGATATAACCCATCCCTTAAAAGTCGCTTCACCATATCTCGTGACACCAGCAAAAAT
CAATTTTATCTTCAACTCAATTCAGTGACAGCCGAGGATACAGCTGTTTACTATTGTGTGACAGCTGGCAGAGGATT
TCCCTATTGGGGACAGGGAACCCTTGTAACGGTCTCCAGC
>Seq ID 231: ABT-806 V2 VL amino acid sequence
DIVMTQSPSSLSVSPGDRVTITCHSSQDINSNIGWLQQKPGKAPKGLIYHGTNLDDGVPSRFSGSGSGADYTLTISS
LEPEDFAVYYCVQYAQFPWTFGQGTKVEIK
>Seq ID 232: ABT-806 V2 VL nucleotide sequence
GACATAGTGATGACACAGTCTCCAAGTTCTTTGAGCGTATCCCCAGGCGACCGCGTAACAATTACCTGTCACAGCTC
CCAGGATATTAATAGTAATATTGGCTGGCTTCAACAGAAGCCAGGGAAGGCACCAAAAGGACTGATCTACCACGGAA
CTAATCTGGACGATGGGGTTCCTTCACGGTTCTCCGGGTCCGGCTCTGGTGCCGACTACACTCTGACAATTAGCTCC
CTGGAGCCCGAGGACTTCGCAGTGTACTATTGCGTGCAGTACGCACAATTTCCCTGGACATTTGGTCAAGGCACCAA
AGTCGAAATTAAA
>Seq ID 233: ABT-806 V3 VH amino acid sequence
QVQLQESGPSLVKPSQTLSLTCTVSGYSITSDFAWNWIRQFPGRKLEWMGYISYSGNTRYNPSLKSRFTISRDTSKN
QFYLKLRSVTIADTAVYYCVTAGRGFPYWGQGTLVTVSS
>Seq ID 234: ABT-806 V3 VH nucleotide sequence
CAAGTGCAGCTCCAGGAGTCAGGCCCTAGCCTCGTGAAGCCCTCCCAGACCCTGAGCCTGACCTGCACCGTATCTGG
TTATAGTATTACAAGTGACTTCGCCTGGAACTGGATTAGGCAGTTTCCAGGTCGCAAGCTGGAGTGGATGGGGTATA
TCTCCTACTCCGGTAACACTCGATACAACCCTTCCCTCAAAAGCAGATTTACCATTTCCAGAGACACATCTAAGAAC
CAATTTTATCTGAAGTTGAGATCTGTTACCATCGCTGATACTGCTGTGTACTATTGCGTTACCGCCGGACGCGGCTT
CCCCTATTGGGGGCAGGGAACCCTGGTGACTGTATCATCC
>Seq ID 235: ABT-806 V3 VL amino acid sequence
DIVMTQSPSSVSLSLGDRVTITCHSSQDINSNIGWLQQKPGKAFKGLIYHGTNLDDGVPSRFSGSGSGADYTLTISS
LQAEDFATYYCVQYAQFPWTFGPGTKLEIK
>Seq ID 236: ABT-806 V3 VL nucleotide sequence
GACATCGTGATGACCCAATCTCCAAGCTCAGTGAGTCTGTCACTAGGCGACCGTGTCACCATTACATGTCATAGTTC
CCAGGATATCAACAGTAATATTGGGTGGCTTCAGCAAAAGCCTGGTAAAGCCTTTAAAGGACTGATTTATCACGGGA
CCAACTTGGACGATGGAGTTCCCTCCCGGTTTTCTGGCTCCGGGTCCGGTGCAGACTATACTTTGACAATTAGCTCT
CTCCAGGCAGAGGACTTCGCTACATATTACTGCGTCCAGTATGCCCAGTTCCCTTGGACCTTTGGACCCGGCACTAA
ACTGGAAATTAAA
>Seq ID 237: SI-95m5 (humanized Cetuximab) VH amino acid sequence
QVQLQQSGPGLVKPSETLSITCTVSGFSLTNYGVHWIRQAPGKGLEWLGVIWSGGNTDYNTPFTSRFTITKDNSKNQ
VYFKLRSVRADDTAIYYCARALTYYDYEFAYWGQGTLVTVSS
>Seq ID 238: SI-95m5 (humanized Cetuximab) VH nucleotide sequence
CAAGTTCAGTTGCAGCAGTCTGGCCCTGGCCTGGTCAAGCCTTCTGAGACACTGTCCATCACCTGTACCGTGTCCGG
CTTCTCCCTGACCAATTACGGCGTGCACTGGATCAGACAGGCCCCTGGCAAAGGACTGGAATGGCTGGGAGTGATTT
GGAGCGGCGGCAACACCGACTACAACACCCCTTTCACCAGCCGGTTCACCATCACCAAGGACAACTCCAAGAACCAG
GTGTACTTCAAGCTGCGGAGCGTGCGGGCTGATGACACCGCCATCTACTACTGTGCTCGGGCCCTGACCTACTACGA
CTACGAGTTTGCTTACTGGGGCCAGGGCACCCTGGTCACAGTTTCTTCT
>Seq ID 239: SI-95m5 (humanized Cetuximab) VL amino acid sequence
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVL
>Seq ID 240: SI-95m5 (humanized Cetuximab) VL nucleotide sequence
GAGATCGTGCTGACCCAGTCTCCTTCCACACTGTCTGTGTCTCCCGGCGAGAGAGCCACCTTCAGCTGTAGAGCCTC
TCAGTCCATCGGCACCAACATCCACTGGTATCAGCAGAAGCCCGGCAAGCCTCCTCGGCTGCTGATTAAGTACGCCT
CCGAGTCCATCAGCGGCATCCCTGACAGATTCTCCGGCTCTGGCTCTGGCACCGAGTTTACCCTGACCATCTCCTCC
GTGCAGTCCGAGGATTTCGCCGTGTACTACTGCCAGCAGAACAACAACTGGCCCACCACCTTTGGACCCGGCACCAA
GCTGACCGTGCTG
>Seq ID 241: Cetuximab N92F VL amino acid sequence
EIVLTQSPSTLSVSPGERATFSCRASQSIGTNIHWYQQKPGKPPRLLIKYASESISGIPDRFSGSGSGTEFTLTISS
VQSEDFAVYYCQQNFNWPTTFGPGTKLTVL
>Seq ID 242: Cetuximab N92F VL nucleotide sequence
GAGATCGTGCTGACCCAGTCTCCTTCCACACTGTCTGTGTCTCCCGGCGAGAGAGCCACCTTCAGCTGTAGAGCCTC
TCAGTCCATCGGCACCAACATCCACTGGTATCAGCAGAAGCCCGGCAAGCCTCCTCGGCTGCTGATTAAGTACGCCT
CCGAGTCCATCAGCGGCATCCCTGACAGATTCTCCGGCTCTGGCTCTGGCACCGAGTTTACCCTGACCATCTCCTCC
GTGCAGTCCGAGGATTTCGCCGTGTACTACTGCCAGCAGAACTTCAACTGGCCCACCACCTTTGGACCCGGCACCAA
GCTGACCGTGCTG
>Seq ID 243: Cetuximab N92K VL amino acid sequence
DILLTQSPVILSVSPGERVSFSCRASQSIGTNIHWYQQRTNGSPRLLIKYASESISGIPSRFSGSGSGTDFTLSINS
VESEDIADYYCQQNKNWPTTFGAGTKLELK
>Seq ID 244: Cetuximab N92K VL nucleotide sequence
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATAAGAACTGGCCAACCACGTTCGGTGCTGGGACCAA
GCTGGAGCTGAAA
>Seq ID 245: Humanized Cetuximab Y101A VH amino acid sequence
QVQLQQSGPGLVKPSETLSITCTVSGFSLTNYGVHWIRQAPGKGLEWLGVIWSGGNTDYNTPFTSRFTITKDNSKNQ
VYFKLRSVRADDTAIYYCARALTAYDYEFAYWGQGTLVTVSS
>Seq ID 246: Humanized Cetuximab Y101A VH nucleotide sequence
CAAGTTCAGTTGCAGCAGTCTGGCCCTGGCCTGGTCAAGCCTTCTGAGACACTGTCCATCACCTGTACCGTGTCCGG
CTTCTCCCTGACCAATTACGGCGTGCACTGGATCAGACAGGCCCCTGGCAAAGGACTGGAATGGCTGGGAGTGATTT
GGAGCGGCGGCAACACCGACTACAACACCCCTTTCACCAGCCGGTTCACCATCACCAAGGACAACTCCAAGAACCAG
GTGTACTTCAAGCTGCGGAGCGTGCGGGCTGATGACACCGCCATCTACTACTGTGCTCGGGCCCTGACCGCCTACGA
CTACGAGTTTGCTTACTGGGGCCAGGGCACCCTGGTCACAGTTTCTTCT
>Seq ID 247: Cetuximab Y101W VH amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTWYDYEFAYWGQGTLVTVSS
>Seq ID 248: Cetuximab Y101W VH nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTGGTATGA
TTACGAGTTTGCTTACTGGGGCCAAGGGACTCTGGTCACTGTCTCTAGC
>Seq ID 249: Humanized Cetuximab Y102A VH amino acid sequence
QVQLQQSGPGLVKPSETLSITCTVSGFSLTNYGVHWIRQAPGKGLEWLGVIWSGGNTDYNTPFTSRFTITKDNSKNQ
VYFKLRSVRADDTAIYYCARALTYADYEFAYWGQGTLVTVSS
>Seq ID 250: Humanized Cetuximab Y102A VH nucleotide sequence
CAAGTTCAGTTGCAGCAGTCTGGCCCTGGCCTGGTCAAGCCTTCTGAGACACTGTCCATCACCTGTACCGTGTCCGG
CTTCTCCCTGACCAATTACGGCGTGCACTGGATCAGACAGGCCCCTGGCAAAGGACTGGAATGGCTGGGAGTGATTT
GGAGCGGCGGCAACACCGACTACAACACCCCTTTCACCAGCCGGTTCACCATCACCAAGGACAACTCCAAGAACCAG
GTGTACTTCAAGCTGCGGAGCGTGCGGGCTGATGACACCGCCATCTACTACTGTGCTCGGGCCCTGACCTACGCCGA
CTACGAGTTTGCTTACTGGGGCCAGGGCACCCTGGTCACAGTTTCTTCT
>Seq ID 251: Cetuximab D103F VH amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTYYFYEFAYWGQGTLVTVSS
>Seq ID 252: Cetuximab D103F VH nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTACTATTT
CTACGAGTTTGCTTACTGGGGCCAAGGGACTCTGGTCACTGTCTCTAGC
>Seq ID 253: Cetuximab D103W VH amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTYYWYEFAYWGQGTLVTVSS
>Seq ID 254: Cetuximab D103W VH nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTACTATTG
GTACGAGTTTGCTTACTGGGGCCAAGGGACTCTGGTCACTGTCTCTAGC
>Seq ID 255: Humanized Cetuximab D103Y VH amino acid sequence
QVQLQQSGPGLVKPSETLSITCTVSGFSLTNYGVHWIRQAPGKGLEWLGVIWSGGNTDYNTPFTSRFTITKDNSKNQ
VYFKLRSVRADDTAIYYCARALTYYYYEFAYWGQGTLVTVSS
>Seq ID 256: Humanized Cetuximab D103Y VH nucleotide sequence
CAAGTTCAGTTGCAGCAGTCTGGCCCTGGCCTGGTCAAGCCTTCTGAGACACTGTCCATCACCTGTACCGTGTCCGG
CTTCTCCCTGACCAATTACGGCGTGCACTGGATCAGACAGGCCCCTGGCAAAGGACTGGAATGGCTGGGAGTGATTT
GGAGCGGCGGCAACACCGACTACAACACCCCTTTCACCAGCCGGTTCACCATCACCAAGGACAACTCCAAGAACCAG
GTGTACTTCAAGCTGCGGAGCGTGCGGGCTGATGACACCGCCATCTACTACTGTGCTCGGGCCCTGACCTACTACTA
CTACGAGTTTGCTTACTGGGGCCAGGGCACCCTGGTCACAGTTTCTTCT
>Seq ID 257: Humanized Cetuximab Y104L VH amino acid sequence
QVQLQQSGPGLVKPSETLSITCTVSGFSLTNYGVHWIRQAPGKGLEWLGVIWSGGNTDYNTPFTSRFTITKDNSKNQ
VYFKLRSVRADDTAIYYCARALTYYDLEFAYWGQGTLVTVSS
>Seq ID 258: Humanized Cetuximab Y104L VH nucleotide sequence
CAAGTTCAGTTGCAGCAGTCTGGCCCTGGCCTGGTCAAGCCTTCTGAGACACTGTCCATCACCTGTACCGTGTCCGG
CTTCTCCCTGACCAATTACGGCGTGCACTGGATCAGACAGGCCCCTGGCAAAGGACTGGAATGGCTGGGAGTGATTT
GGAGCGGCGGCAACACCGACTACAACACCCCTTTCACCAGCCGGTTCACCATCACCAAGGACAACTCCAAGAACCAG
GTGTACTTCAAGCTGCGGAGCGTGCGGGCTGATGACACCGCCATCTACTACTGTGCTCGGGCCCTGACCTACTACGA
CCTCGAGTTTGCTTACTGGGGCCAGGGCACCCTGGTCACAGTTTCTTCT
>Seq ID 259: Cetuximab Y101A VH amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTAYDYEFAYWGQGTLVTVSS
>Seq ID 260: Cetuximab Y101A VH nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCGCCTATGA
TTACGAGTTTGCTTACTGGGGCCAAGGGACTCTGGTCACTGTCTCTAGC
>Seq ID 261: Cetuximab Y102A VH amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTYADYEFAYWGQGTLVTVSS
>Seq ID 262: Cetuximab Y102A VH nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTACGCCGA
TTACGAGTTTGCTTACTGGGGCCAAGGGACTCTGGTCACTGTCTCTAGC
>Seq ID 263: Cetuximab Y102V VH amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTYVDYEFAYWGQGTLVTVSS
>Seq ID 264: Cetuximab Y102V VH nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTACGTGGA
TTACGAGTTTGCTTACTGGGGCCAAGGGACTCTGGTCACTGTCTCTAGC
>Seq ID 265: Cetuximab D103Y VH amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTYYYYEFAYWGQGTLVTVSS
>Seq ID 266: Cetuximab D103Y VH nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTACTATTA
CTACGAGTTTGCTTACTGGGGCCAAGGGACTCTGGTCACTGTCTCTAGC
>Seq ID 267: Cetuximab Y104L VH amino acid sequence
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTYYDLEFAYWGQGTLVTVSS
>Seq ID 268: Cetuximab Y104L VH nucleotide sequence
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTACTATGA
TCTCGAGTTTGCTTACTGGGGCCAAGGGACTCTGGTCACTGTCTCTAGC
>Seq ID 269: Cetuximab N92F VL amino acid sequence
DILLTQSPVILSVSPGERVSFSCRASQSIGTNIHWYQQRINGSPRLLIKYASESISGIPSRFSGSGSGTDFTLSINS
VESEDIADYYCQQNFNWPTTFGAGTKLELK
>Seq ID 270: Cetuximab N92F VL nucleotide sequence
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATTTCAACTGGCCAACCACGTTCGGTGCTGGGACCAA
GCTGGAGCTGAAA

What is claimed is:

1. A biepitopic tetravalent antibody having a binding affinity to at least two epitopes of EGFR, comprising,
an antibody backbone, comprising
an antibody light chain having an antibody light chain variable (VL) domain,
an antibody heavy chain having an antibody heavy chain variable (VH) domain,
wherein the antibody VL domain and the antibody VH domain forms a Fab region, and
a scFv domain having a scFv light chain variable (VL) domain and scFv heavy chain variable (VH) domain, wherein the scFv domain is linked to at least one end of the antibody light chain or the antibody heavy chain through an inter-domain linker.

2. The biepitopic tetravalent antibody of claim 1, wherein the two epitopes of EGFR comprise an EGFR wild-type (EGFRwt) epitope and an EGFRvIII epitope, and wherein the antibody has a stronger binding affinity to the EGFRvIII epitope than to the EGFRwt epitope.

3. The biepitopic tetravalent antibody of claim 2, wherein the antibody has a binding affinity to the EGFRwt epitope with a first K_{D}, and a binding affinity to the EGFRvIII epitope with a second K_{D}, wherein the first K_{D} is higher than the second K_{D}, and wherein the first K_{D} is not less than 1E-11 M, and the second K_{D} is not more than 1E-06 M.

4. The biepitopic tetravalent antibody of claim 2, wherein the EGFRwt epitope comprises the epitope having a binding affinity to Cetuximab, or wherein the EGFRvIII epitope comprises the epitope having a binding affinity to ABT-806.

5. The biepitopic tetravalent antibody of claim 2,
wherein the scFv domain has the binding affinity to the EGFRwt epitope and the Fab domain has the binding affinity to the EGFRvIII epitope, or
wherein the scFv domain has the binding affinity to the EGFRvIII epitope and the Fab domain has the binding affinity to the EGFRwt epitope.

6. The biepitopic tetravalent antibody of claim 1, wherein the scFv domain is linked to the antibody light chain at its C-terminus, the antibody light chain at its N-terminus, the antibody heavy chain at its C-terminus, or the antibody heavy chain at its N-terminus.

7. The biepitopic tetravalent antibody of claim 1, wherein the antibody backbone comprises Cetuximab, humanized Cetuximab, dematured Cetuximab, or humanized dematured Cetuximab, and wherein the scFv domain comprises a binding domain derived from the variable region of ABT-806 or humanized ABT-806.

8. The biepitopic tetravalent antibody of claim 7, wherein the antibody backbone comprises a Cetuximab dematuration mutation (sequential numbering) comprising VH-Y101A, VH-Y101W, VH-Y102A, VH-D103F, VH-D103W, VH-D103Y, VH-Y104L, VL-N92F, VL-N92K, or a combination thereof.

9. The biepitopic tetravalent antibody of claim 7, wherein the antibody heavy chain comprises an amino acid sequence having at least 99% sequence identity to SEQ ID NO. 1, 13, 79, 97, 101, 103, 105, 107, 123, 127, 129, 131, or 133, or wherein the antibody light chain comprises an amino acid sequence having at least 99% sequence identity to SEQ ID NO. 3, 81, 83, 99, 109, 125, or 135.

10. The biepitopic tetravalent antibody of claim 7, wherein the antibody backbone comprises humanized Cetuximab, wherein the antibody VH domain comprises an amino acid sequence having at least 99% sequence identity to SEQ ID NO. 237, or wherein the antibody VL domain comprises an amino acid sequence having at least 99% sequence identity to SEQ ID NO. 239.

11. The biepitopic tetravalent antibody of claim 7, wherein the antibody backbone comprises dematured Cetuximab, and wherein the antibody VH domain comprises an amino acid sequence having at least 99% sequence identity to SEQ ID NO. 247, 251, 253, 259, 261, 263, 265, or 267, or wherein the antibody VL domain comprises an amino acid sequence having at least 99% sequence identity to SEQ ID NO. 243 or 269.

12. The biepitopic tetravalent antibody of claim 7, wherein the antibody backbone comprises the humanized dematured Cetuximab, and wherein the antibody VH domain comprises an amino acid sequence having at least 99% sequence identity to SEQ ID NO. 245, 249, 255, or 257, or wherein the antibody VL domain comprises an amino acid sequence having at least 99% sequence identity to SEQ ID NO. 241.

13. The biepitopic tetravalent antibody of claim 7, wherein the scFv domain comprises the variable regions of ABT-806 or humanized ABT-806.

14. The biepitopic tetravalent antibody of claim 7, wherein the scFv VH domain comprises an amino acid sequence having at least 99% sequence identity to SEQ ID NO. 209, 225, 229, or 233, or wherein the scFv VL domain comprises an amino acid sequence having at least 99% sequence identity to SEQ ID NO. 211, 227, 231, or 235.

15. The biepitopic tetravalent antibody of claim 7, wherein the antibody VH domain comprises 3 complementary determining regions (CDRs) of SEQ ID NO. 201, 237, 245, 247, 249, 251, 253, 255, 257, 259, 261, 263, 265, or 267;
wherein the antibody VL domain comprises 3 CDRs of SEQ ID NO. 203, 239, 241, 243, or 269;
wherein the scFv VH domain comprises 3 CDRs of SEQ ID NO. 209; and
wherein the scFv VL domain comprises 3 CDRs of SEQ ID NO. 211.

16. The biepitopic tetravalent antibody of claim 1, wherein the antibody backbone comprises ABT0806 or humanized ABT-806, and wherein the scFv domain comprises a binding domain derived from the variable region of Cetuximab, humanized Cetuximab, dematured Cetuximab, humanized dematured Cetuximab, or Nimotuzumab.

17. The biepitopic tetravalent antibody of claim 16, wherein the antibody heavy chain comprises an amino acid sequence having at least 99% sequence identity to SEQ ID NO. 9, 17, 19, 21, 23, 25, 27, 29, 31, 75, 93, 113, 115, 117, 119, 121, 137, 139, 141, 143, 145, 147, or 179, or wherein the antibody light chain comprises an amino acid sequence having at least 99% sequence identity to SEQ ID NO. 11, 33, 35, 37, 39, 41, 43, 77, 91, 95, or 111.

18. The biepitopic tetravalent antibody of claim 16, wherein the antibody backbone comprises the ABT-806, and wherein the antibody VH domain comprises an amino acid sequence having at least 99% sequence identity to SEQ ID NO. 209, or wherein antibody the VL domain comprises an amino acid sequence having at least 99% sequence identity to SEQ ID NO. 211.

19. The biepitopic tetravalent antibody of claim 16, wherein the antibody backbone comprises the humanized ABT-806, and wherein the antibody VH domain comprises an amino acid sequence having at least 99% sequence identity to SEQ ID NO. 225, 229, or 233, or wherein the antibody backbone comprises the humanized ABT-806, and wherein the antibody VL domain comprises an amino acid sequence having at least 99% sequence identity to SEQ ID NO. 227, 231, or 235.

20. The biepitopic tetravalent antibody of claim 16, wherein the scFv VH domain comprises an amino acid sequence having at least 99% sequence identity to SEQ ID NO. 201, 205, 237, 245, 247, 249, 251, 253, 255, 257, 259, 261, 263, 265, or 267, or wherein the scFv VL domain comprises an amino acid sequence having at least 99% sequence identity to SEQ ID NO. 203, 207, 239, 241, 243, or 269.

21. The biepitopic tetravalent antibody of claim 16, wherein the scFv domain comprises a binding domain derived from the variable region of the dematured Cetuximab or the humanized dematured Cetuximab, wherein the dematured Cetuximab or the humanized dematured Cetuximab comprises a Cetuximab dematuration mutation (sequential numbering) comprising VH-Y101A, VH-Y101W, VH-Y102A, VH-D103F, VH-D103W, VH-D103Y, VH-Y104L, VL-N92F, VL-N92K, or a combination thereof.

22. The biepitopic tetravalent antibody of claim 16,
wherein the antibody VH domain comprises 3 complementary determining regions (CDRs) of SEQ ID NO. 209;
wherein the antibody VL domain comprises 3 CDRs of SEQ ID NO. 211;
wherein the scFv VH domain comprises 3 CDRs of SEQ ID NO. 201, 205, 237, 245, 247, 249, 251, 253, 255, 257, 259, 261, 263, 265, or 267; and
wherein the scFv VL domain comprises 3 CDRs of SEQ ID NO. 203, 207, 239, 241, 243, or 269.

23. The biepitopic tetravalent antibody of claim 1, wherein the antibody backbone comprises Nimotuzumab, and wherein the scFv domain comprises a binding domain derived from the variable region of ABT-806 or humanized ABT-806.

24. The biepitopic tetravalent antibody of claim 23, wherein the antibody heavy chain comprises an amino acid sequence having at 99% sequence identity to SEQ ID NO. 5, 15, 45, 47, 49, 51, 53, 55, or 85, or wherein the antibody light chain comprises an amino acid sequence having at least 99% sequence identity to SEQ ID NO. 7, 57, 59, 61, 63, 65, 67, 69, 71, or 73.

25. The biepitopic tetravalent antibody of claim 23, wherein the antibody VH domain comprises an amino acid sequence having at least 99% sequence identity to SEQ ID NO. 205, or wherein the antibody VL domain comprises an amino acid sequence having at least 99% sequence identity to SEQ ID NO. 207.

26. The biepitopic tetravalent antibody of claim 23, wherein the scFv VH domain comprises an amino acid sequence having at least 99% sequence identity to SEQ ID NO. 209, or wherein the scFv VL domain comprises an amino acid sequence having at least 99% sequence identity to SEQ ID NO. 211.

27. A dematured Cetuximab monoclonal antibody, comprising a light chain having a light chain variable (VL) domain, and a heavy chain having a heavy chain variable (VH) domain, wherein the VL domain and the VH domain forms a Fab region, and wherein the monoclonal antibody comprises a Cetuximab dematuration mutation (sequential numbering) selected from VH-Y101A, VH-Y101W, VH-Y102A, VH-D103F, VH-D103W, VH-D103Y, VH-Y104L, VL-N92F, VL-N92K, or a combination thereof.

28. A humanized anti-EGFRvIII monoclonal antibody, comprising a light chain having a light chain variable (VL) domain, and a heavy chain having a heavy chain variable (VH) domain, wherein the VL domain and the VH domain forms a Fab region, and wherein the heavy chain comprises an amino acid sequence having at least 99% of the sequence identity to SEQ ID NO. 171, 149, or 177 or wherein the light chain comprises an amino acid sequence having at least 99% of the sequence identity to SEQ ID NO. 111, 173, or 175.

29. An isolated nucleic acid sequence encoding the biepitopic tetravalent antibody of claim 1.

30. A method of producing a biepitopic tetravalent antibody, comprising culturing a host cell so that the biepitopic antibody is produced, wherein the host cell comprises the nucleic acid of claim 1.

31. An immunoconjugate comprising the biepitopic tetravalent antibody of claim 1 and a cytotoxic agent, wherein the cytotoxic agent comprises a chemotherapeutic agent, a growth inhibitory agent, a toxin, or a radioactive isotope.

32. A pharmaceutical composition, comprising the biepitopic tetravalent antibody of claim 1 and a pharmaceutically acceptable carrier.

33. The pharmaceutical composition of claim 32, further comprising radioisotope, radionuclide, a toxin, a therapeutic agent, a chemotherapeutic agent, or a combination thereof.

34. A pharmaceutical composition, comprising the immunoconjugate of claim 31 and a pharmaceutically acceptable carrier.

35. A method of treating a subject with a cancer, comprising administering to the subject an effective amount of the biepitopic tetravalent antibody of claim 1, wherein the cancer comprises cells expressing EGFR, EGFRvIII, or both.

36. The method of claim 35, further comprising co-administering an effective amount of a therapeutic agent, wherein the therapeutic agent comprises an antibody, a chemotherapy agent, an enzyme, or a combination thereof.


SEQUENCE LISTING (250 sequences)

SEQ ID NO: 1 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - DNA, 1347 bp, synthetic construct
  source 1..1347: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttaa

SEQ ID NO: 3 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 645 bp, synthetic construct
  source 1..645: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 5 - protein, 452 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - DNA, 1359 bp, synthetic construct
  source 1..1359: mol_type = other DNA
caggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttaa

SEQ ID NO: 7 - protein, 219 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 660 bp, synthetic construct
  source 1..660: mol_type = other DNA
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattactcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 9 - protein, 446 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1341 bp, synthetic construct
  source 1..1341: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 11 - protein, 214 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 645 bp, synthetic construct
  source 1..645: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 13 - protein, 701 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 14 - protein, 2106 aa, synthetic construct
CAGGTGCAGCTGAAGCAGTCAGGACCTGGCCTAGTGCAGCCCTCACAGAGCCTGTCCATCACCTGCACAGTCTCTGGTTTCTCATTAACTAACTATGGTGTACACTGGGTTCGCCAGTCTCCAGGAAAGGGTCTGGAGTGGCTGGGAGTGATATGGAGTGGTGGAAACACAGACTATAATACACCTTTCACATCCAGACTGAGCATCAACAAGGACAATTCCAAGAGCCAAGTTTTCTTTAAAATGAACAGTCTGCAATCTAATGACACAGCCATATATTACTGTGCCAGAGCCCTCACCTACTATGATTACGAGTTTGCTTACTGGGGCCAAGGGACTCTGGTCACTGTCTCTAGCGCTAGCACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAACTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAGAGTTGAGCCCAAATCTTGTGACAAAACTCACACATGCCCACCGTGCCCAGCACCTGAACTCCTGGGGGGACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCAAGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGATGAGCTGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTATAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGTGGCGGTGGAGGGTCCGGCGGTGGTGGATCCGATGTGCAGCTTCAGGAGTCGGGACCTAGCCTGGTGAAACCTTCTCAGTCTCTGTCCCTCACCTGCACTGTCACTGGCTACTCAATCACCAGTGATTTTGCCTGGAACTGGATTCGGCAGTTTCCAGGAAACAAGCTGGAGTGGATGGGCTACATAAGTTATAGTGGTAACACTAGGTACAACCCATCTCTCAAAAGTCGAATCTCTATCACTCGCGACACATCCAAGAACCAATTCTTCCTGCAGTTGAACTCTGTGACTATTGAGGACACAGCCACATATTACTGTGTAACGGCGGGACGCGGGTTTCCTTATTGGGGCCAAGGGACTCTGGTCACTGTCTCTGCAGGCGGTGGCGGTAGTGGGGGAGGCGGTTCTGGCGGCGGAGGGTCCGGCGGTGGAGGATCAGACATCCTGATGACCCAATCTCCATCCTCCATGTCTGTATCTCTGGGAGACACAGTCAGCATCACTTGCCATTCAAGTCAGGACATTAACAGTAATATAGGGTGGTTGCAGCAGAGACCAGGGAAATCATTTAAGGGCCTGATCTATCATGGAACCAACTTGGACGATGAAGTTCCATCAAGGTTCAGTGGCAGTGGATCTGGAGCCGATTATTCTCTCACCATCAGCAGCCTGGAATCTGAAGATTTTGCAGACTATTACTGTGTACAGTATGCTCAGTTTCCGTGGACGTTCGGTGGAGGCACCAAGCTGGAAATCAAATGA

SEQ ID NO: 15 - protein, 705 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 16 - DNA, 2118 bp, synthetic construct
  source 1..2118: mol_type = other DNA
caggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggcggtggcggtagtgggggaggcggttctggcggcggagggtccggcggtggaggatcagacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaatga

SEQ ID NO: 17 - protein, 701 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 18 - DNA, 2106 bp, synthetic construct
  source 1..2106: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcggcggtggcggtagtgggggaggcggttctggcggcggagggtccggcggtggaggatcagacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaatga

SEQ ID NO: 19 - protein, 710 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQIT

SEQ ID NO: 20 - DNA, 2133 bp, synthetic construct
  source 1..2133: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggtggtggaggatccggcggcggtggtagcggcggcggtgggagcggcggtggtggcagcgacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcacctga

SEQ ID NO: 21 - protein, 716 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - DNA, 2151 bp, synthetic construct
  source 1..2151: mol_type = other DNA
caagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggtggaggaggcagtggtggtggaggaagcggaggtggtggcagcgacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcacctccggatccggttctggaggaggcggtggaagcggaggcggtggctctggaggaggcggttcggatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 23 - protein, 720 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITGGGGSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - DNA, 2163 bp, synthetic construct
  source 1..2163: mol_type = other DNA
caagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggtggaggaggcagtggtggtggaggaagcggaggtggtggcagcgacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcaccggtggcggtggctccggatccggttctggaggaggcggtggaagcggaggcggtggctctggaggaggcggttcggatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 25 - protein, 725 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITGGGGSGGGGSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - DNA, 2178 bp, synthetic construct
  source 1..2178: mol_type = other DNA
caagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggtggaggaggcagtggtggtggaggaagcggaggtggtggcagcgacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcaccggaggcggtggtagcggtggcggtggctccggatccggttctggaggaggcggtggaagcggaggcggtggctctggaggaggcggttcggatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 27 - protein, 716 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - DNA, 2151 bp, synthetic construct
  source 1..2151: mol_type = other DNA
gacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcaccggtggaggaggcagtggtggtggaggaagcggaggtggtggcagccaagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttcctccggatccggttctggaggaggcggtggaagcggaggcggtggctctggaggaggcggttcggatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 29 - protein, 720 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - DNA, 2163 bp, synthetic construct
  source 1..2163: mol_type = other DNA
gacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcaccggtggaggaggcagtggtggtggaggaagcggaggtggtggcagccaagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggtggcggtggctccggatccggttctggaggaggcggtggaagcggaggcggtggctctggaggaggcggttcggatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 31 - protein, 725 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGGGGSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - DNA, 2178 bp, synthetic construct
  source 1..2178: mol_type = other DNA
gacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcaccggtggaggaggcagtggtggtggaggaagcggaggtggtggcagccaagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggaggcggtggtagcggtggcggtggctccggatccggttctggaggaggcggtggaagcggaggcggtggctctggaggaggcggttcggatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 33 - protein, 485 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITSGSGSGGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - DNA, 1458 bp, synthetic construct
  source 1..1458: mol_type = other DNA
caagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggtggaggaggcagtggtggtggaggaagcggaggtggtggcagcgacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcacctccggatccggttctggaggaggcggtggaagcggaggcggtggctctggaggaggcggttcggacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 35 - protein, 489 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITGGGGSGSGSGGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 1470 bp, synthetic construct
  source 1..1470: mol_type = other DNA
caagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggtggaggaggcagtggtggtggaggaagcggaggtggtggcagcgacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcaccggtggcggtggctccggatccggttctggaggaggcggtggaagcggaggcggtggctctggaggaggcggttcggacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 37 - protein, 494 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITGGGGSGGGGSGSGSGGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 1485 bp, synthetic construct
  source 1..1485: mol_type = other DNA
caagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggtggaggaggcagtggtggtggaggaagcggaggtggtggcagcgacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcaccggaggcggtggtagcggtggcggtggctccggatccggttctggaggaggcggtggaagcggaggcggtggctctggaggaggcggttcggacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 39 - protein, 485 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSSGSGSGGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 1458 bp, synthetic construct
  source 1..1458: mol_type = other DNA
gacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcaccggtggaggaggcagtggtggtggaggaagcggaggtggtggcagccaagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttcctccggatccggttctggaggaggcggtggaagcggaggcggtggctctggaggaggcggttcggacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 41 - protein, 489 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGSGSGGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - DNA, 1470 bp, synthetic construct
  source 1..1470: mol_type = other DNA
gacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcaccggtggaggaggcagtggtggtggaggaagcggaggtggtggcagccaagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggtggcggtggctccggatccggttctggaggaggcggtggaagcggaggcggtggctctggaggaggcggttcggacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 43 - protein, 494 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSGGGGSGGGGSGSGSGGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 1485 bp, synthetic construct
  source 1..1485: mol_type = other DNA
gacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcaccggtggaggaggcagtggtggtggaggaagcggaggtggtggcagccaagtgcagctgcagcagagcggcgccgaggtgaagaagcctggcagcagcgtgaaggtgagctgcaaggctagcggctacaccttcaccaactactacatctactgggtgagacaagcccctggccaaggcctggagtggatcggcggcatcaaccctacaagcggcggcagcaacttcaacgagaagttcaagacaagagttaccataactgccgacgagagcagcaccaccgcctacatggagctgagcagcctgagaagcgaggacaccgccttctacttctgcacaagacaaggcctgtggttcgacagcgacggcagaggcttcgacttttggggccaaggaaccacagttactgtgtcttccggaggcggtggtagcggtggcggtggctccggatccggttctggaggaggcggtggaagcggaggcggtggctctggaggaggcggttcggacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 45 - protein, 711 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKSGSGSGGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - DNA, 2136 bp, synthetic construct
  source 1..2136: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggaggaggcagtggtggtggaggaagcggaggtggtggcagcgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaatccggatccggttctggaggaggcggtggaagcggaggcggtggctctggaggaggcggttcgcaggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 47 - protein, 715 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGSGSGGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - DNA, 2148 bp, synthetic construct
  source 1..2148: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggaggaggcagtggtggtggaggaagcggaggtggtggcagcgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggtggcggtggctccggatccggttctggaggaggcggtggaagcggaggcggtggctctggaggaggcggttcgcaggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 49 - protein, 720 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGGGGSGSGSGGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - DNA, 2163 bp, synthetic construct
  source 1..2163: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggaggaggcagtggtggtggaggaagcggaggtggtggcagcgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggaggcggtggtagcggtggcggtggctccggatccggttctggaggaggcggtggaagcggaggcggtggctctggaggaggcggttcgcaggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 51 - protein, 711 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSASGSGSGGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - DNA, 2136 bp, synthetic construct
  source 1..2136: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggtggaggaggcagtggtggtggaggaagcggaggtggtggcagcgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcatccggatccggttctggaggaggcggtggaagcggaggcggtggctctggaggaggcggttcgcaggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 53 - protein, 715 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGSGSGGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - DNA, 2148 bp, synthetic construct
  source 1..2148: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggtggaggaggcagtggtggtggaggaagcggaggtggtggcagcgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggcggtggctccggatccggttctggaggaggcggtggaagcggaggcggtggctctggaggaggcggttcgcaggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 55 - protein, 720 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGSGSGGGGGSGGGGSGGGGSQVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - DNA, 2163 bp, synthetic construct
  source 1..2163: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggtggaggaggcagtggtggtggaggaagcggaggtggtggcagcgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggaggcggtggtagcggtggcggtggctccggatccggttctggaggaggcggtggaagcggaggcggtggctctggaggaggcggttcgcaggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 57 - protein, 478 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKSGSGSGGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - DNA, 1437 bp, synthetic construct
  source 1..1437: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggaggaggcagtggtggtggaggaagcggaggtggtggcagcgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaatccggatccggttctggaggaggcggtggaagcggaggcggtggctctggaggaggcggttcggacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcacccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 59 - protein, 482 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGSGSGGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - DNA, 1449 bp, synthetic construct
  source 1..1449: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggaggaggcagtggtggtggaggaagcggaggtggtggcagcgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggtggcggtggctccggatccggttctggaggaggcggtggaagcggaggcggtggctctggaggaggcggttcggacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcacccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 61 - protein, 487 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGGGGSGSGSGGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - DNA, 1464 bp, synthetic construct
  source 1..1464: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggaggaggcagtggtggtggaggaagcggaggtggtggcagcgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggaggcggtggtagcggtggcggtggctccggatccggttctggaggaggcggtggaagcggaggcggtggctctggaggaggcggttcggacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcacccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 63 - protein, 478 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSASGSGSGGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - DNA, 1437 bp, synthetic construct
  source 1..1437: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggtggaggaggcagtggtggtggaggaagcggaggtggtggcagcgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcatccggatccggttctggaggaggcggtggaagcggaggcggtggctctggaggaggcggttcggacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcacccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 65 - protein, 482 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGSGSGGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - DNA, 1449 bp, synthetic construct
  source 1..1449: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggtggaggaggcagtggtggtggaggaagcggaggtggtggcagcgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggcggtggctccggatccggttctggaggaggcggtggaagcggaggcggtggctctggaggaggcggttcggacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcacccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 67 - protein, 487 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGSGSGGGGGSGGGGSGGGGSDIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - DNA, 1464 bp, synthetic construct
  source 1..1464: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggtggaggaggcagtggtggtggaggaagcggaggtggtggcagcgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggaggcggtggtagcggtggcggtggctccggatccggttctggaggaggcggtggaagcggaggcggtggctctggaggaggcggttcggacattcagatgacacagagccctagcagcctgagcgctagcgtgggcgacagagtgaccatcacctgtagaagctctcagaacatcgtgcacagcaacggcaacacctacctggactggtatcagcagacccctggcaaggcccctaagctgctgatctacaaggtgagcaacagatttagtggtgtccctagcagattcagcggcagcggcagcggcaccgacttcaccttcaccatcagcagcctgcagcctgaggacatcgccacctactactgctttcagtacagccacgtgccttggaccttcggacaaggcacgaaactgcagatcacccgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 69 - protein, 478 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 70 - DNA, 1437 bp, synthetic construct
  source 1..1437: mol_type = other DNA
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattactcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttccggatccggttctggaggaggcggtggaagcggaggcggtggctctggaggaggcggttcggatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggaggaggcagtggtggtggaggaagcggaggtggtggcagcgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaatag

SEQ ID NO: 71 - protein, 482 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 72 - DNA, 1449 bp, synthetic construct
  source 1..1449: mol_type = other DNA
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattactcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggtggcggtggctccggatccggttctggaggaggcggtggaagcggaggcggtggctctggaggaggcggttcggatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggaggaggcagtggtggtggaggaagcggaggtggtggcagcgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaatag

SEQ ID NO: 73 - protein, 487 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 74 - protein, 1464 aa, synthetic construct
GATATTCAAATGACTCAATCTCCTTCTTCTCTTTCTGCTTCTGTTGGTGATCGTGTTACTATTACTTGTCGTTCTTCTCAAAATATTGTTCATTCTAATGGTAATACTTATCTTGATTGGTATCAACAAACTCCTGGTAAAGCTCCTAAACTTCTTATTTATAAAGTTTCTAATCGTTTTTCTGGTGTTCCTTCTCGTTTTTCTGGTTCTGGTTCTGGTACTGATTTTACTTTTACTATTTCTTCTCTTCAACCTGAAGATATTGCTACTTATTATTGTTTTCAATATTCTCATGTTCCTTGGACTTTTGGTCAAGGTACTAAACTTCAAATTACTCGTACGGTGGCTGCACCATCTGTCTTCATCTTCCCGCCATCTGATGAGCAGTTGAAATCTGGAACTGCCTCTGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACCCATCAGGGCCTGAGCTCGCCCGTCACAAAGAGCTTCAACAGGGGAGAGTGTGGAGGCGGTGGTAGCGGTGGCGGTGGCTCCGGATCCGGTTCTGGAGGAGGCGGTGGAAGCGGAGGCGGTGGCTCTGGAGGAGGCGGTTCGGATGTGCAGCTTCAGGAGTCGGGACCTAGCCTGGTGAAACCTTCTCAGTCTCTGTCCCTCACCTGCACTGTCACTGGCTACTCAATCACCAGTGATTTTGCCTGGAACTGGATTCGGCAGTTTCCAGGAAACAAGCTGGAGTGGATGGGCTACATAAGTTATAGTGGTAACACTAGGTACAACCCATCTCTCAAAAGTCGAATCTCTATCACTCGCGACACATCCAAGAACCAATTCTTCCTGCAGTTGAACTCTGTGACTATTGAGGACACAGCCACATATTACTGTGTAACGGCGGGACGCGGGTTTCCTTATTGGGGCCAAGGGACTCTGGTCACTGTCTCTGCAGGTGGAGGAGGCAGTGGTGGTGGAGGAAGCGGAGGTGGTGGCAGCGACATCCTGATGACCCAATCTCCATCCTCCATGTCTGTATCTCTGGGAGACACAGTCAGCATCACTTGCCATTCAAGTCAGGACATTAACAGTAATATAGGGTGGTTGCAGCAGAGACCAGGGAAATCATTTAAGGGCCTGATCTATCATGGAACCAACTTGGACGATGAAGTTCCATCAAGGTTCAGTGGCAGTGGATCTGGAGCCGATTATTCTCTCACCATCAGCAGCCTGGAATCTGAAGATTTTGCAGACTATTACTGTGTACAGTATGCTCAGTTTCCGTGGACGTTCGGTGGAGGCACCAAGCTGGAAATCAAATAG

SEQ ID NO: 75 - protein, 712 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - DNA, 2136 bp, synthetic construct
  source 1..2136: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaaggcggtggcggtagtgggggaggcggttctggcggcggagggtccggcggtggaggatcacaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctcgagctccggatccggttctggaggaggcggtggaagcggaggcggtggctctggaggaggcggttcggatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 77 - protein, 481 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSSGSGSGGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - DNA, 1443 bp, synthetic construct
  source 1..1443: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaaggcggtggcggtagtgggggaggcggttctggcggcggagggtccggcggtggaggatcacaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctcgagctccggatccggttctggaggaggcggtggaagcggaggcggtggctctggaggaggcggttcggacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 79 - protein, 707 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSASGSGSGGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - DNA, 2124 bp, synthetic construct
  source 1..2124: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggtggaggaggcagtggtggtggaggaagcggaggtggtggcagcgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcatccggatccggttctggaggaggcggtggaagcggaggcggtggctctggaggaggcggttcgcaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttaa

SEQ ID NO: 81 - protein, 473 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSASGSGSGGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - DNA, 1422 bp, synthetic construct
  source 1..1422: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaaggtggaggaggcagtggtggtggaggaagcggaggtggtggcagcgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcatccggatccggttctggaggaggcggtggaagcggaggcggtggctctggaggaggcggttcggacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 83 - protein, 473 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGSGSGGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 84 - DNA, 1419 bp, synthetic construct
  source 1..1419: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttccggatccggttctggaggaggcggtggaagcggaggcggtggctctggaggaggcggttcggatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggtggaggaggcagtggtggtggaggaagcggaggtggtggcagcgacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaa

SEQ ID NO: 85 - protein, 708 aa, synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 86 - DNA, 2124 bp, synthetic construct
  source 1..2124: mol_type = other DNA
caggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcggcggtggcggtagtgggggaggcggttctggcggcggagggtccggcggtggaggatcagacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaa

SEQ ID NO: 87 - protein, 698 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 88 - DNA, 2094 bp, synthetic construct
  source 1..2094: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggcggtggcggtagtgggggaggcggttctggcggcggagggtccggcggtggaggatcagacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaa

SEQ ID NO: 89 - protein, 704 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 90 - DNA, 2112 bp, synthetic construct
  source 1..2112: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcggcggtggcggtagtgggggaggcggttctggcggcggagggtccggcggtggaggatcagacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaa

SEQ ID NO: 91 - protein, 475 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 92 - DNA, 1425 bp, synthetic construct
  source 1..1425: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcggcggtggcggtagtgggggaggcggttctggcggcggagggtccggcggtggaggatcagacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaa

SEQ ID NO: 93 - protein, 711 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSGGGGSGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - DNA, 2133 bp, synthetic construct
  source 1..2133: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaaggcggtggcggtagtgggggaggcggttctggcggcggagggtccggcggtggaggatcacaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctcgagcggcggtggcggtagtgggggaggcggttctggcggcggagggtccggcggtggaggatcagatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 95 - protein, 214 aa, synthetic construct; identical to SEQ ID NO: 11

SEQ ID NO: 96 - DNA, 642 bp, synthetic construct
  source 1..642: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 97 - protein, 702 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYYYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 98 - DNA, 2106 bp, synthetic construct
  source 1..2106: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactactactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggtggcggtggagggtccggcggtggtggatccgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggcggtggcggtagtgggggaggcggttctggcggcggagggtccggcggtggaggatcagacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaa

SEQ ID NO: 99 - protein, 214 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - DNA, 642 bp, synthetic construct
  source 1..642: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgaccgtgctgcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 101 - protein, 702 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDLEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 102 - DNA, 2106 bp, synthetic construct
  source 1..2106: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgacctcgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggtggcggtggagggtccggcggtggtggatccgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggcggtggcggtagtgggggaggcggttctggcggcggagggtccggcggtggaggatcagacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaa

SEQ ID NO: 103 - protein, 702 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTAYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 104 - DNA, 2106 bp, synthetic construct
  source 1..2106: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgaccgcctacgactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggtggcggtggagggtccggcggtggtggatccgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggcggtggcggtagtgggggaggcggttctggcggcggagggtccggcggtggaggatcagacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaa

SEQ ID NO: 105 - protein, 702 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYADYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 106 - DNA, 2106 bp, synthetic construct
  source 1..2106: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctacgccgactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggtggcggtggagggtccggcggtggtggatccgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggcggtggcggtagtgggggaggcggttctggcggcggagggtccggcggtggaggatcagacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaa

SEQ ID NO: 107 - protein, 701 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSGGGGSGGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 108 - DNA, 2103 bp, synthetic construct
  source 1..2103: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcaggcggtggcggtagtgggggaggcggttctggcggcggagggtccggcggtggaggatcagacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaa

SEQ ID NO: 109 - protein, 642 aa, synthetic construct
GAGATCGTGCTGACCCAGTCTCCTTCCACACTGTCTGTGTCTCCCGGCGAGAGAGCCACCTTCAGCTGTAGAGCCTCTCAGTCCATCGGCACCAACATCCACTGGTATCAGCAGAAGCCCGGCAAGCCTCCTCGGCTGCTGATTAAGTACGCCTCCGAGTCCATCAGCGGCATCCCTGACAGATTCTCCGGCTCTGGCTCTGGCACCGAGTTTACCCTGACCATCTCCTCCGTGCAGTCCGAGGATTTCGCCGTGTACTACTGCCAGCAGAACTTCAACTGGCCCACCACCTTTGGACCCGGCACCAAGCTGACCGTGCTGCGTACGGTGGCTGCACCATCTGTCTTCATCTTCCCGCCATCTGATGAGCAGTTGAAATCTGGAACTGCCTCTGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACCCATCAGGGCCTGAGCTCGCCCGTCACAAAGAGCTTCAACAGGGGAGAGTGT

SEQ ID NO: 110 - DNA, 642 bp, synthetic construct
  source 1..642: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacttcaactggcccaccacctttggacccggcaccaagctgaccgtgctgcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 111 - protein, 214 aa, synthetic construct
DIVMTQSPSSVSLSLGDRVTITCHSSQDINSNIGWLQQKPGKAFKGLIYHGTNLDDGVPSRFSGSGSGADYTLTISSLQAEDFATYYCVQYAQFPWTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - DNA, 642 bp, synthetic construct
  source 1..642: mol_type = other DNA
gacatcgtgatgacccaatctccaagctcagtgagtctgtcactaggcgaccgtgtcaccattacatgtcatagttcccaggatatcaacagtaatattgggtggcttcagcaaaagcctggtaaagcctttaaaggactgatttatcacgggaccaacttggacgatggagttccctcccggttttctggctccgggtccggtgcagactatactttgacaattagctctctccaggcagaggacttcgctacatattactgcgtccagtatgcccagttcccttggacctttggacccggcactaaactggaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 113 - protein, 711 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNFNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSGGGGSGGGGSQVQLQESGPSLVKPSQTLSLTCTVSGYSITSDFAWNWIRQFPGRKLEWMGYISYSGNTRYNPSLKSRFTISRDTSKNQFYLKLRSVTIADTAVYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - DNA, 2133 bp, synthetic construct
  source 1..2133: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacttcaactggcccaccacctttggacccggcaccaagctgacagttcttggcggtggcggtagtgggggaggcggttctggcggcggagggtccggcggtggaggatcacaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctggcggtggcggtagtgggggaggcggttctggcggcggagggtccggcggtggaggatcacaagtgcagctccaggagtcaggccctagcctcgtgaagccctcccagaccctgagcctgacctgcaccgtatctggttatagtattacaagtgacttcgcctggaactggattaggcagtttccaggtcgcaagctggagtggatggggtatatctcctactccggtaacactcgatacaacccttccctcaaaagcagatttaccatttccagagacacatctaagaaccaattttatctgaagttgagatctgttaccatcgctgatactgctgtgtactattgcgttaccgccggacgcggcttcccctattgggggcagggaaccctggtgactgtatcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 115 - protein, 711 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTAYDYEFAYWGQGTLVTVSSGGGGSGGGGSGGGGSGGGGSQVQLQESGPSLVKPSQTLSLTCTVSGYSITSDFAWNWIRQFPGRKLEWMGYISYSGNTRYNPSLKSRFTISRDTSKNQFYLKLRSVTIADTAVYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - DNA, 2133 bp, synthetic construct
  source 1..2133: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttcttggcggtggcggtagtgggggaggcggttctggcggcggagggtccggcggtggaggatcacaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgaccgcctacgactacgagtttgcttactggggccagggcaccctggtcacagtttcttctggcggtggcggtagtgggggaggcggttctggcggcggagggtccggcggtggaggatcacaagtgcagctccaggagtcaggccctagcctcgtgaagccctcccagaccctgagcctgacctgcaccgtatctggttatagtattacaagtgacttcgcctggaactggattaggcagtttccaggtcgcaagctggagtggatggggtatatctcctactccggtaacactcgatacaacccttccctcaaaagcagatttaccatttccagagacacatctaagaaccaattttatctgaagttgagatctgttaccatcgctgatactgctgtgtactattgcgttaccgccggacgcggcttcccctattgggggcagggaaccctggtgactgtatcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 117 - protein, 711 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYADYEFAYWGQGTLVTVSSGGGGSGGGGSGGGGSGGGGSQVQLQESGPSLVKPSQTLSLTCTVSGYSITSDFAWNWIRQFPGRKLEWMGYISYSGNTRYNPSLKSRFTISRDTSKNQFYLKLRSVTIADTAVYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - DNA, 2133 bp, synthetic construct
  source 1..2133: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttcttggcggtggcggtagtgggggaggcggttctggcggcggagggtccggcggtggaggatcacaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctacgccgactacgagtttgcttactggggccagggcaccctggtcacagtttcttctggcggtggcggtagtgggggaggcggttctggcggcggagggtccggcggtggaggatcacaagtgcagctccaggagtcaggccctagcctcgtgaagccctcccagaccctgagcctgacctgcaccgtatctggttatagtattacaagtgacttcgcctggaactggattaggcagtttccaggtcgcaagctggagtggatggggtatatctcctactccggtaacactcgatacaacccttccctcaaaagcagatttaccatttccagagacacatctaagaaccaattttatctgaagttgagatctgttaccatcgctgatactgctgtgtactattgcgttaccgccggacgcggcttcccctattgggggcagggaaccctggtgactgtatcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 119 - protein, 711 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYYYEFAYWGQGTLVTVSSGGGGSGGGGSGGGGSGGGGSQVQLQESGPSLVKPSQTLSLTCTVSGYSITSDFAWNWIRQFPGRKLEWMGYISYSGNTRYNPSLKSRFTISRDTSKNQFYLKLRSVTIADTAVYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - DNA, 2133 bp, synthetic construct
  source 1..2133: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttcttggcggtggcggtagtgggggaggcggttctggcggcggagggtccggcggtggaggatcacaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactactactacgagtttgcttactggggccagggcaccctggtcacagtttcttctggcggtggcggtagtgggggaggcggttctggcggcggagggtccggcggtggaggatcacaagtgcagctccaggagtcaggccctagcctcgtgaagccctcccagaccctgagcctgacctgcaccgtatctggttatagtattacaagtgacttcgcctggaactggattaggcagtttccaggtcgcaagctggagtggatggggtatatctcctactccggtaacactcgatacaacccttccctcaaaagcagatttaccatttccagagacacatctaagaaccaattttatctgaagttgagatctgttaccatcgctgatactgctgtgtactattgcgttaccgccggacgcggcttcccctattgggggcagggaaccctggtgactgtatcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 121 - protein, 711 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDLEFAYWGQGTLVTVSSGGGGSGGGGSGGGGSGGGGSQVQLQESGPSLVKPSQTLSLTCTVSGYSITSDFAWNWIRQFPGRKLEWMGYISYSGNTRYNPSLKSRFTISRDTSKNQFYLKLRSVTIADTAVYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - DNA, 2133 bp, synthetic construct
  source 1..2133: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttcttggcggtggcggtagtgggggaggcggttctggcggcggagggtccggcggtggaggatcacaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgacctcgagtttgcttactggggccagggcaccctggtcacagtttcttctggcggtggcggtagtgggggaggcggttctggcggcggagggtccggcggtggaggatcacaagtgcagctccaggagtcaggccctagcctcgtgaagccctcccagaccctgagcctgacctgcaccgtatctggttatagtattacaagtgacttcgcctggaactggattaggcagtttccaggtcgcaagctggagtggatggggtatatctcctactccggtaacactcgatacaacccttccctcaaaagcagatttaccatttccagagacacatctaagaaccaattttatctgaagttgagatctgttaccatcgctgatactgctgtgtactattgcgttaccgccggacgcggcttcccctattgggggcagggaaccctggtgactgtatcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 123 - protein, 448 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - DNA, 1347 bp, synthetic construct
  source 1..1347: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 125 - protein, 473 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGGSGGGGSGGGGSQVQLQESGPSLVKPSQTLSLTCTVSGYSITSDFAWNWIRQFPGRKLEWMGYISYSGNTRYNPSLKSRFTISRDTSKNQFYLKLRSVTIADTAVYYCVTAGRGFPYWGQGTLVTVSSGGGGSGGGGSGGGGSDIVMTQSPSSVSLSLGDRVTITCHSSQDINSNIGWLQQKPGKAFKGLIYHGTNLDDGVPSRFSGSGSGADYTLTISSLQAEDFATYYCVQYAQFPWTFGPGTKLEIK

SEQ ID NO: 126 - DNA, 1419 bp, synthetic construct
  source 1..1419: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttcttcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggatccggaggaggcggtggaagcggaggcggtggctctggaggaggcggttcgcaagtgcagctccaggagtcaggccctagcctcgtgaagccctcccagaccctgagcctgacctgcaccgtatctggttatagtattacaagtgacttcgcctggaactggattaggcagtttccaggtcgcaagctggagtggatggggtatatctcctactccggtaacactcgatacaacccttccctcaaaagcagatttaccatttccagagacacatctaagaaccaattttatctgaagttgagatctgttaccatcgctgatactgctgtgtactattgcgttaccgccggacgcggcttcccctattgggggcagggaaccctggtgactgtatcatccggtggaggaggcagtggtggtggaggaagcggaggtggtggcagcgacatcgtgatgacccaatctccaagctcagtgagtctgtcactaggcgaccgtgtcaccattacatgtcatagttcccaggatatcaacagtaatattgggtggcttcagcaaaagcctggtaaagcctttaaaggactgatttatcacgggaccaacttggacgatggagttccctcccggttttctggctccgggtccggtgcagactatactttgacaattagctctctccaggcagaggacttcgctacatattactgcgtccagtatgcccagttcccttggacctttggacccggcactaaactggaaattaaa

SEQ ID NO: 127 - protein, 448 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTAYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - DNA, 1347 bp, synthetic construct
  source 1..1347: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgaccgcctacgactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 129 - protein, 448 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYADYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - DNA, 1347 bp, synthetic construct
  source 1..1347: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctacgccgactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 131 - protein, 448 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYYYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - DNA, 1347 bp, synthetic construct
  source 1..1347: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactactactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 133 - protein, 448 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDLEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - DNA, 1347 bp, synthetic construct
  source 1..1347: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgacctcgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttag

SEQ ID NO: 135 - protein, 473 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNFNWPTTFGPGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGGSGGGGSGGGGSQVQLQESGPSLVKPSQTLSLTCTVSGYSITSDFAWNWIRQFPGRKLEWMGYISYSGNTRYNPSLKSRFTISRDTSKNQFYLKLRSVTIADTAVYYCVTAGRGFPYWGQGTLVTVSSGGGGSGGGGSGGGGSDIVMTQSPSSVSLSLGDRVTITCHSSQDINSNIGWLQQKPGKAFKGLIYHGTNLDDGVPSRFSGSGSGADYTLTISSLQAEDFATYYCVQYAQFPWTFGPGTKLEIK

SEQ ID NO: 136 - DNA, 1419 bp, synthetic construct
  source 1..1419: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacttcaactggcccaccacctttggacccggcaccaagctgaccgtgctgcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggatccggaggaggcggtggaagcggaggcggtggctctggaggaggcggttcgcaagtgcagctccaggagtcaggccctagcctcgtgaagccctcccagaccctgagcctgacctgcaccgtatctggttatagtattacaagtgacttcgcctggaactggattaggcagtttccaggtcgcaagctggagtggatggggtatatctcctactccggtaacactcgatacaacccttccctcaaaagcagatttaccatttccagagacacatctaagaaccaattttatctgaagttgagatctgttaccatcgctgatactgctgtgtactattgcgttaccgccggacgcggcttcccctattgggggcagggaaccctggtgactgtatcatccggtggaggaggcagtggtggtggaggaagcggaggtggtggcagcgacatcgtgatgacccaatctccaagctcagtgagtctgtcactaggcgaccgtgtcaccattacatgtcatagttcccaggatatcaacagtaatattgggtggcttcagcaaaagcctggtaaagcctttaaaggactgatttatcacgggaccaacttggacgatggagttccctcccggttttctggctccgggtccggtgcagactatactttgacaattagctctctccaggcagaggacttcgctacatattactgcgtccagtatgcccagttcccttggacctttggacccggcactaaactggaaattaaa

SEQ ID NO: 137 - protein, 701 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVL

SEQ ID NO: 138 - DNA, 2106 bp, synthetic construct
  source 1..2106: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctggcggtggcggtagtgggggaggcggttctggcggcggagggtccggcggtggaggatcagagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttctttga

SEQ ID NO: 139 - protein, 701 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNFNWPTTFGPGTKLTVL

SEQ ID NO: 140 - DNA, 2103 bp, synthetic construct
  source 1..2103: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctggcggtggcggtagtgggggaggcggttctggcggcggagggtccggcggtggaggatcagagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacttcaactggcccaccacctttggacccggcaccaagctgacagttctt

SEQ ID NO: 141 - protein, 701 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTAYDYEFAYWGQGTLVTVSSGGGGSGGGGSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVL

SEQ ID NO: 142 - DNA, 2103 bp, synthetic construct
  source 1..2103: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgaccgcctacgactacgagtttgcttactggggccagggcaccctggtcacagtttcttctggcggtggcggtagtgggggaggcggttctggcggcggagggtccggcggtggaggatcagagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttctt

SEQ ID NO: 143 - protein, 701 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYADYEFAYWGQGTLVTVSSGGGGSGGGGSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVL

SEQ ID NO: 144 - DNA, 2103 bp, synthetic construct
  source 1..2103: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctacgccgactacgagtttgcttactggggccagggcaccctggtcacagtttcttctggcggtggcggtagtgggggaggcggttctggcggcggagggtccggcggtggaggatcagagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttctt

SEQ ID NO: 145 - protein, 701 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYYYEFAYWGQGTLVTVSSGGGGSGGGGSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVL

SEQ ID NO: 146 - DNA, 2103 bp, synthetic construct
  source 1..2103: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactactactacgagtttgcttactggggccagggcaccctggtcacagtttcttctggcggtggcggtagtgggggaggcggttctggcggcggagggtccggcggtggaggatcagagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttctt

SEQ ID NO: 147 - protein, 701 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDLEFAYWGQGTLVTVSSGGGGSGGGGSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVL

SEQ ID NO: 148 - DNA, 2103 bp, synthetic construct
  source 1..2103: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatcccaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgacctcgagtttgcttactggggccagggcaccctggtcacagtttcttctggcggtggcggtagtgggggaggcggttctggcggcggagggtccggcggtggaggatcagagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttctt

SEQ ID NO: 149 - protein, 446 aa, synthetic construct
QVQLQESGPSLVKPSQTLSLTCTVSGYSITSDFAWNWIRQFPGRKLEWMGYISYSGNTRYNPSLKSRFTISRDTSKNQFYLKLRSVTIADTAVYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - DNA, 1338 bp, synthetic construct
  source 1..1338: mol_type = other DNA
caagtgcagctccaggagtcaggccctagcctcgtgaagccctcccagaccctgagcctgacctgcaccgtatctggttatagtattacaagtgacttcgcctggaactggattaggcagtttccaggtcgcaagctggagtggatggggtatatctcctactccggtaacactcgatacaacccttccctcaaaagcagatttaccatttccagagacacatctaagaaccaattttatctgaagttgagatctgttaccatcgctgatactgctgtgtactattgcgttaccgccggacgcggcttcccctattgggggcagggaaccctggtgactgtatcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 151 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNFNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - DNA, 642 bp, synthetic construct
  source 1..642: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaatttcaactggccaaccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 153 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNKNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - DNA, 642 bp, synthetic construct
  source 1..642: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataagaactggccaaccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 155 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTAYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - DNA, 1344 bp, synthetic construct
  source 1..1344: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcaccgcctatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 157 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTWYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 158 - DNA, 1344 bp, synthetic construct
  source 1..1344: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctggtatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 159 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYADYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - DNA, 1344 bp, synthetic construct
  source 1..1344: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctacgccgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 161 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYVDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - DNA, 1344 bp, synthetic construct
  source 1..1344: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctacgtggattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 163 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYFYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - DNA, 1344 bp, synthetic construct
  source 1..1344: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatttctacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 165 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYWYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - DNA, 1344 bp, synthetic construct
  source 1..1344: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactattggtacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 167 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYYYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - DNA, 1344 bp, synthetic construct
  source 1..1344: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactattactacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 169 - protein, 448 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDLEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 170 - DNA, 1344 bp, synthetic construct
  source 1..1344: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgatctcgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 171 - protein, 446 aa, synthetic construct
QVQLQESGPGLVKPSQSLSLTCTVSGYSITSDFAWNWIRQPPGKGLEWIGYISYSGNTRYNPSLKSRVTISRDTSKNQFSLQLNSVTAEDTAVYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - DNA, 1338 bp, synthetic construct
  source 1..1338: mol_type = other DNA
caagtgcagttgcaggagagtggtccgggcctggtgaagccttctcagtcactgagcctcacgtgcaccgtaagcggttatagtatcaccagtgattttgcctggaactggattcggcagccgcccgggaagggactggagtggatcggatacatttcctacagtggaaacactcgatataatcctagtcttaaaagtcgggtaaccatctctagggataccagcaagaatcagttctcattgcaactcaattcagtgaccgcagaagacaccgcagtctactattgcgtcacagccggacgcggtttcccgtactggggccaaggaacactagtcaccgtgtcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 173 - protein, 214 aa, synthetic construct
DIQMTQSPSSVSASVGDRVTITCHSSQDINSNIGWLQQKPGKAPKGLIYHGTNLDDGVPSRFSGSGSGADYTLTISSLQPEDFATYYCVQYAQFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - DNA, 642 bp, synthetic construct
  source 1..642: mol_type = other DNA
gacattcagatgacacagagcccaagtagcgtttctgccagcgtgggggaccgagtcaccattacttgtcacagttcacaagatattaactctaacatcggctggctgcagcaaaaaccaggtaaagcaccaaaagggctcatttatcatgggaccaacctggatgacggagtcccgagtcgattcagcgggagtggaagcggagctgattataccctgacgatctcaagcttgcagccagaggatttcgccacatactattgtgtccagtatgcccagttcccctggacatttggccagggtacgaaggtggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 175 - protein, 214 aa, synthetic construct
DIVMTQSPSSLSVSPGDRVTITCHSSQDINSNIGWLQQKPGKAPKGLIYHGTNLDDGVPSRFSGSGSGADYTLTISSLEPEDFAVYYCVQYAQFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - DNA, 642 bp, synthetic construct
  source 1..642: mol_type = other DNA
gacatagtgatgacacagtctccaagttctttgagcgtatccccaggcgaccgcgtaacaattacctgtcacagctcccaggatattaatagtaatattggctggcttcaacagaagccagggaaggcaccaaaaggactgatctaccacggaactaatctggacgatggggttccttcacggttctccgggtccggctctggtgccgactacactctgacaattagctccctggagcccgaggacttcgcagtgtactattgcgtgcagtacgcacaatttccctggacatttggtcaaggcaccaaagtcgaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 177 - protein, 446 aa, synthetic construct
QVQLQESGPGLVKPSQSLSLTCTVSGYSITSDFAWNWIRQAPGKGLEWVGYISYSGNTRYNPSLKSRFTISRDTSKNQFYLQLNSVTAEDTAVYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - DNA, 1338 bp, synthetic construct
  source 1..1338: mol_type = other DNA
caagtccagctgcaggaaagtggccctggactcgttaagcctagtcagagcttgtcattgacttgcacagtcagtggttattccatcacaagtgactttgcctggaattggattcggcaggctcccgggaaggggctggaatgggtcgggtatatttcctacagcggaaatactcgatataacccatcccttaaaagtcgcttcaccatatctcgtgacaccagcaaaaatcaattttatcttcaactcaattcagtgacagccgaggatacagctgtttactattgtgtgacagctggcagaggatttccctattggggacagggaacccttgtaacggtctccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 179 - protein, 711 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSGGGGSGGGGSQVQLQESGPSLVKPSQTLSLTCTVSGYSITSDFAWNWIRQFPGRKLEWMGYISYSGNTRYNPSLKSRFTISRDTSKNQFYLKLRSVTIADTAVYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 180 - DNA, 2133 bp, synthetic construct
  source 1..2133: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttcttggcggtggcggtagtgggggaggcggttctggcggcggagggtccggcggtggaggatcacaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctggcggtggcggtagtgggggaggcggttctggcggcggagggtccggcggtggaggatcacaagtgcagctccaggagtcaggccctagcctcgtgaagccctcccagaccctgagcctgacctgcaccgtatctggttatagtattacaagtgacttcgcctggaactggattaggcagtttccaggtcgcaagctggagtggatggggtatatctcctactccggtaacactcgatacaacccttccctcaaaagcagatttaccatttccagagacacatctaagaaccaattttatctgaagttgagatctgttaccatcgctgatactgctgtgtactattgcgttaccgccggacgcggcttcccctattgggggcagggaaccctggtgactgtatcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 181 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 182 - DNA, 357 bp, synthetic construct
  source 1..357: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagc

SEQ ID NO: 183 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 184 - DNA, 321 bp, synthetic construct
  source 1..321: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaa

SEQ ID NO: 185 - protein, 123 aa, synthetic construct [sequence omitted]

SEQ ID NO: 186 - DNA, 369 bp, synthetic construct
  source 1..369: mol_type = other DNA
caggtgcagctgcagcagagcggcgccgaggtgaagaagcccggcagcagcgtgaaggtgagctgcaaggccagcggctacaccttcaccaactactacatctactgggtgcggcaggcccccggccagggcctggagtggatcggcggcatcaaccccaccagcggcggcagcaacttcaacgagaagttcaagacccgggtgaccatcaccgccgacgagagcagcaccaccgcctacatggagctgagcagcctgcggagcgaggacaccgccttctacttctgcacccggcagggcctgtggttcgacagcgacggccggggcttcgacttctggggccagggcaccaccgtgaccgtgagcagc

SEQ ID NO: 187 - protein, 112 aa, synthetic construct [sequence omitted]

SEQ ID NO: 188 - DNA, 336 bp, synthetic construct
  source 1..336: mol_type = other DNA
gatattcaaatgactcaatctccttcttctctttctgcttctgttggtgatcgtgttactattacttgtcgttcttctcaaaatattgttcattctaatggtaatacttatcttgattggtatcaacaaactcctggtaaagctcctaaacttcttatttataaagtttctaatcgtttttctggtgttccttctcgtttttctggttctggttctggtactgattttacttttactatttcttctcttcaacctgaagatattgctacttattattgttttcaatattctcatgttccttggacttttggtcaaggtactaaacttcaaattact

SEQ ID NO: 189 - protein, 116 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 190 - DNA, 348 bp, synthetic construct
  source 1..348: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgca

SEQ ID NO: 191 - protein, 107 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 192 - DNA, 321 bp, synthetic construct
  source 1..321: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaa

SEQ ID NO: 193 - protein, 10 aa, synthetic construct
GGGGSGGGGS

SEQ ID NO: 194 - DNA, 30 bp, synthetic construct
  source 1..30: mol_type = other DNA
ggcggtggagggtccggcggtggtggatcc

SEQ ID NO: 195 - protein, 15 aa, synthetic construct
GGGGSGGGGSGGGGS

SEQ ID NO: 196 - DNA, 45 bp, synthetic construct
  source 1..45: mol_type = other DNA
ggtggaggaggcagtggtggtggaggaagcggaggtggtggcagc

SEQ ID NO: 197 - protein, 25 aa, synthetic construct
GGGGSGSGSGGGGGSGGGGSGGGGS

SEQ ID NO: 198 - DNA, 75 bp, synthetic construct
  source 1..75: mol_type = other DNA
ggtggcggtggctccggatccggttctggaggaggcggtggaagcggaggcggtggctctggaggaggcggttcg

SEQ ID NO: 199 - protein, 30 aa, synthetic construct
GGGGSGGGGSGSGSGGGGGSGGGGSGGGGS

SEQ ID NO: 200 - DNA, 90 bp, synthetic construct
  source 1..90: mol_type = other DNA
ggaggcggtggtagcggtggcggtggctccggatccggttctggaggaggcggtggaagcggaggcggtggctctggaggaggcggttcg

SEQ ID NO: 201 - protein, 359 aa, synthetic construct
SEQIDHUMANIGGAMINOACIDSEQUENCEASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 202 - DNA, 987 bp, synthetic construct
  source 1..987: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 203 - protein, 107 aa, synthetic construct [sequence omitted]

SEQ ID NO: 204 - DNA, 321 bp, synthetic construct
  source 1..321: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 205 - protein, 116 aa, synthetic construct
QVQLQESGPGLVKPSQSLSLTCTVSGYSITSDFAWNWIRQPPGKGLEWIGYISYSGNTRYNPSLKSRVTISRDTSKNQFSLQLNSVTAEDTAVYYCVTAGRGFPYWGQGTLVTVSS

SEQ ID NO: 206 - DNA, 348 bp, synthetic construct
  source 1..348: mol_type = other DNA
caagtgcagttgcaggagagtggtccgggcctggtgaagccttctcagtcactgagcctcacgtgcaccgtaagcggttatagtatcaccagtgattttgcctggaactggattcggcagccgcccgggaagggactggagtggatcggatacatttcctacagtggaaacactcgatataatcctagtcttaaaagtcgggtaaccatctctagggataccagcaagaatcagttctcattgcaactcaattcagtgaccgcagaagacaccgcagtctactattgcgtcacagccggacgcggtttcccgtactggggccaaggaacactagtcaccgtgtcttcc

SEQ ID NO: 207 - protein, 107 aa, synthetic construct
DIQMTQSPSSVSASVGDRVTITCHSSQDINSNIGWLQQKPGKAPKGLIYHGTNLDDGVPSRFSGSGSGADYTLTISSLQPEDFATYYCVQYAQFPWTFGQGTKVEIK

SEQ ID NO: 208 - DNA, 321 bp, synthetic construct
  source 1..321: mol_type = other DNA
gacattcagatgacacagagcccaagtagcgtttctgccagcgtgggggaccgagtcaccattacttgtcacagttcacaagatattaactctaacatcggctggctgcagcaaaaaccaggtaaagcaccaaaagggctcatttatcatgggaccaacctggatgacggagtcccgagtcgattcagcgggagtggaagcggagctgattataccctgacgatctcaagcttgcagccagaggatttcgccacatactattgtgtccagtatgcccagttcccctggacatttggccagggtacgaaggtggagattaag

SEQ ID NO: 209 - protein, 116 aa, synthetic construct
QVQLQESGPGLVKPSQSLSLTCTVSGYSITSDFAWNWIRQAPGKGLEWVGYISYSGNTRYNPSLKSRFTISRDTSKNQFYLQLNSVTAEDTAVYYCVTAGRGFPYWGQGTLVTVSS

SEQ ID NO: 210 - DNA, 348 bp, synthetic construct
  source 1..348: mol_type = other DNA
caagtccagctgcaggaaagtggccctggactcgttaagcctagtcagagcttgtcattgacttgcacagtcagtggttattccatcacaagtgactttgcctggaattggattcggcaggctcccgggaaggggctggaatgggtcgggtatatttcctacagcggaaatactcgatataacccatcccttaaaagtcgcttcaccatatctcgtgacaccagcaaaaatcaattttatcttcaactcaattcagtgacagccgaggatacagctgtttactattgtgtgacagctggcagaggatttccctattggggacagggaacccttgtaacggtctccagc

SEQ ID NO: 211 - protein, 107 aa, synthetic construct
DIVMTQSPSSLSVSPGDRVTITCHSSQDINSNIGWLQQKPGKAPKGLIYHGTNLDDGVPSRFSGSGSGADYTLTISSLEPEDFAVYYCVQYAQFPWTFGQGTKVEIK

SEQ ID NO: 212 - DNA, 321 bp, synthetic construct
  source 1..321: mol_type = other DNA
gacatagtgatgacacagtctccaagttctttgagcgtatccccaggcgaccgcgtaacaattacctgtcacagctcccaggatattaatagtaatattggctggcttcaacagaagccagggaaggcaccaaaaggactgatctaccacggaactaatctggacgatggggttccttcacggttctccgggtccggctctggtgccgactacactctgacaattagctccctggagcccgaggacttcgcagtgtactattgcgtgcagtacgcacaatttccctggacatttggtcaaggcaccaaagtcgaaattaaa

SEQ ID NO: 213 - protein, 116 aa, synthetic construct
QVQLQESGPSLVKPSQTLSLTCTVSGYSITSDFAWNWIRQFPGRKLEWMGYISYSGNTRYNPSLKSRFTISRDTSKNQFYLKLRSVTIADTAVYYCVTAGRGFPYWGQGTLVTVSS

SEQ ID NO: 214 - DNA, 348 bp, synthetic construct
  source 1..348: mol_type = other DNA
caagtgcagctccaggagtcaggccctagcctcgtgaagccctcccagaccctgagcctgacctgcaccgtatctggttatagtattacaagtgacttcgcctggaactggattaggcagtttccaggtcgcaagctggagtggatggggtatatctcctactccggtaacactcgatacaacccttccctcaaaagcagatttaccatttccagagacacatctaagaaccaattttatctgaagttgagatctgttaccatcgctgatactgctgtgtactattgcgttaccgccggacgcggcttcccctattgggggcagggaaccctggtgactgtatcatcc

SEQ ID NO: 215 - protein, 107 aa, synthetic construct
DIVMTQSPSSVSLSLGDRVTITCHSSQDINSNIGWLQQKPGKAFKGLIYHGTNLDDGVPSRFSGSGSGADYTLTISSLQAEDFATYYCVQYAQFPWTFGPGTKLEIK

SEQ ID NO: 216 - DNA, 321 bp, synthetic construct
  source 1..321: mol_type = other DNA
gacatcgtgatgacccaatctccaagctcagtgagtctgtcactaggcgaccgtgtcaccattacatgtcatagttcccaggatatcaacagtaatattgggtggcttcagcaaaagcctggtaaagcctttaaaggactgatttatcacgggaccaacttggacgatggagttccctcccggttttctggctccgggtccggtgcagactatactttgacaattagctctctccaggcagaggacttcgctacatattactgcgtccagtatgcccagttcccttggacctttggacccggcactaaactggaaattaaa

SEQ ID NO: 217 - protein, 119 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSS

SEQ ID NO: 218 - DNA, 357 bp, synthetic construct
  source 1..357: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttct

SEQ ID NO: 219 - protein, 107 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVL

SEQ ID NO: 220 - DNA, 321 bp, synthetic construct
  source 1..321: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgaccgtgctg

SEQ ID NO: 221 - protein, 107 aa, synthetic construct
EIVLTQSPSTLSVSPGERATFSCRASQSIGTNIHWYQQKPGKPPRLLIKYASESISGIPDRFSGSGSGTEFTLTISSVQSEDFAVYYCQQNFNWPTTFGPGTKLTVL

SEQ ID NO: 222 - DNA, 321 bp, synthetic construct
  source 1..321: mol_type = other DNA
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacttcaactggcccaccacctttggacccggcaccaagctgaccgtgctg

SEQ ID NO: 223 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNKNWPTTFGAGTKLELK

SEQ ID NO: 224 - DNA, 321 bp, synthetic construct
  source 1..321: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataagaactggccaaccacgttcggtgctgggaccaagctggagctgaaa

SEQ ID NO: 225 - protein, 119 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTAYDYEFAYWGQGTLVTVSS

SEQ ID NO: 226 - DNA, 357 bp, synthetic construct
  source 1..357: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgaccgcctacgactacgagtttgcttactggggccagggcaccctggtcacagtttcttct

SEQ ID NO: 227 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTWYDYEFAYWGQGTLVTVSS

SEQ ID NO: 228 - DNA, 357 bp, synthetic construct
  source 1..357: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctggtatgattacgagtttgcttactggggccaagggactctggtcactgtctctagc

SEQ ID NO: 229 - protein, 119 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYADYEFAYWGQGTLVTVSS

SEQ ID NO: 230 - DNA, 357 bp, synthetic construct
  source 1..357: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctacgccgactacgagtttgcttactggggccagggcaccctggtcacagtttcttct

SEQ ID NO: 231 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYFYEFAYWGQGTLVTVSS

SEQ ID NO: 232 - DNA, 357 bp, synthetic construct
  source 1..357: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatttctacgagtttgcttactggggccaagggactctggtcactgtctctagc

SEQ ID NO: 233 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYWYEFAYWGQGTLVTVSS

SEQ ID NO: 234 - DNA, 357 bp, synthetic construct
  source 1..357: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactattggtacgagtttgcttactggggccaagggactctggtcactgtctctagc

SEQ ID NO: 235 - protein, 119 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYYYEFAYWGQGTLVTVSS

SEQ ID NO: 236 - DNA, 357 bp, synthetic construct
  source 1..357: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactactactacgagtttgcttactggggccagggcaccctggtcacagtttcttct

SEQ ID NO: 237 - protein, 119 aa, synthetic construct
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDLEFAYWGQGTLVTVSS

SEQ ID NO: 238 - DNA, 357 bp, synthetic construct
  source 1..357: mol_type = other DNA
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgacctcgagtttgcttactggggccagggcaccctggtcacagtttcttct

SEQ ID NO: 239 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTAYDYEFAYWGQGTLVTVSS

SEQ ID NO: 240 - DNA, 357 bp, synthetic construct
  source 1..357: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcaccgcctatgattacgagtttgcttactggggccaagggactctggtcactgtctctagc

SEQ ID NO: 241 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYADYEFAYWGQGTLVTVSS

SEQ ID NO: 242 - DNA, 357 bp, synthetic construct
  source 1..357: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctacgccgattacgagtttgcttactggggccaagggactctggtcactgtctctagc

SEQ ID NO: 243 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYVDYEFAYWGQGTLVTVSS

SEQ ID NO: 244 - DNA, 357 bp, synthetic construct
  source 1..357: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctacgtggattacgagtttgcttactggggccaagggactctggtcactgtctctagc

SEQ ID NO: 245 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYYYEFAYWGQGTLVTVSS

SEQ ID NO: 246 - DNA, 357 bp, synthetic construct
  source 1..357: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactattactacgagtttgcttactggggccaagggactctggtcactgtctctagc

SEQ ID NO: 247 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDLEFAYWGQGTLVTVSS

SEQ ID NO: 248 - DNA, 357 bp, synthetic construct
  source 1..357: mol_type = other DNA
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgatctcgagtttgcttactggggccaagggactctggtcactgtctctagc

SEQ ID NO: 249 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNFNWPTTFGAGTKLELK

SEQ ID NO: 250 - protein, 321 aa, synthetic construct
GACATCTTGCTGACTCAGTCTCCAGTCATCCTGTCTGTGAGTCCAGGAGAAAGAGTCAGTTTCTCCTGCAGGGCCAGTCAGAGTATTGGCACAAACATACACTGGTATCAGCAAAGAACAAATGGTTCTCCAAGGCTTCTCATAAAGTATGCTTCTGAGTCTATCTCTGGGATTCCTTCCAGGTTTAGTGGCAGTGGATCAGGGACAGATTTTACTCTTAGCATCAACAGTGTGGAGTCTGAAGATATTGCAGATTATTACTGTCAACAAAATTTCAACTGGCCAACCACGTTCGGTGCTGGGACCAAGCTGGAGCTGAAA